(19) United States
(12) Patent Application Publication
(10) Pub. No.: US 2026/0365690 A1
(43) Pub. Date: Nov. 19, 2026

(54) COMPOUND FOR RNA CAPPING AND USE THEREOF

(71) Applicant: GUANGZHOU HENOVCOM BIOSCIENCE CO., LTD., Guangdong (CN)

(72) Inventors: Jiancun ZHANG, Guangdong (CN); Lijun ZHANG, Guangdong (CN); Yiqian ZHOU, Guangdong (CN); Jiafeng CHEN, Guangdong (CN); Jufu ZHANG, Guangdong (CN); Suyong LI, Guangdong (CN); Yanhui LIU, Guangdong (CN); Chen GUO, Guangdong (CN); Wanjun TANG, Guangdong (CN); Feng WU, Guangdong (CN); Xiaoxi HE, Guangdong (CN); Qi GUO, Guangdong (CN); Huixuan CHEN, Guangdong (CN); Kun WANG, Guangdong (CN); Deyao LI, Guangdong (CN)

(21) Appl. No.: 19/107,893

(22) PCT Filed: Sep. 5, 2023
(86) PCT No.: PCT/CN2023/117043

§ 371 Date: Feb. 28, 2025

(30) Foreign Application Priority Data
Sep. 5, 2022 (CN) 202211077883.7

(51) Int. Cl. C07H 21/02; A61K 31/7105; C12P 19/34

(52) U.S. Cl. CPC C07H 21/02; A61K 31/7105; C12P 19/34

(57) ABSTRACT

The present invention relates to a compound for RNA capping and use thereof and belongs to the technical field of genetic engineering. The compound has a structure represented by formula I. The compound is used for capping an mRNA5′ end, and the capping efficiency is good. The capped mRNA can stably express a protein with a high yield. By using the compound of the present invention as a cap structure to prepare an RNA vaccine or an RNA medicament, the cost can be greatly reduced. The compound of the present invention has wide application prospects in preparing RNA vaccines or RNA medicaments.

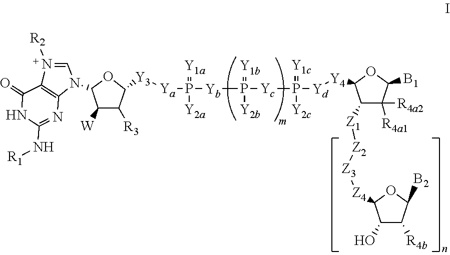

### TECHNICAL FIELD

The present disclosure relates to the technical field of genetic engineering, in particular to a compound for RNA capping and use thereof.

### BACKGROUND

The 5′ cap structure (five-prime cap) (m7GpppN) of messenger ribonucleic acid (mRNA) was discovered in the 1970s, and its presence confers stability to the mRNA and enables efficient translation. There are usually three types of cap structures (m7G5′ppp5′Np, m7G5′ppp5′NmpNp, m7G5′ppp5′NmpNmpNp), which are referred to as type O (m7G5′ppp5′Np), type I (m7G5′ppp5′NmpNp) and type II (m7G5′ppp5′NmpNmpNp), respectively. Type O means that the ribose of the terminal nucleotide is unmethylated, type I means that the ribose of one terminal nucleotide is methylated, and type II means that the ribose of two terminal nucleotides is methylated.

In eukaryotic cells, in addition to recognizing the initiation of protein synthesis, the 5′ cap structure also serves as a protective group for exonuclease cleavage from 5′ to 3′, that is, it is resistant to degradation by 5′-exonucleases. Additionally, during protein synthesis, the cap structure is also a unique identifier that recruits protein factors involved in pre-mRNA splicing, polyadenylation and nuclear export. It also functions as an anchor for recruiting initiation factors, facilitating ribosomes recognition and binding to mRNA, thereby ensuring proper translation initiation.

Studies have shown that the cap structure of mRNA is closely related to RNA quality control and the body's innate immunity. Therefore, it is of great application value to provide a system that can improve the capping efficiency and increase the expression of mRNA after capping.

### CONTENT OF THE PRESENT INVENTION

Based on this, it is necessary to provide a compound for RNA capping in response to the above problems. Using this compound for mRNA capping at the 5′ end has good capping efficiency, and the obtained mRNA expression after capping is also improved.

A compound having a structure represented by formula I, or a stereoisomer, pharmaceutically acceptable salt or solvate thereof:

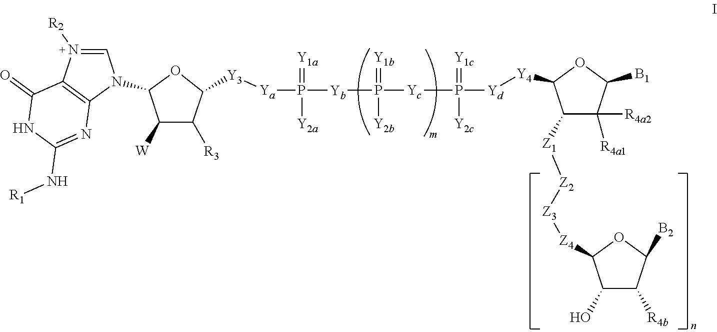

wherein
R₁ is H, C₁-C₆ alkyl, C₂-C₆ alkenyl, C₂-C₆ alkynyl, C₃-C₆ cycloalkyl, C₃-C₆ cycloalkenyl, benzyl, aryl, heteroaryl, R₅-substituted benzyl-C₁-C₆ alkyl, R₅-substituted C₂-C₆ alkenyl, R₅-substituted C₂-C₆ alkynyl, R₅-substituted C₃-C₆ cycloalkyl, R₅-substituted benzyl, R₅-substituted aryl, C₁-C₆ alkylcarbonyl, C₁-C₆ alkoxycarbonyl or sulfonyl;
R₂ is C₁-C₆ alkyl, C₂-C₆ alkenyl, C₂-C₆ alkynyl, C₃-C₆ cycloalkyl, C₃-C₆ cycloalkenyl, benzyl, R₅-substituted C₁-C₆ alkyl, R₅-substituted C₂-C₆ alkenyl, R₅-substituted C₂-C₆ alkynyl, R₅-substituted C₃-C₆ cycloalkyl, R₅-substituted C₃-C₆ cycloalkenyl or R₅-substituted benzyl;
R₃ is H, OH, halogen, CN, N₃, C₁-C₆ alkyl, C₁-C₆ alkoxy, C₂-C₆ alkenyl, C₂-C₆ alkynyl, C₃-C₆ cycloalkyl, C₃-C₆ cycloalkenyl, benzyl, benzyloxy, R₈-substituted C₁-C₆ alkyl, R₈-substituted C₁-C₆ alkoxy, R₈-substituted C₂-C₆ alkenyl, R₈-substituted C₂-C₆ alkynyl, R₈-substituted C₃-C₆ cycloalkyl, R₈-substituted C₃-C₆ cycloalkenyl, R₈-substituted benzyl, R₈-substituted benzyloxy, —(CH₂)ₙ₁C(O)NR₇R₈, —(CH₂)ₙ₁OC(O)NR₇R₈, —(CH₂)ₙ₁NR₇C(O)R₈ or —(CH₂)ₙ₁NR₇SO₂R₈;
R₄ₐ₁, R₄ₐ₂ and R_{4b} are independently H, OH, C₁-C₆ alkyl, C₁-C₆ alkoxy, C₂-C₆ alkenyl, C₂-C₆ alkynyl, C₃-C₆ cycloalkyl, C₃-C₆ heterocycloalkyl, R₅-substituted C₁-C₆ alkyl, R₅-substituted alkoxy, R₅-substituted C₂-C₆ alkenyl, R₅-substituted C₂-C₆ alkynyl, R₅-substituted cycloalkyl, R₅-substituted C₃-C₆ heterocycloalkyl or halogen, or R₄ₐ₁, together with the attached carbon atom, forms a cycloalkyl, halocycloalkyl or heterocycloalkyl ring; the heteroatom in the C₃-C₆ heterocycloalkyl or heterocycloalkyl is O or S;
W is H, OH, halogen, C₁-C₄ alkoxy or C₁-C₄ haloalkoxy;
Yₐ, Y_{b}, Y_{c} and Y_{d} are independently 0, S, CH₂, CCl₂, CF₂ or NH;
Y₁ₐ, Y_{1b} and Y_{1c} are independently O or S;
Y₂ₐ, Y_{2b} and Y_{2c} are independently OH, SH or BH₃;
Y₃ and Y₄ are independently CH₂ or O;
Z₁ is O, OH, CH₂, S, NR₆, CO or SO₂;
Z₂ and Z₃ are independently O, NR₆, CHR₇, CHCOOR₇, CHCONR₇R₇, S, CO, SO₂, PO(OH), PS(OH), PO(SH), P(O)VCO₂H or absent;
Z₄ is O, CH₂, S, NR₆, CO, SO₂ or absent;
B₁ and B₂ are independently a natural or modified pyrimidine nucleotide base or a natural or modified purine nucleotide base;
R₅ is C₁-C₆ alkyl, C₁-C₆ haloalkyl, C₂-C₆ alkenyl, C₂-C₆ haloalkenyl, C₂-C₆ alkynyl, C₂-C₆ haloalkynyl, OR₇, SR₇, NR₇R₇, COR₇, COOR₇, OCOOR₇, CONR₇R₇, NHCOR₇, OCONR₇R₇, halogen, CN, SO₂, NO₂, D, N₃, aryl or heteroaryl;
R₆ is H, C₁-C₆ alkyl, COR₇ or SO₂R₇;
R₇ is H, C₁-C₈ alkyl, C₂-C₈ alkenyl, C₂-C₈ alkynyl or absent;
R₈ is H, halogen, C₁-C₈ alkyl, C₁-C₈ alkoxy, C₂-C₈ alkenyl, C₂-C₈ alkenyloxy, C₂-C₈ alkynyl, C₂-C₈ alkynyloxy, phenyl, benzyl, C₁-C₈ haloalkyl, C₁-C₈ haloalkoxy, C₂-C₈ haloalkenyl, C₂-C₈ haloalkenyloxy, C₂-C₈ haloalkynyl, C₂-C₈ haloalkynyloxy, halophenyl or halobenzyl;
V is C₁-C₄ alkyl;
m is 1, 2 or 3;
n is 0, 1 or 2;
n1 is 0, 1, 2, 3 or 4;
wherein halo means that one or more hydrogens are substituted by one or more of fluorine, chlorine, bromine or iodine.

In some embodiments, the compound has a structure represented by formula I-a below:

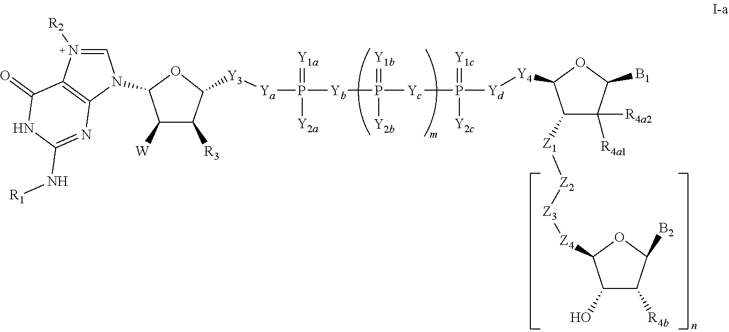

in formula I-a, W and R₃ are on the same side;
R₃ is H, OH, halogen, CN, N₃, C₁-C₆ alkyl, C₂-C₆ alkenyl, C₂-C₆ alkynyl, C₃-C₆ cycloalkyl, C₃-C₆ cycloalkenyl, benzyl, R₈-substituted C₁-C₆ alkyl, R₈-substituted C₂-C₆ alkenyl, R₈-substituted C₂-C₆ alkynyl, R₈-substituted C₃-C₆ cycloalkyl, R₈-substituted C₃-C₆ cycloalkenyl, R₈-substituted benzyl, —(CH₂)ₙ₁C(O)NR₇R₈, —(CH₂)ₙ₁OC(O)NR₇R₈, —(CH₂)ₙ₁NR₇C(O)R₈ or —(CH₂)ₙ₁NR₇SO₂R₈;
n1 is 0;
R₈ is H, halogen, C₁-C₈ alkyl, C₂-C₈ alkenyl, C₂-C₈ alkynyl, phenyl, benzyl, C₁-C₈ haloalkyl, C₂-C₈ haloalkenyl, C₂-C₈ haloalkynyl, halophenyl or halobenzyl.

When n is 1, the compound has a structure represented by formula I-a0:

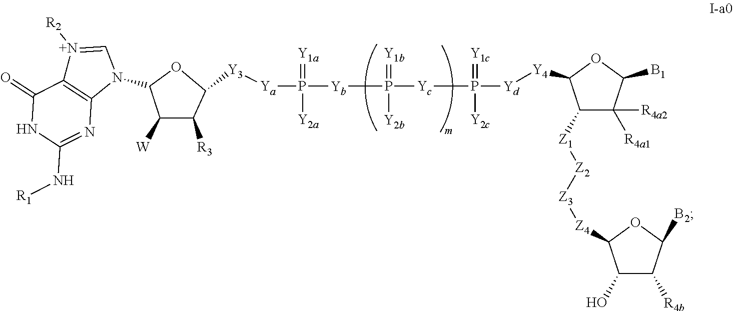

in formula I-a0, W and R₃ are on the same side;
R₁ is H, C₁-C₆ alkyl, C₂-C₆ alkenyl, C₂-C₆ alkynyl, C₃-C₆ cycloalkyl, C₃-C₆ cycloalkenyl, phenyl, benzyl, R₅-substituted C₁-C₆ alkyl, R₅-substituted C₂-C₆ alkenyl, R₅-substituted C₂-C₆ alkynyl, R₅-substituted C₃-C₆ cycloalkyl, R₅-substituted phenyl or R₅-substituted benzyl;
R₂ is C₁-C₆ alkyl, C₂-C₆ alkenyl, C₂-C₆ alkynyl, C₃-C₆ cycloalkyl, C₃-C₆ cycloalkenyl, phenyl, benzyl, R₅-substituted C₁-C₆ alkyl, R₅-substituted C₂-C₆ alkenyl, R₅-substituted C₂-C₆ alkynyl, R₅-substituted C₃-C₆ cycloalkyl, R₅-substituted C₃-C₆ cycloalkenyl, R₅-substituted phenyl or R₅-substituted benzyl;
R₃ is halogen, CN, N₃, C₁-C₆ alkyl, C₂-C₆ alkenyl, C₂-C₆ alkynyl, C₃-C₆ cycloalkyl, C₃-C₆ cycloalkenyl, benzyl, R₈-substituted C₁-C₆ alkyl, R₈-substituted C₂-C₆ alkenyl, R₈-substituted C₂-C₆ alkynyl, R₈-substituted C₃-C₆ cycloalkyl, R₈-substituted C₃-C₆ cycloalkenyl, R₈-substituted benzyl, —(CH₂)ₙ₁C(O)NR₇R₈, —(CH₂)ₙ₁OC(O)NR₇R₈, —(CH₂)ₙ₁NR₇C(O)R₈ or —(CH₂)ₙ₁NR₇SO₂R₈;
R₄ₐ₁, R₄ₐ₂ and R_{4b} are each independently H, OH, C₁-C₆ alkyl, C₁-C₆ alkoxy, C₂-C₆ alkenyl, C₂-C₆ alkynyl, C₃-C₆ cycloalkyl, R₈-substituted C₁-C₆ alkyl, R₈-substituted C₁-C₆ alkoxy, R₈-substituted C₂-C₆ alkenyl, R₈-substituted C₂-C₆ alkynyl, R₈-substituted C₃-C₆ cycloalkyl or halogen;
W is OH, halogen, C₁-C₄ alkoxy or C₁-C₄ haloalkoxy;
Yₐ, Y_{b}, Y_{c} and Y_{d} are independently 0, S, CH₂, CCl₂, CF₂ or NH;
Y₁ₐ, Y_{1b} and Y_{1c} are independently O or S;
Y₂ₐ, Y_{2b} and Y_{2c} are independently OH, SH or BH₃;
Y₃ and Y₄ are independently CH₂, O or S;
Z₁ is O, CH₂, S, NR₆, CO or SO₂;
Z₂ and Z₃ are independently O, NR₆, CHR₇, CHCOOR₇, CHCONR₇R₇, CO, SO₂, PO(OH), PS(OH), PO(SH) or absent;
Z₄ is O, CH₂, S, NR₆, CO, SO₂ or absent;
B₁ and B₂ are independently a natural or modified pyrimidine nucleotide base or a natural or modified purine nucleotide base;
R₅ is C₁-C₆ alkyl, C₁-C₆ haloalkyl, C₂-C₆ alkenyl, C₂-C₆ haloalkenyl, C₂-C₆ alkynyl, C₂-C₆ haloalkynyl, OR₇, SR₇, NR₇R₇, halogen, CN, D or N₃;
R₆ is H or C₁-C₆ alkyl;

R₇ is H, C₁-C₈ alkyl, C₂-C₈ alkenyl or C₂-C₈ alkynyl;

R₈ is H, halogen, C₁-C₈ alkyl, C₂-C₈ alkenyl, C₂-C₈ alkynyl, phenyl, benzyl, C₁-C₈ haloalkyl, C₂-C₈ haloalkenyl, C₂-C₈ haloalkynyl, halophenyl or halobenzyl;
m is 1, 2 or 3;
n1 is 0, 1, 2, 3 or 4;
wherein halo means that one or more hydrogens are substituted by one or more of fluorine, chlorine, bromine or iodine.

In some embodiments, W is H, OH, halogen, methoxy, ethoxy, propoxy, isopropoxy, n-butoxy, sec-butoxy, tert-butoxy, halomethoxy, haloethoxy, halopropoxy, haloisopropoxy, halo-n-butoxy, halo-sec-butoxy or halo-tert-butoxy, wherein halo means that one or more hydrogens are substituted by one or more of fluorine, chlorine, bromine or iodine.

More preferably, W is H, OH, fluorine, chlorine, methoxy, ethoxy, propoxy, isopropoxy, n-butoxy, sec-butoxy, tert-butoxy, monofluoromethoxy, difluoromethoxy, trifluoromethoxy, monochloromethoxy, dichloromethoxy, trichloromethoxy, monofluoroethoxy, difluoroethoxy, trifluoroethoxy, monochloroethoxy, dichloroethoxy, trichloroethoxy, monofluoropropoxy, difluoropropoxy, trifluoropropoxy, monochloropropoxy, dichloropropoxy, trichloropropoxy, monofluoroisopropoxy, difluoroisopropoxy, trifluoroisopropoxy, monochloroisopropoxy, dichloroisopropoxy, trichloroisopropoxy, monofluoro-n-butoxy, difluoro-n-butoxy, trifluoro-n-butoxy, monochloro-n-butoxy, dichloro-n-butoxy, trichloro-n-butoxy, monofluoro-tert-butoxy, difluoro-tert-butoxy, trifluoro-tert-butoxy, monochloro-tert-butoxy, dichloro-tert-butoxy or trichloro-tert-butoxy.

More preferably, W is H, OH, fluorine, chlorine, methoxy, ethoxy, propoxy, isopropoxy, monofluoromethoxy, difluoromethoxy, trifluoromethoxy, monofluoropropoxy, difluoropropoxy or trifluoropropoxy.

More preferably, W is OH, fluorine, chlorine, methoxy, ethoxy, monofluoromethoxy, difluoromethoxy, trifluoromethoxy, monofluoroethoxy, difluoroethoxy or trifluoroethoxy.

More preferably, W is OH, fluorine, chlorine, methoxy, ethoxy, monofluoromethoxy, trifluoromethoxy, monofluoroethoxy or trifluoroethoxy.

More preferably, W is OH, fluorine, chlorine, methoxy, ethoxy, trifluoromethoxy or trifluoroethoxy.

More preferably, W is OH, fluorine, methoxy or trifluoromethoxy.

More preferably, when W is one of H, OH, fluorine, chlorine, methoxy, ethoxy, propoxy or isopropoxy, then in formula I-a, R₃ is H, OH, halogen, CN, N₃, C₁-C₆ alkyl, C₂-C₆ alkenyl, C₂—C₆ alkynyl, C₃-C₆ cycloalkyl, C₃-C₆ cycloalkenyl, benzyl, R₈-substituted C₁-C₆ alkyl, R₈-substituted C₂-C₆ alkenyl, R₈-substituted C₂-C₆ alkynyl, R₈-substituted C₃-C₆ cycloalkyl, R₈-substituted C₃-C₆ cycloalkenyl, R₈-substituted benzyl, —(CH₂)ₙ₁C(O)NR₇R₈, —(CH₂)ₙ₁OC(O)NR₇R₈, —(CH₂)ₙ₁NR₇C(O)R₈ or —(CH₂)ₙ₁NR₇SO₂R₈;
n1 is 0;
R₈ is H, halogen, C₁-C₈ alkyl, C₂-C₈ alkenyl, C₂-C₈ alkynyl, phenyl, benzyl, C₁-C₈ haloalkyl, C₂-C₈ haloalkenyl, C₂-C₈ haloalkynyl, halophenyl or halobenzyl.

In some embodiments, when W is OH and R₄ₐ₂ is H, then the compound has a structure represented by formula I-a1:

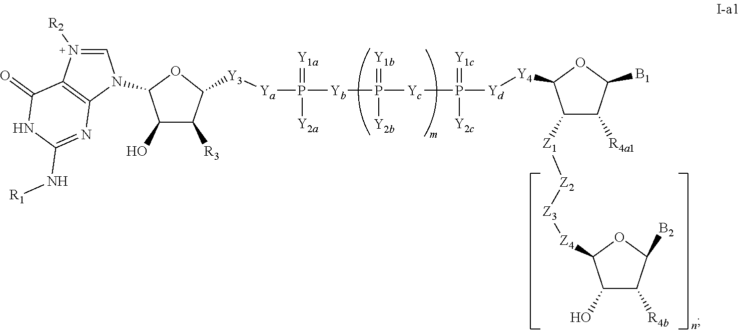

in formula I-a1, R₃ is H, OH, halogen, CN, N₃, C₁-C₆ alkyl, C₂-C₆ alkenyl, C₂-C₆ alkynyl, C₃-C₆ cycloalkyl, C₃-C₆ cycloalkenyl, benzyl, R₈-substituted C₁-C₆ alkyl, R₈-substituted C₂-C₆ alkenyl, R₈-substituted C₂-C₆ alkynyl, R₈-substituted C₃-C₆ cycloalkyl, R₈-substituted C₃-C₆ cycloalkenyl, R₈-substituted benzyl, —C(O)NR₇R₈ or —NR₇C(O)R₈;
R₈ is H, halogen, C₁-C₈ alkyl, C₂-C₈ alkenyl, C₂-C₈ alkynyl, phenyl, benzyl, C₁-C₈ haloalkyl, C₂-C₈ haloalkenyl, C₂-C₈ haloalkynyl, halophenyl or halobenzyl.

In some embodiments, when n is 1, the compound has a structure represented by formula I-a11:

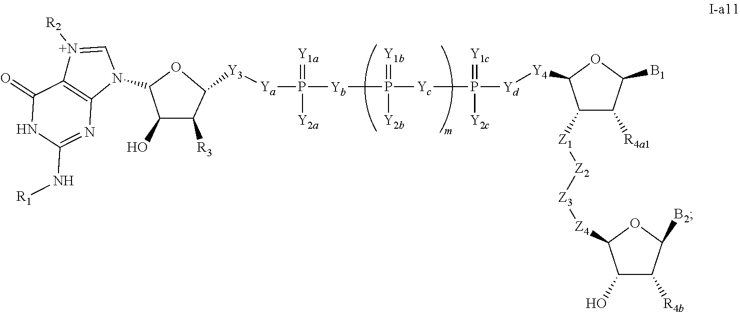

wherein R₃ is halogen, CN, N₃, C₁-C₆ alkyl, C₂-C₆ alkenyl, C₂-C₆ alkynyl, C₃-C₆ cycloalkyl, C₃-C₆ cycloalkenyl, benzyl, R₈-substituted C₁-C₆ alkyl, R₈-substituted C₂-C₆ alkenyl, R₈-substituted C₂-C₆ alkynyl, R₈-substituted C₃-C₆ cycloalkyl, R₈-substituted C₃-C₆ cycloalkenyl, R₈-substituted benzyl, —C(O)NR₇R₈ or —NR₇C(O)R₈;
R₈ is H, halogen, C₁-C₈ alkyl, C₂-C₈ alkenyl, C₂-C₈ alkynyl, phenyl, benzyl, C₁-C₈ haloalkyl, C₂-C₈ haloalkenyl, C₂-C₈ haloalkynyl, halophenyl or halobenzyl.

In some embodiments, in formula I-a1, R₃ is halogen, CN, N₃, C₁-C₄ alkyl, C₂-C₆ alkenyl, C₂-C₆ alkynyl, C₃-C₆ cycloalkyl, benzyl, C₁-C₆ haloalkyl, C₂-C₆ haloalkenyl, C₂-C₆ haloalkynyl, C₃-C₆ halocycloalkyl, halobenzyl, —C(O)NR₇R₈ or —NR₇C(O)R₈;
R₈ is H, halogen, C₁-C₆ alkyl, C₂-C₆ alkenyl, C₂-C₆ alkynyl, phenyl, benzyl, C₁-C₆ haloalkyl, C₂-C₆ haloalkenyl, C₂-C₆ haloalkynyl, halophenyl or halobenzyl.

In some embodiments, R₃ is represented by a1 below:
(a1) R₃ is halogen, CN, N₃, C₁-C₄ alkyl, C₂-C₆ alkenyl, C₂-C₆ alkynyl, C₃-C₆ cycloalkyl, benzyl, C₁-C₆ haloalkyl, C₂-C₆ haloalkenyl, C₂-C₆ haloalkynyl, C₃-C₆ halocycloalkyl or halobenzyl.

In some embodiments, R₃ is represented by a2 below:
(a2) R₃ is C₁-C₄ alkyl, C₂-C₆ alkenyl, C₂-C₆ alkynyl, C₃-C₆ cycloalkyl, benzyl, C₁-C₆ haloalkyl, C₂-C₆ haloalkenyl, C₂-C₆ haloalkynyl, C₃-C₆ halocycloalkyl or halobenzyl.

In some embodiments, R₃ is represented by a3 below:
(a3) R₃ is C₁-C₄ alkyl, C₂-C₄ alkenyl, C₂-C₄ alkynyl, C₃-C₆ cycloalkyl, benzyl, C₁-C₄ haloalkyl, C₂-C₄ haloalkenyl, C₂-C₄ haloalkynyl, C₃-C₆ halocycloalkyl or halobenzyl.

In some embodiments, R₃ is represented by a4 below:
(a4) R₃ is C₁-C₄ alkyl, C₂-C₄ alkenyl, C₃-C₆ cycloalkyl, C₁-C₄ haloalkyl, C₂-C₄ haloalkenyl or C₃-C₆ halocycloalkyl.

In some embodiments, R₃ is represented by a5 below:
(a5) R₃ is C₁-C₄ alkyl, C₃-C₆ cycloalkyl, C₁-C₄ haloalkyl or C₃-C₆ halocycloalkyl.

In some embodiments, R₃ is represented by a6 below:
(a6) R₃ is C₁-C₄ alkyl, C₃-C₆ cycloalkyl or C₁-C₄ haloalkyl.

In some embodiments, in formula I-a1, R₃ is halogen, CN, N₃, methyl, ethyl, propyl, isopropyl, cyclopropyl, n-butyl, sec-butyl, tert-butyl, propenyl, butenyl, propynyl, butynyl, halomethyl, haloethyl, halopropyl, haloisopropyl, halocyclopropyl, halo-n-butyl, halo-sec-butyl, halo-tert-butyl, halopropenyl, halobutenyl, halopropynyl, halobutynyl, benzyl, halobenzyl, C(O)NR₇R₈ or —NR₇C(O)R₈; R₇ is H, methyl, ethyl, propyl, isopropyl, cyclopropyl, n-butyl, sec-butyl, tert-butyl, propenyl, butenyl, methoxymethyl, methoxyethyl, methoxypropyl, methoxyisopropyl, methoxy-n-butyl, ethoxymethyl, ethoxyethyl, ethoxypropyl, ethoxyisopropyl, ethoxy-n-butyl, propoxymethyl, propoxyethyl, propoxypropyl, propoxyisopropyl or propoxy-n-butyl;
R₈ is H, methyl, ethyl, propyl, isopropyl, cyclopropyl, n-butyl, sec-butyl, tert-butyl, methoxy, ethoxy, propoxy, isopropoxy, cyclopropoxy, n-butoxy, sec-butoxy, tert-butoxy, propenyl, butenyl, methoxymethyl, methoxyethyl, methoxypropyl, methoxyisopropyl, methoxy-n-butyl, ethoxymethyl, ethoxyethyl, ethoxypropyl, ethoxyisopropyl, ethoxy-n-butyl, propoxymethyl, propoxyethyl, propoxypropyl, propoxyisopropyl, propoxy-n-butyl, phenyl or benzyl.

In some embodiments, R₃ is represented by b1 below: (b1) R₃ is halogen, CN, N₃, methyl, ethyl, propyl, isopropyl, cyclopropyl, n-butyl, sec-butyl, tert-butyl, propenyl, butenyl, propynyl, butynyl, halomethyl, haloethyl, halopropyl, haloisopropyl, halocyclopropyl, halo-n-butyl, halo-sec-butyl, halo-tert-butyl, halopropenyl, halobutenyl, halopropynyl, halobutynyl, benzyl or halobenzyl.

In some embodiments, R₃ is represented by b2 below: (b2) R₃ is methyl, ethyl, propyl, isopropyl, cyclopropyl, n-butyl, sec-butyl, tert-butyl, propenyl, butenyl, halomethyl, haloethyl, halopropyl, haloisopropyl, halocyclopropyl, halo-n-butyl, halo-sec-butyl, halo-tert-butyl, halopropenyl, halobutenyl, benzyl or halobenzyl.

In some embodiments, R₃ is represented by b3 below: (b3) R₃ is methyl, ethyl, propyl, isopropyl, cyclopropyl, n-butyl, sec-butyl, tert-butyl, halomethyl, haloethyl, halopropyl, haloisopropyl, halocyclopropyl, halo-n-butyl, halo-sec-butyl, halo-tert-butyl, benzyl or halobenzyl.

In some embodiments, R₃ is represented by b4 below: (b4) R₃ is methyl, ethyl, propyl, isopropyl, cyclopropyl, n-butyl, sec-butyl, tert-butyl, halomethyl, haloethyl, halopropyl, haloisopropyl, halocyclopropyl, halo-n-butyl, halo-sec-butyl or halo-tert-butyl.

In some embodiments, R₃ is represented by b5 below: (b5) R₃ is methyl, ethyl, propyl, isopropyl, cyclopropyl, n-butyl, sec-butyl or tert-butyl.

In some embodiments, R₃ is represented by b6 below: (b6) R₃ is halomethyl, haloethyl, halopropyl, haloisopropyl, halocyclopropyl, halo-n-butyl, halo-sec-butyl or halo-tert-butyl.

In some embodiments, in formula I-a1, R₃ is C(O)NHR₈, C(O)NR₇R₈, —NHC(O)R₈ or —NR₇C(O)R₈.

In some embodiments, in formula I-a1, R₃ is —C(O)NHCH₃, —C(O)NHCH₂CH₃, —C(O)NH(CH₂)₂CH₃, —C(O)NHCH(CH₃)₂, —C(O)NH(CH₂)₃CH₃, —C(O)NHCH₂CH(CH₃)₂, —C(O)NHCH₂OCH₃, —C(O)NH(CH₂)₂OCH₃, —C(O)NHCH₂OCH₂CH₃, —C(O)N(CH₃)₂, —C(O)N(CH₂CH₃)₂, —C(O)N(CH₂CH₂CH₃)₂, —C(O)N(CH₃)CH₂CH₂CH₃, —C(O)N(CH₂CH₃)—CH(CH₃)₂, —C(O)N(CH₃)—CH₂OCH₃, —C(O)N(CH₃)—CHOCH₂CH₃, —C(O)N(CHOCH₂CH₃)—CHOCH₂CH₃, —C(O)NHC₆H₅, —C(O)NHBn, —NHC(O)CH₃, —NHC(O)CH₂CH₃, —NHC(O)(CH₂)₂CH₃, —NHC(O)CH(CH₃)₂, —NHC(O)OCH₃, —NHC(O)OCH₂CH₃, —N(CH₃)C(O)CH₃, —N(CH₃)C(O)CH₂CH₃, —N(CH₃)C(O)(CH₂)₂CH₃, —N(CH₃)C(O)CH(CH₃)₂, —N(CH₂CH₃)C(O)CH₃, —N(CH₂CH₃)C(O)CH₂CH₃, —N(CH₂CH₃)C(O)(CH₂)₂CH₃, —N(CH₂CH₃)C(O)OCH₃, —N(CH₂OCH₃)C(O)OCH₂CH₃, —N(CH₂OCH₃)C(O)CH₂CH₃ or —N(CH₂OCH₃)C(O)CH₂OCH₂CH₃.

In some embodiments, R₁ is H, C₁-C₄ alkyl, C₂-C₄ alkenyl, C₂-C₄ alkynyl, C₃-C₆ cycloalkyl, C₃-C₆ cycloalkenyl, benzyl, C₁-C₄ haloalkyl, C₂-C₄ haloalkenyl, C₂-C₄ haloalkynyl, C₃-C₆ halocycloalkyl, benzyl, halobenzyl, C₁-C₄ alkylcarbonyl, C₁-C₄ alkoxycarbonyl or sulfonyl;
R₂ is H, C₁-C₄ alkyl, C₂-C₄ alkenyl, C₂-C₄ alkynyl, C₃-C₆ cycloalkyl, C₃-C₆ cycloalkenyl, C₁-C₄ haloalkyl, C₂-C₄ haloalkenyl, C₂-C₄ haloalkynyl, C₃-C₆ halocycloalkyl, C₃-C₆ halocycloalkenyl, benzyl or halobenzyl.

In some embodiments, R₁ is H, methyl, ethyl, n-propyl, isopropyl, butyl, vinyl, propenyl, butenyl, ethynyl, propynyl, butynyl, cyclopropyl, cyclobutyl, halomethyl, haloethyl, halo-n-propyl, haloisopropyl, halobutyl, haloethenyl, halopropenyl, halobutenyl, haloethynyl, halopropynyl, halobutynyl, halocyclopropyl, halocyclobutyl, benzyl, halobenzyl, —COCH₃, —COCH₂CH₃, —COCH₂CH₂CH₃, —COCH(CH₃)CH₃, —COOCH₃, —COOCH₂CH₃, —COOCH₂CH₂CH₃, —COOCH(CH₃)CH₃ or sulfonyl;

R₂ is H, methyl, ethyl, n-propyl, isopropyl, butyl, vinyl, propenyl, butenyl, ethynyl, propynyl, butynyl, cyclopropyl, cyclobutyl, halomethyl, haloethyl, halo-n-propyl, haloisopropyl, halobutyl, halovinyl, halopropenyl, halobutenyl, haloethynyl, halopropynyl, halobutynyl, halocyclopropyl, halocyclobutyl, benzyl or halobenzyl.

In some embodiments, R₁ is represented by c1 below: (c1) R₁ is H, C₁-C₆ alkyl, C₂-C₆ alkenyl, C₂-C₆ alkynyl, C₃-C₆ cycloalkyl, benzyl, C₁-C₆ haloalkyl, C₂-C₆ haloalkenyl, C₂-C₆ haloalkynyl, C₃-C₆ halocycloalkyl or halobenzyl.

In some embodiments, R₁ is represented by c2 below: (c2) R₁ is H, C₁-C₆ alkyl, C₂-C₆ alkenyl, C₃-C₆ cycloalkyl, benzyl, C₁-C₆ haloalkyl, C₂-C₆ haloalkenyl, C₃-C₆ halocycloalkyl or halobenzyl.

In some embodiments, R₁ is represented by c3 below: (c3) R₁ is H, C₁-C₆ alkyl, C₃-C₆ cycloalkyl, benzyl, C₁-C₆ haloalkyl, C₃-C₆ halocycloalkyl or halobenzyl.

In some embodiments, R₁ is represented by c4 below: (c4) R₁ is H, C₁-C₆ alkyl, C₃-C₆ cycloalkyl, benzyl, C₁-C₆ haloalkyl or halobenzyl.

In some embodiments, R₁ is represented by c5 below: (c5) R₁ is H, C₁-C₆ alkyl, C₃-C₆ cycloalkyl or C₁-C₄ haloalkyl.

In some embodiments, R₁ is represented by c6 below: (c6) R₁ is H, C₁-C₆ alkyl, C₃-C₆ cycloalkyl or benzyl.

In some embodiments, R₁ is represented by c7 below: (c7) R₁ is H, C₁-C₄ alkyl, C₃0.4 cycloalkyl, benzyl, C₁-C₄ haloalkyl or halobenzyl.

In some embodiments, R₁ is represented by c8 below: (c8) R₁ is H, C₁-C₄ alkyl, C₃0.4 cycloalkyl or C₁-C₄ haloalkyl.

In some embodiments, R₁ is represented by c9 below: (c9) R₁ is H, C₁-C₄ alkyl, C₃-C₄ cycloalkyl or benzyl.

In some embodiments, R₂ is represented by d1 below: (d1) R₂ is C₁-C₆ alkyl, C₂-C₆ alkenyl, C₂-C₆ alkynyl, C₃-C₆ cycloalkyl, benzyl, C₁-C₆ haloalkyl, C₂-C₆ haloalkenyl, C₃-C₆ halocycloalkyl or halobenzyl.

In some embodiments, R₂ is represented by d2 below: (d2) R₂ is C₁-C₆ alkyl, C₂-C₆ alkenyl, C₂-C₆ alkynyl, benzyl, C₁-C₆ haloalkyl, C₂₋₆ haloalkenyl or halobenzyl.

In some embodiments, R₂ is represented by d3 below: (d3) R₂ is C₁-C₆ alkyl, C₂-C₆ alkenyl, C₂-C₆ alkynyl, benzyl, C₁-C₆ haloalkyl or halobenzyl.

In some embodiments, R₂ is represented by d4 below: (d4) R₂ is C₁-C₆ alkyl, C₂-C₆ alkenyl, C₂-C₆ alkynyl, benzyl or halobenzyl.

In some embodiments, R₂ is represented by d5 below: (d5) R₂ is C₁-C₆ alkyl, C₂-C₆ alkenyl, C₂-C₆ alkynyl or benzyl.

In some embodiments, R₂ is represented by d6 below: (d6) R₂ is C₁-C₄ alkyl, C₂-C₄ alkenyl, C₂-C₄ alkynyl or benzyl.

In some embodiments, Z₁ is O, CH₂, S or NH;
Z₂ and Z₃ are independently NR₆, CHR₇, CHCOOR₇, CHCONR₇R₇, S, CO, SO₂, PO(OH), PO(SH) or absent;
Z₄ is O, CH₂, S, NH or absent;
B₁ and B₂ are independently a natural or modified cytosine nucleotide base, a natural or modified uracil nucleotide base, a natural or modified adenine nucleotide base or a natural or modified guanine nucleotide base;
R₆ is H, methyl, ethyl, propyl or isopropyl;
R₇ is H, C₁-C₄ alkyl, C₂-C₄ alkenyl or C₁-C₄ alkoxy-substituted C₁-C₄ alkyl.

In some embodiments, Yₐ, Y_{b}, Y_{c} and Y_{d} are all O or at most one is S, CH₂, CCl₂, CF₂ or NH;
Y₁ₐ, Y_{1b} and Y_{1c} are all O or at most one is S;
Y₂ₐ, Y_{2b} and Y_{2c} are all OH or at most one is SH or BH₃;
Y₃ and Y₄ are independently CH₂;
Z₂ is CH₂, CH₂CH₂, CO, SO₂, PO(OH) or absent;
Z₃ is O, CH₂ or NH;
Z₄ is CH₂ or NH.

In some embodiments, Z₁ is O, CH₂, S or NR₆;
Z₂ is CHR₇, CO, PO(OH) or PO(SH);
Z₃ is O, NR₆, CHR₇ or CO;
Z₄ is O, CH₂ or S.

In some embodiments, Z₁ is O, Z₂ is PO(OH) or PO(SH), Z₃ is O, and Z₄ is CH₂.

More preferably, Yₐ and Y_{d} are all O or at most one is S or CH₂.

More preferably, Y_{b} and Y_{c} are all O or at most one is S, CH₂, CCl₂, CF₂ or NH.

More preferably, Z₂ is CH₂, SO₂ or PO(OH).

In some embodiments, R₄ₐ₁, R₄ₐ₂ and R_{4b} are independently H, OH, C₁-C₄ alkyl, C₁-C₄ alkoxy, C₂-C₆ alkenyl, C₂-C₆ alkynyl, C₃-C₆ cycloalkyl, O-containing C₃-C₆ cycloalkyl, C₁-C₆ haloalkyl, C₁-C₆ haloalkoxy, C₂-C₆ haloalkenyl, C₂-C₆ haloalkynyl, C₃-C₆ halocycloalkyl, C₁-C₄ alkoxy-substituted C₁-C₄ alkyl or halogen, or R₄ₐ₁, together with the attached carbon atom, forms a C₃-C₆ cycloalkyl, C₃-C₆ halocycloalkyl or C₃-C₆ heterocycloalkyl ring; the heteroatom in the C₃-C₆ heterocycloalkyl is O or S.

In some embodiments, one of R₄ₐ₁ and R₄ₐ₂ is H, and the other substituent and R_{4b} are each independently e1 below: (e1) R₄ₐ₁ or R₄ₐ₂, R_{4b} are independently H, OH, halogen, C₁-C₆ alkyl, C₁-C₆ alkoxy, C₁-C₆ haloalkyl or C₁-C₆ haloalkoxy.

In some embodiments, one of R₄ₐ₁ and R₄ₐ₂ is H, and the other substituent and R_{4b} are each independently e2 below: (e2) R₄ₐ₁ or R₄ₐ₂, R_{4b} are independently H, OH, halogen, C₁-C₄ alkyl, C₁-C₄ alkoxy, C₁-C₄ haloalkyl or C₁-C₄ haloalkoxy.

In some embodiments, one of R₄ₐ₁ and R₄ₐ₂ is H, and the other substituent and R_{4b} are each independently e3 below: (e3) R₄ₐ₁ or R₄ₐ₂, R_{4b} are independently H, OH, halogen, C₁-C₄ alkyl or C₁-C₄ alkoxy.

In some embodiments, one of R₄ₐ₁ and R₄ₐ₂ is H, and the other substituent and R_{4b} are each independently e4 below: (e4) R₄ₐ₁ or R₄ₐ₂, R_{4b} are independently OH, halogen, methyl, ethyl, propyl, isopropyl, methoxy, ethoxy, propoxy, isopropoxy or n-butoxy.

In some embodiments, R₄ₐ₁, R₄ₐ₂ and R_{4b} are each independently H, OH, halogen, methyl, ethyl, n-propyl, isopropyl, propenyl, propynyl, cyclopropyl, cyclobutyl, cyclopentyl, cyclohexyl, halomethyl, haloethyl, halo-n-propyl, haloisopropyl, 0-containing cyclopropyl, 0-containing cyclopentyl, 0-containing cyclohexyl, halocyclopropyl, halocyclopentyl, halocyclohexyl, methoxymethyl, methoxyethyl, methoxypropyl, methoxyisopropyl, methoxy-n-butyl, ethoxymethyl, ethoxyethyl, ethoxypropyl, ethoxyisopropyl, ethoxy-n-butyl, propoxymethyl, propoxyethyl, propoxypropyl, propoxyisopropyl or propoxy-n-butyl.

More preferably, R₄ₐ₁ and R_{4b} are each independently H, OH, halogen, methyl, ethyl, n-propyl, isopropyl, propenyl, propynyl, cyclopropyl, cyclopentyl, cyclohexyl, fluoromethyl, difluoromethyl, trifluoromethyl, trifluoroethyl, trifluoropropyl, trifluoroisopropyl, fluorocyclopropyl, difluorocyclopropyl, fluorocyclopentyl or difluorocyclopentyl.

More preferably, each R₄ₐ₂ is independently H, methyl, ethyl, n-propyl, isopropyl, fluoromethyl, difluoromethyl, trifluoromethyl or trifluoroethyl.

In some embodiments, when R₄ₐ₁, together with the attached carbon atom, forms a ring, R₄ₐ₁ is cyclopropyl, halocyclopropyl, cyclobutyl, halocyclobutyl, cyclopentyl, halocyclopentyl, cyclohexyl, halocyclohexyl, epoxypropyl, epoxybutyl or epoxypentyl.

More preferably, when R₄ₐ₁, together with the attached carbon atom, forms a ring, R₄ₐ₁ is cyclopropyl, fluorocyclopropyl, difluorocyclopropyl, cyclobutyl, fluorocyclobutyl, difluorocyclobutyl, cyclopentyl, fluorocyclopentyl, difluorocyclopentyl, cyclohexyl, fluorocyclohexyl, difluorocyclohexyl, epoxypropyl, epoxybutyl or epoxypentyl.

In some embodiments, in all of the above general formulas, B₁ and B₂ are independently a natural or modified cytosine nucleotide base, a natural or modified uracil nucleotide base, a natural or modified adenine nucleotide base or a natural or modified guanine nucleotide base.

In some embodiments, when B₁ and B₂ are independently natural adenine nucleotide bases, their structure is represented by formula X:

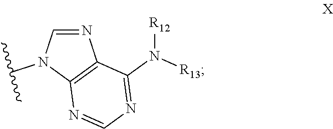

wherein R₁₂ and R₁₃ are independently H, C₁₋₄ alkyl or C₁₋₄ haloalkyl.

Preferably, R₁₂ and R₁₃ are independently H or C₁₋₄ alkyl.

In some embodiments, the compound is the following compound:

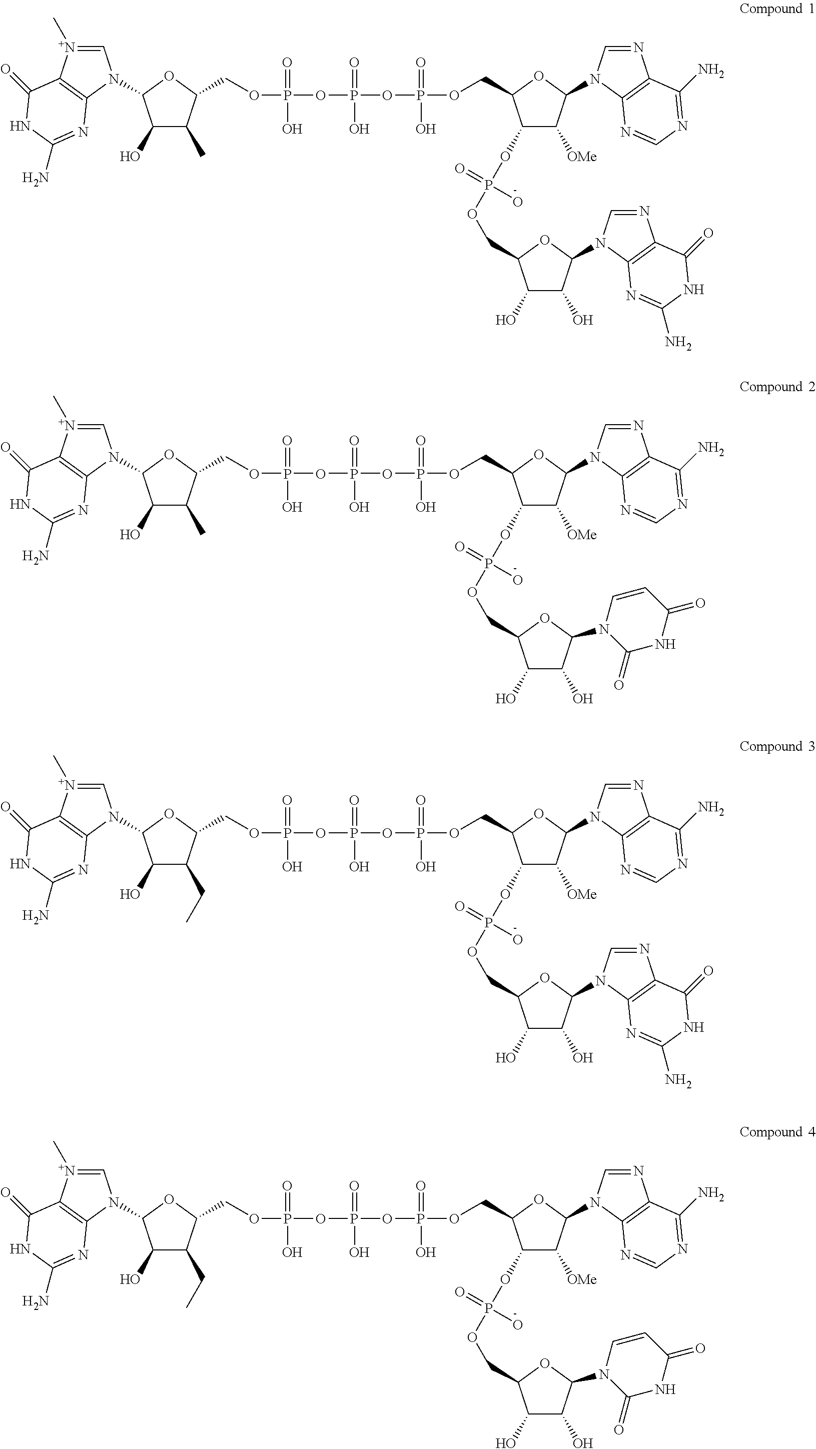

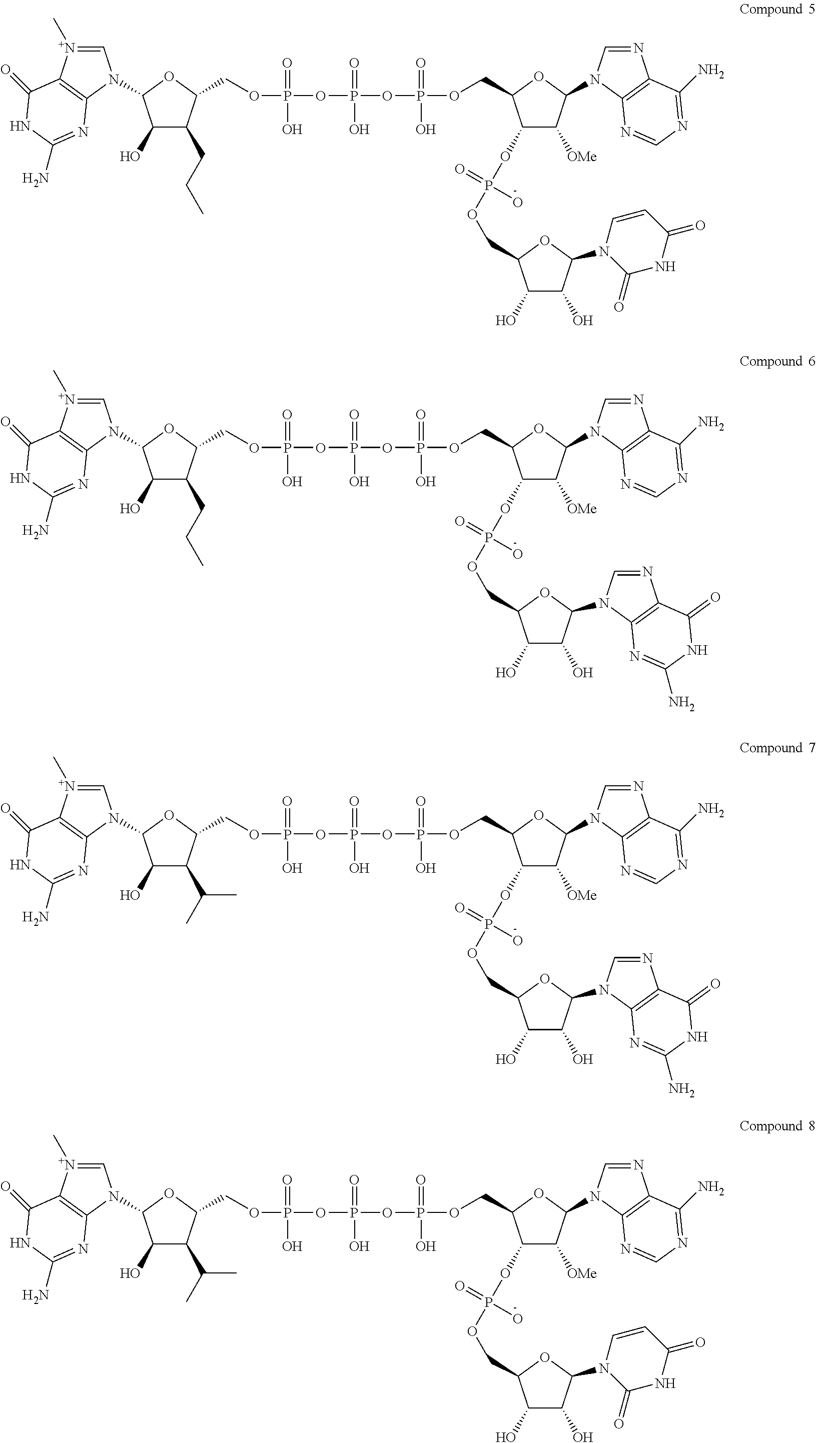

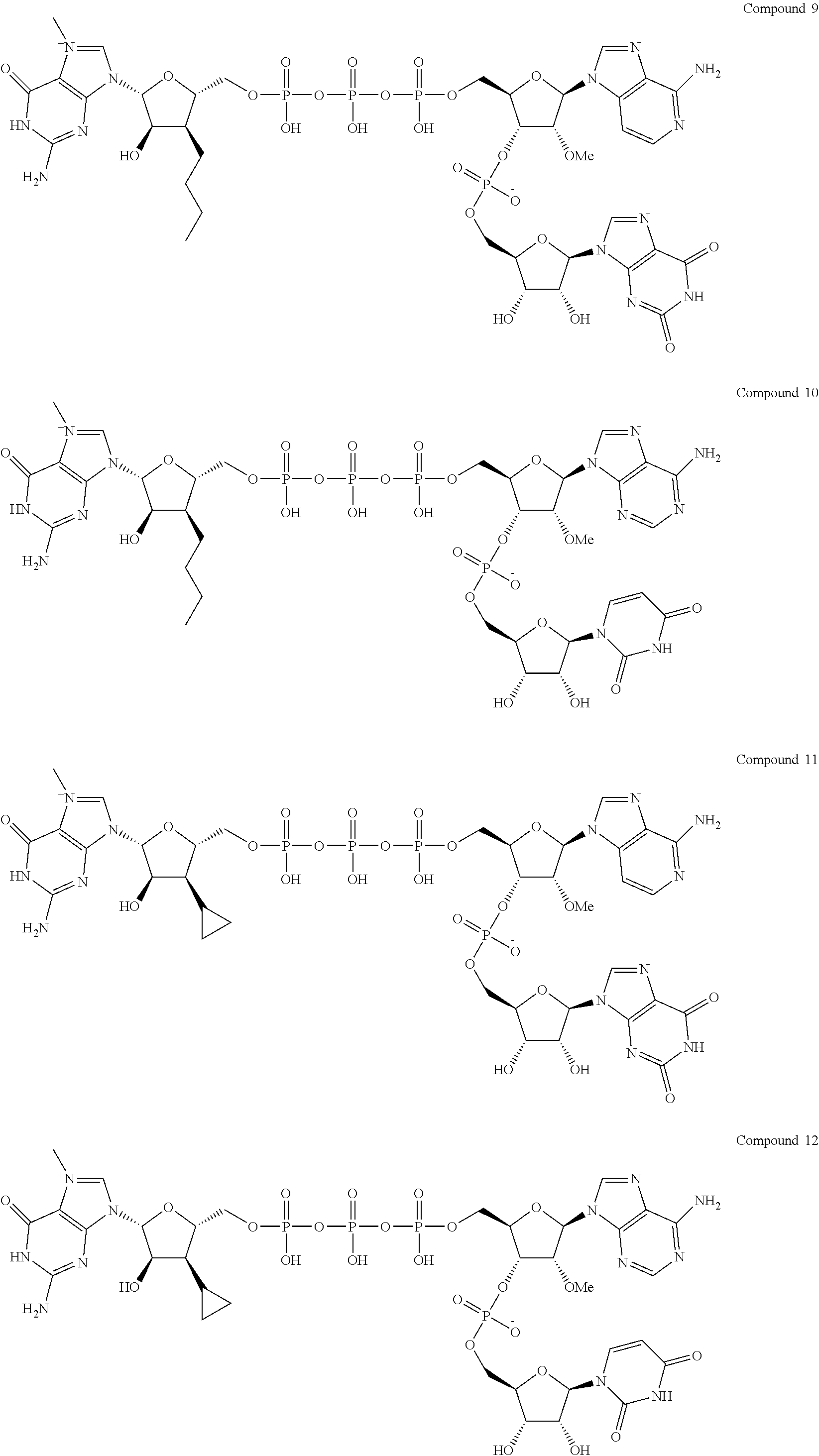

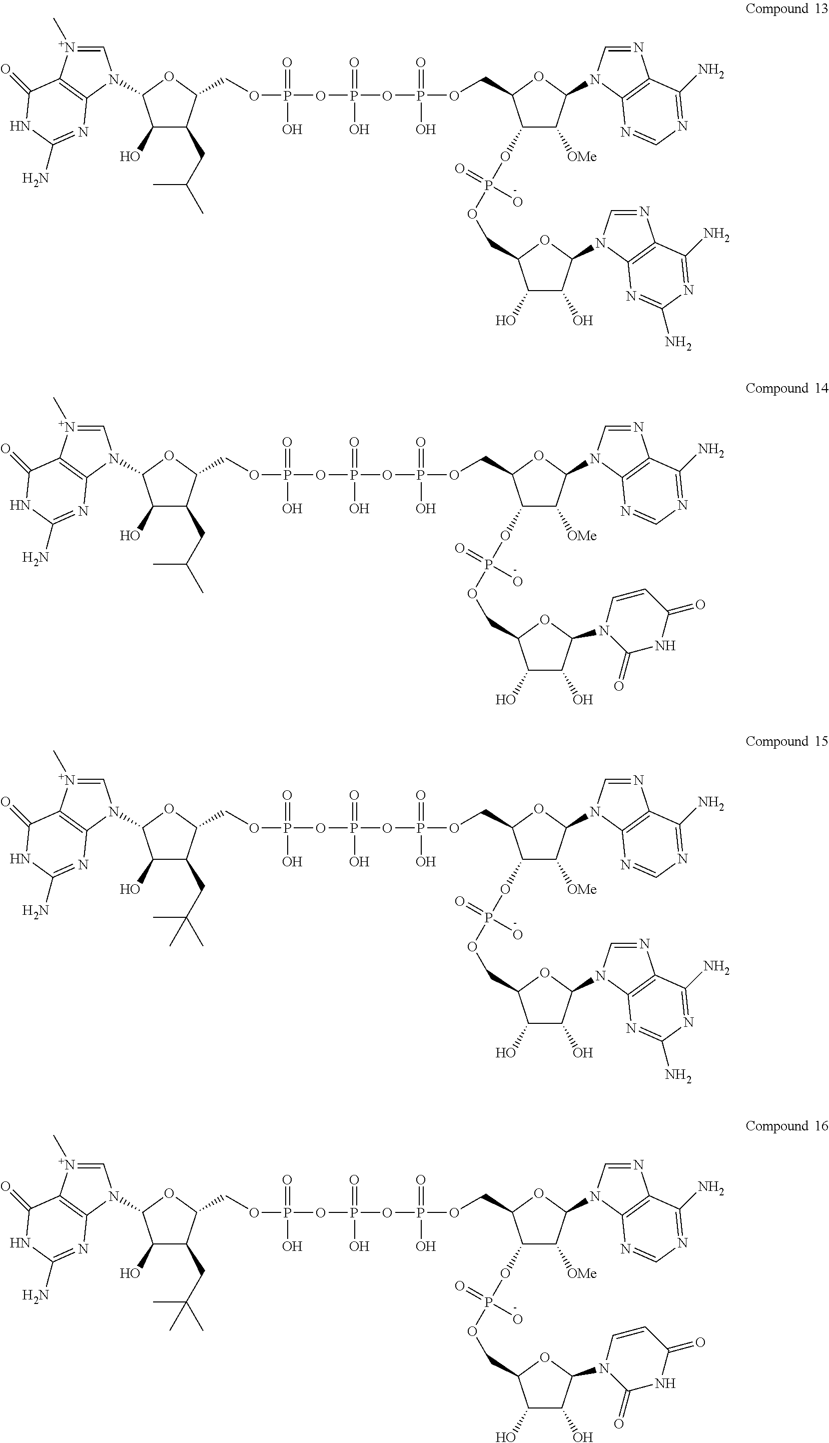

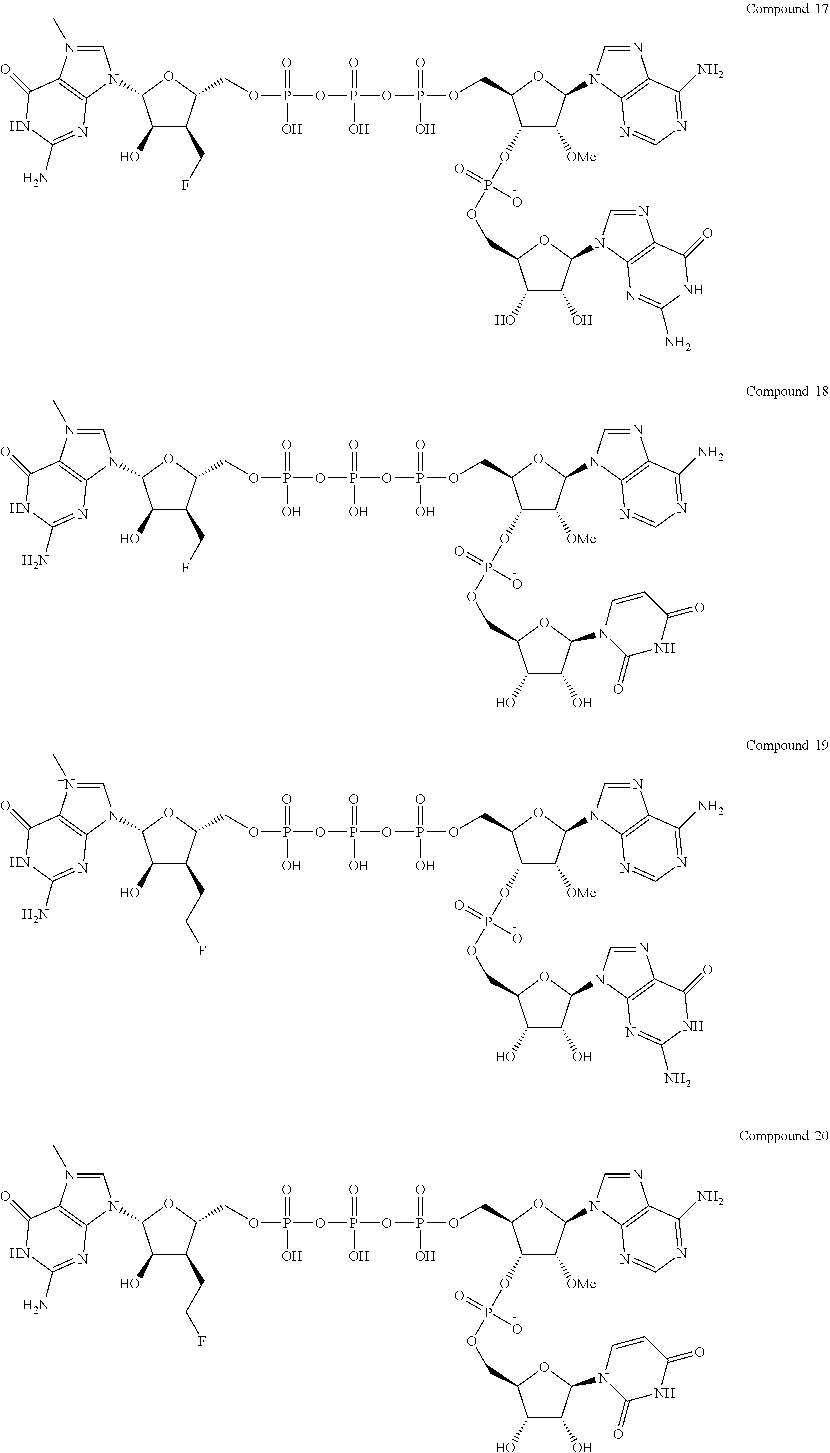

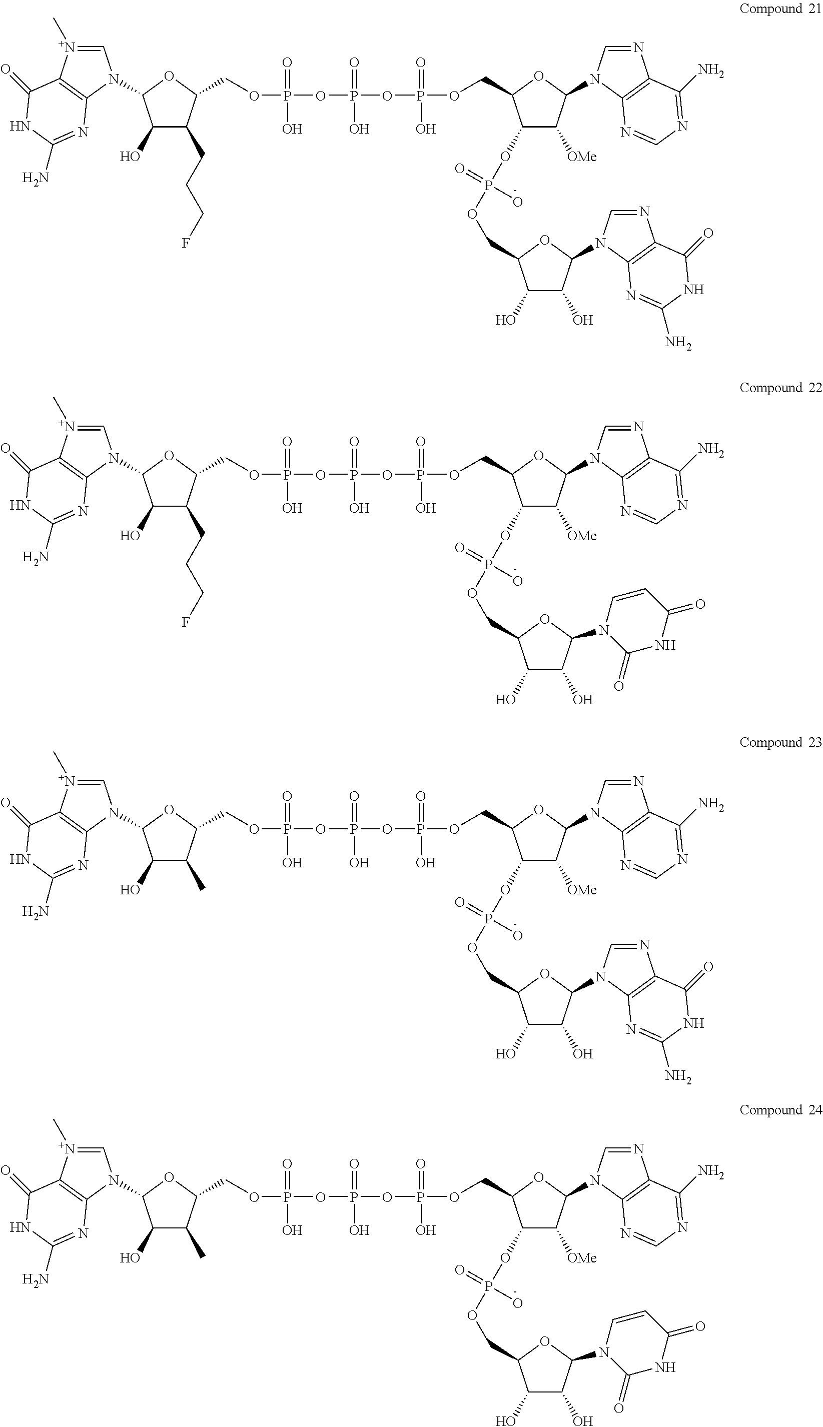

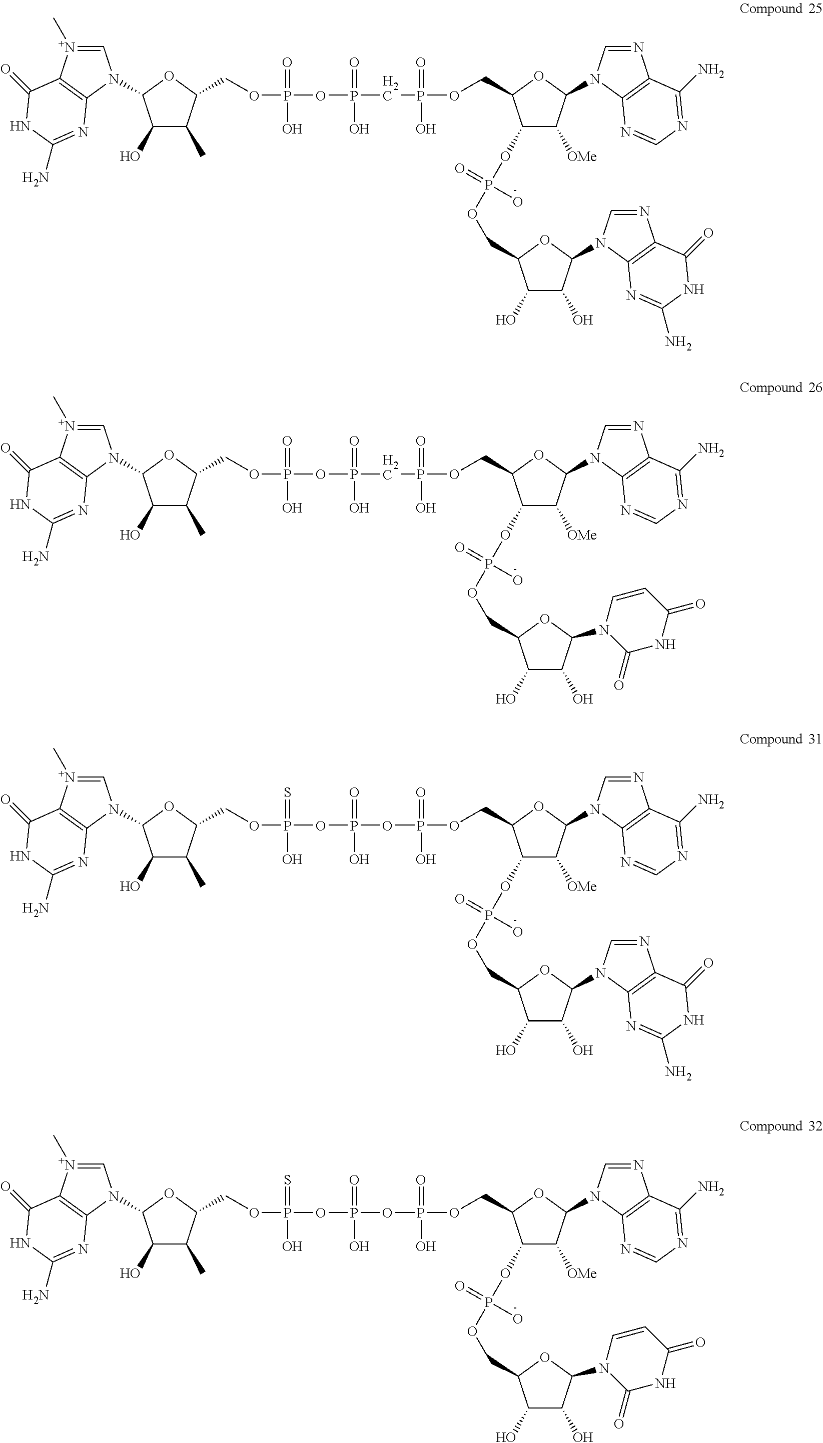

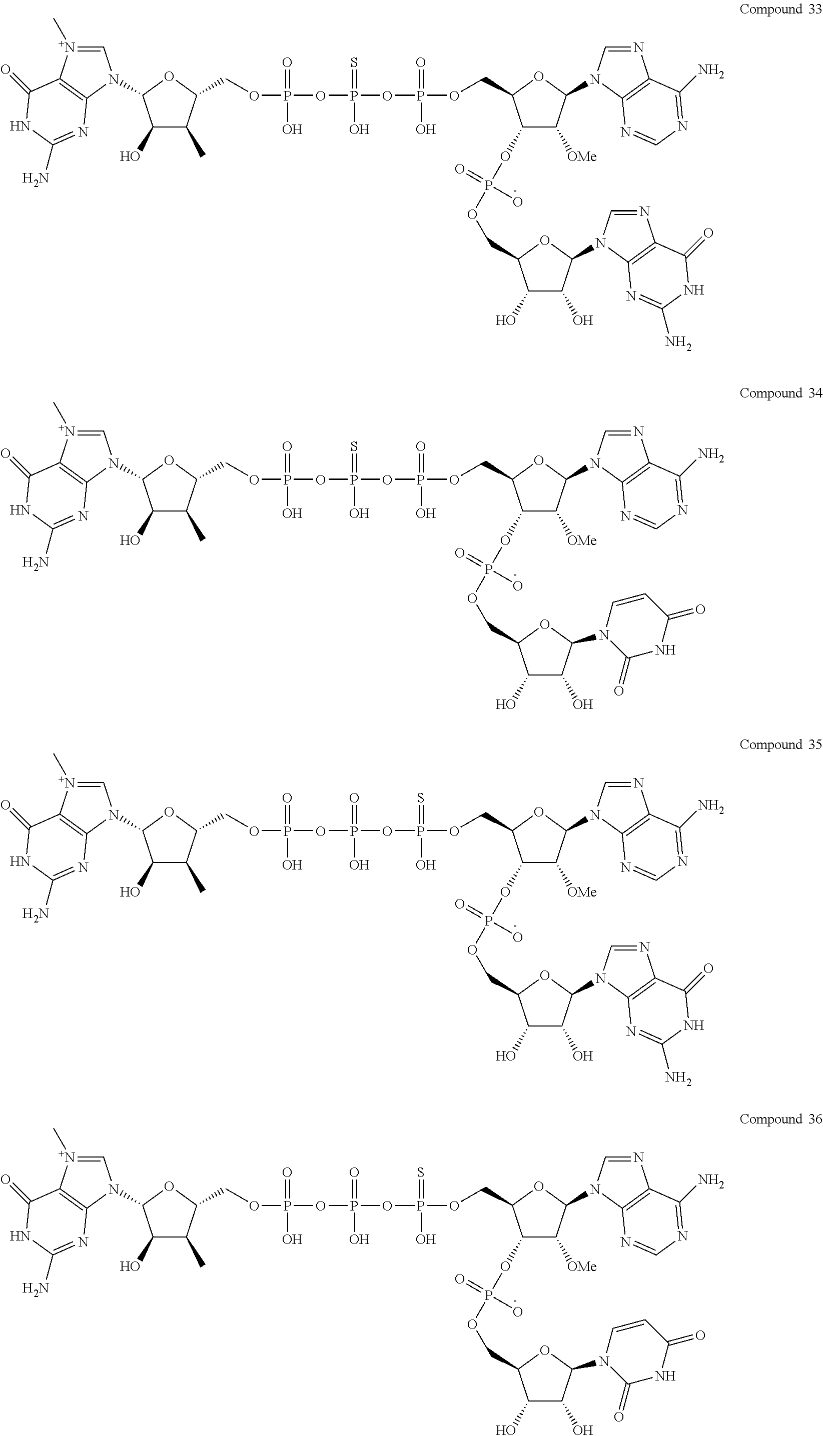

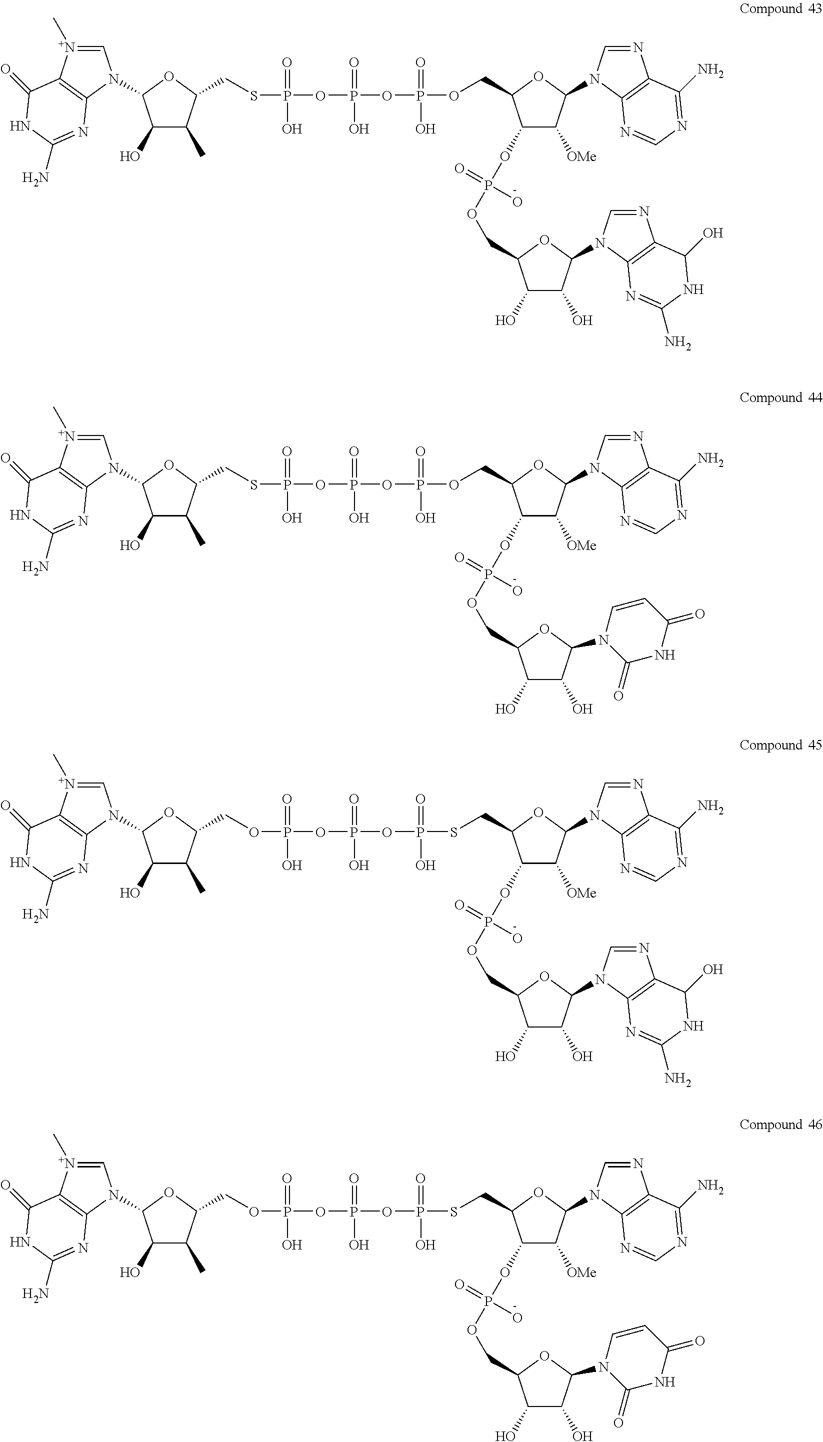

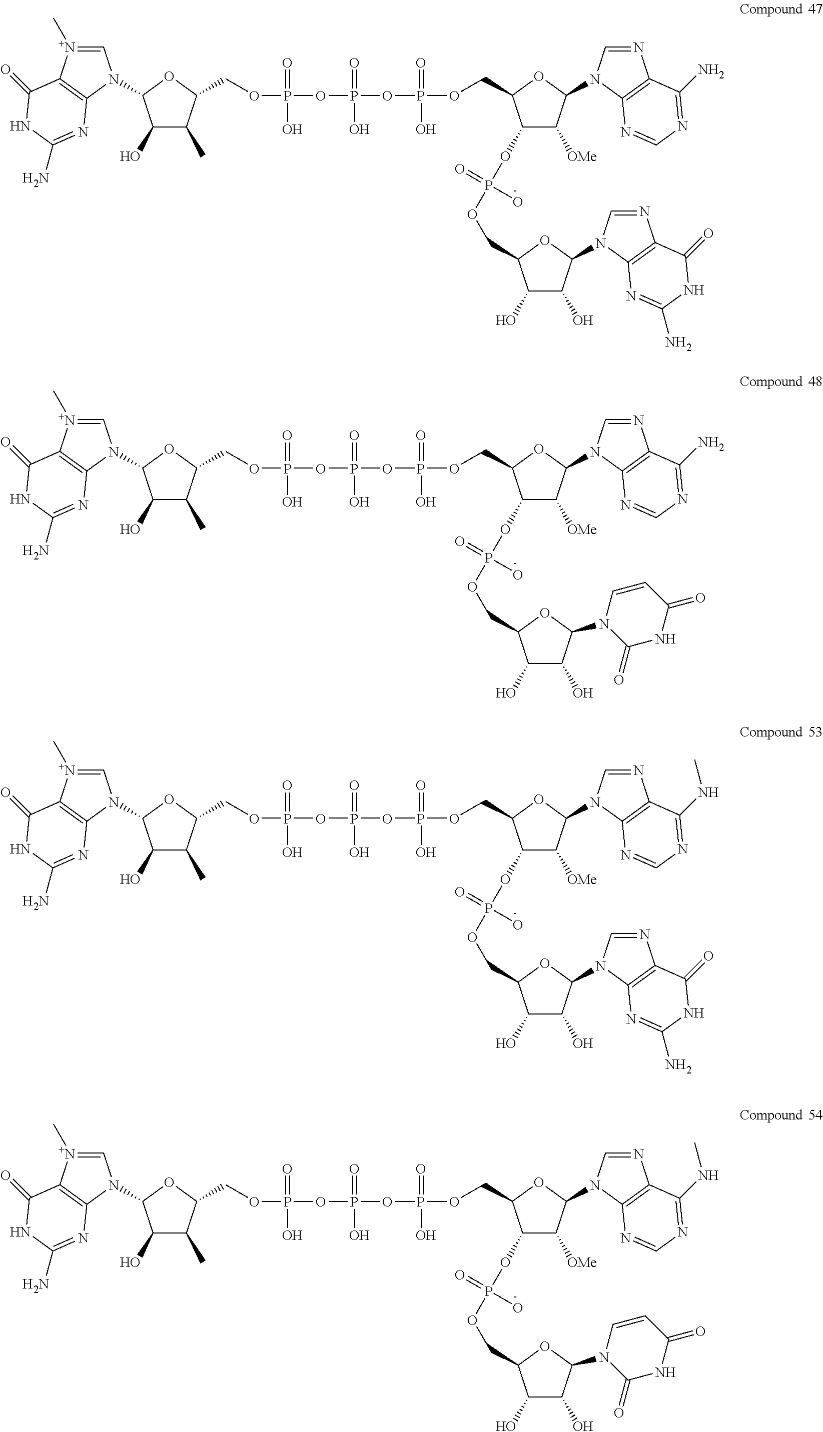

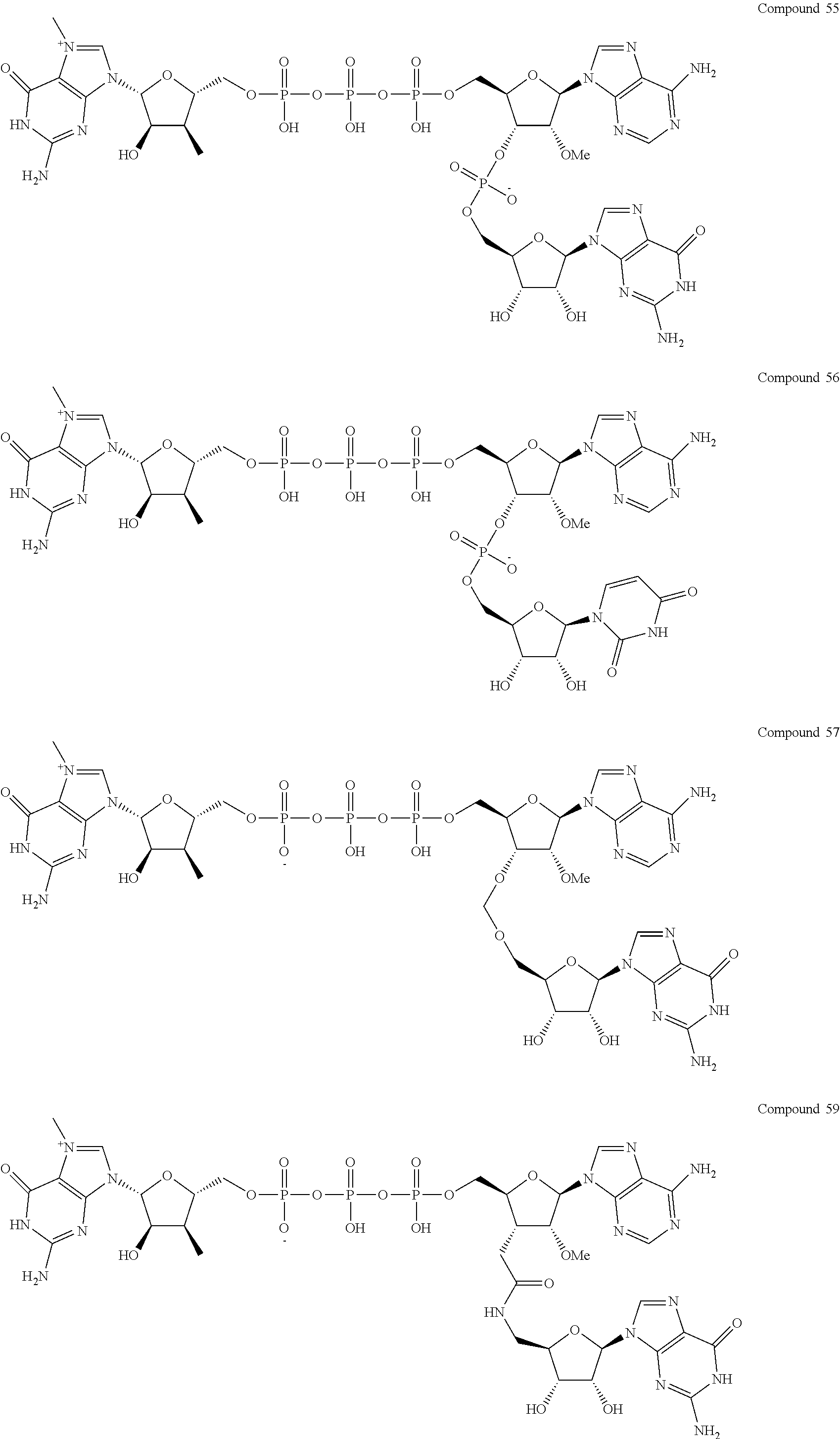

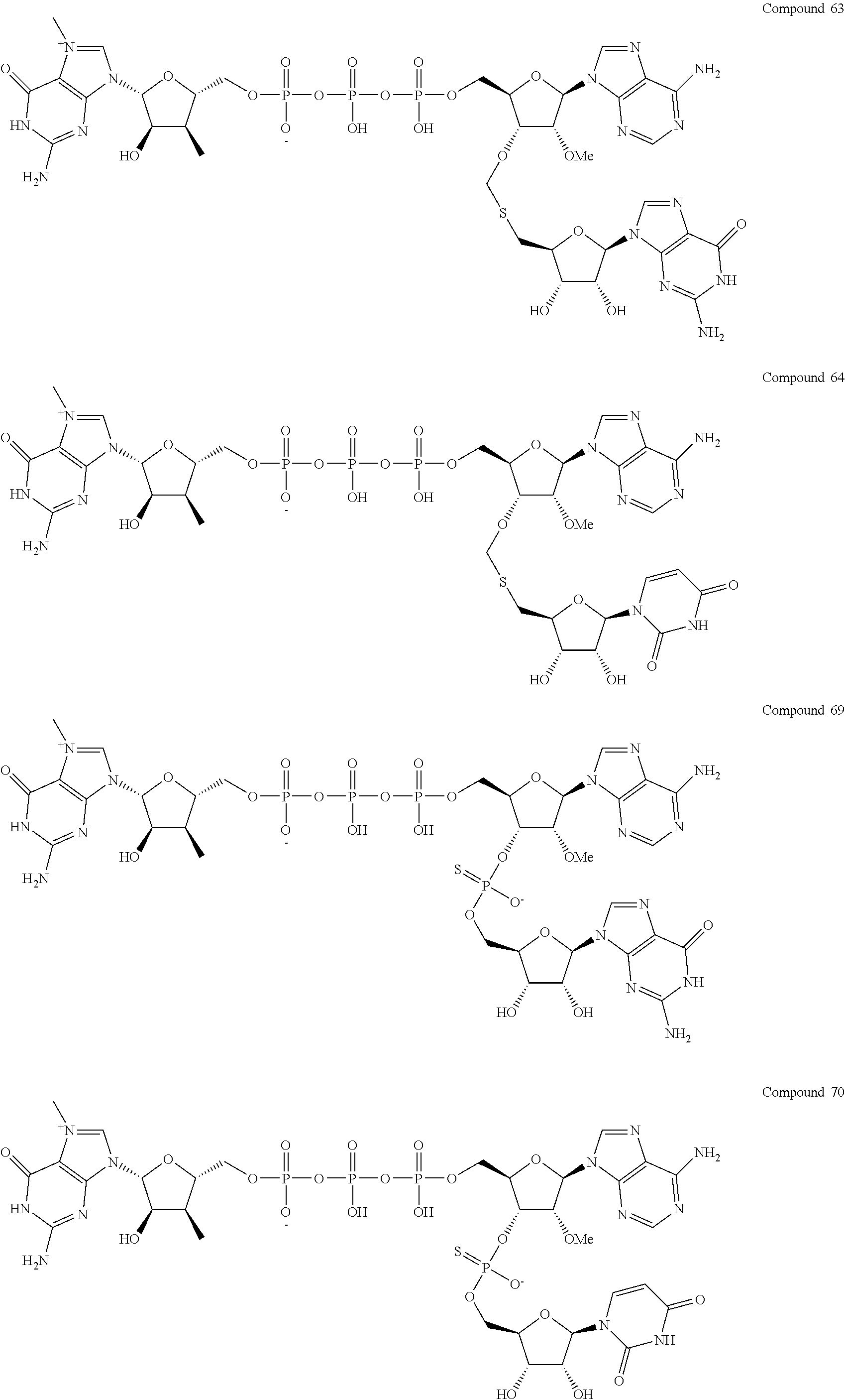

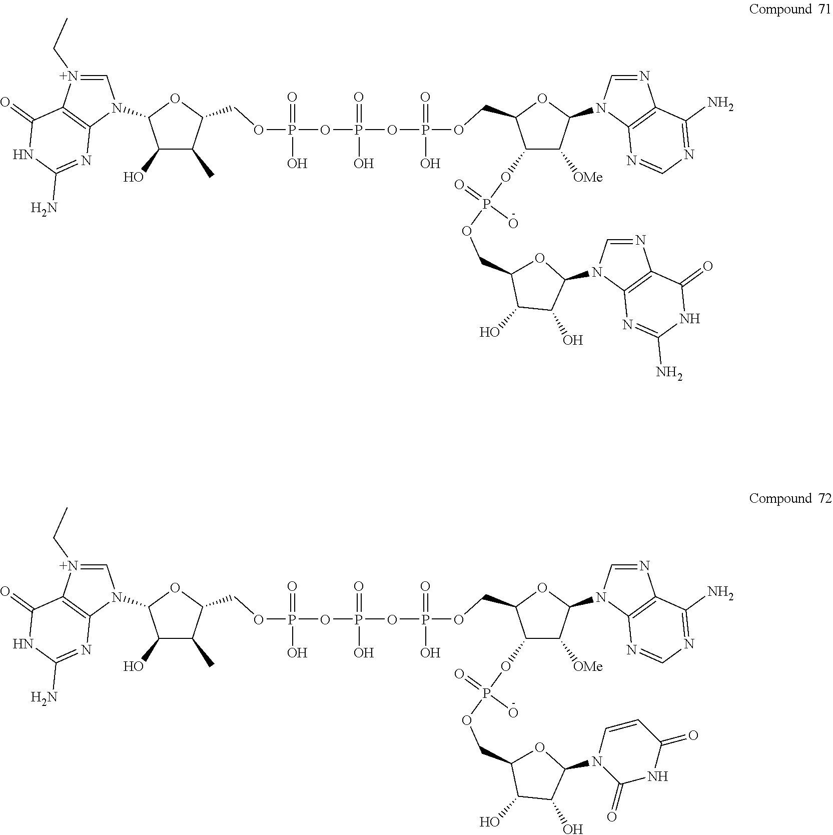

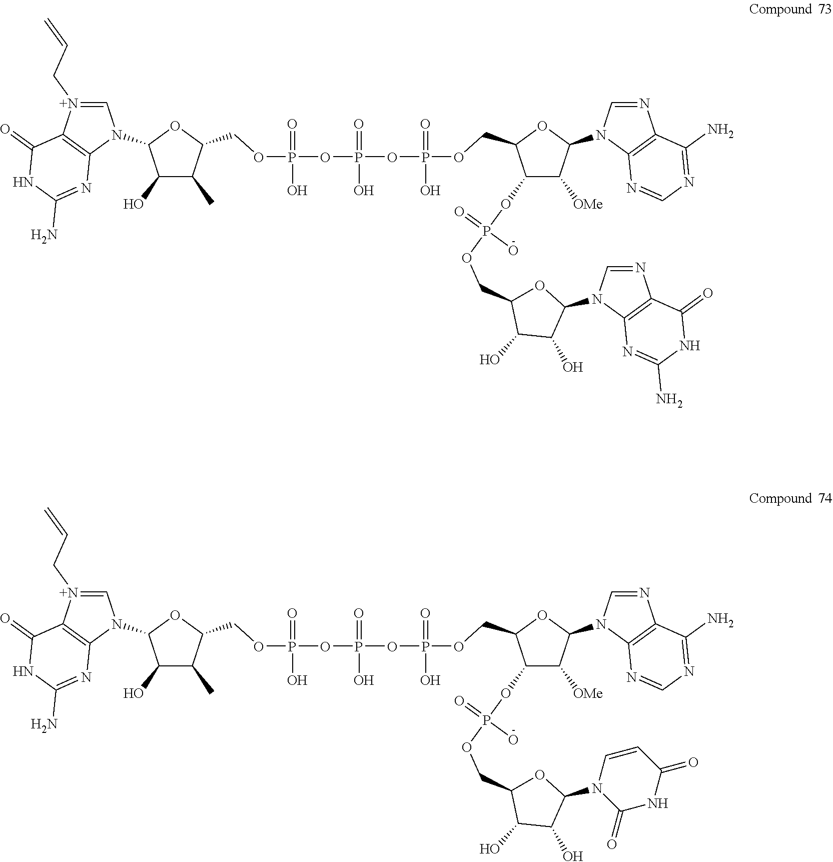

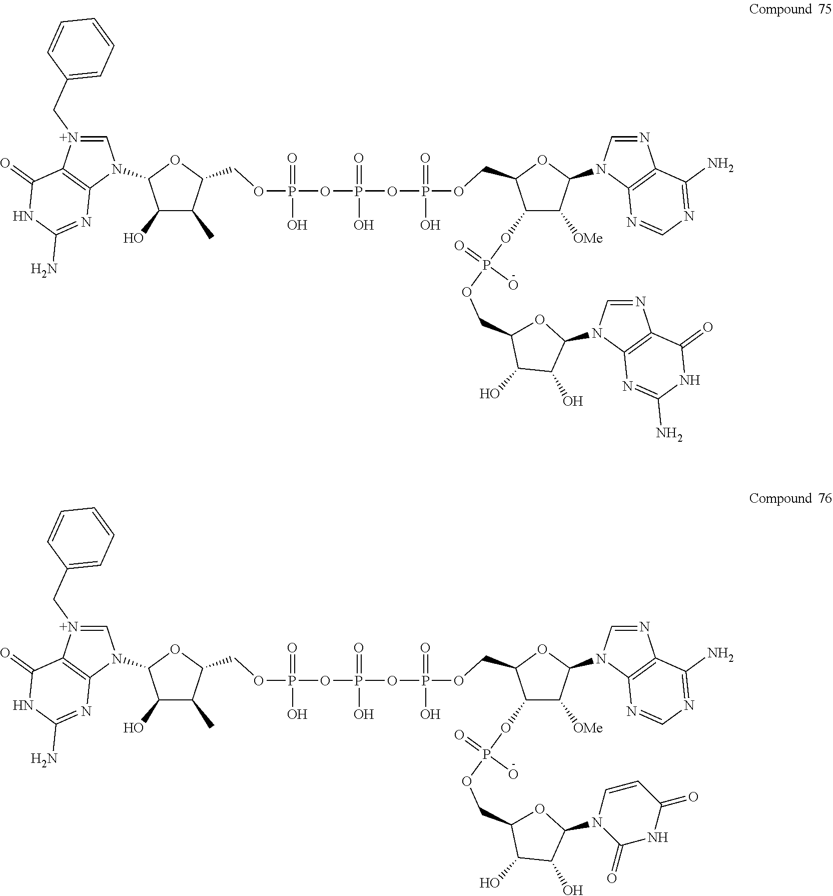

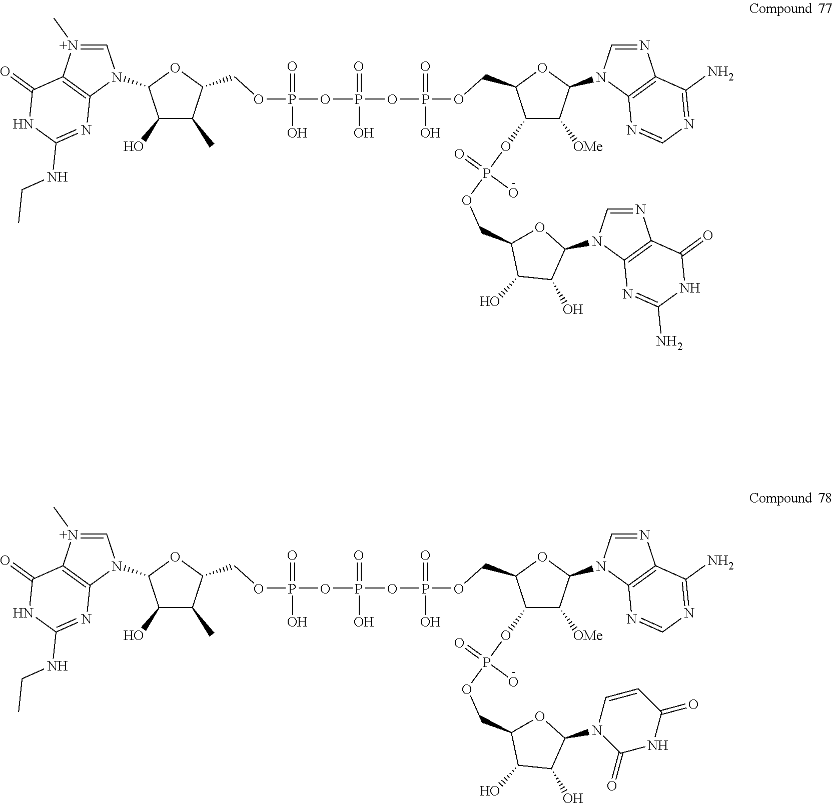

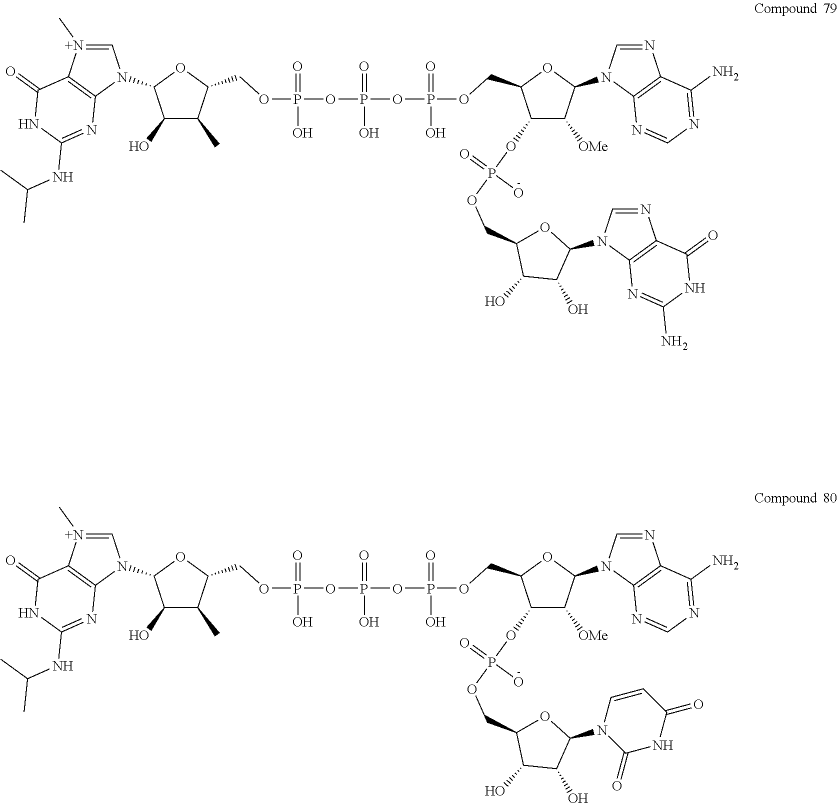

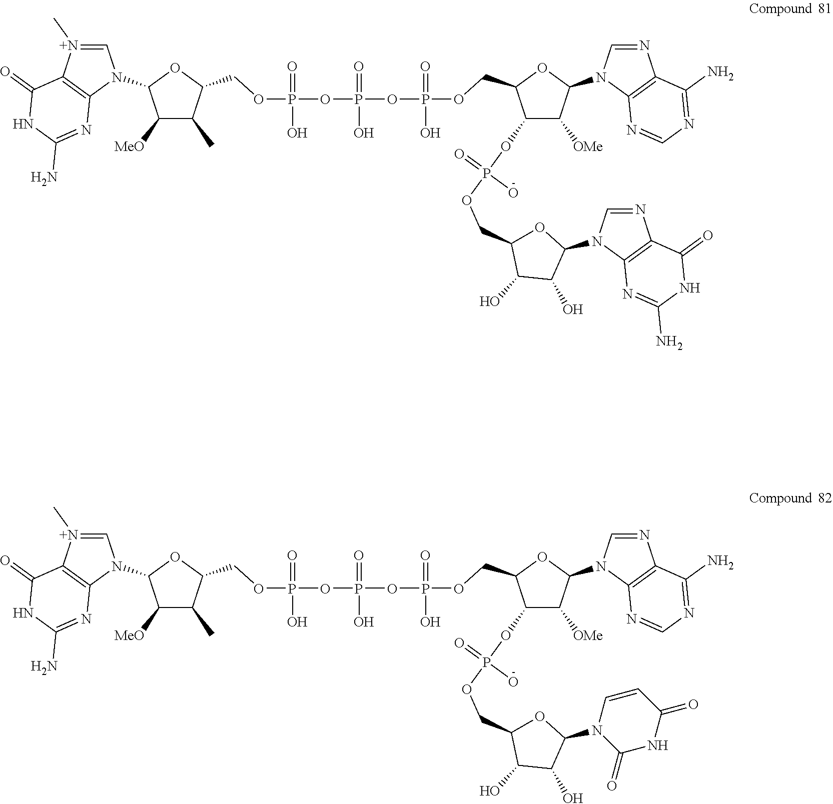

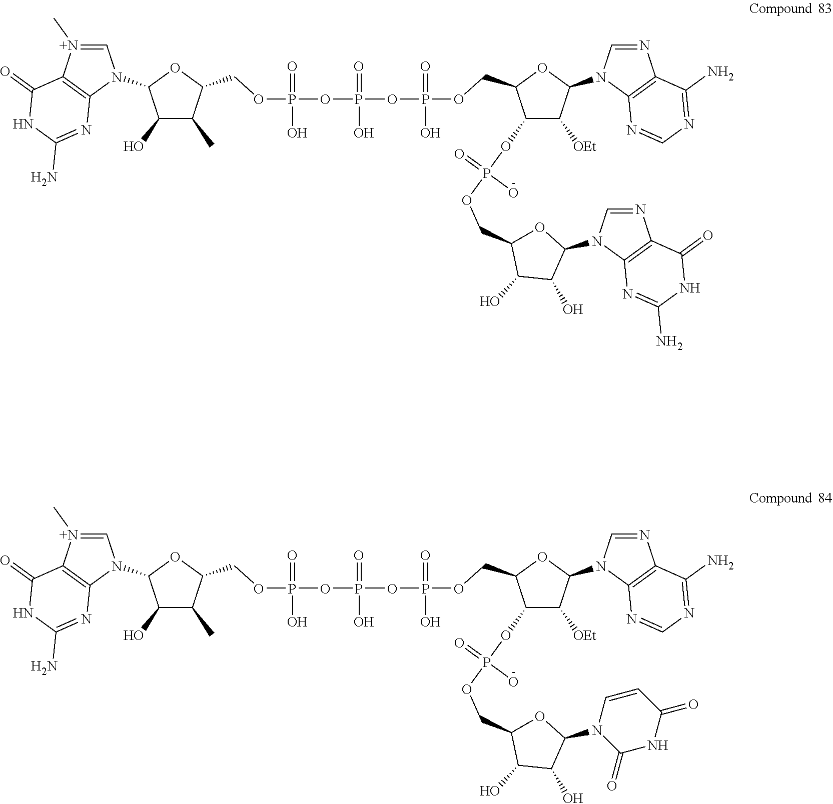

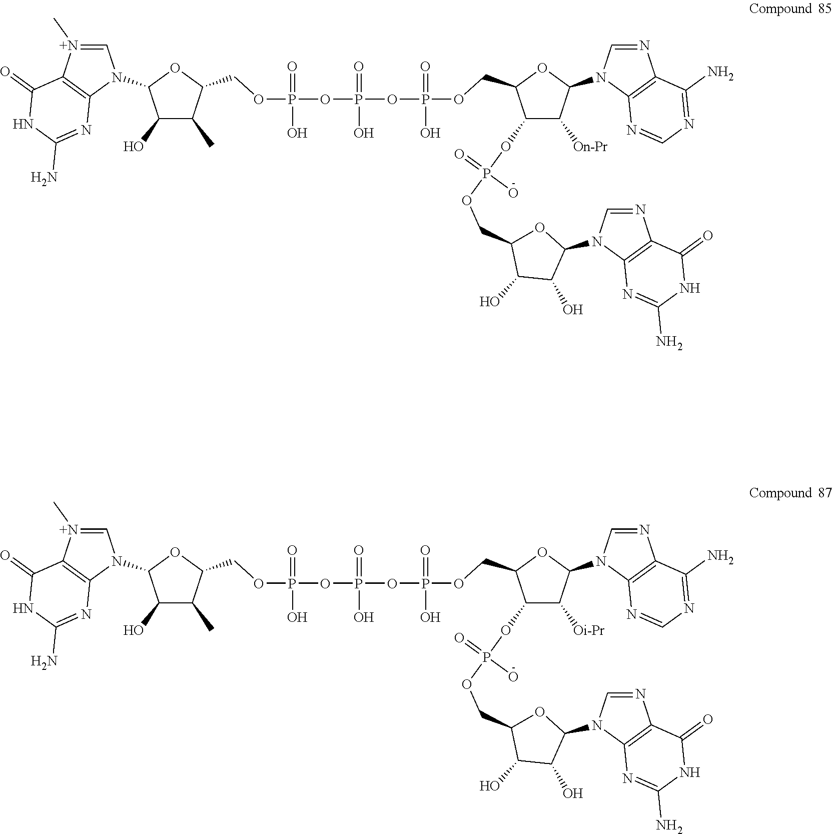

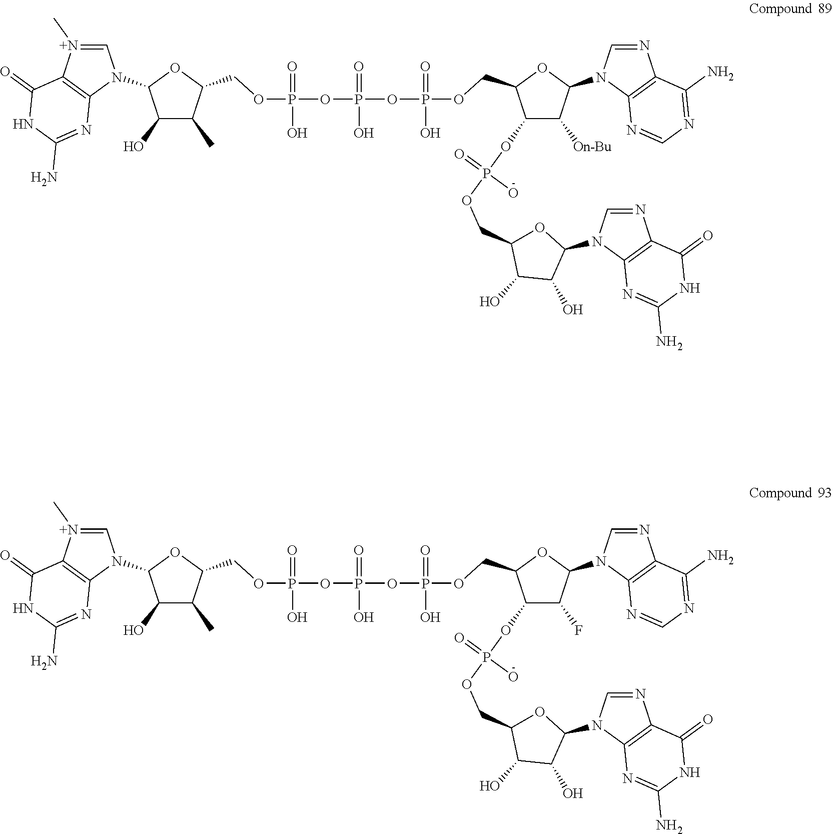

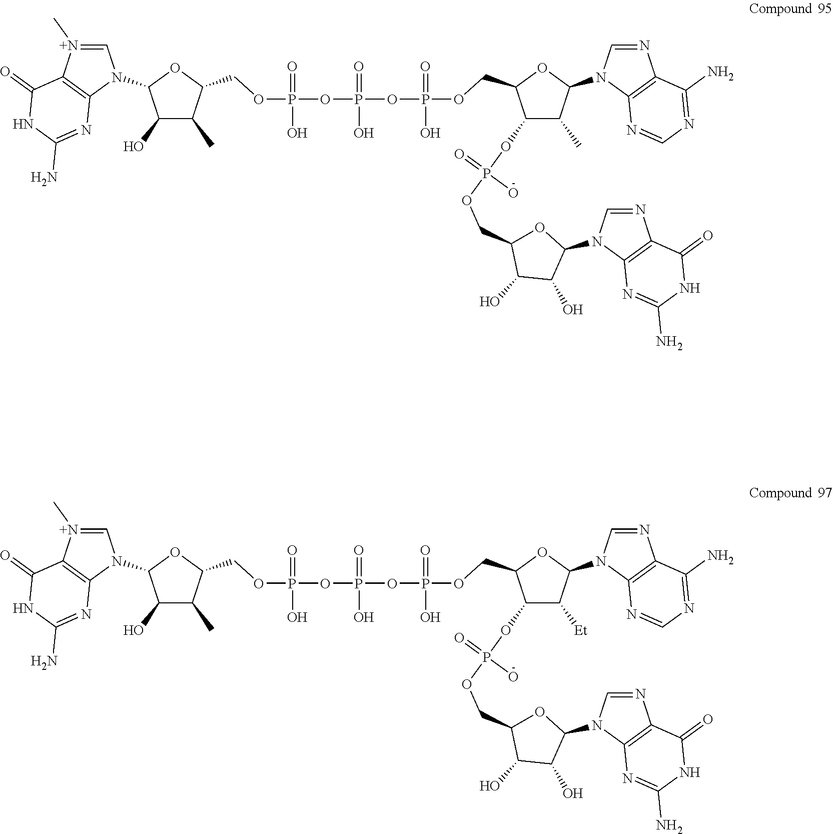

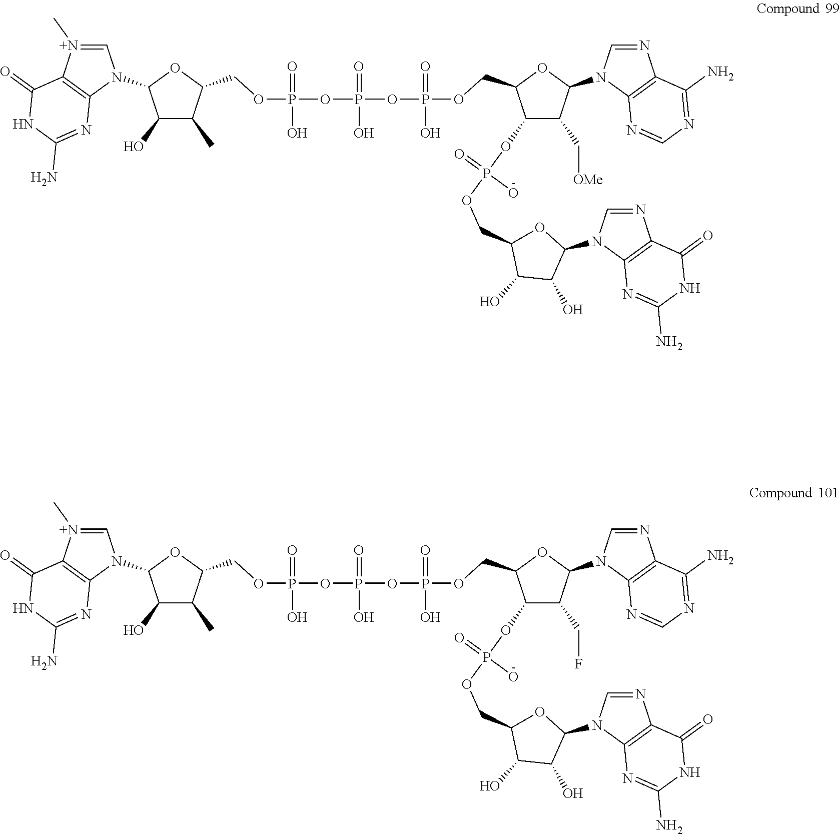

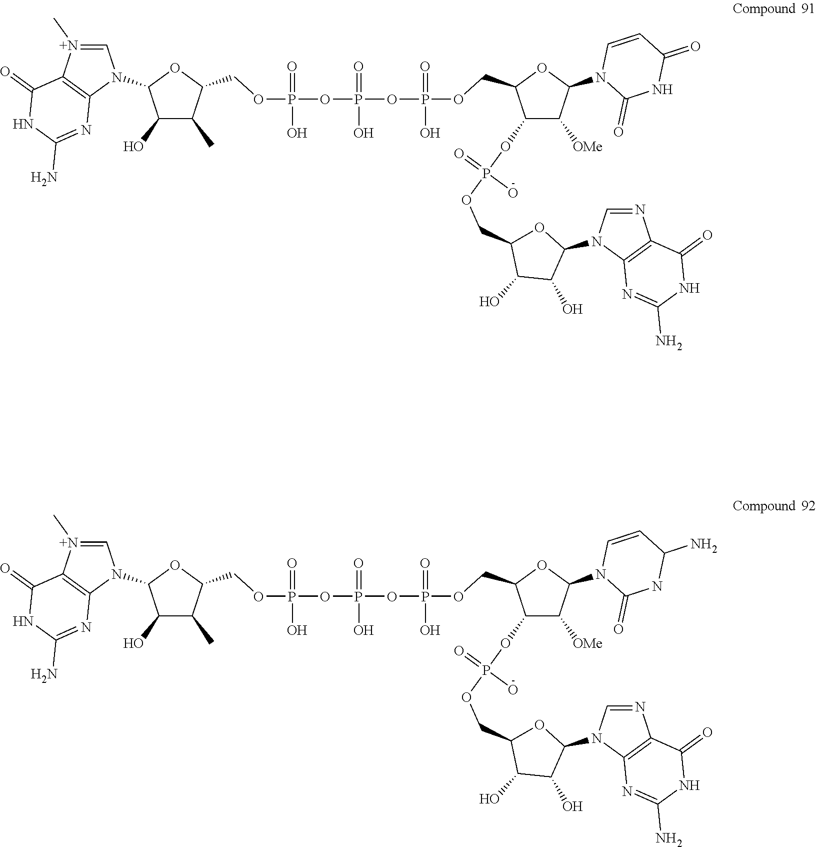

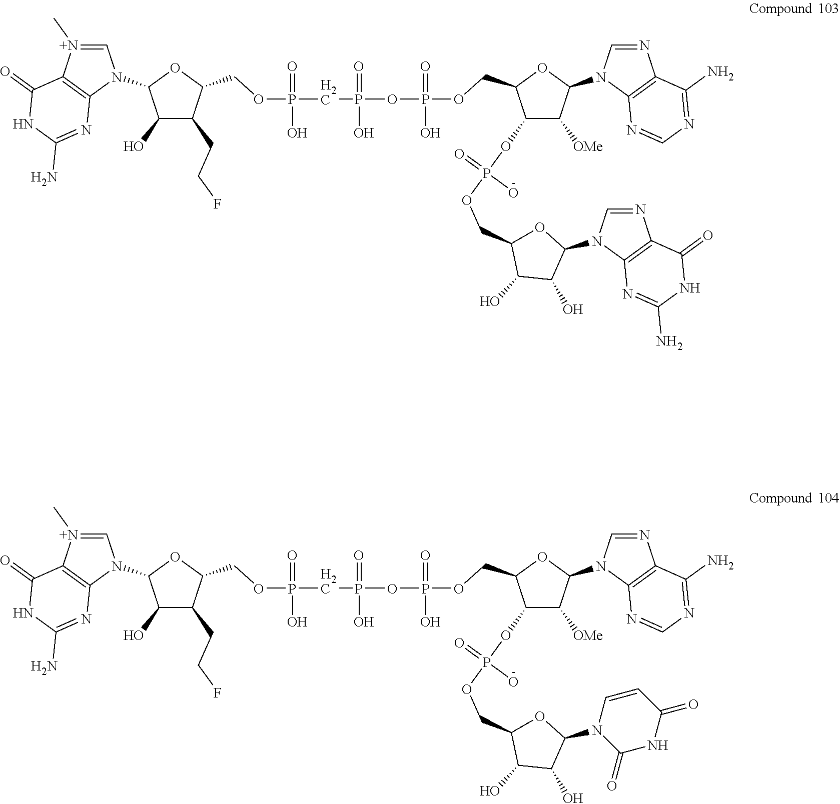

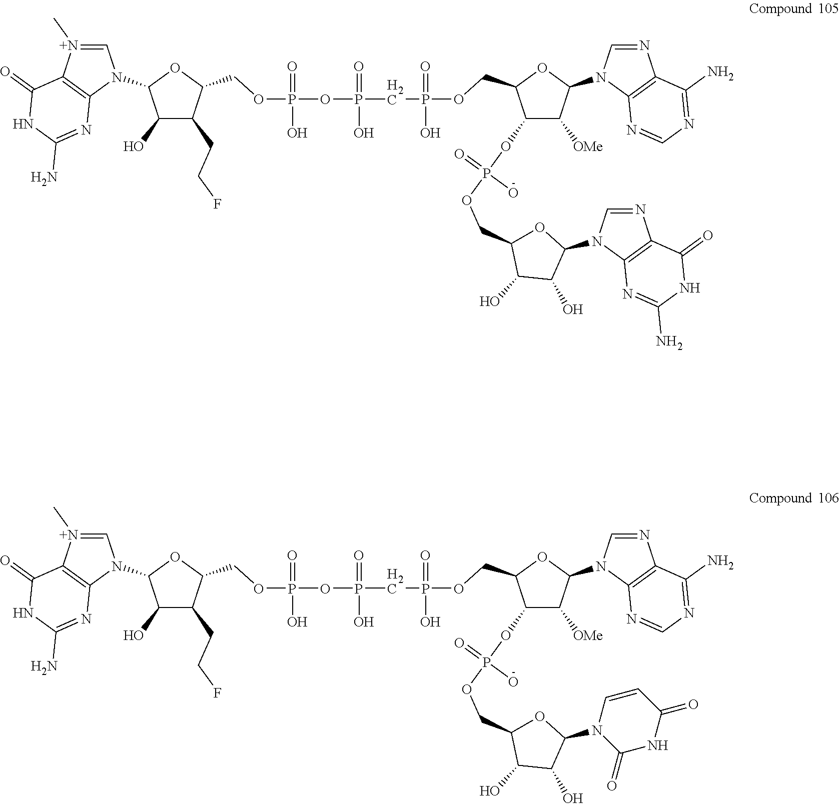

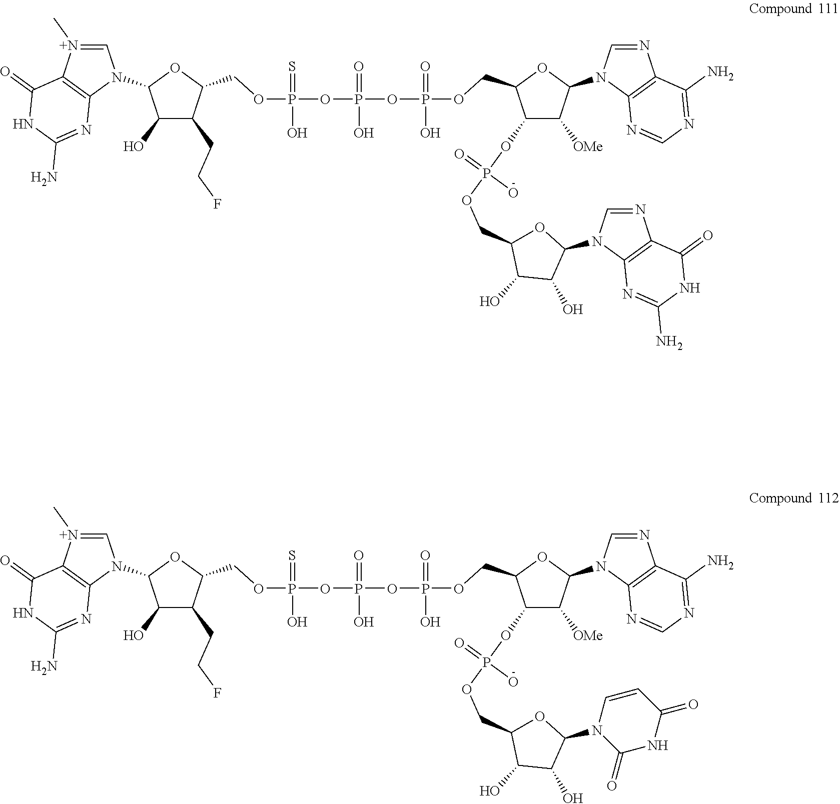

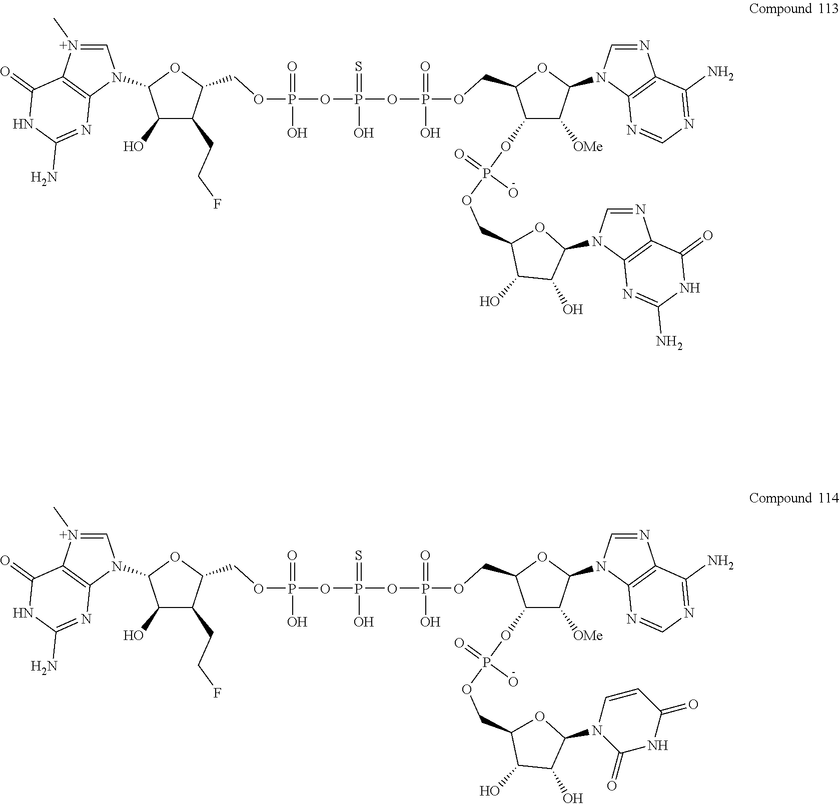

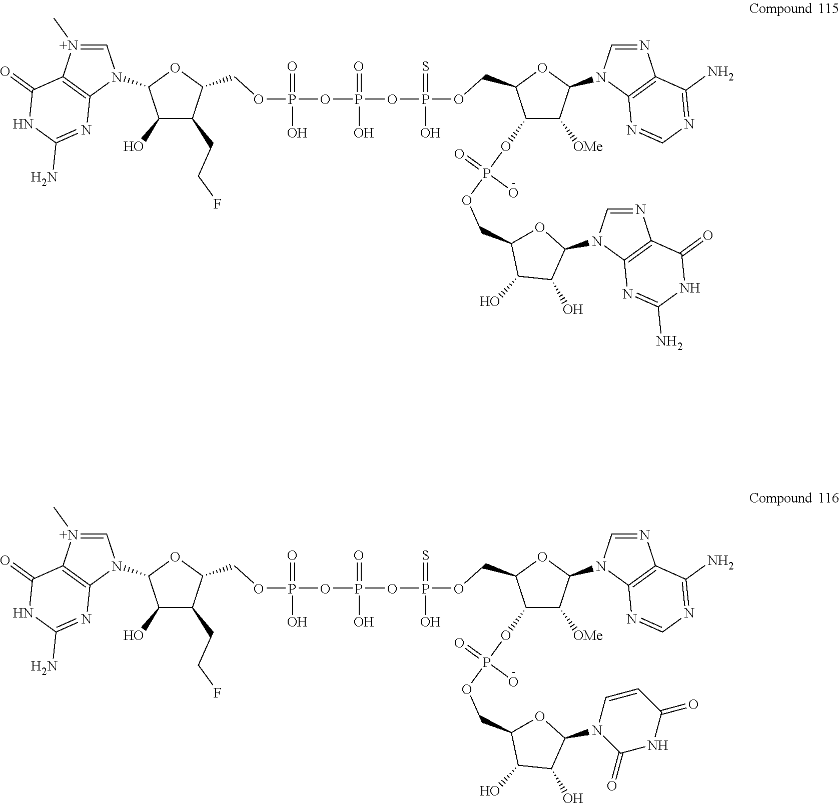

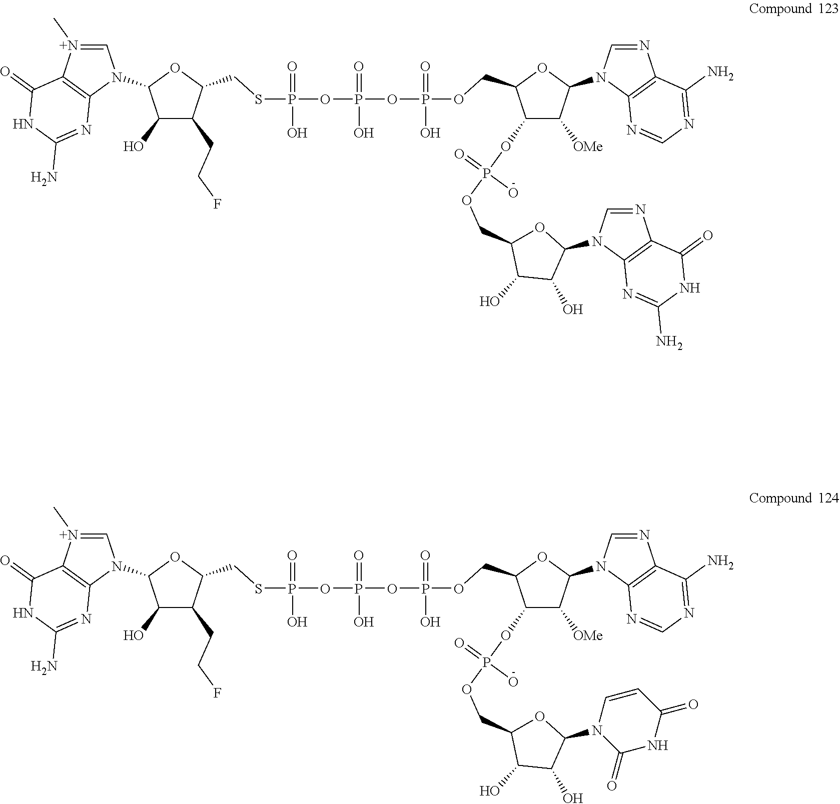

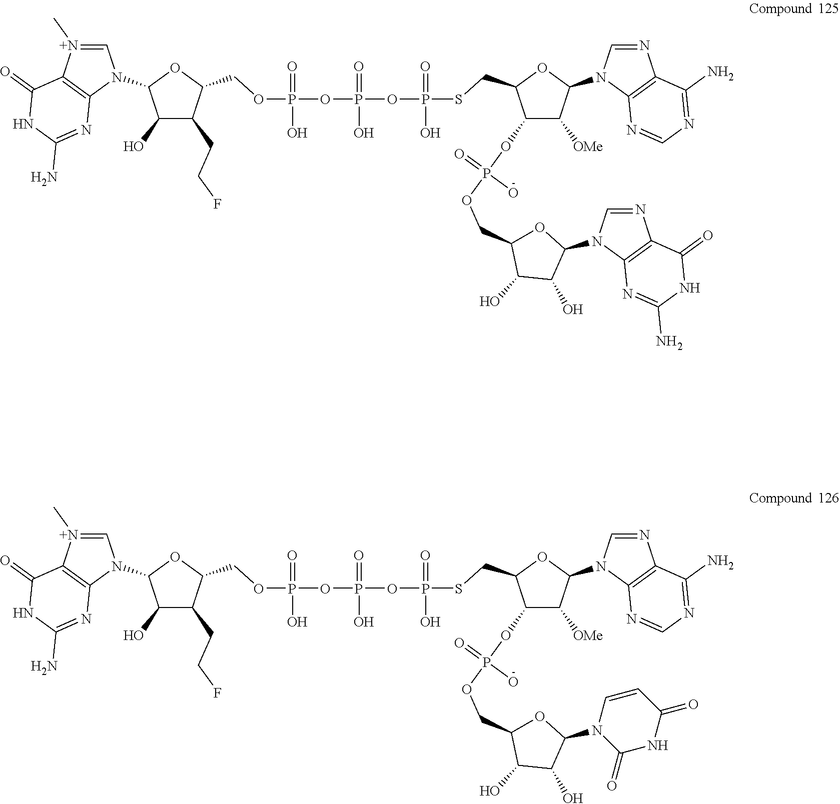

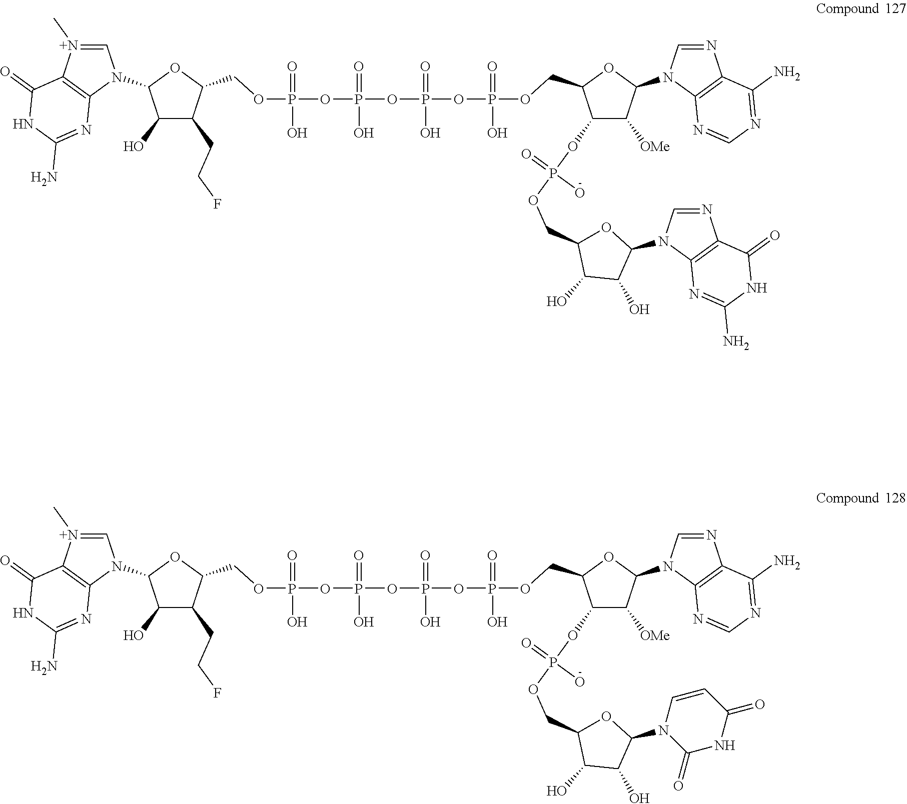

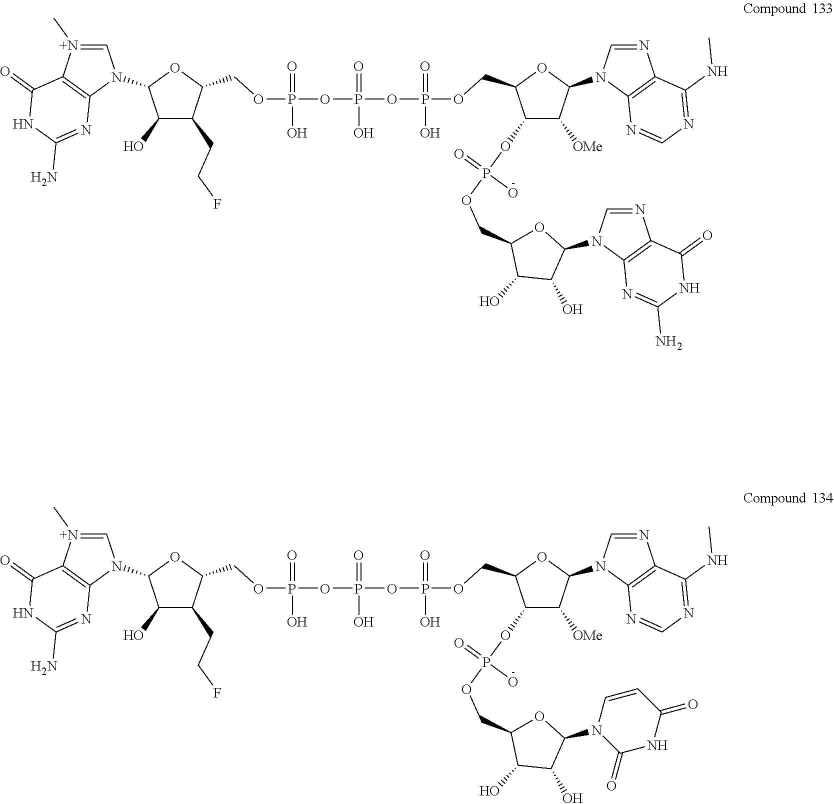

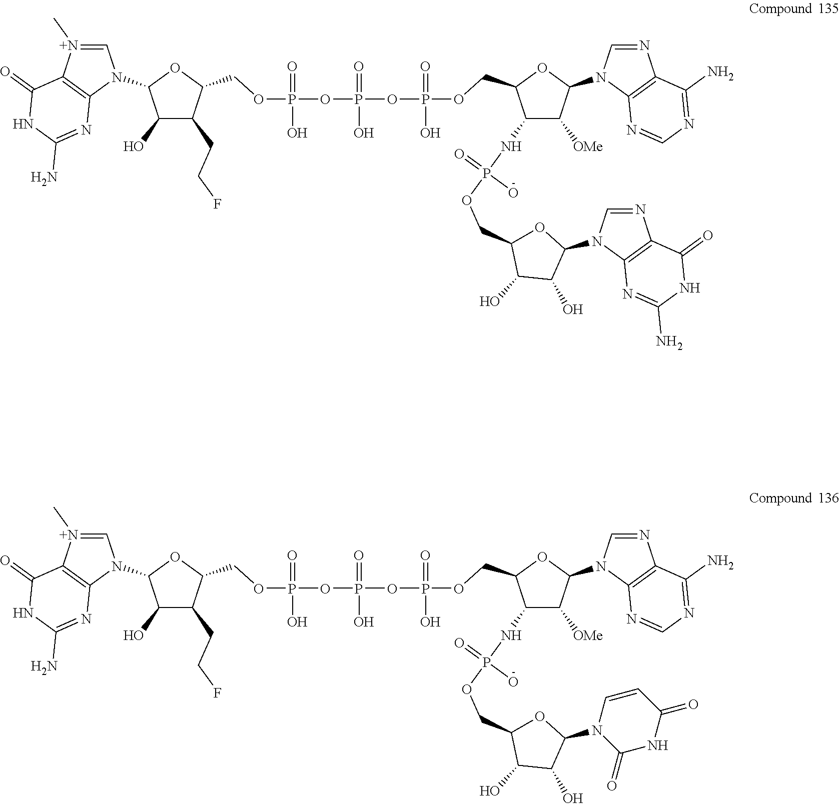

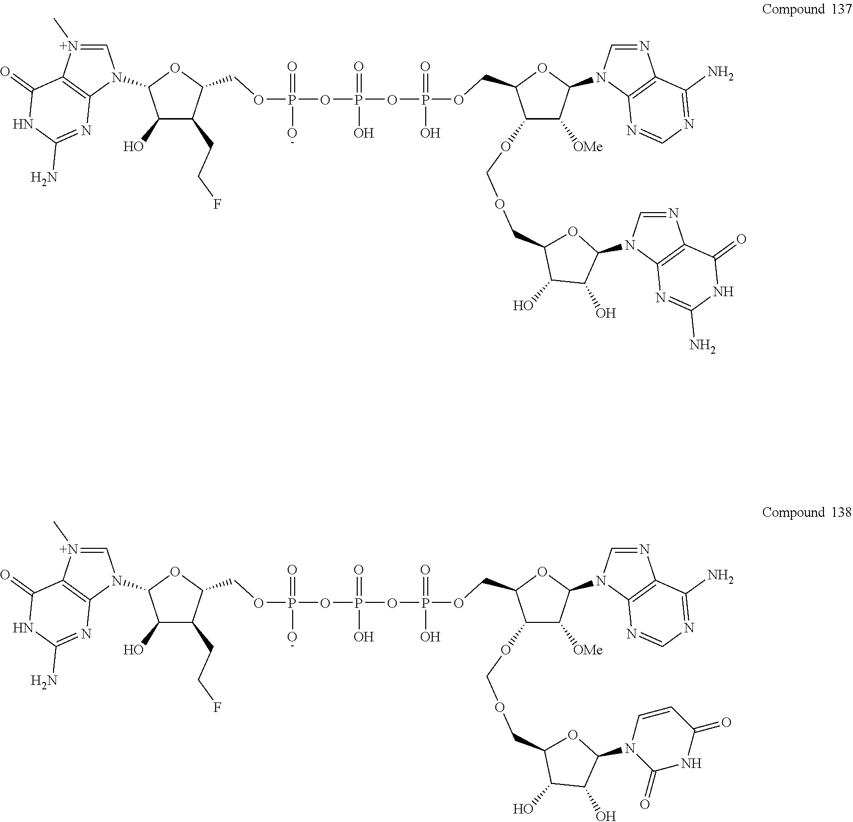

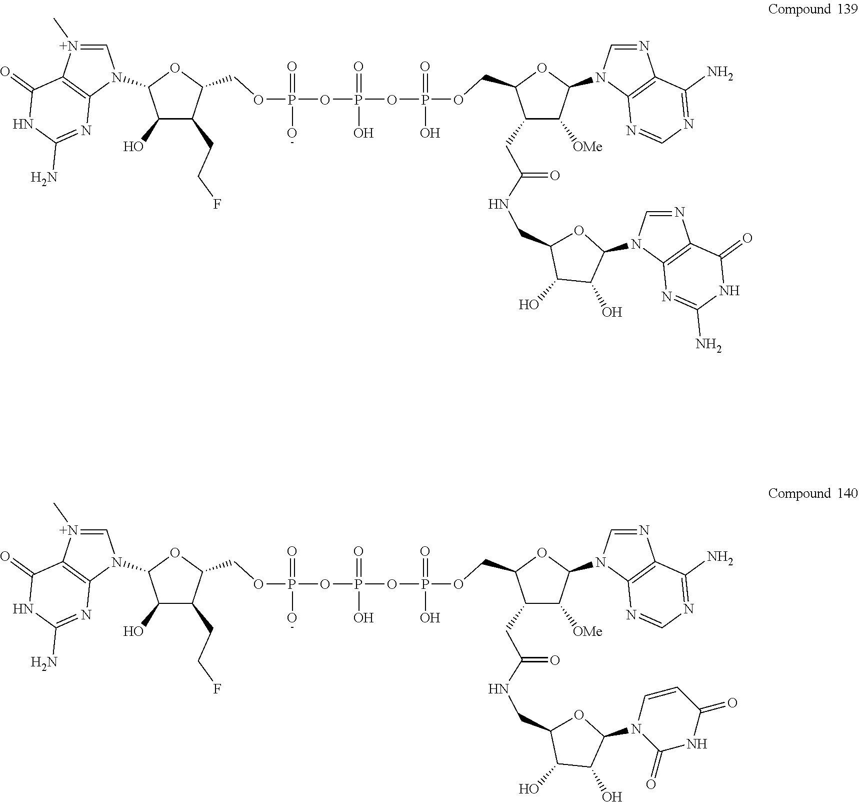

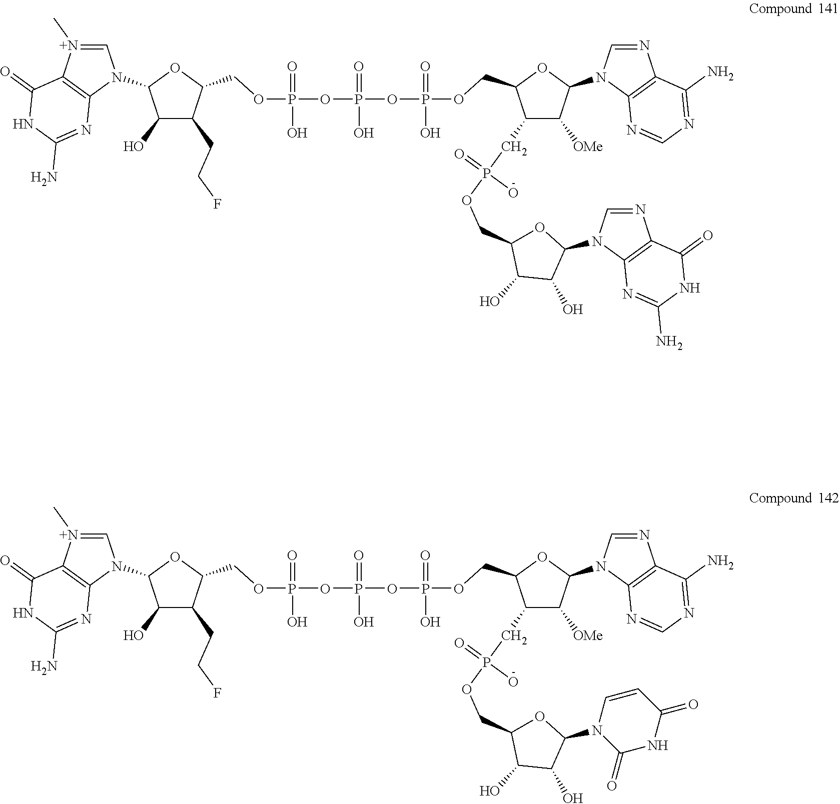

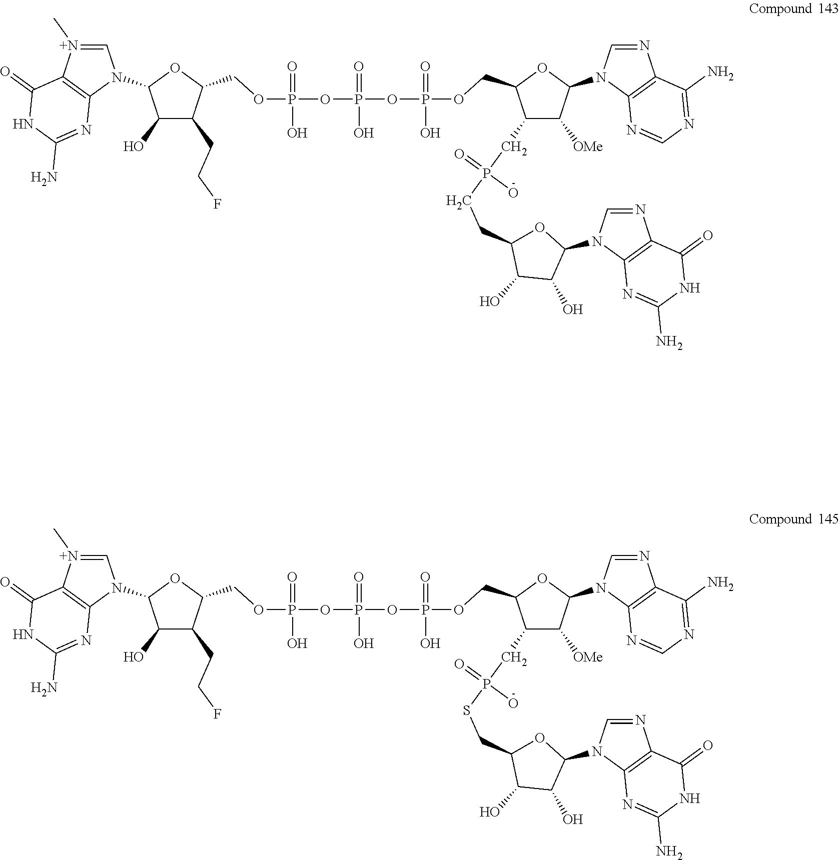

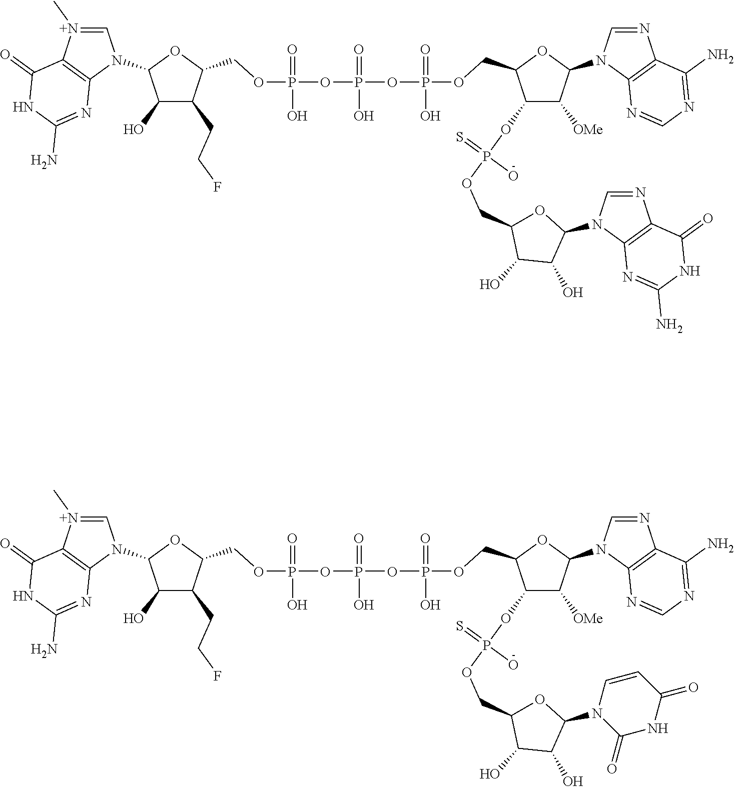

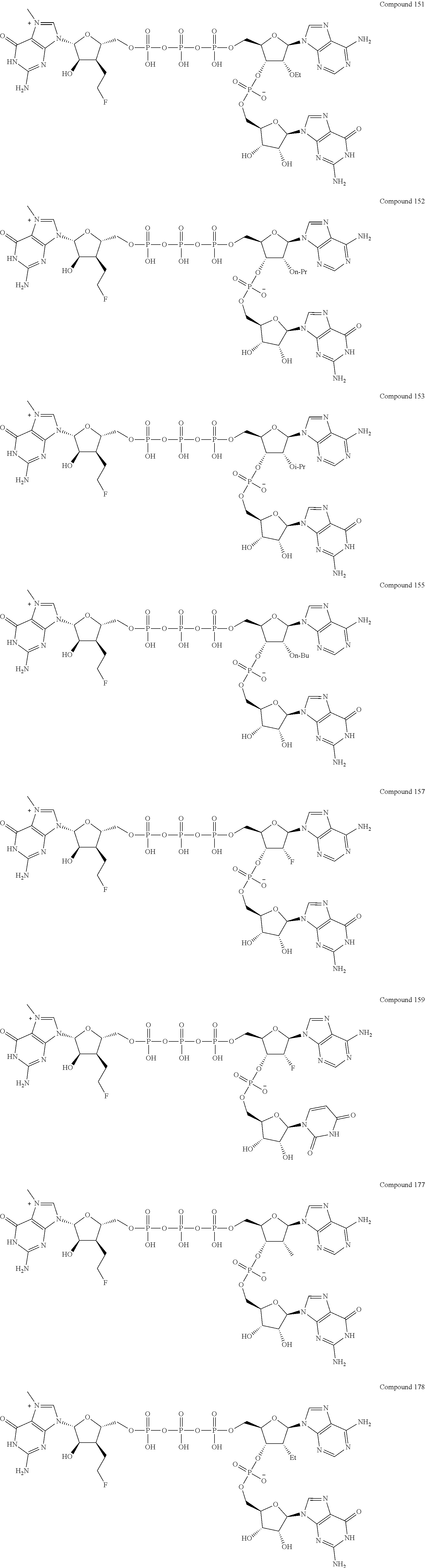

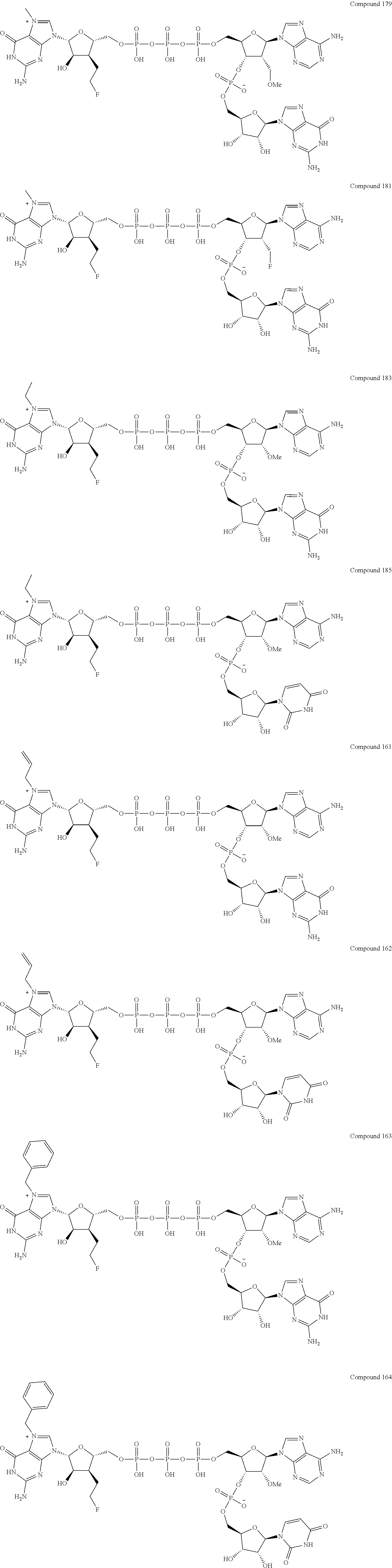

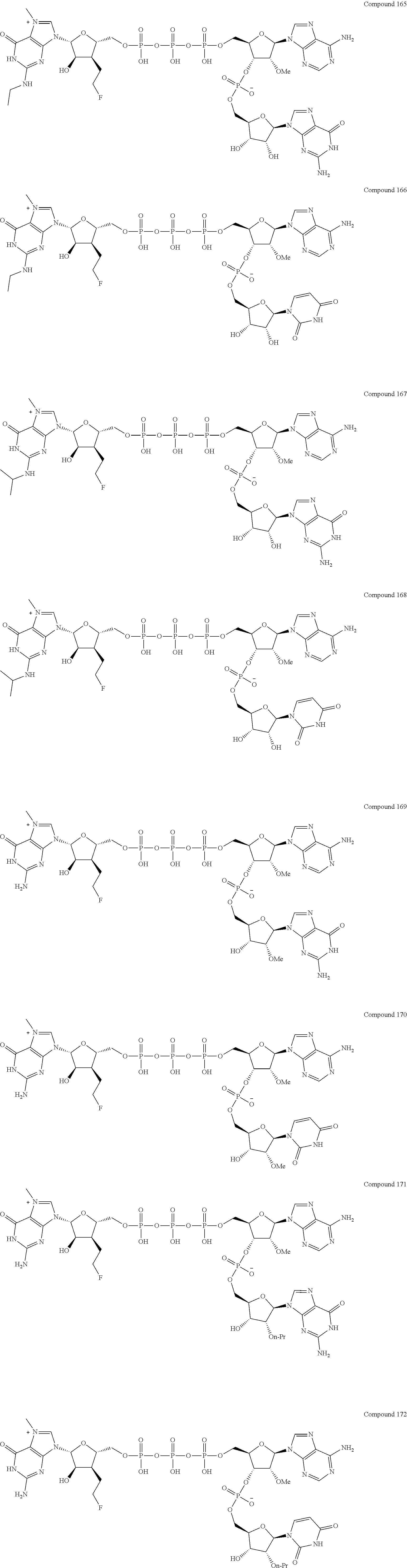

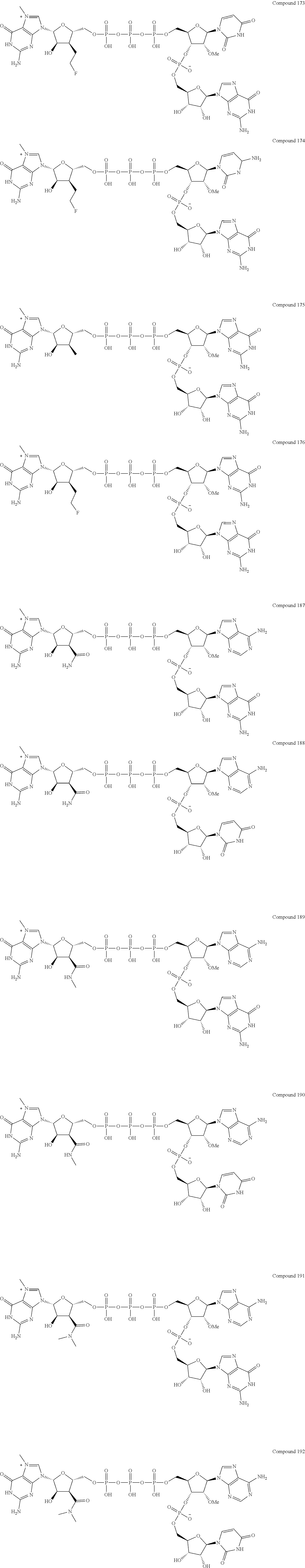

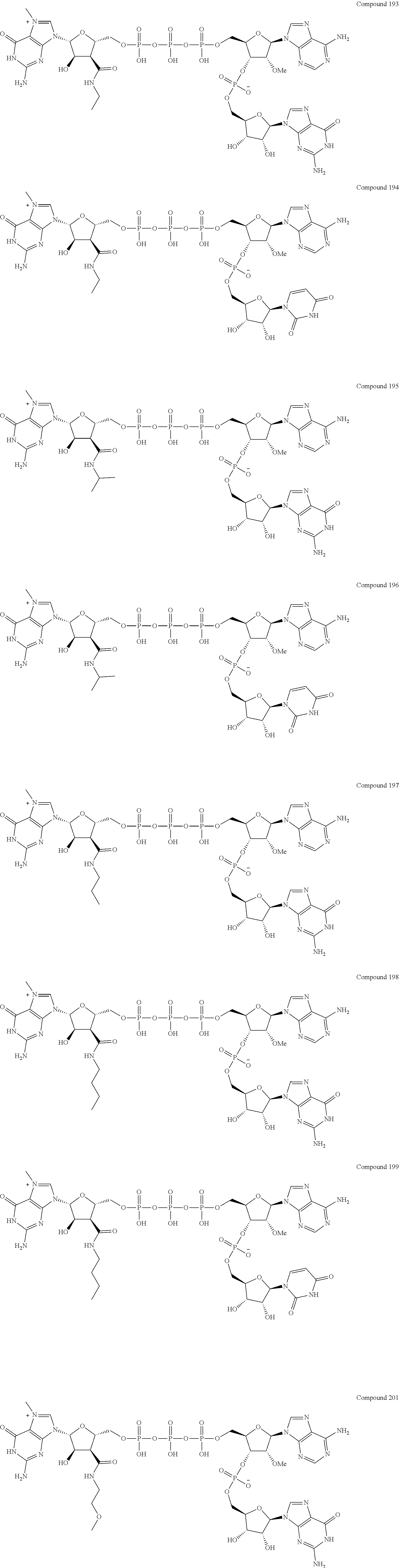

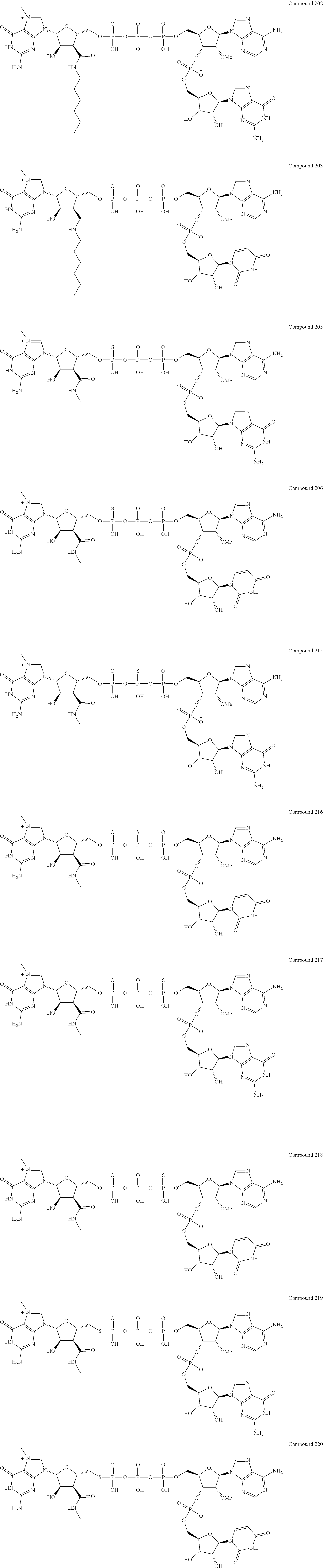

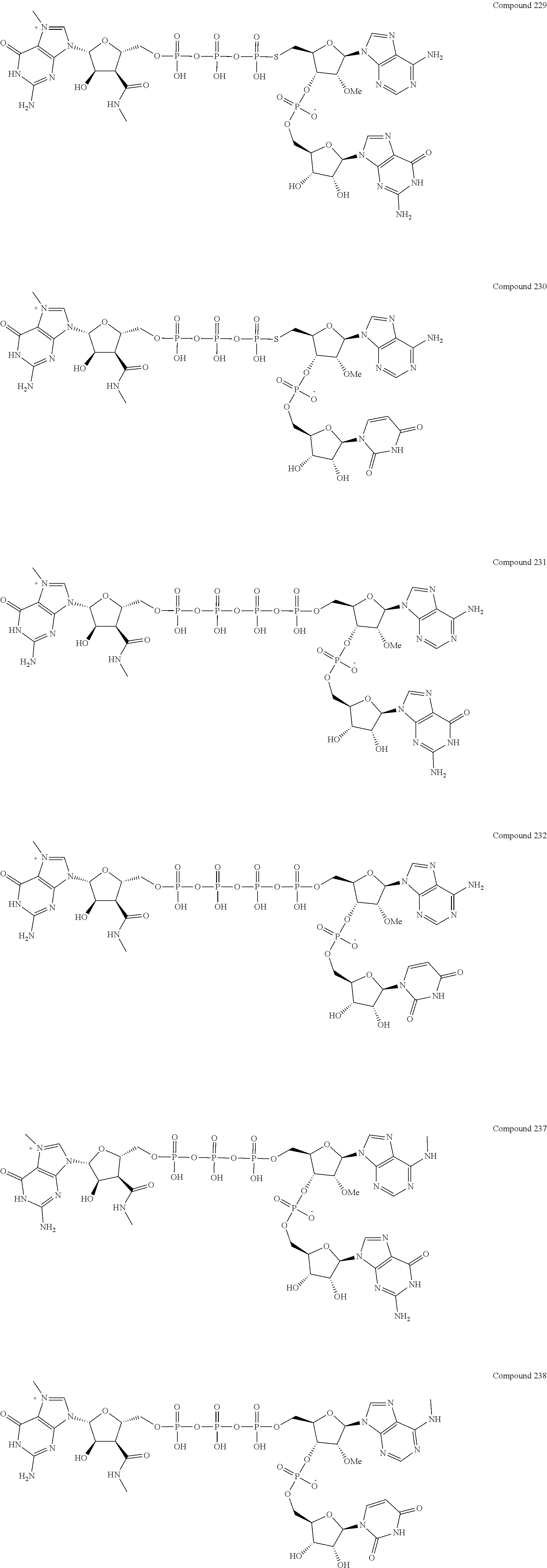

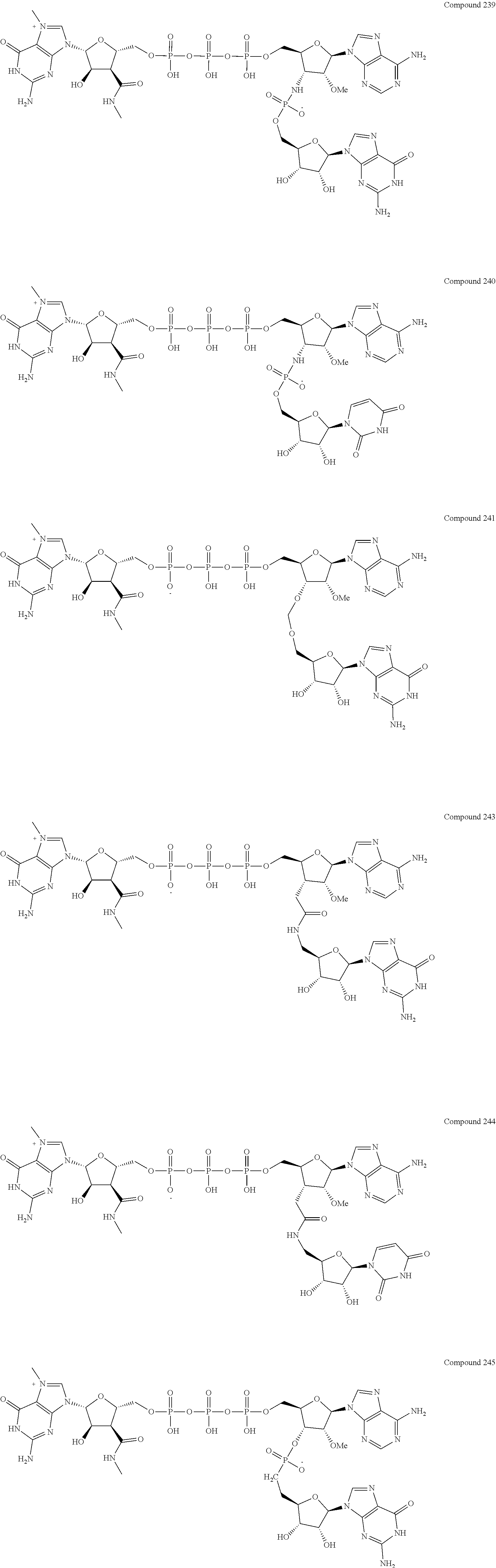

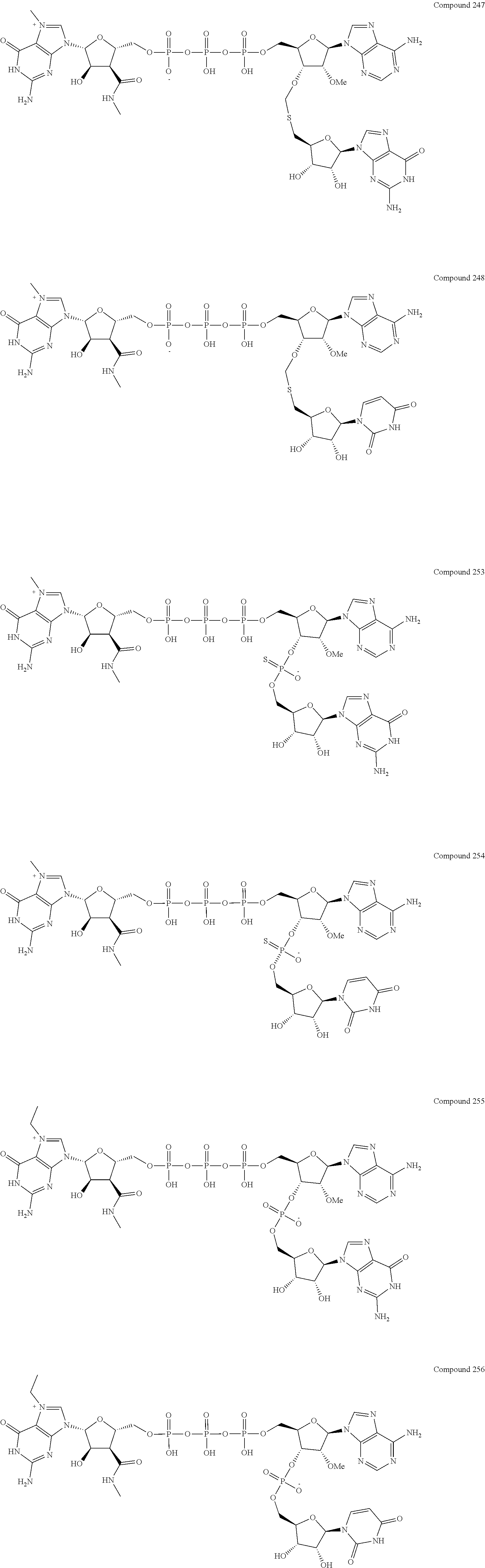

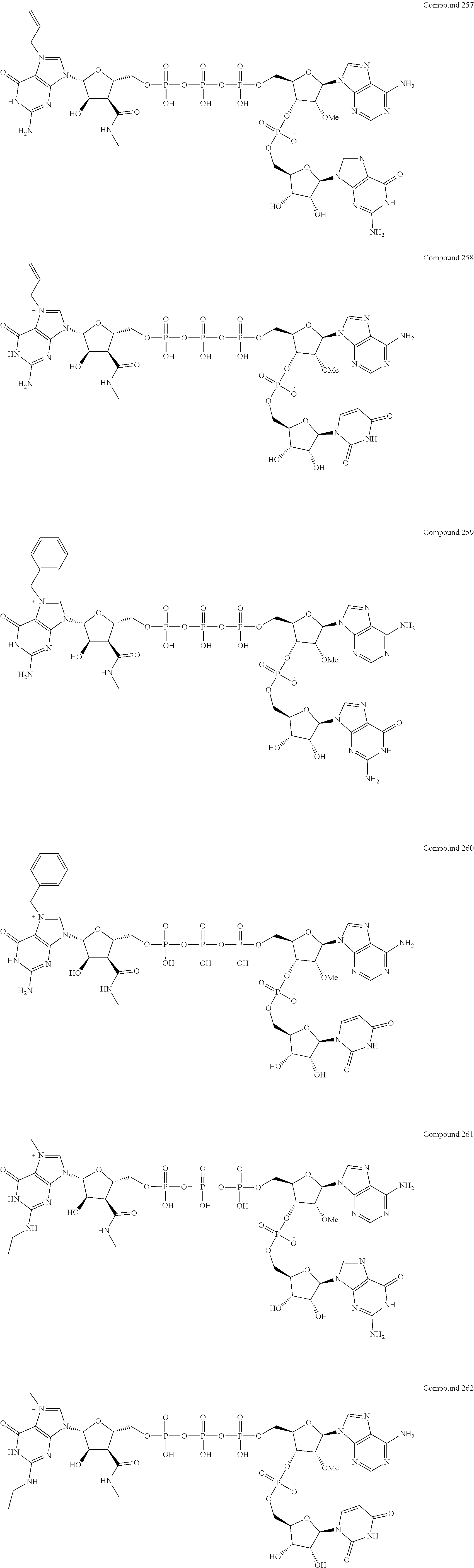

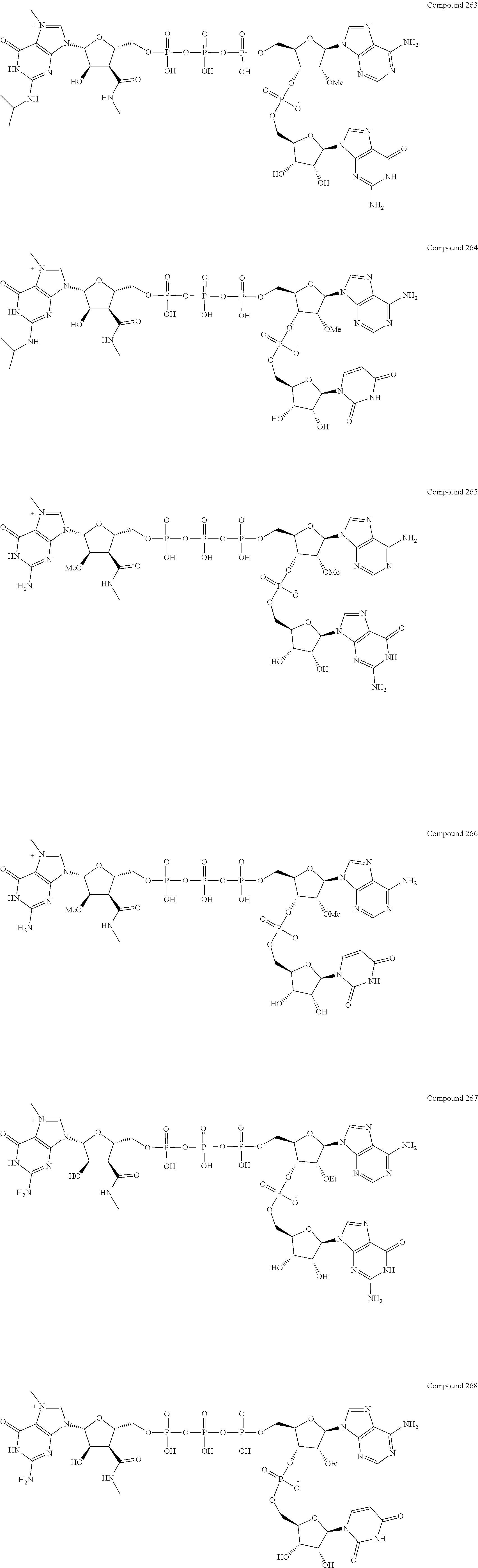

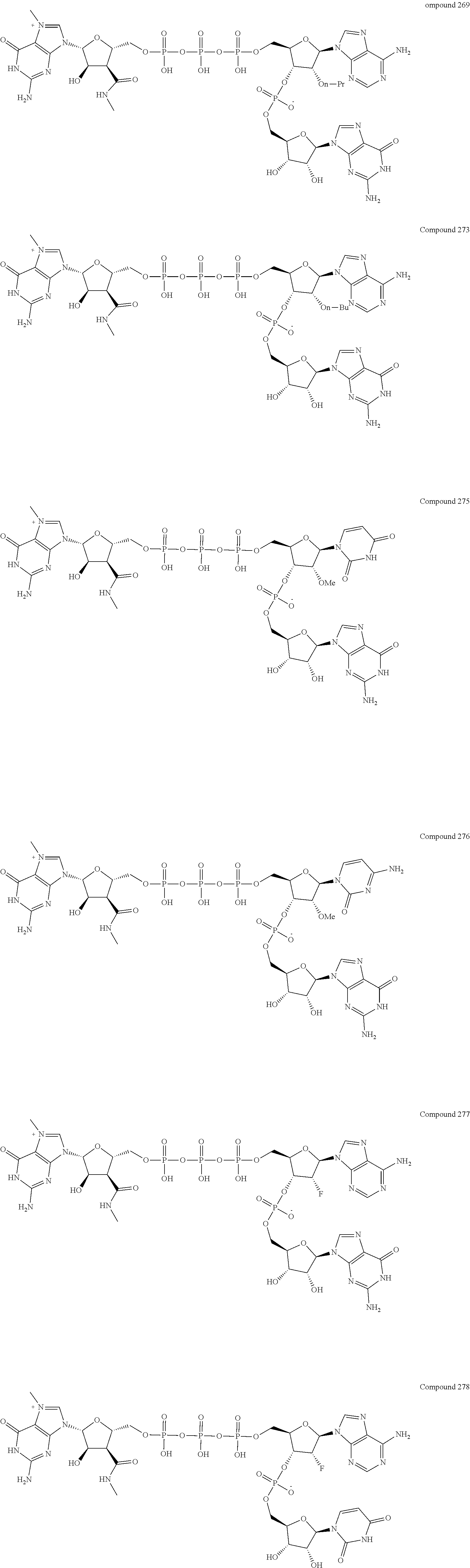

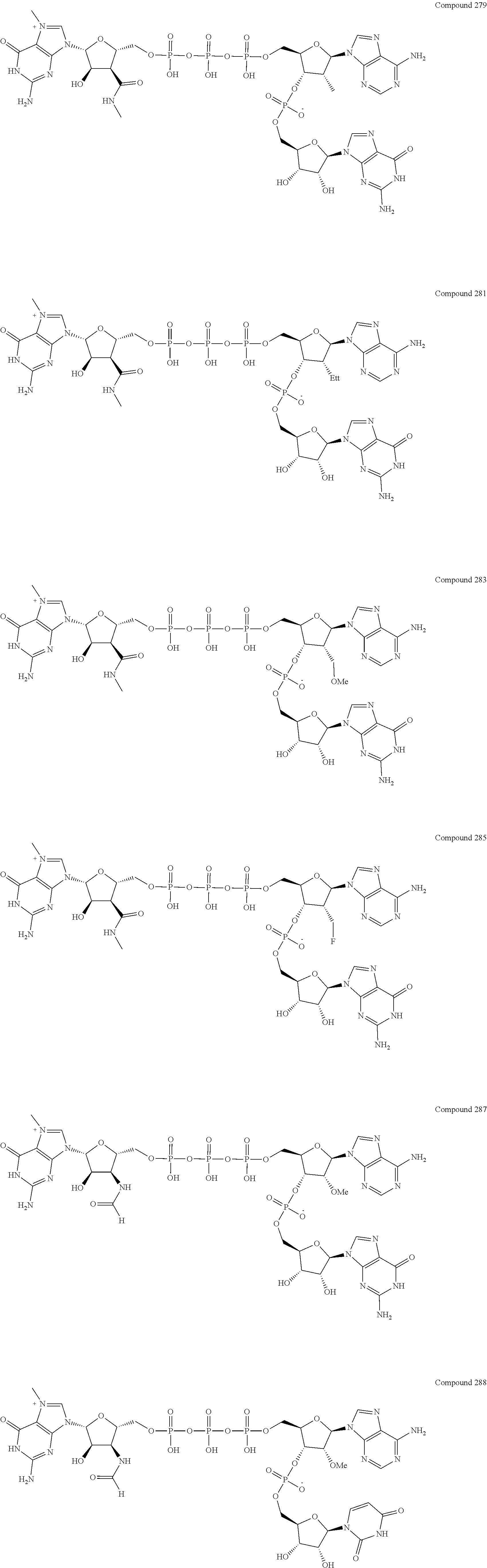

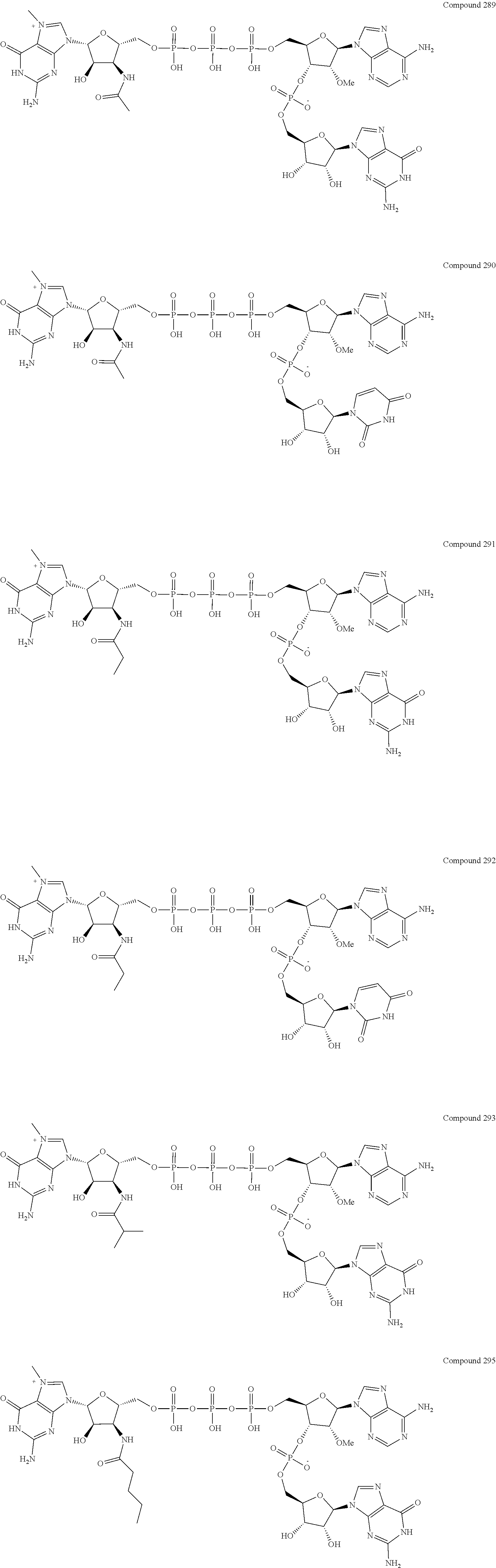

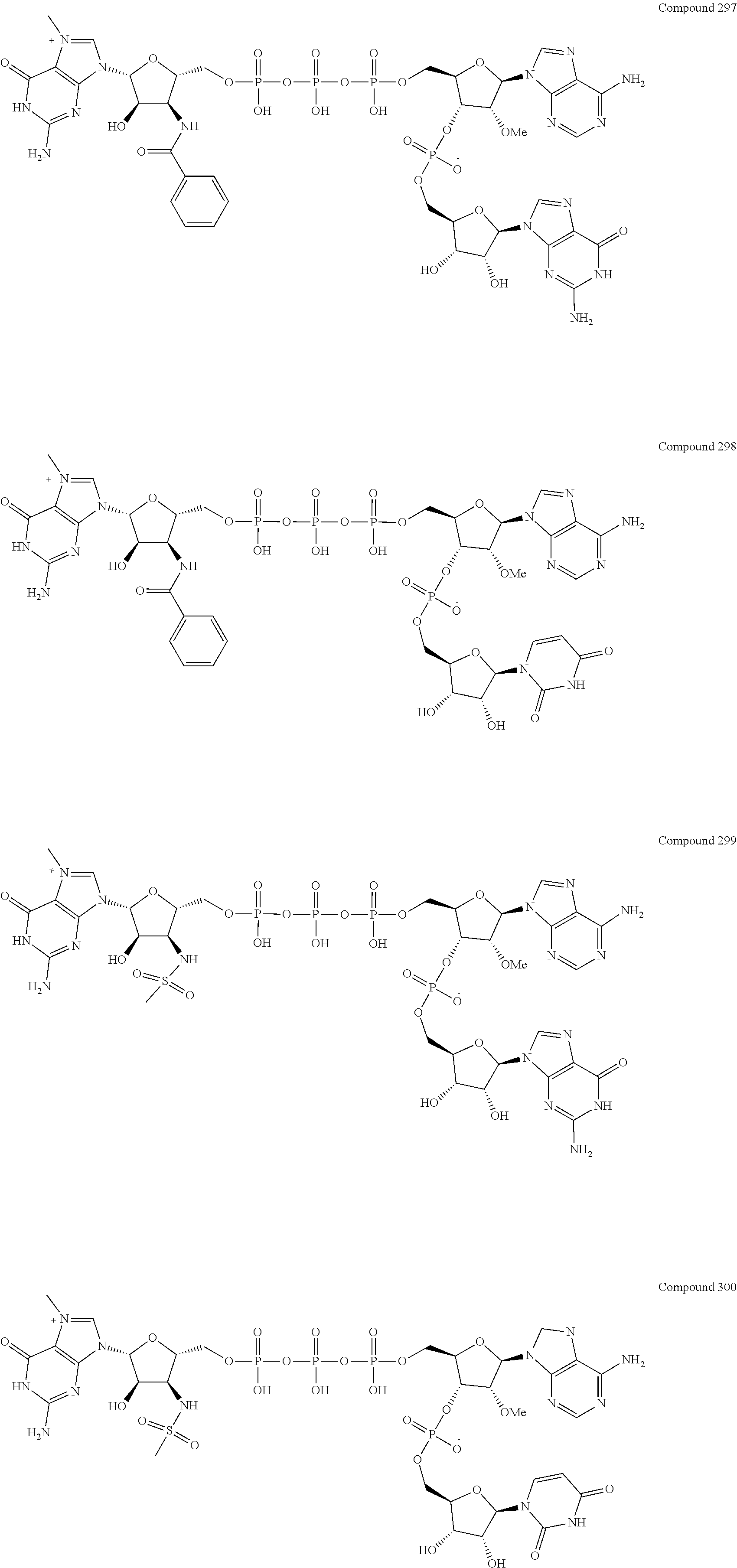

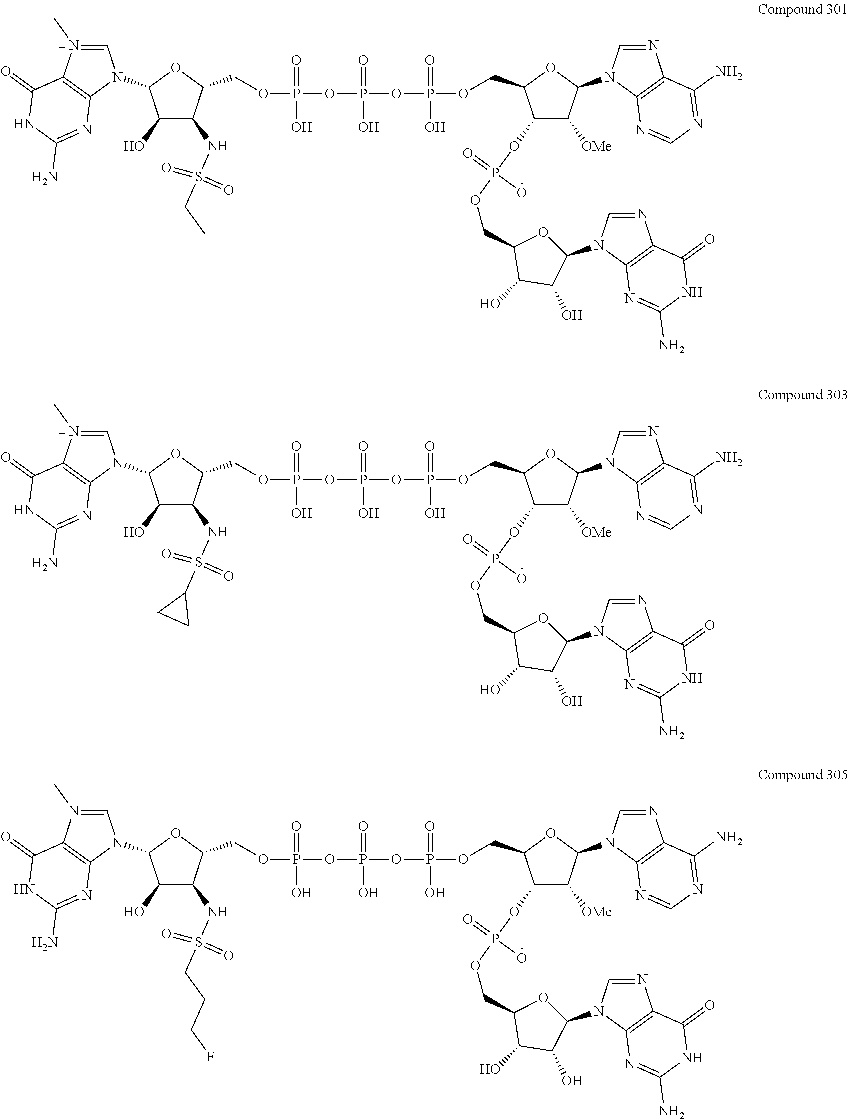

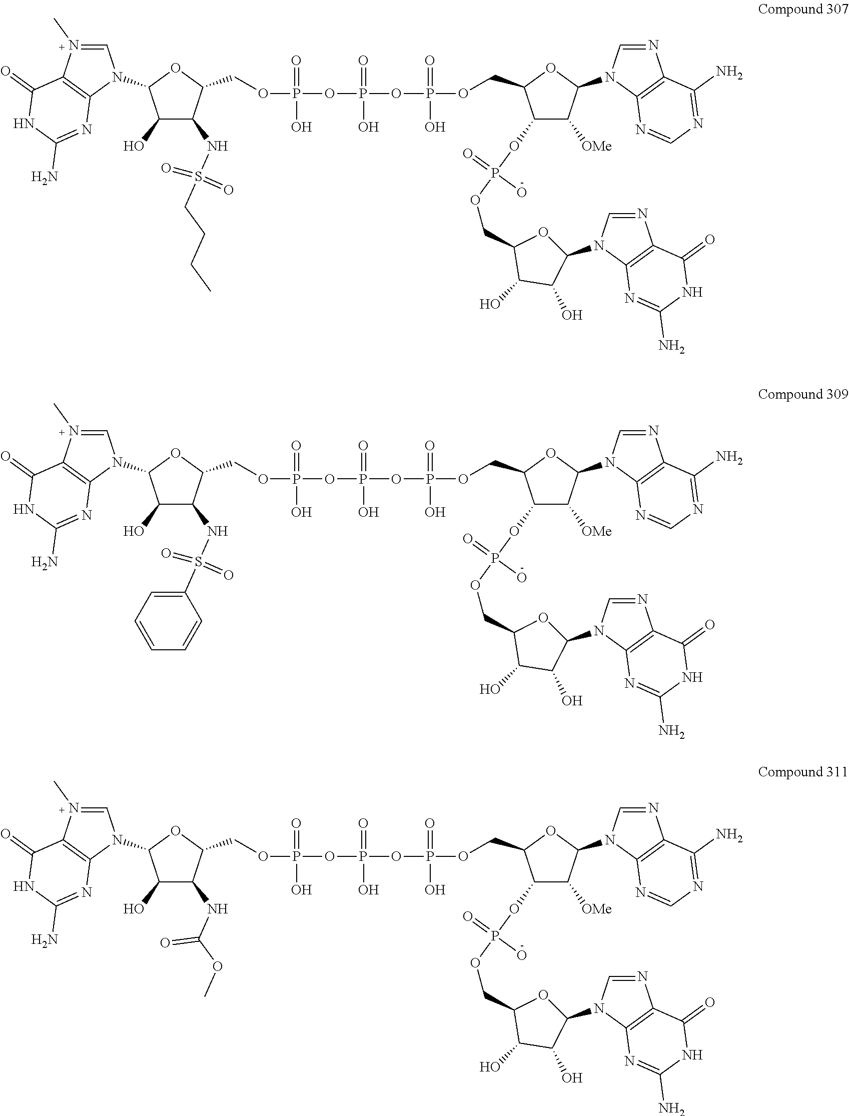

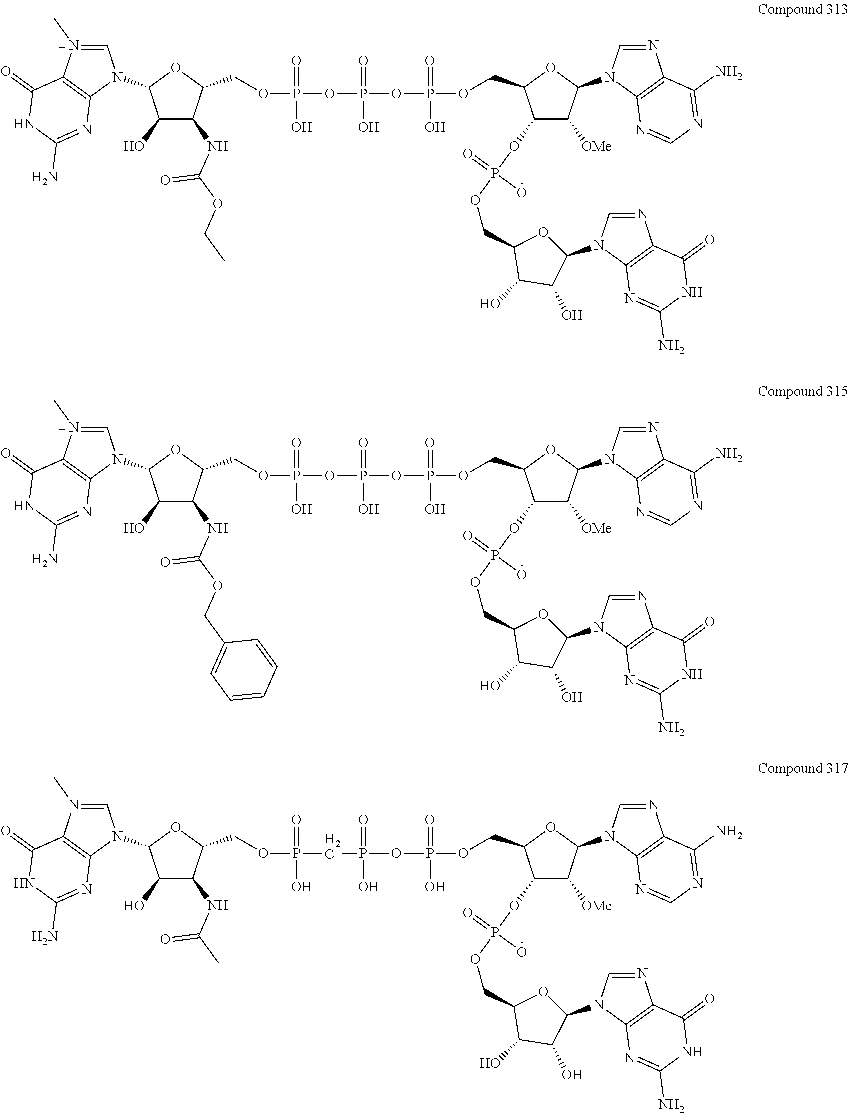

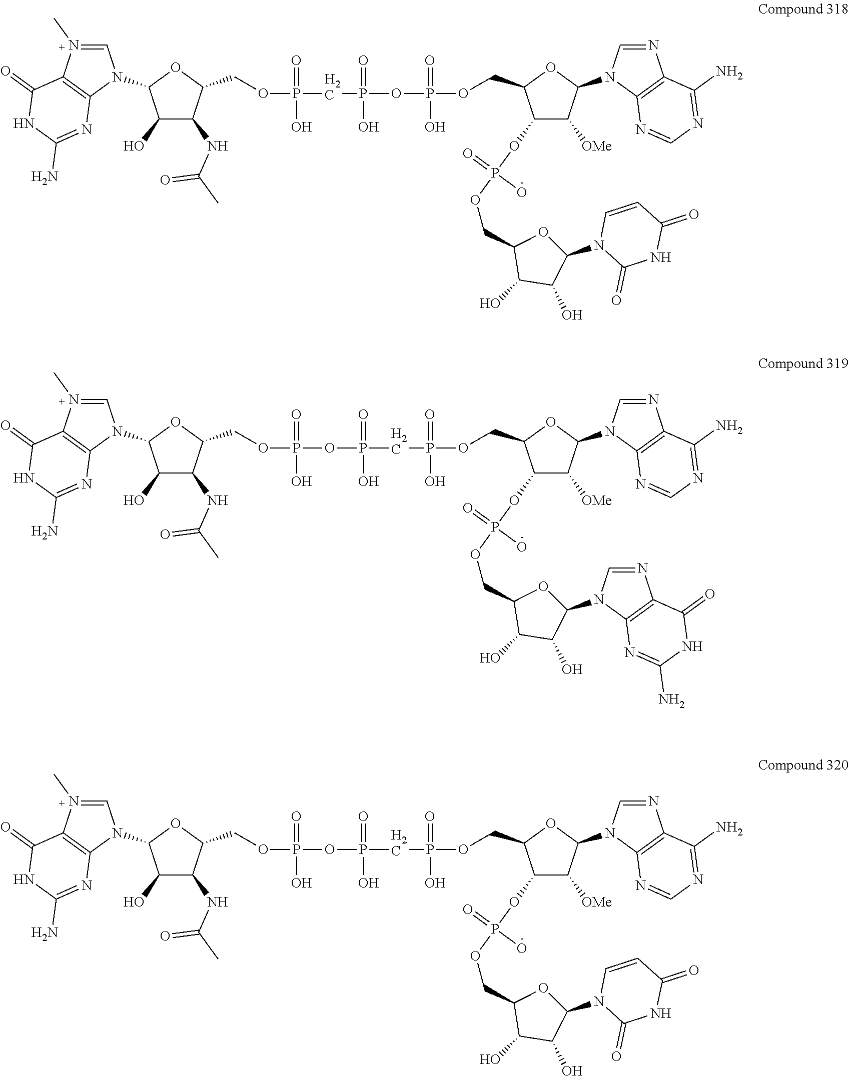

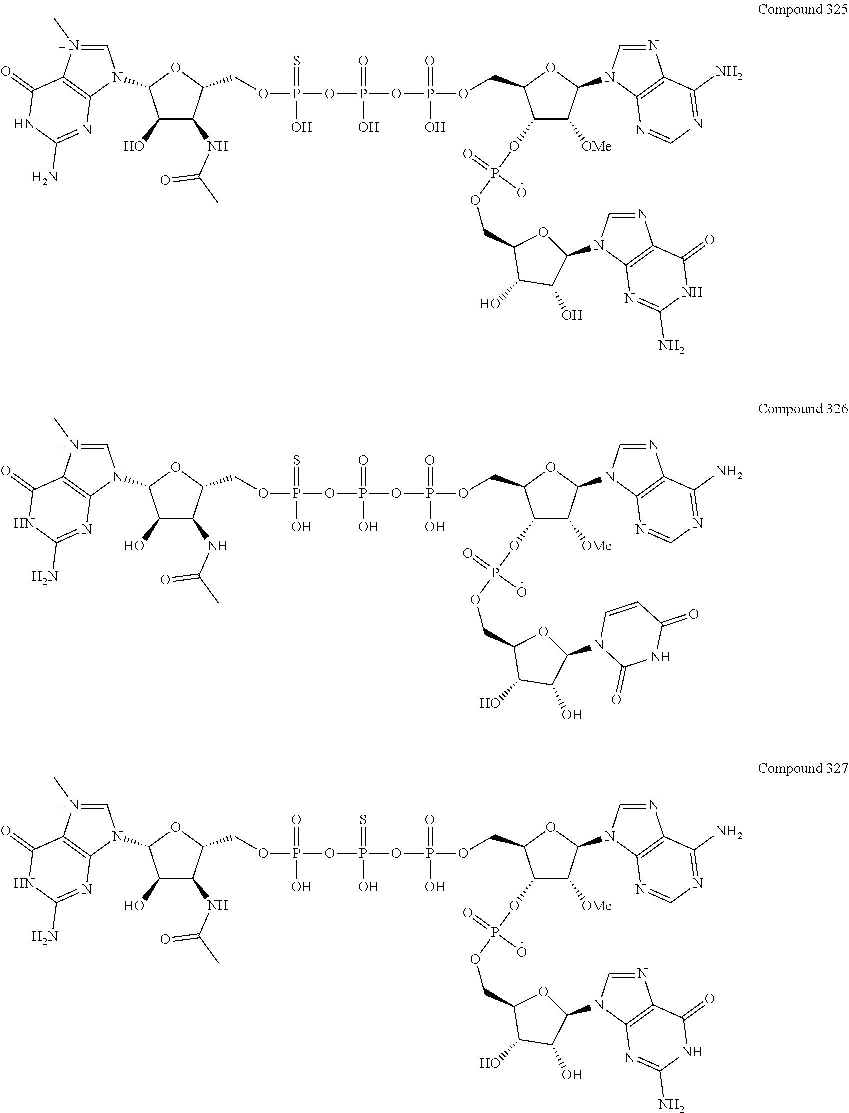

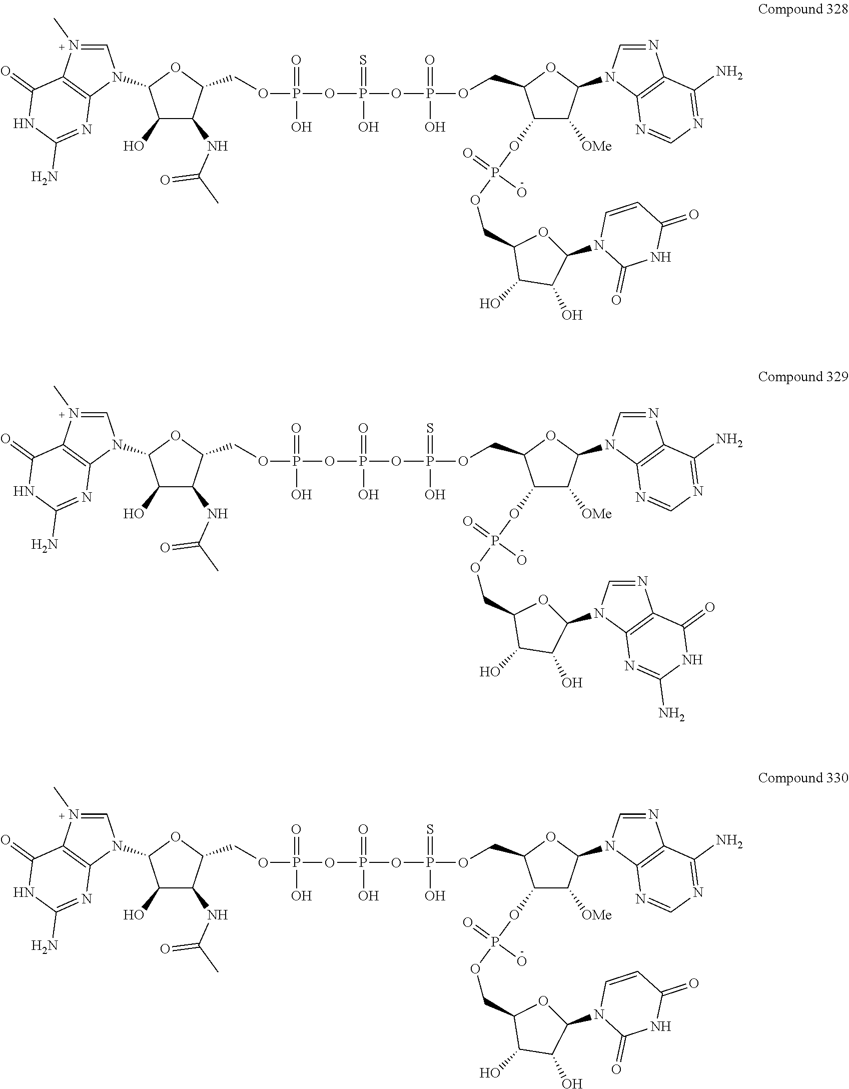

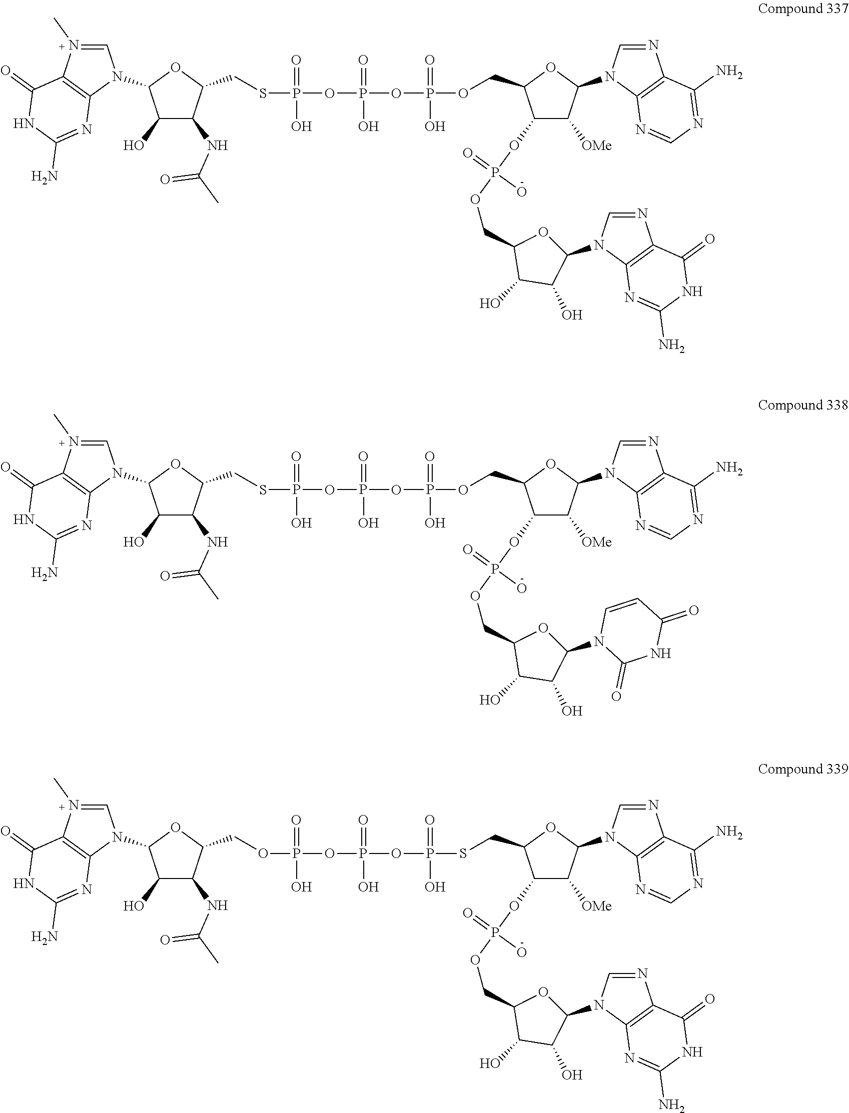

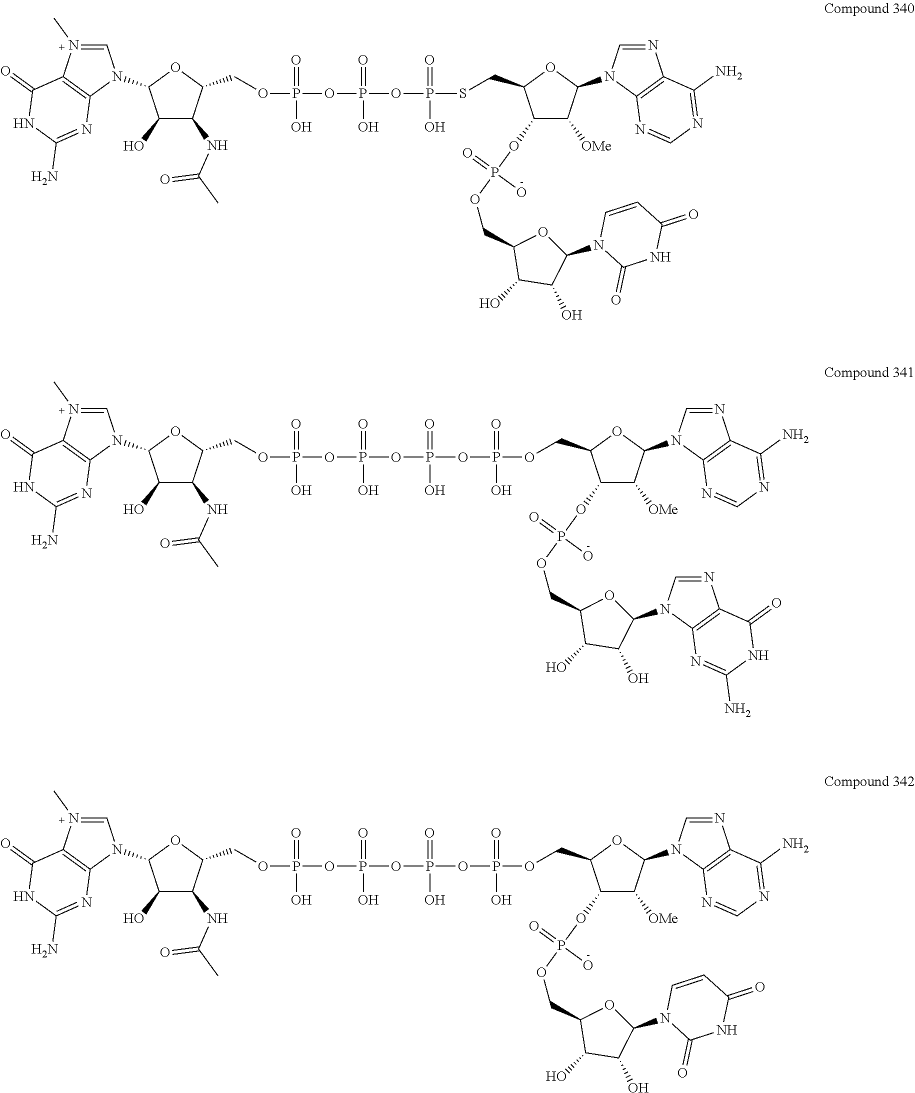

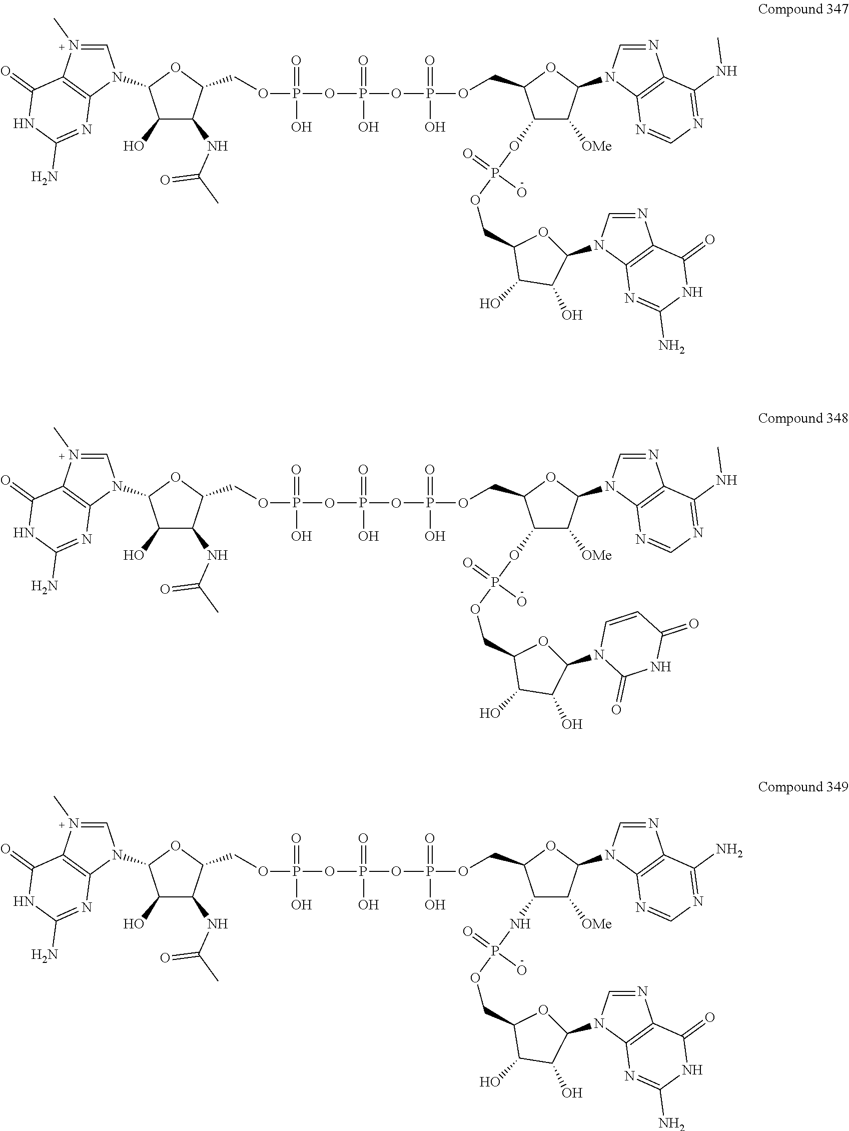

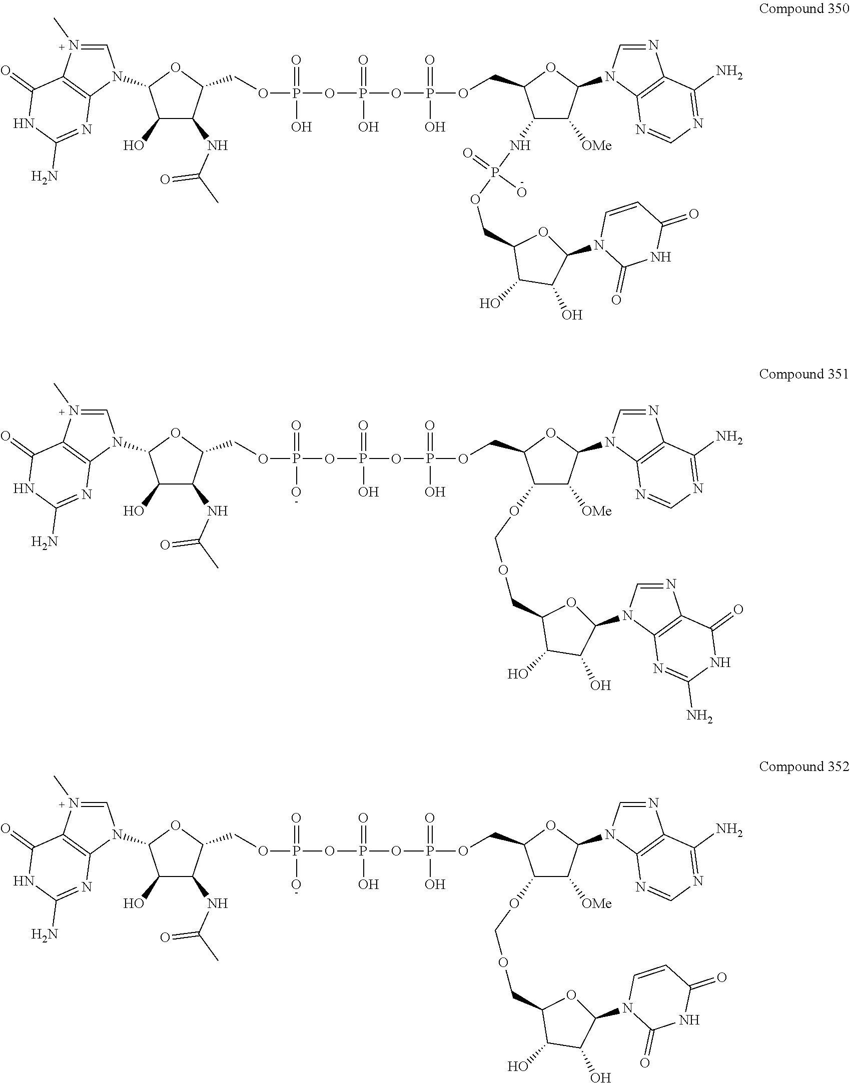

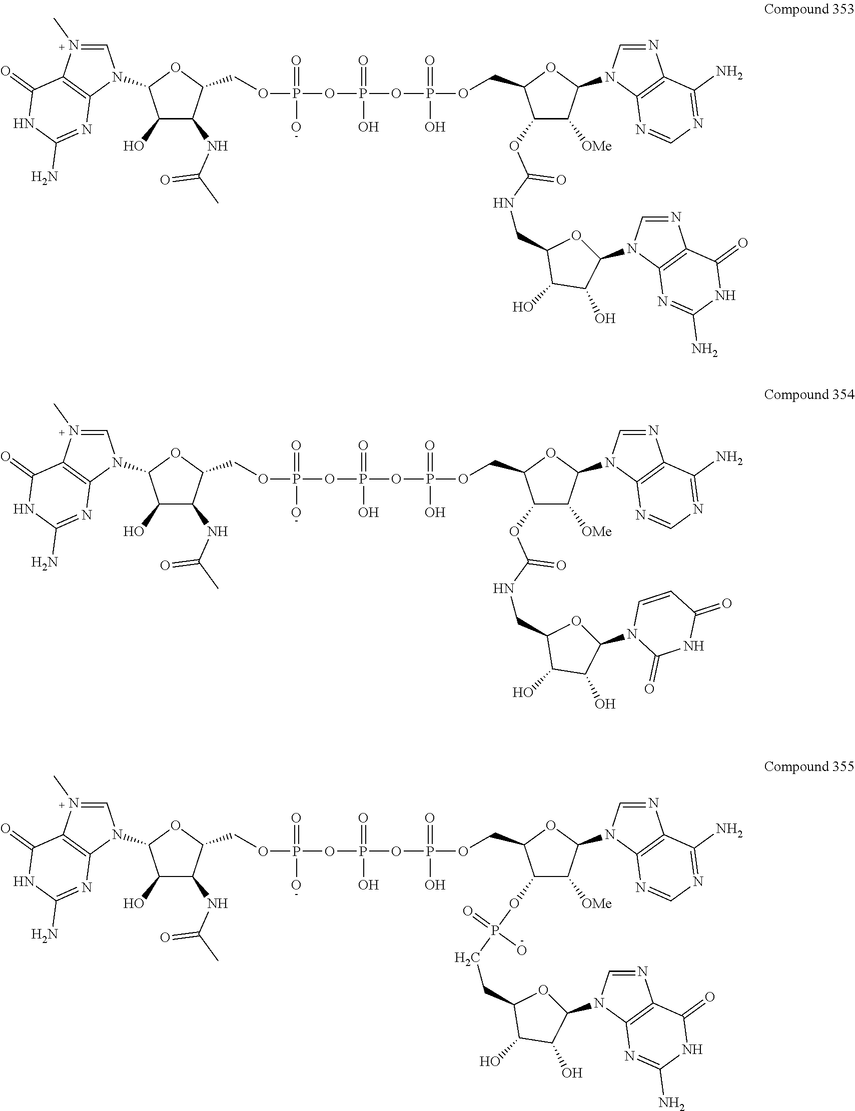

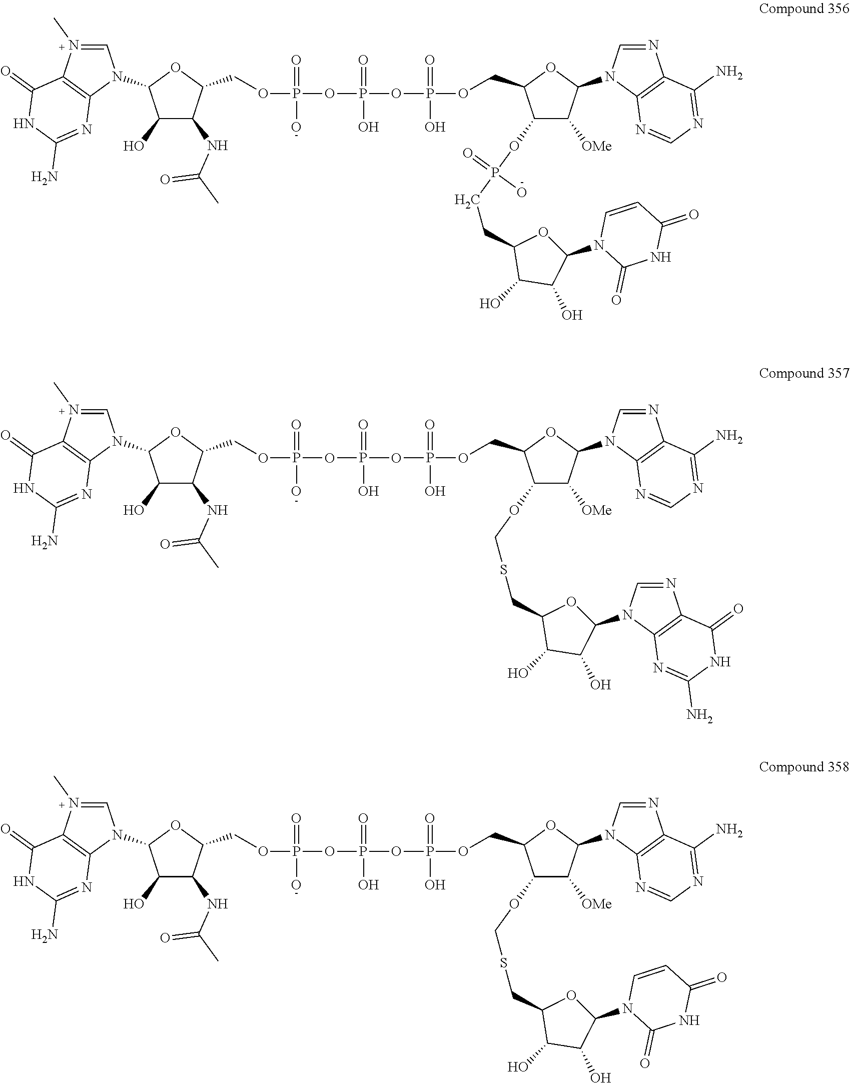

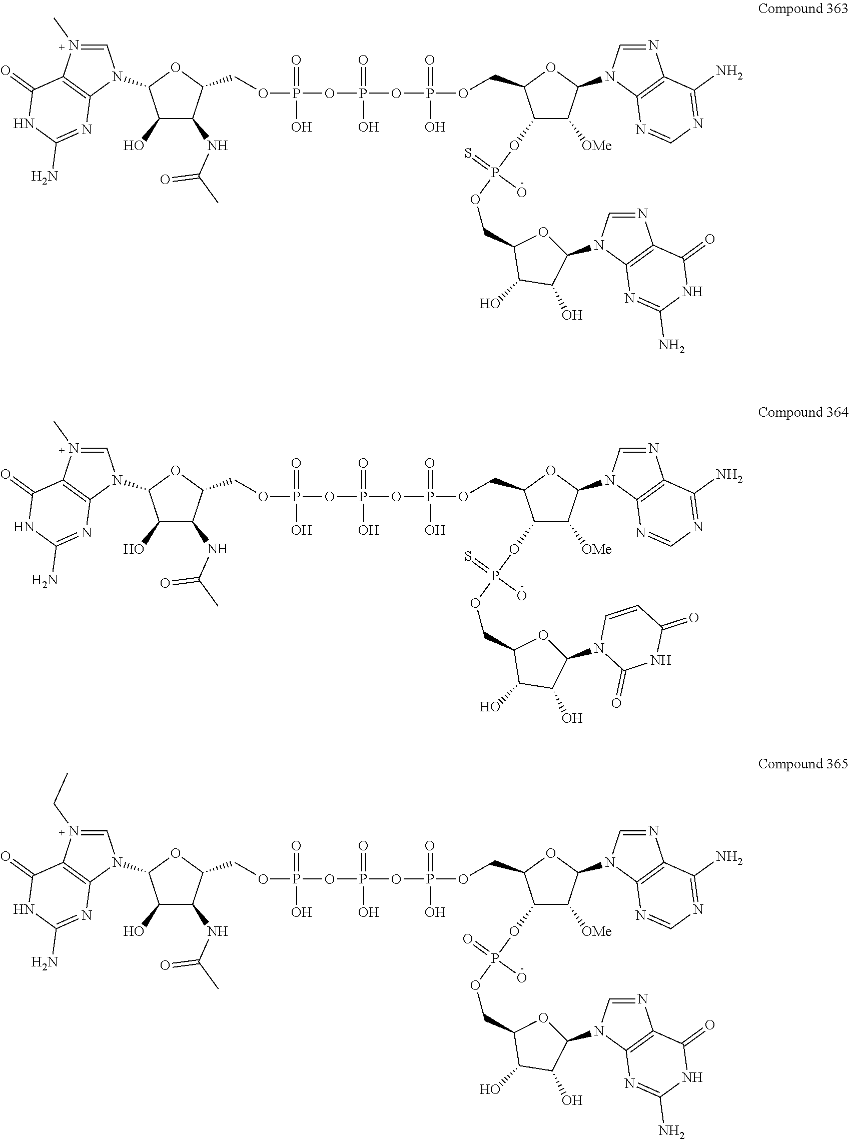

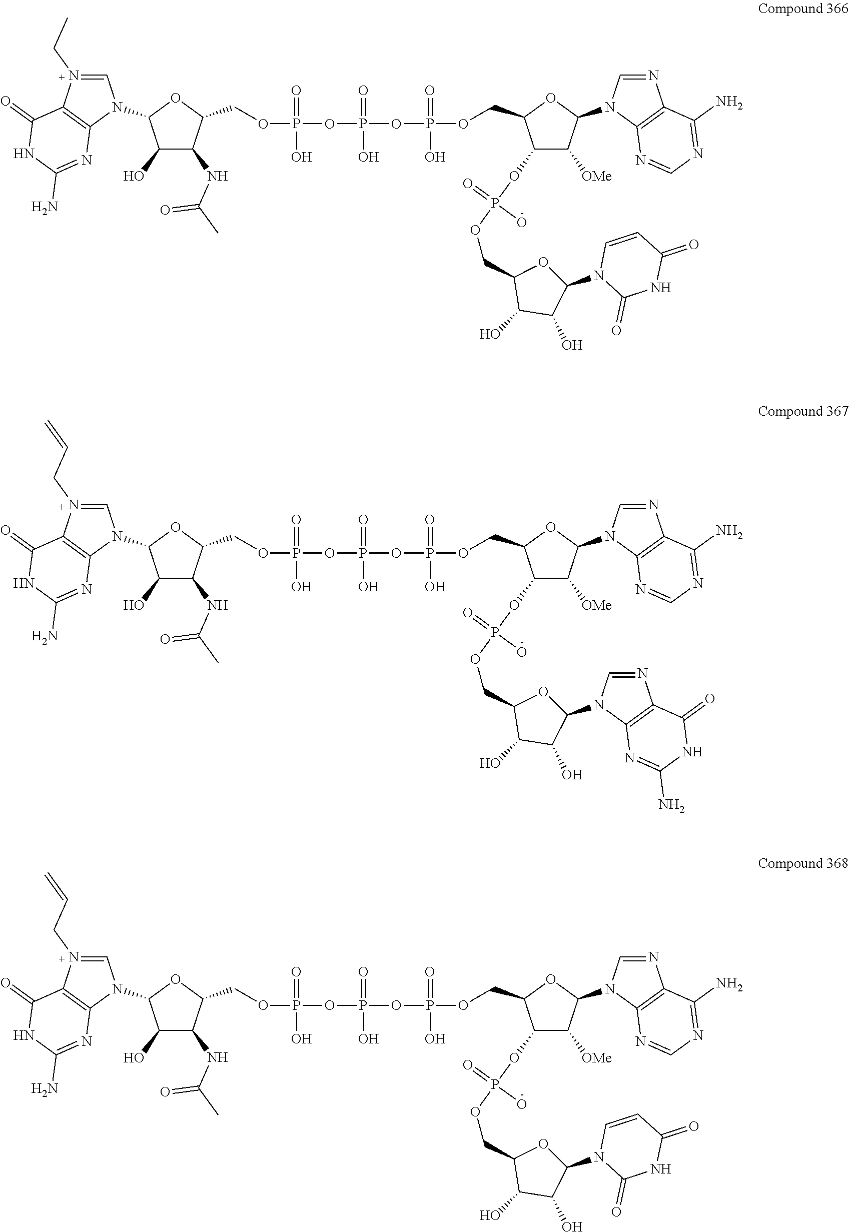

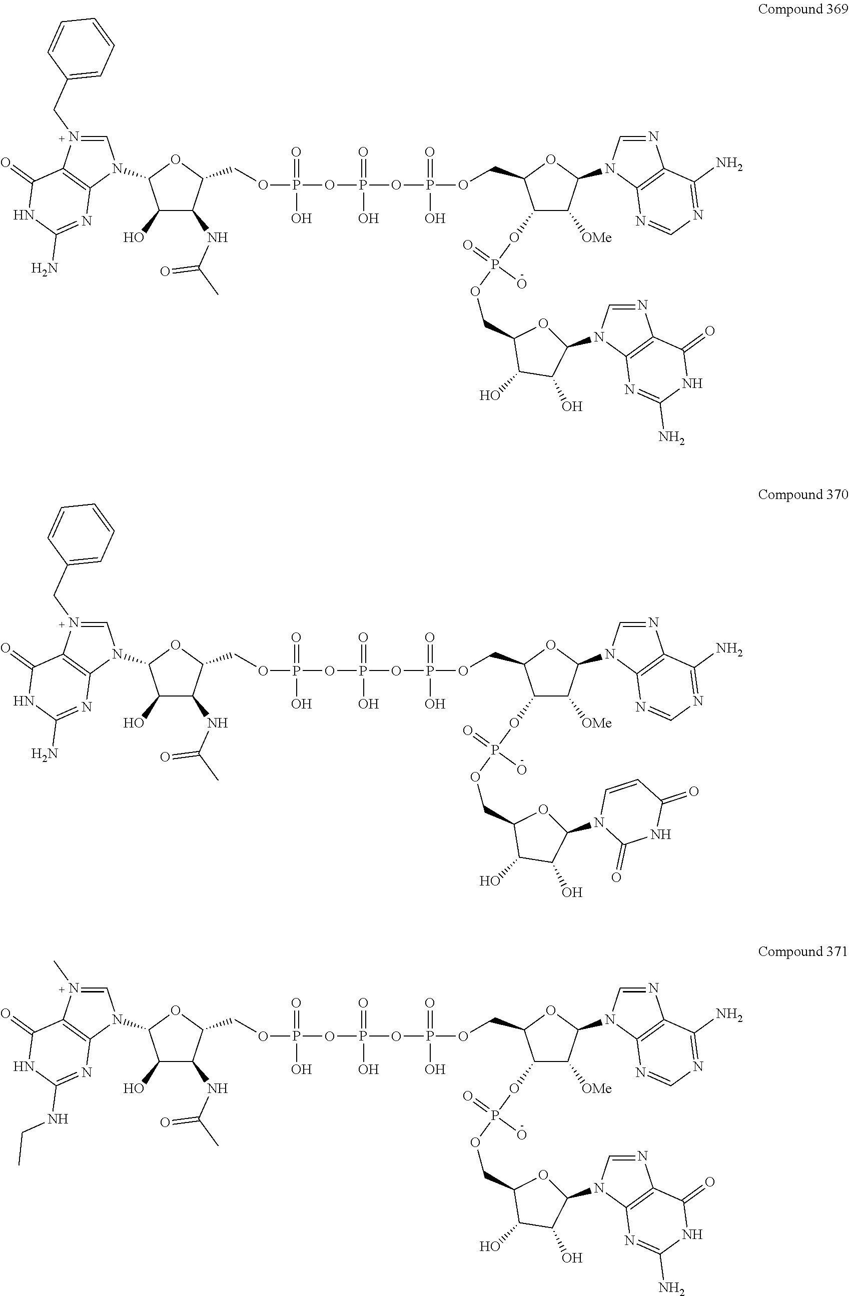

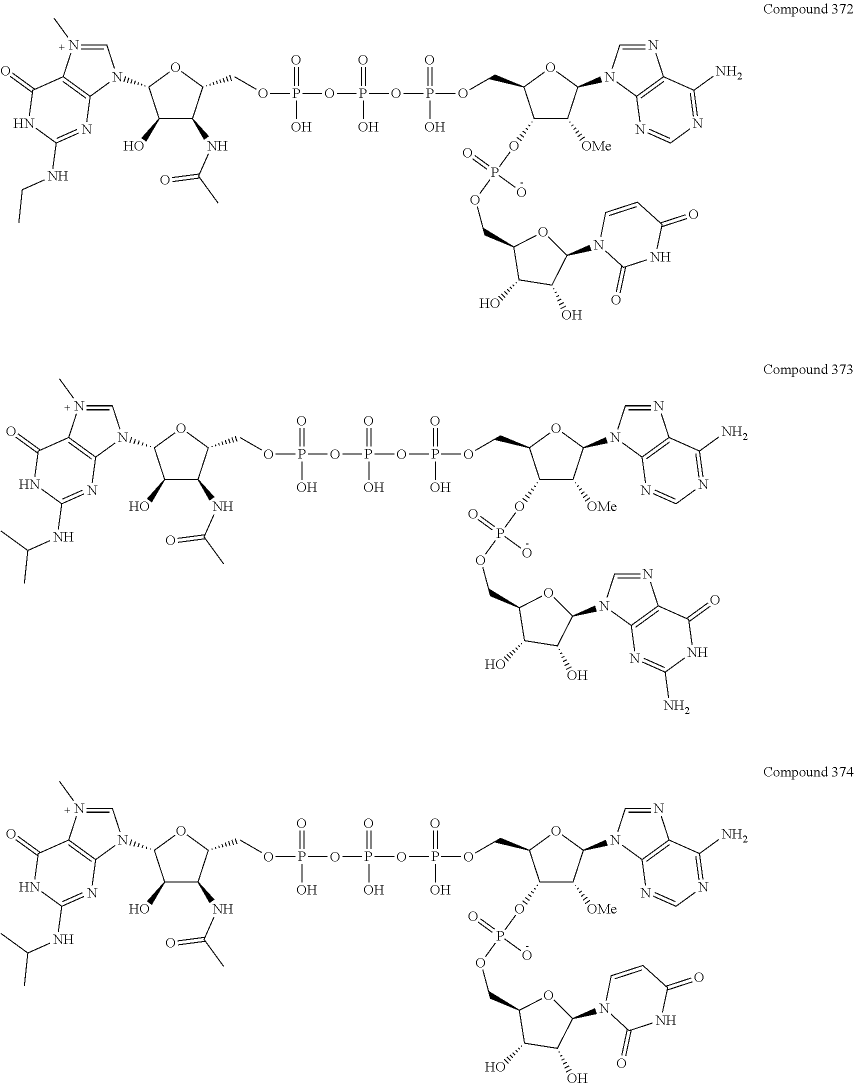

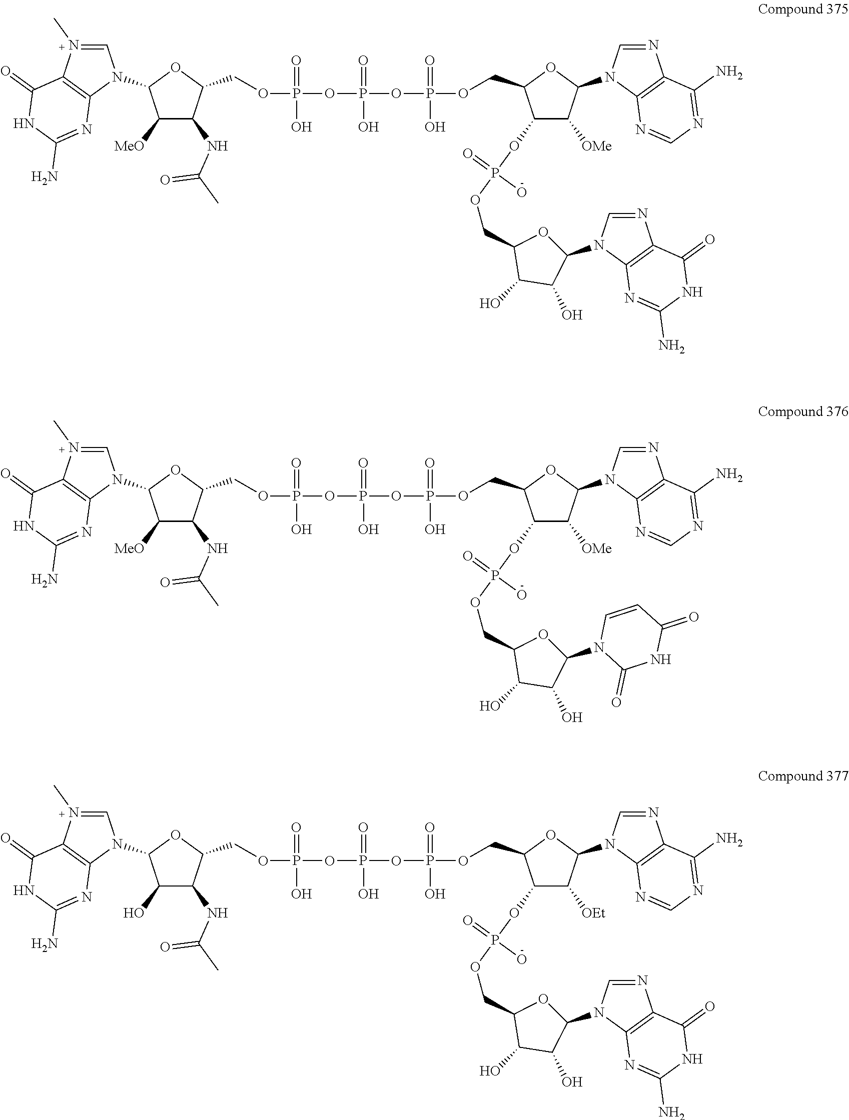

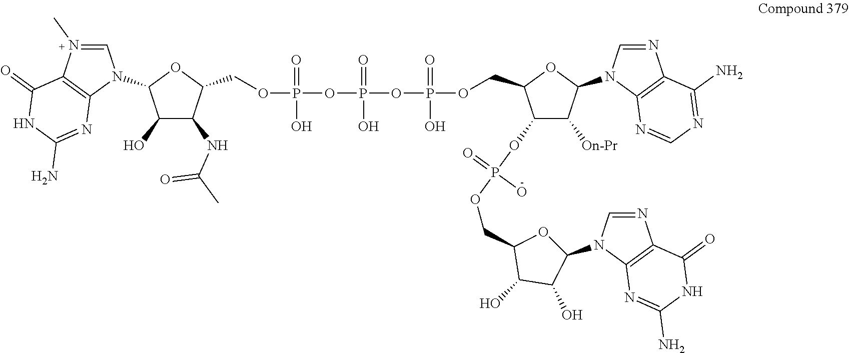

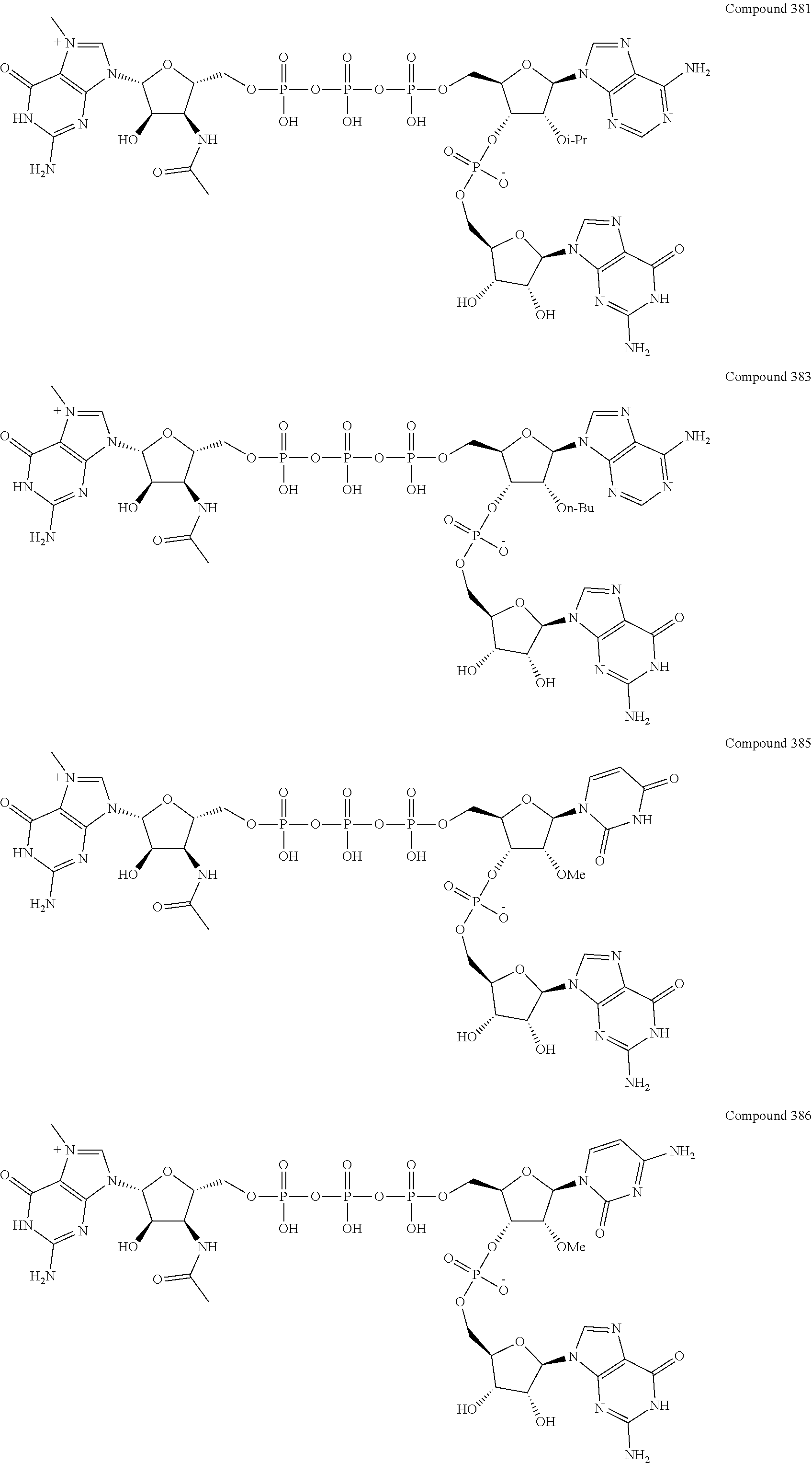

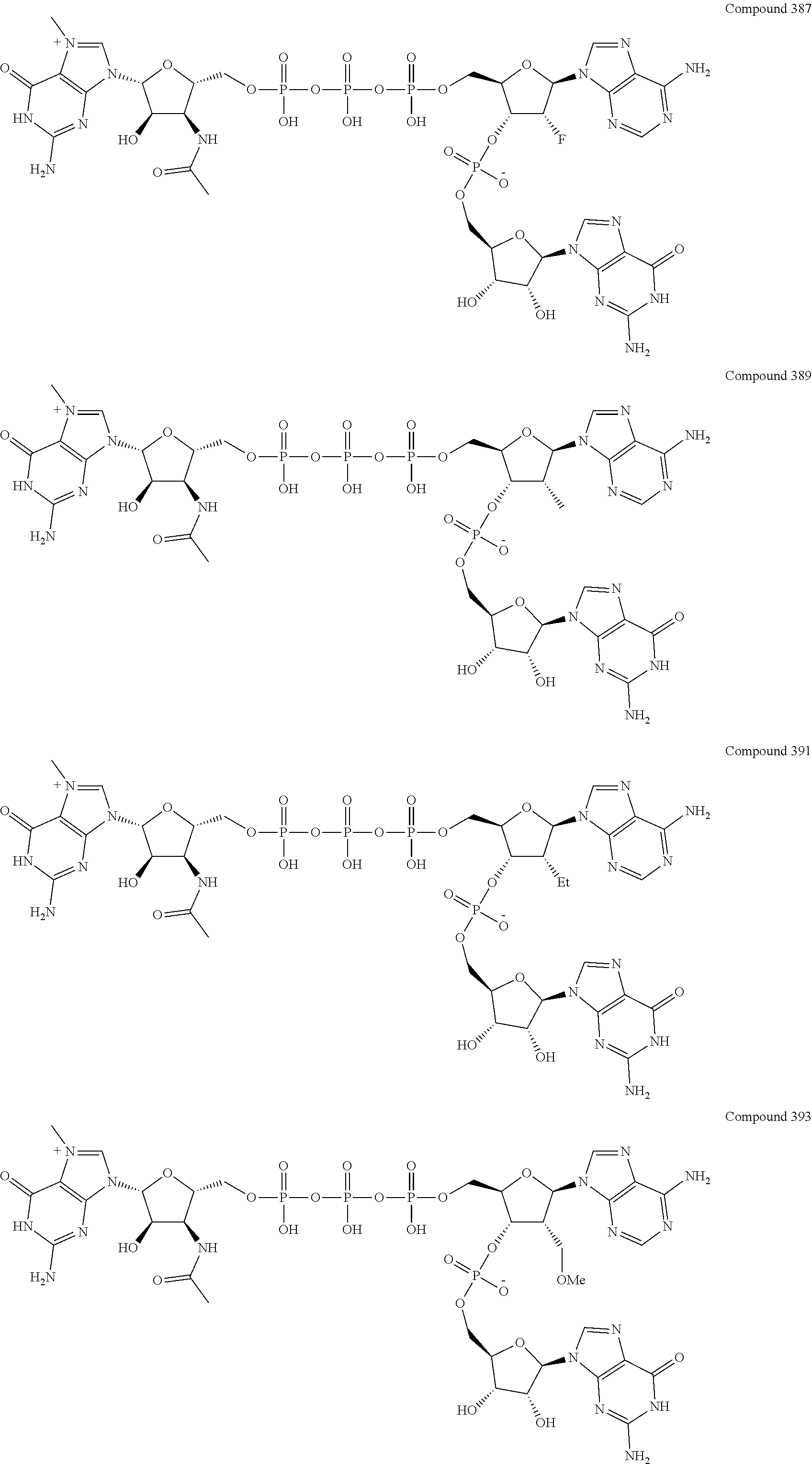

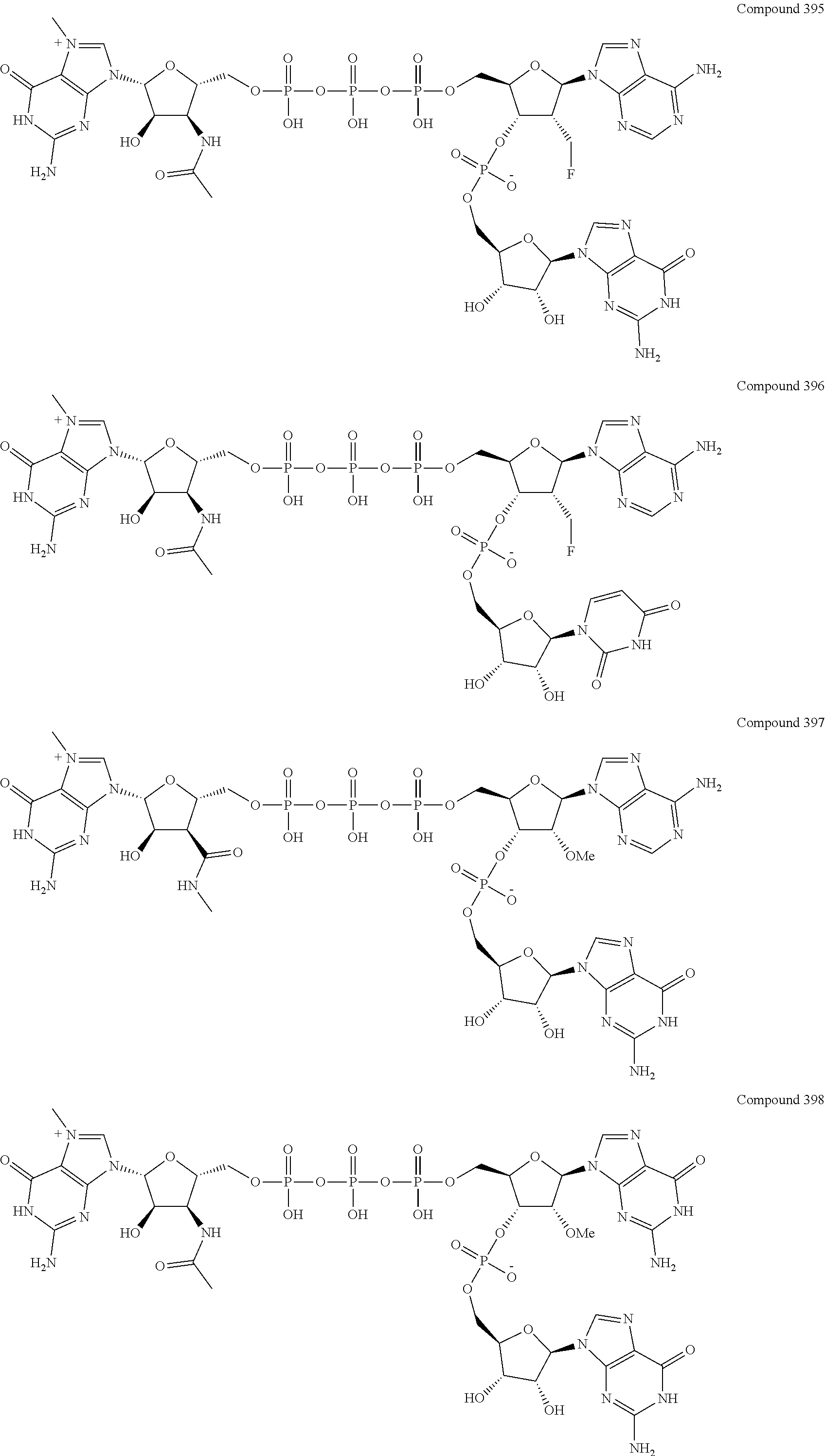

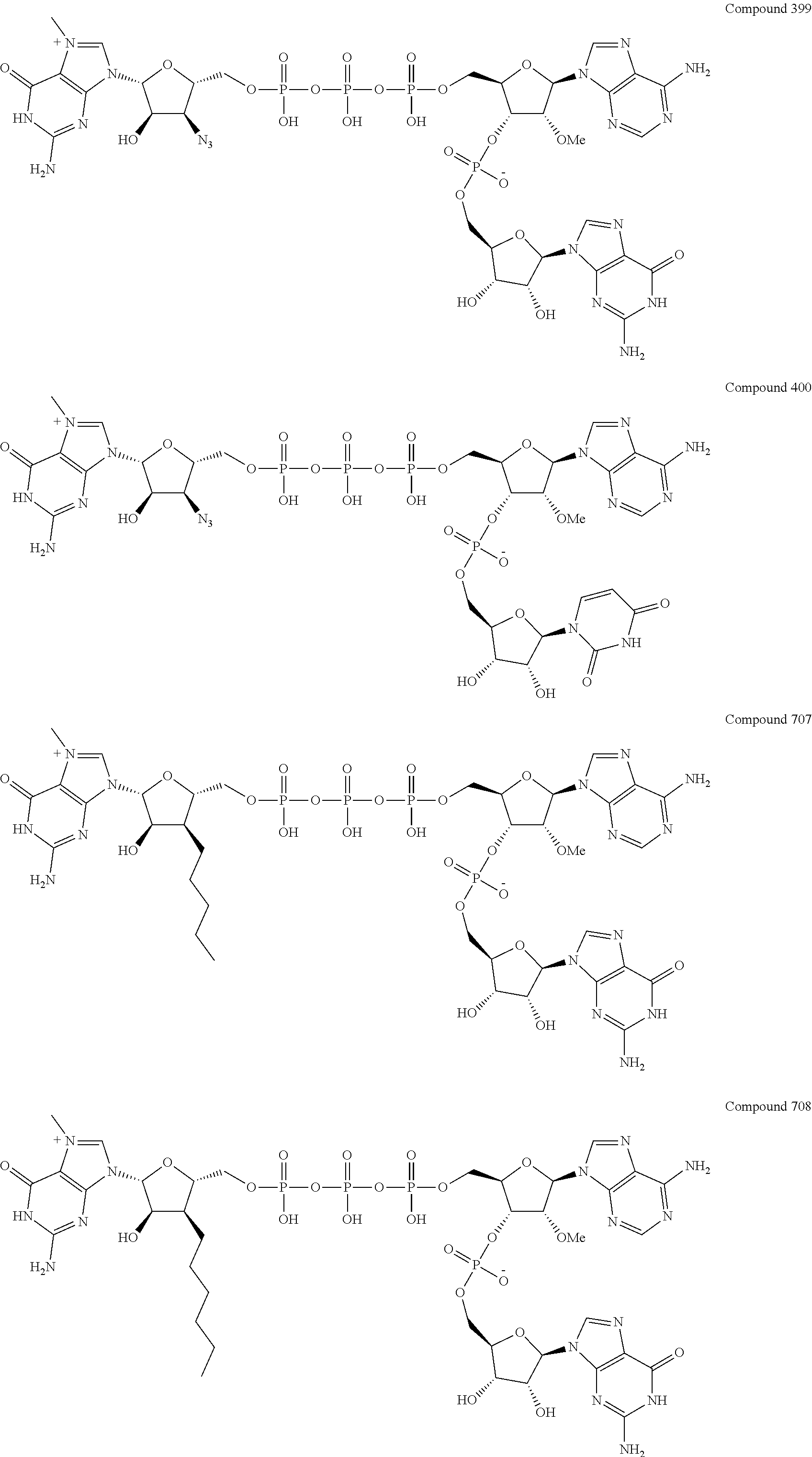

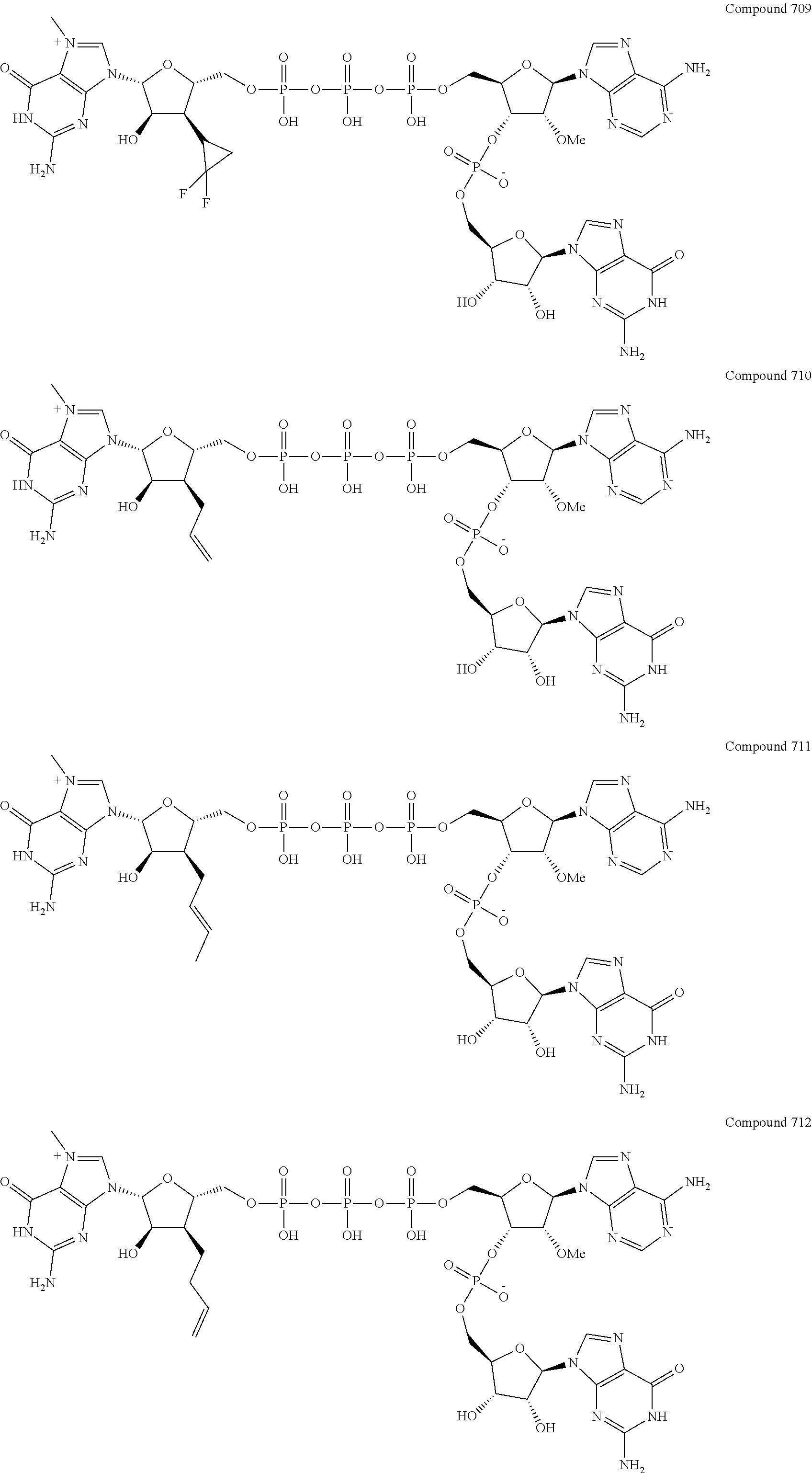

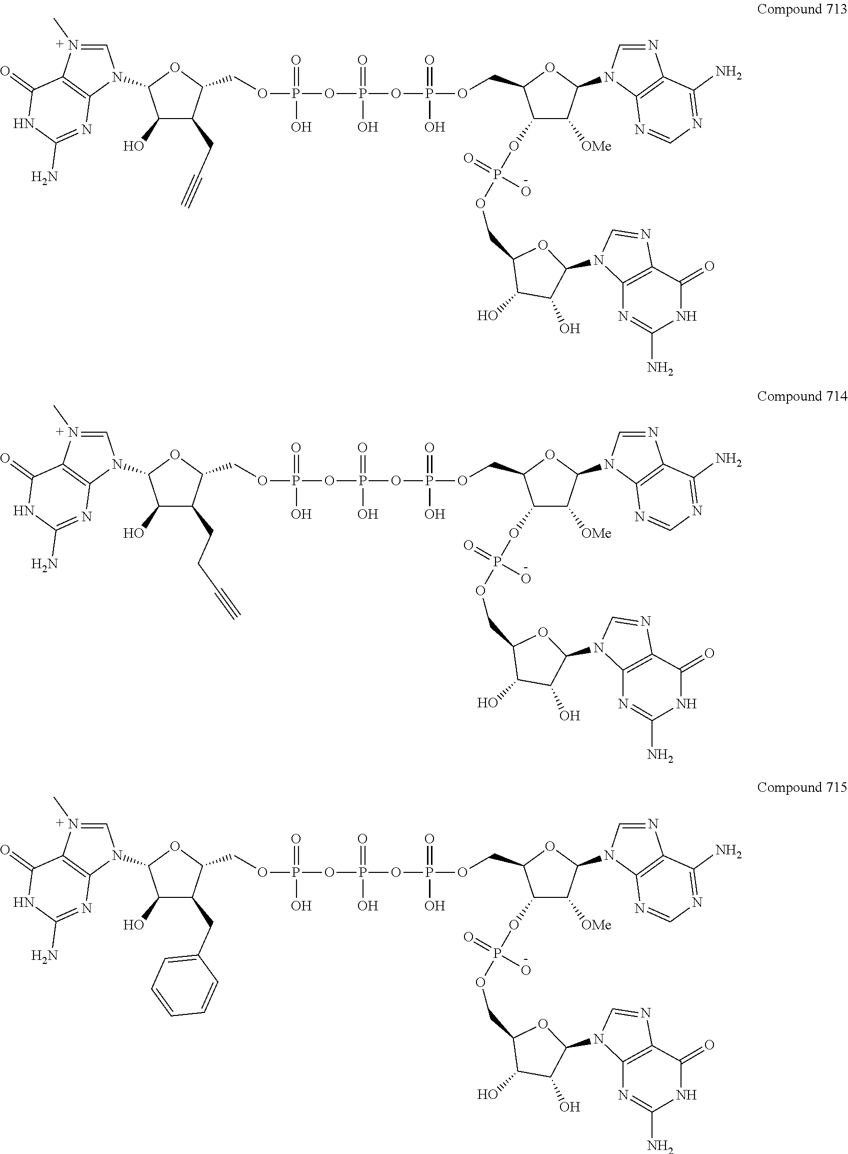

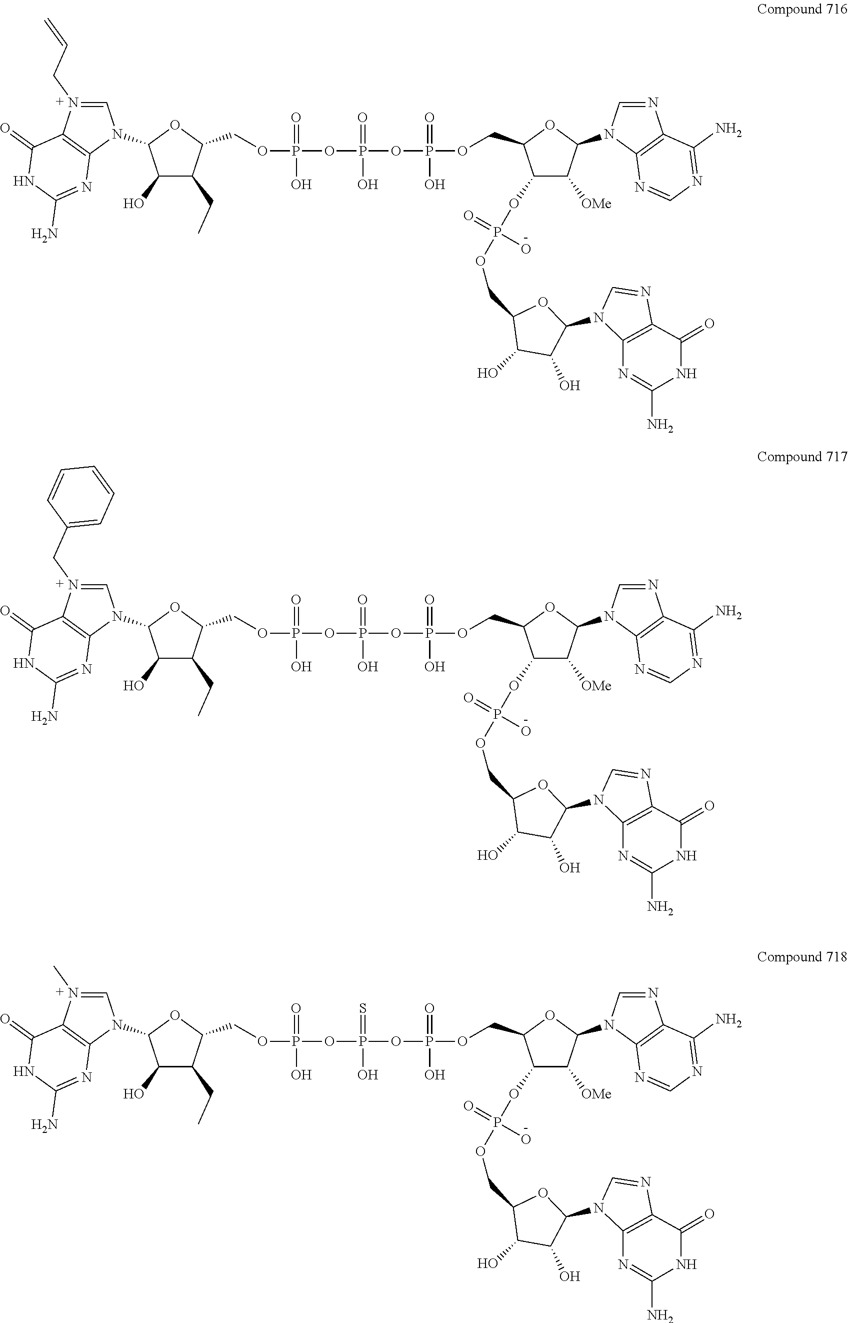

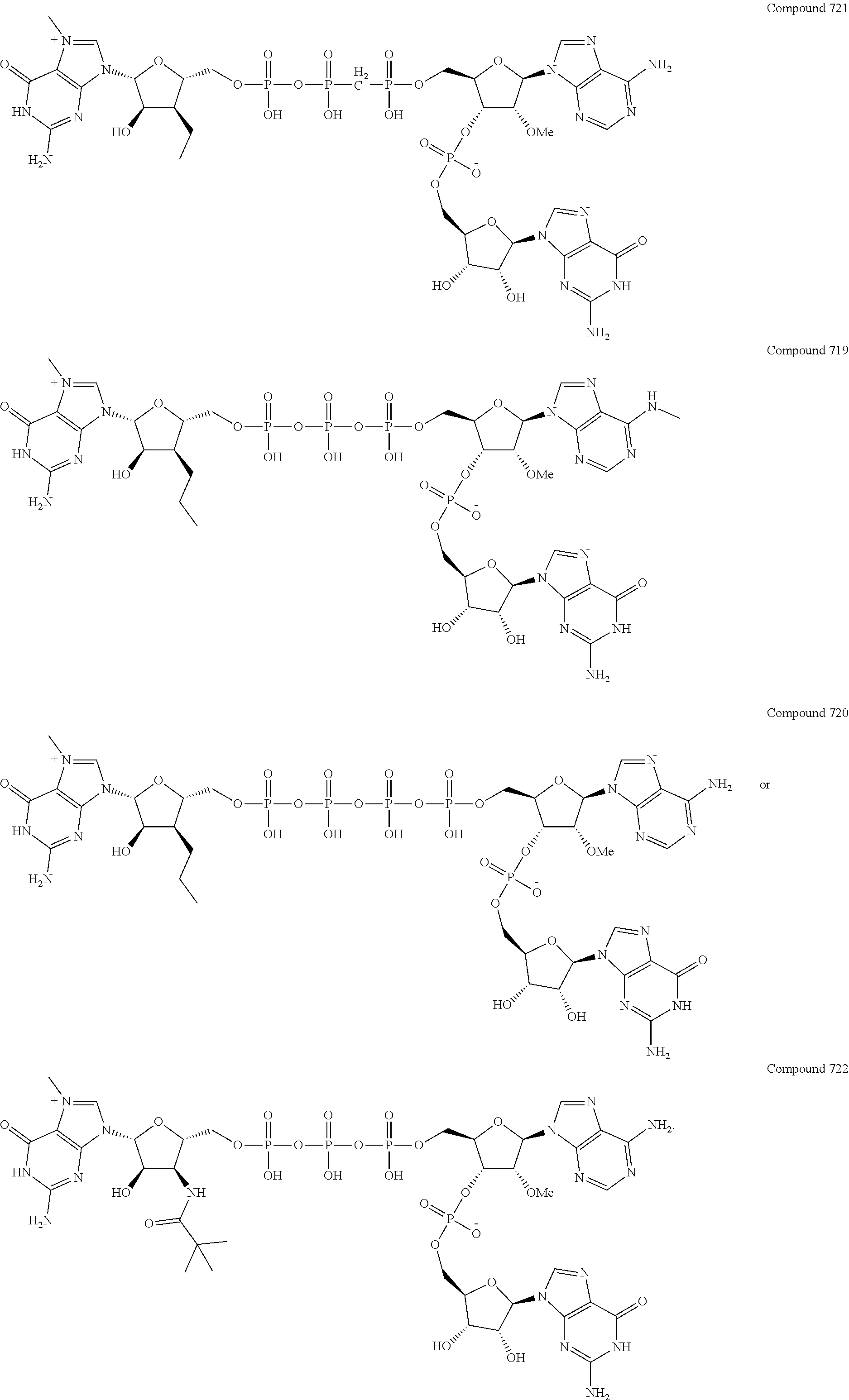

The present disclosure further discloses use of the above compound as an in vino co-transcriptional RNA capping reagent.

The present disclosure further discloses an RNA molecule comprising the above compound as a cap structure or a cap structure fragment.

It can be understood that the above RNA molecule can be used as an mRNA vaccine, an RNA drug, or in cell therapy of precision medicine.

The present disclosure further discloses a pharmaceutical composition comprising the above RNA molecule and a pharmaceutically acceptable carrier.

The present disclosure further discloses a method for synthesizing an RNA molecule, comprising the following step:
co-incubating the above compound with a polynucleotide template for template transcription.

The present disclosure further discloses a capped RNA transcription reaction system comprising: a polynucleotide template, the above compound, NTPs and an RNA polymerase.

Compared with the prior art, the present disclosure has the following beneficial effects:

The compound for RNA capping of the present disclosure can be used as an initial capping oligonucleotide primer to cap the 5′ end of mRNA, and has good capping efficiency and protein expression; the capped mRNA can express protein stably and in high yield; using the compound of the present disclosure as a cap structure for synthetisizing RNA vaccines or drugs can greatly reduce costs; the compound of the present disclosure has broad application prospects in the preparation of RNA vaccines or drugs.

### DETAILED DESCRIPTION OF THE PREFERRED EMBODIMENT

In order to facilitate understanding of the present disclosure, the present disclosure will be described more fully below with reference to the relevant drawings. Preferred examples of the present disclosure are shown in the accompanying drawings. However, the present disclosure may be embodied in many different forms and is not limited to the examples described herein. On the contrary, these examples are provided for the purpose of providing a more thorough and comprehensive understanding of the present disclosure.

Unless otherwise defined, all technical and scientific terms used herein have the same meaning as commonly understood by one of ordinary skill in the art to which the present disclosure belongs. The terms used in the specification of the present disclosure herein are for the purpose of describing specific examples only and are not intended to limit the present disclosure.

### Terms:

Pyrimidine nucleotide bases include, but are not limited to: uracil, thymine, cytosine, 5-methylcytosine, 5-fluorouracil, 5-fluorocytosine, etc.

Purine nucleotide derivatives include, but are not limited to, adenine, guanine, 6-N-methyladenine, 6-N,N-dimethyladenine, 2-N-methylguanine, 2-N,N-dimethylguanine, etc.

“Together as a single bond or a double bond” indicates that this structure is a chemical bond, and specifically optionally a single bond or a double bond, e.g., “—X₄— together as a single bond or a double bond” means that when optionally —X₄— is a single bond or a double bond, the ring of the parent nucleus of formula I is a five-membered ring, with the groups on both side of X₄ directly connected.

“Connected to form a ring by a chemical bond” means that two groups are connected by a carbon-carbon bond, a carbon-oxygen bond, a carbon-nitrogen bond, a carbon-sulfur bond, or the like to form a ring structure, with the corresponding groups reducing 1 to 2 hydrogen atoms when necessary.

“Stereoisomer” refers to a compound with the same chemical composition but differing in the spatial arrangement of atoms or groups. Stereoisomers include enantiomers, diastereomers, conformational isomers (rotational isomers), geometric isomers (cis/trans isomers), atropisomers, etc.

The term “substituted” indicates that one or more hydrogen atoms in the given structure are substituted by specific substituents. Unless otherwise indicated, a substituted group may have a substituent substituted at each substitutable site of the group. When more than one site in a given structural formula can be substituted by one or more substituents selected from specific groups, the substituents may be identically or differently substituted at each site.

The term “be independently” should be understood in a broad sense, either to mean that the specific options expressed between the same symbols in different groups do not affect each other, or that the specific options expressed between the same symbols in the same groups do not affect each other.

In various sections of this specification, the substituents of the compounds disclosed in the present disclosure are disclosed according to the type or range of groups. It is specifically noted that the present disclosure includes each individual member of every independent secondary combination of these types and ranges of groups. For example, the term “C1-6 alkyl” specifically refers to the independently disclosed methyl, ethyl, C3 alkyl, C4 alkyl, C5 alkyl and C6 alkyl.

In various sections of the present disclosure, the connection of substituents is described. When the structure explicitly requires a connecting group, the Markush variables listed for the group should be understood as connecting groups. For example, if the structure requires a connecting group and the Markush group definition for the variable lists “alkyl” or “aryl,” it should be understood that the “alkyl” or “aryl” respectively represent a connected alkylene group or a connected arylene group.

The term “alkyl” or “alkyl group” as used in the present disclosure refers to a saturated straight-chain or branched monovalent hydrocarbon group, wherein the alkyl group may be optionally substituted by one or more substituents described in the present disclosure. The alkyl group may be optionally substituted by one or more substituents described in the present disclosure.

Examples of alkyl groups include, but are not limited to, methyl (Me, —CH₃), ethyl (Et, —CH₂CH₃), n-propyl (n-Pr, —CH₂CH₂CH₃), isopropyl (i-Pr, —CH(CH₃)₂), n-butyl (n-Bu, —CH₂CH₂CH₂CH₃), isobutyl (i-Bu, —CH₂CH(CH₃)₂), sec-butyl (s-Bu, —CH(CH₃)CH₂CH₃), tert-butyl (t-Bu, —C(CH₃)₃), n-pentyl (—CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), n-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), n-heptyl, n-octyl, etc.

The term “alkenyl” refers to a straight-chain or branched monovalent hydrocarbon group containing 2 to 30 carbon atoms, with at least one unsaturated site, i.e., one carbon-carbon sp2 double bond. Such alkenyl groups include a “cis” and “trans” orientation, or an “E” and “Z” orientation. Examples of alkenyl groups include, but are not limited to, vinyl (—CH═CH₂), allyl (—CH₂CH═CH₂), etc. The alkenyl group may be optionally substituted by one or more substituents described in the present disclosure.

The term “alkynyl” means that there is at least one unsaturated site, that is, one carbon-carbon sp triple bond. Examples of alkynyl groups include, but are not limited to, ethynyl (—C≡CH), propargyl (—CH₂C≡CH), 1-propynyl (—C≡C—CH₃), etc. The alkynyl group may be optionally substituted by one or more substituents described in the present disclosure.

The term “cycloalkyl” as used in the present disclosure, unless otherwise specified, refers to a monovalent saturated or partially unsaturated (but non-aromatic) monocyclic or polycyclic hydrocarbon. In some embodiments, the cycloalkyl group may be bridged or non-bridged, spiro or non-spiro, and/or fused or non-fused bicyclic groups. In some embodiments, the cycloalkyl group contains 3 to 10 carbon atoms, i.e., C3 to C10 cycloalkyl. In some embodiments, the cycloalkyl group has 3-15 (C3-15), 3-10 (C3-10) or 3-7 (C3-7) carbon atoms. In some embodiments, the cycloalkyl group is monocyclic or bicyclic. In some embodiments, the cycloalkyl group is monocyclic. In some embodiments, the cycloalkyl group is bicyclic. In some embodiments, the cycloalkyl group is cyclopropyl, cyclobutyl, cyclopentyl, cyclohexyl, cycloheptyl, bicyclo[2.1.1]hexyl, bicyclo[2.2.1]heptyl, decahydronaphthyl or adamantyl. When the cycloalkyl group is substituted, it may be substituted independently by one or more substituents described in the present disclosure, on any ring, i.e., any aromatic or non-aromatic ring contained within the cycloalkyl.

The terms “heterocyclyl/heterocyclic group” and “heterocycle” are used interchangeably in the present disclosure, unless otherwise specified, to refer to a monovalent monocyclic non-aromatic ring system and/or a polycyclic system comprising at least one non-aromatic ring, wherein one or more (in some embodiments, 1, 2, 3 or 4) atoms in the monocyclic non-aromatic ring are independently selected from O, S(O)₀₋₂ and N heteroatoms, with the remaining ring atoms being carbon atoms; and wherein in the polycyclic system, one or more (in some embodiments, 1, 2, 3 or 4) ring atoms are independently selected from O, S(O)₀₋₂ and N heteroatoms, with the remaining ring atoms being carbon atoms. In some embodiments, the heterocycle contains 1 or 2 heteroatoms, where the heteroatoms are nitrogen atoms. In some embodiments, the heterocyclic group is polycyclic and contains one heteroatom in a non-aromatic ring, or one heteroatom in an aromatic ring, or two heteroatoms, with one in the aromatic ring and the other in the non-aromatic ring. In some embodiments, the heterocyclic group has 3 to 20, 3 to 15, 3 to 10, 3 to 8, 4 to 7 or 5 to 6 ring atoms. In some embodiments, the heterocyclic group is a monocyclic, bicyclic, tricyclic or tetracyclic system.

In one embodiment, the heterocyclic group is a heterocyclic group composed of 3 to 8 atoms, which refers to a saturated or partially unsaturated monocyclic ring containing 3 to 8 ring atoms, wherein at least one ring atom is selected from nitrogen, sulfur and oxygen atoms. Unless otherwise specified, the heterocyclic group composed of 3 to 8 atoms may be connected to other parts through carbon atoms or nitrogen atoms, and the —CH2- group may be optionally replaced by —C(═O)—. The sulfur atom in the ring may be optionally oxidized to form an S-oxide. The nitrogen atom in the ring may be optionally oxidized to form an N-oxide. Examples of heterocyclic groups composed of 3 to 8 atoms include, but are not limited to: azetidinyl, oxetanyl, thietanyl, pyrrolidinyl, 2-pyrrolinyl, 3-pyrrolinyl, pyrazolinyl, pyrazolidinyl, imidazolinyl, imidazolidinyl, tetrahydrofuranyl, dihydrofuranyl, tetrahydrothiophenyl, dihydrothiophenyl, 1,3-dioxolanyl, dithiolanyl, tetrahydropyranyl, dihydropyranyl, 2H-pyranyl, 4H-pyranyl, tetrahydrothiopyranyl, piperidyl, morpholinyl, thiomorpholinyl, piperazinyl, dioxanyl, dithianyl, thioxanyl, homopiperazinyl, homopiperidinyl, oxacycloheptyl, thiocycloheptyl, oxazepinyl, diazepinyl and thiazepinyl. Examples of the —CH₂— group in the heterocyclic group substituted with —C(═O)— include, but are not limited to, 2-oxopyrrolidinyl, oxo-1,3-thiazolidinyl, 2-piperidinonyl, 3,5-dioxopiperidinyl and pyrimidinedionyl. Examples of heterocyclic groups in which a sulfur atom is oxidized include, but are not limited to, cyclobutanesulfonyl and 1,1-dioxothiomorpholinyl. The heterocyclic groups composed of 3 to 8 atoms may be optionally substituted by one or more substituents as described in the present disclosure.

In one embodiment, the heterocyclic group is a heterocyclic group composed of 3 to 6 atoms, which refers to a saturated or partially unsaturated monocyclic ring containing 3 to 6 ring atoms, wherein at least one ring atom is selected from nitrogen, sulfur and oxygen atoms. Unless otherwise specified, the heterocyclic group composed of 3 to 6 atoms may be connected to other parts through carbon atoms or nitrogen atoms, and the —CH2- group may be optionally replaced by —C(═O)—. The sulfur atom in the ring may be optionally oxidized to form an S-oxide. The nitrogen atom in the ring may be optionally oxidized to form an N-oxide. The heterocyclic groups composed of 3 to 6 atoms may be optionally substituted by one or more substituents as described in the present disclosure.

In another embodiment, the heterocyclic group is a heterocyclic group composed of 5 to 6 atoms, which refers to a saturated or partially unsaturated monocyclic ring containing 5 to 6 ring atoms, where at least one ring atom is selected from nitrogen, sulfur and oxygen atoms. Unless otherwise specified, the heterocyclic group composed of 5 to 6 atoms may be connected to other parts through carbon atoms or nitrogen atoms, and the —CH2- group may be optionally replaced by —C(═O)—. The sulfur atom in the ring may be optionally oxidized to form an S-oxide. The nitrogen atom in the ring may be optionally oxidized to form an N-oxide. Examples of heterocyclic groups composed of 5 to 6 atoms include, but are not limited to: pyrrolidinyl, 2-pyrrolinyl, 3-pyrrolinyl, pyrazolinyl, pyrazolidinyl, imidazolinyl, imidazolidinyl, tetrahydrofuranyl, dihydrofuranyl, tetrahydrothiophenyl, dihydrothiophenyl, 1,3-dioxolanyl, dithiolanyl, 2-oxo-pyrrolidinyl, oxo-1,3-thiazolidinyl, cyclobutanesulfonyl, tetrahydropyranyl, dihydropyranyl, 2H-pyranyl, 4H-pyranyl, tetrahydrothiopyranyl, piperidyl, morpholinyl, thiomorpholinyl, piperazinyl, dioxanyl, dithianyl, thioxanyl, 2-piperidone, 3,5-dioxopiperidinyl, pyrimidinedione and 1,1-dioxothiomorpholinyl. The heterocyclic group composed of 5 to 6 atoms may be optionally substituted by one or more substituents as described in the present disclosure.

The term “aryl” as used in the present disclosure, unless otherwise specified, refers to a monovalent C6-C14 carbocyclic ring system containing at least one aromatic ring, where the aromatic ring system may be monocyclic, bicyclic or tricyclic. The aryl group may be connected to the main structure via any of its rings, i.e., through any aromatic or non-aromatic ring. In some embodiments, the aryl group is phenyl, naphthyl, bicyclo[4.2.0]oct-1,3,5-trienyl, indanyl, fluorenyl or tetrahydronaphthyl. When the aryl group is substituted, it may be substituted on any of the rings, i.e., on any aromatic or non-aromatic ring contained in the aryl group. In some or any embodiment, the aryl group is phenyl, naphthyl, tetrahydronaphthyl, fluorenyl, or indanyl. The aryl group may be independently and optionally substituted by one or more substituents as described in the present disclosure.

The term “heteroaryl” as used in the present disclosure, unless otherwise specified, refers to a monovalent monocyclic or polycyclic aromatic group, where at least one (in some embodiments, 1, 2, 3 or 4) ring atom is independently selected from 0, S(O)₀₋₂ and N heteroatoms in the ring. The heteroaryl group is connected to the rest of the molecule via any atom in the ring system, provided that the valency rules for the atom allow such a connection. In some embodiments, each ring in the heteroaryl group may contain 1 or 2 O atoms, 1 or 2 S atoms, and/or 1 to 4 N atoms, or combinations thereof, provided that the total number of heteroatoms in each ring is 4 or fewer, and each ring contains at least one carbon atom. In some embodiments, the heteroaryl group contains 5 to 20, 5 to 15 or 5 to 10 ring atoms. When the heteroaryl group is substituted, substitution may occur on any of the rings.

The “solvate” of the present disclosure refers to an adduct formed by one or more solvent molecules and the compound of the present disclosure. Solvents forming solvates include, but are not limited to, water, isopropanol, ethanol, methanol, dimethyl sulfoxide, ethyl acetate, acetic acid and aminoethanol. The term “hydrate” means that the solvent molecule is an adduct formed by water.

When the solvent is water, the term “hydrate” can be used. In some embodiments, one compound molecule of the present disclosure can be combined with one water molecule, such as a monohydrate; in other embodiments, one compound molecule of the present disclosure can be combined with more than one water molecule, such as a dihydrate, and in still other embodiments, one compound molecule of the present disclosure can be combined with less than one water molecule, such as a hemihydrate. It should be noted that the hydrate of the present disclosure retains the bioavailability of the compound in non-hydrated form.

The compounds used in the following examples are all commercially available unless otherwise stated; the methods used in the following examples can be implemented by conventional methods unless otherwise stated.

### Example 1: Synthes of Compound 1

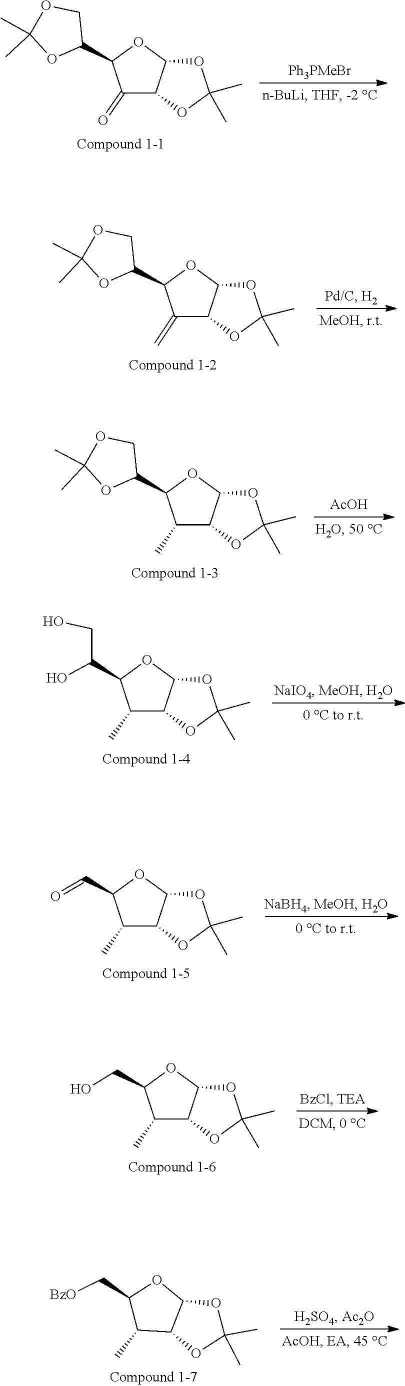

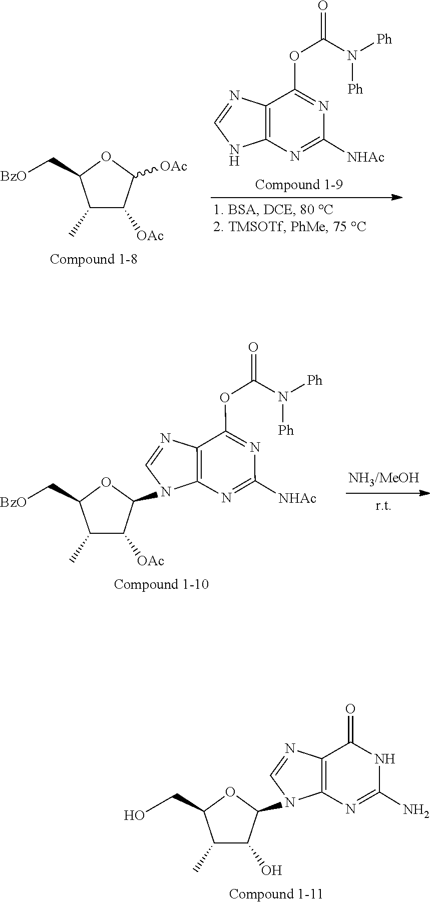

n-Butyllithium (37.2 mL, 92.98 mmol) was slowly added dropwise to a solution of methyltriphenylphosphonium bromide (21.40 g, 92.98 mmol) in tetrahydrofuran (100 mL) at −2 °C under a nitrogen atmosphere, and the reaction mixture was stirred and reacted at −2 °C for 30 minutes. A solution of compound 1-1 (20.00 g, 77.48 mmol) in tetrahydrofuran (20 mL) was slowly added dropwise to the system, and the reaction mixture was stirred and reacted at −2 °C overnight. The reaction was quenched by adding saturated ammonium chloride solution (50 mL) to the system. The mixture was transferred to room temperature and added with ethyl acetate (100 mL) and water (100 mL). The phases were separated, and the organic phase was sequentially washed with water (100 mL×2) and saturated brine, dried over anhydrous sodium sulfate, and concentrated under reduced pressure. The residue was purified by silica gel column chromatography (petroleum ether/ethyl acetate=10/1) to obtain 10.00 g of compound 1-2.

10% palladium on carbon (1.00 g) was added to a solution of compound 1-2 (10.00 g, 39.04 mmol) in methanol (100 mL) at room temperature, and the reaction mixture was stirred and reacted overnight under a hydrogen atmosphere. The reaction mixture was subjected to suction filtration, and the filtrate was concentrated under reduced pressure to obtain 9.00 g of compound 1-3, which was directly used in the next reaction without purification.

At room temperature, 70% acetic acid (acetic acid/water (vN)=7/3, 100 mL) was added to compound 1-3 (9.00 g, 21.00 mmol), and the reaction mixture was heated to 50 °C, stirred and reacted for 2 hours. The reaction mixture was concentrated under reduced pressure to obtain 10.00 of compound 1-4, which was directly used in the next reaction without purification.

Under an ice bath, sodium periodate (10.79 g, 50.43 mmol) was added to a mixed solution of compound 1-4 (10.00 g, 45.85 mmol) in methanol (100 mL) and water (100 mL). The reaction mixture was stirred for 5 minutes, then transferred to room temperature, stirred and reacted for 30 minutes. The reaction mixture was subjected to suction filtration, and the filtrate was collected and cooled to 0 °C. Sodium borohydride (3.47 g, 91.70 mmol) was added to the system in batches, and the reaction mixture was transferred to room temperature, stirred and reacted for 30 minutes after the addition was completed. The reaction was quenched with saturated aqueous ammonium chloride solution (10 mL). Most of the reaction mixture was concentrated under reduced pressure, and the residue was extracted with dichloromethane (100 mL×3), dried over anhydrous sodium sulfate, and concentrated under reduced pressure to obtain 5.50 g of compound 1-6, which was directly used in the next reaction without purification.

In an ice bath, under a nitrogen atmosphere, benzoyl chloride (3.72 mL, 32.15 mmol) was slowly added dropwise to a solution of compound 1-6 (5.50 g, 29.23 mmol) and triethylamine (8.1 mL, 58.48 mmol) in dichloromethane (55 mL). After the addition was completed, the reaction mixture was stirred under the ice bath for 1 hour. Water (100 mL) was added to the system, and the mixture was extracted with dichloromethane (100 mL×3). The organic phases were combined and concentrated under reduced pressure. The residue was purified by silica gel column chromatography (petroleum ether/ethyl acetate=16/1) to obtain 7.90 g of compound 1-7.

At room temperature, concentrated sulfuric acid (0.14 mL, 2.7 mmol) was diluted with ethyl acetate (8 mL) and added to a solution of compound 1-7 (7.90 g, 27.04 mmol), acetic acid (6.2 mL, 108.16 mmol) in ethyl acetate (30 mL). The temperature was raised to 45 °C, and a solution of acetic anhydride (3.8 mL, 40.56 mmol) diluted with ethyl acetate (10 mL) was slowly added dropwise to the system. After the addition was completed, the reaction mixture was stirred at 45 °C for 2 hours. The reaction mixture was cooled to 0 °C, added with triethylamine (0.8 mL, 6.10 mmol), stirred for 5 minutes, then slowly added with saturated aqueous sodium bicarbonate solution (30 mL). The phases were separated, and the aqueous phase was extracted with ethyl acetate (30 mL). The organic phases were combined, and then washed sequentially with saturated aqueous sodium bicarbonate solution (30 mL) and saturated aqueous sodium chloride solution (30 mL), dried over anhydrous sodium sulfate, and concentrated under reduced pressure to obtain 9.09 g of compound 1-8.

At room temperature, under a nitrogen atmosphere, N,O-bis(trimethylsilyl)acetamide (BSA, 13.37 mL, 54.08 mmol) was added to a suspension of compound 1-9 (12.6 g, 32.45 mmol) in 1,2-dichloroethane (135 mL), and the reaction mixture was heated to 80 °C and stirred for 2 hours. The reaction mixture was concentrated under reduced pressure, and toluene (100 mL) was added thereto. Compound 1-8 (9.09 g, 27.04 mmol) was dissolved in toluene (35 mL) and added to the system. Trimethylsilyl trifluoromethanesulfonate (TMSOTf, 6.36 mL, 35.15 mmol) was then added thereto, and the reaction mixture was heated to 75 °C, stirred and reacted for 3 hours. The system was cooled to room temperature, and water (200 mL) was added thereto. The phases were separated, and the aqueous phase was extracted with ethyl acetate (100 mL). The organic phases were combined, washed with water (100 mL), dried over anhydrous sodium sulfate, concentrated under reduced pressure, and the residue was purified by silica gel column chromatography (petroleum ether/ethyl acetate=1/2) to obtain 11.00 g of compound 1-10.

A methanol solution of ammonia (72 mL) was added to compound 1-10 (5.00 g, 7.50 mmol) at room temperature and stirred under airtight conditions overnight. The reaction mixture was concentrated under reduced pressure and purified by slurrying with methanol (30 mL) to obtain 0.90 g of compound 1-11.

The characterization data of compound 1-11 are: MS (m/z): 281.91 [M+H]⁺; ¹H NMR (400 MHz, DMSO): δ 10.56 (s, 1H), 7.97 (s, 1H), 6.43 (s, 2H), 5.70 (s, 1H), 5.52 (d, J=5.3 Hz, 1H), 4.96 (t, J=5.3 Hz, 1H), 4.19 (t, J=5.0 Hz, 1H), 3.77 (d, J=9.3 Hz, 1H), 3.73-3.67 (m, 1H), 3.58-3.45 (m, 1H), 2.38-2.31 (m, 1H), 0.97 (d, J=6.7 Hz, 3H).

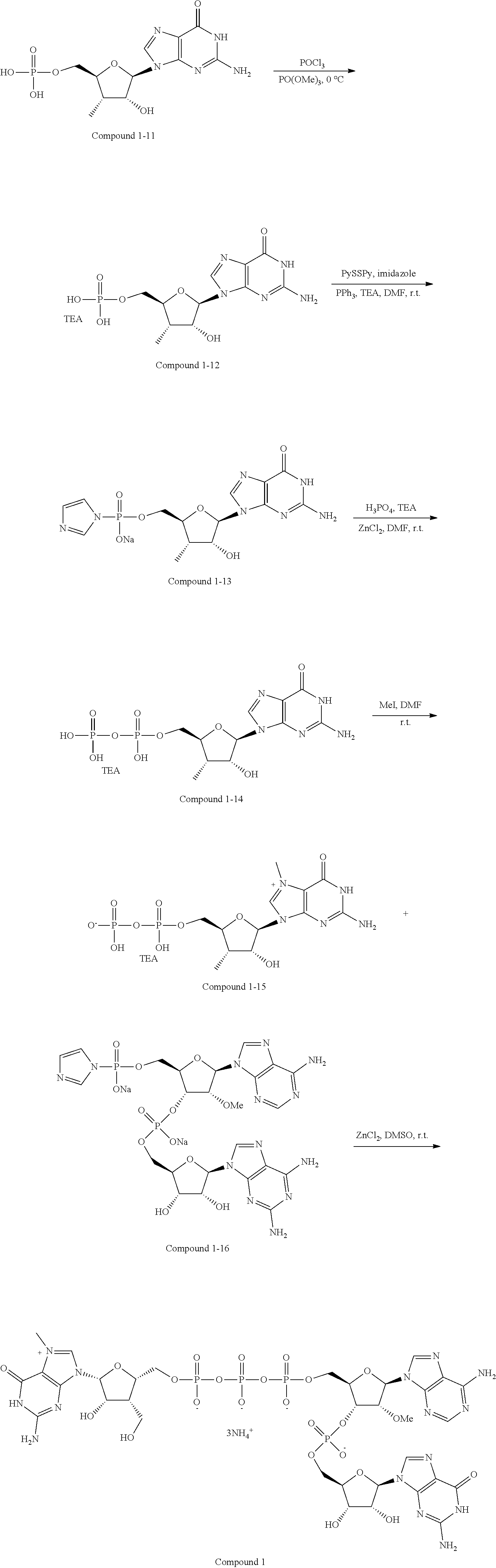

Compound 1-11 (0.5 g, 1.78 mmol) was dissolved in 5 mL of trimethyl phosphate, and phosphorus oxychloride (0.23 mL, 2.67 mmol) was added dropwise at 0 °C. After 2.5 hours, 30 mL of methyl tert-butyl ether was added thereto, and the precipitate was directly purified by an ion exchange column after hydrolysis with water. The product was collected, concentrated and freeze-dried to obtain the triethylamine salt of compound 1-12 (1.13 g, 100%).

The triethylamine salt of compound 1-12 (1.1 g, 3.05 mmol) was dissolved in 10 mL of dimethylformamide, and then 2,2′-dithiodipyridine (1.29 g, 6.1 mmol), imidazole (1 g, 15.25 mmol), triphenylphosphine (1.5 g, 6.1 mmol) and triethylamine (0.4 mL, 3.05 mmol) were added thereto, and stirred at room temperature for 3 hours. Sodium perchlorate (2.2 g, 15.86 mmol) was added to 20 mL of acetone and stirred to dissolve, and the above system was added dropwise to acetone and stirred for half an hour. After standing, the reaction mixture was centrifuged, and the supernatant was poured off, and then acetone was added to disperse and centrifuged again, and the solid was vacuum-dried to obtain compound 1-13 (0.7 g, 56%) after repeating the above operations for four times.

Compound 1-13 (0.7 g, 1.7 mmol), 85% phosphoric acid (0.33 mL, 5.1 mmol), triethylamine (0.66 mL, 5.1 mmol) and zinc chloride (0.22 g, 1.7 mmol) were dissolved in 2 mL of dimethyl sulfoxide. The reaction mixture was stirred at room temperature for 5 hours. After the reaction was completed, 30 mL of methyl tert-butyl ether was added thereto. The precipitate was dissolved in water and directly purified by an ion exchange column. The product was collected, concentrated and freeze-dried to obtain the triethylamine salt of compound 1-14 (0.65 g, 93%).

The triethylamine salt of compound 1-14 (0.65 g, 1.47 mmol) was dissolved in 6 mL of dimethylformamide, and iodomethane (0.89 mL, 14.7 mmol) was added and stirred at room temperature overnight. After the reaction was completed, 10 mL of methyl tert-butyl ether was added thereto, and the precipitate was dissolved in water and directly purified by an ion exchange column. The product was collected, concentrated and freeze-dried to obtain the triethylamine salt of compound 1-15 (0.28 g, 70%).

The triethylamine salt of compound 1-15 (280 mg, 0.61 mmol) and compound 1-16 (460 mg, 0.61 mmol) were dissolved in 10 mL of dimethyl sulfoxide, and zinc chloride (800 mg, 6.1 mmol) was added and stirred for three days at room temperature. After the reaction was completed, the reaction mixture was diluted by adding water and directly purified by an ion exchange column, and the product was collected, concentrated and freeze-dried to obtain the ammonium salt of compound 1 (13.1 mg, 4.3%).

The characterization data of the ammonium salt of compound 1 are MS (m/z): 1142.18 [M−1]⁻; ¹H NMR (500 MHz, D₂O) δ 9.02 (s, 1H), 8.41 (s, 1H), 8.15 (s, 1H), 7.96 (s, 1H), 6.00 (d, J=5.6 Hz, 1H), 5.81 (d, J=5.9 Hz, 1H), 5.79 (s, 1H), 4.95-4.91 (m, 1H), 4.51-4.48 (m, 2H), 4.47-4.41 (m, 3H), 4.34 (s, 1H), 4.30-4.25 (m, 2H), 4.19-4.17 (m, 2H), 4.15-4.13 (m, 2H), 3.99 (s, 3H), 3.43 (s, 3H), 2.36-2.32 (m, 1H), 1.02 (d, J=6.7 Hz, 3H); ³¹P NMR (202 MHz, D₂O) δ −0.94 (s, 1P), −11.50 (m, 2P), −22.89 (m, 1P).

### Example 2: Synthes of Compound 3

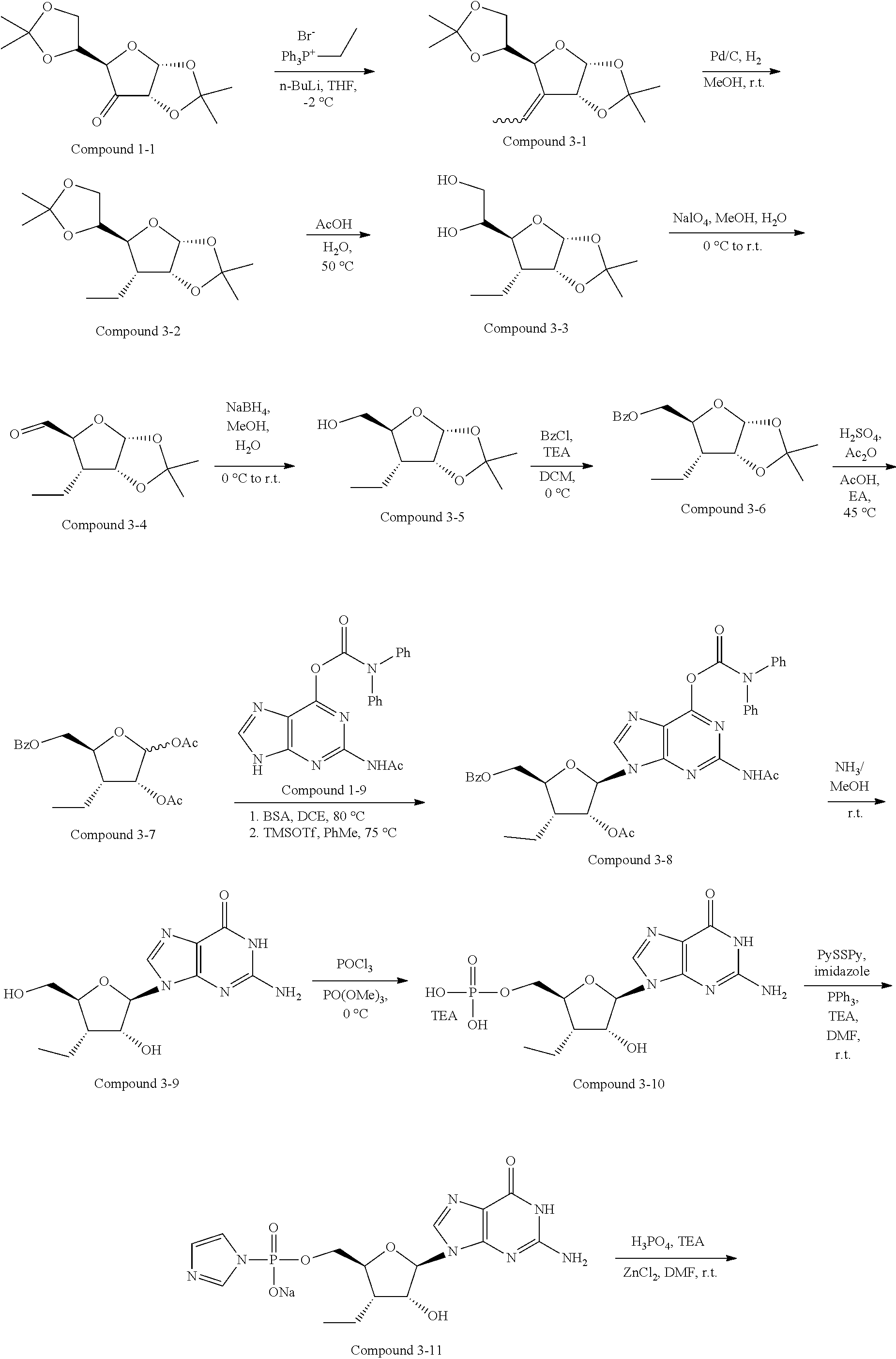

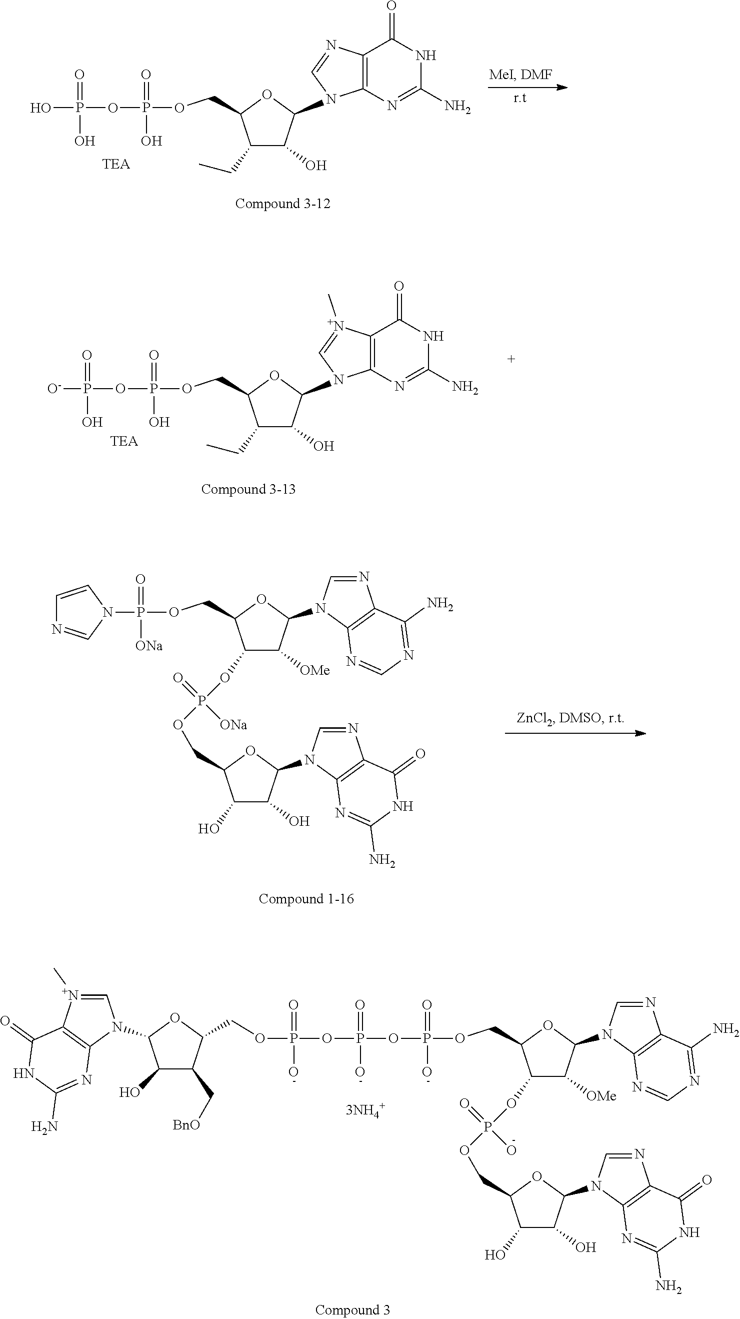

According to the above synthetic route and referring to the synthetic method of compound 1-11, compound 3-9 was synthesized by using ethyltriphenylphosphonium bromide instead of methyltriphenylphosphonium bromide.

The characterization data of compound 3-9 are: MS (m/z): 296.07 [M+H]⁺; ¹H NMR (500 MHz, DMSO) δ 10.58 (s, 1H), 8.02 (s, 1H), 6.44 (s, 2H), 5.72 (s, 1H), 5.58 (d, J=5.6 Hz, 1H), 5.01 (t, J=5.3 Hz, 1H), 4.22 (t, J=5.2 Hz, 1H), 3.85-3.82 (m, 1H), 3.75-3.71 (m, 1H), 3.55-3.50 (m, 1H), 2.17-2.12 (m, 1H), 1.56-1.47 (m, 1H), 1.36-1.28 (m, 1H), 0.91 (t, J=7.3 Hz, 3H).

According to the above synthetic route and referring to the synthetic method of compound 1, compound 3-9 was used instead of compound 1-11 to synthesize the ammonium salt of compound 3.

The characterization data of the ammonium salt of compound 3 are: MS (m/z): 1156.07 [M−1]⁻; ¹H NMR (500 MHz, D₂O) δ 9.01 (s, 1H), 8.37 (s, 1H), 8.06 (s, 1H), 7.95 (s, 1H), 6.02 (d, J=5.5 Hz, 1H), 5.80 (d, J=5.3 Hz, 1H), 5.76 (s, 1H), 4.96-4.92 (m, 1H), 4.70 (t, J=5.2 Hz, 1H), 4.54 (d, J=4.2 Hz, 1H), 4.50-4.48 (m, 2H), 4.46-4.41 (m, 2H), 4.34-4.30 (m, 2H), 4.26-4.18 (m, 3H), 4.16-4.09 (m, 2H), 3.98 (s, 3H), 3.47 (s, 3H), 2.15-2.09 (m, 1H), 1.43-1.39 (m, 2H), 0.88 (t, J=7.3 Hz, 3H); ³¹P NMR (202 MHz, D₂O) δ −0.96 (s, 1P), −11.50 (m, 2P), −22.73 (m, 1P).

### Example 3: Synthes of Compound 5

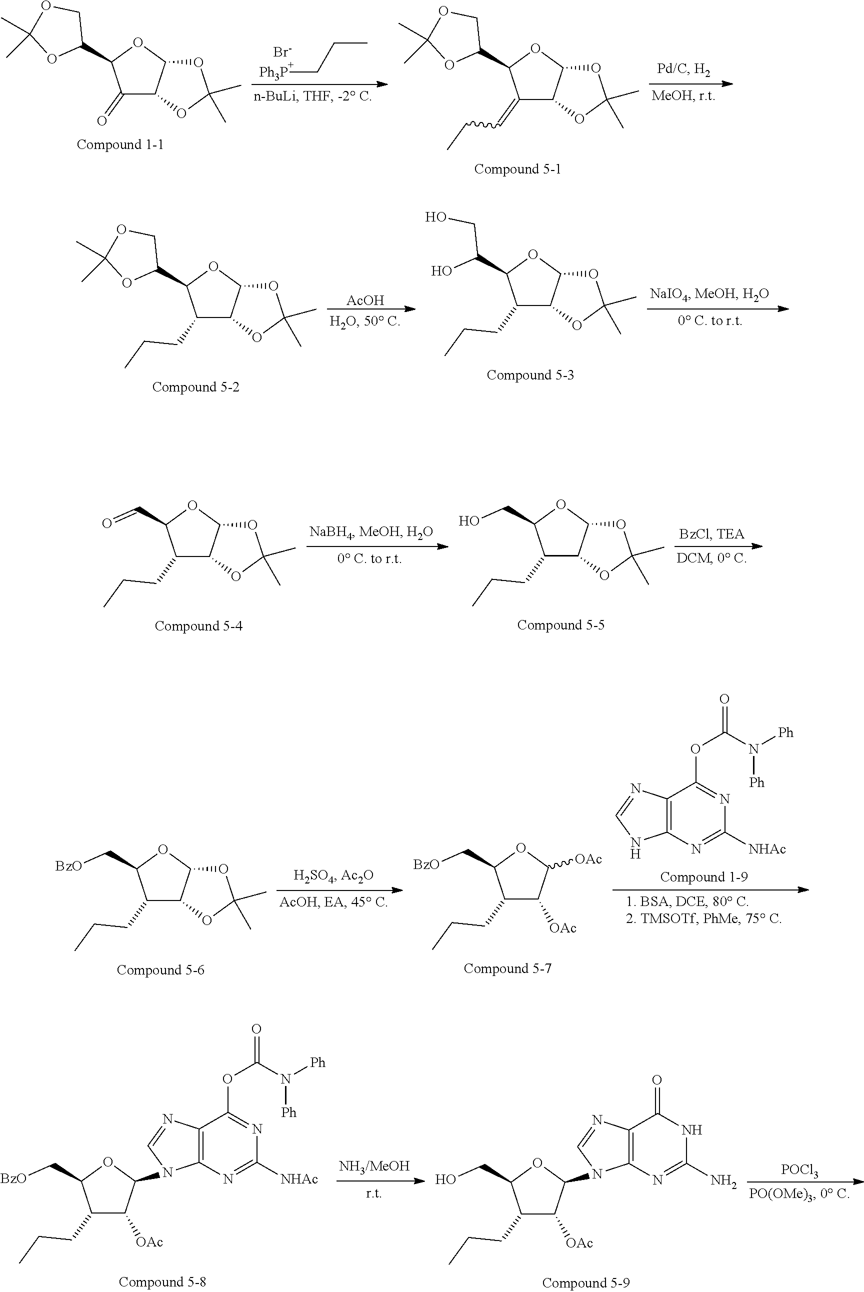

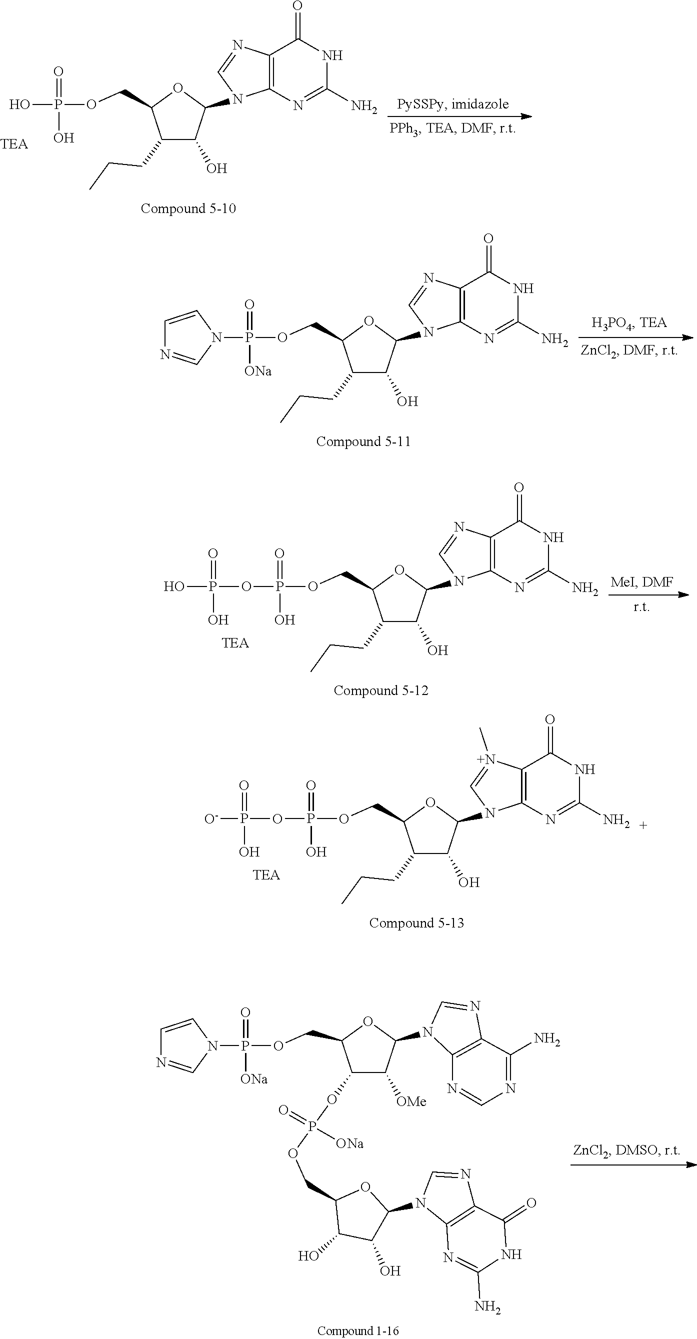

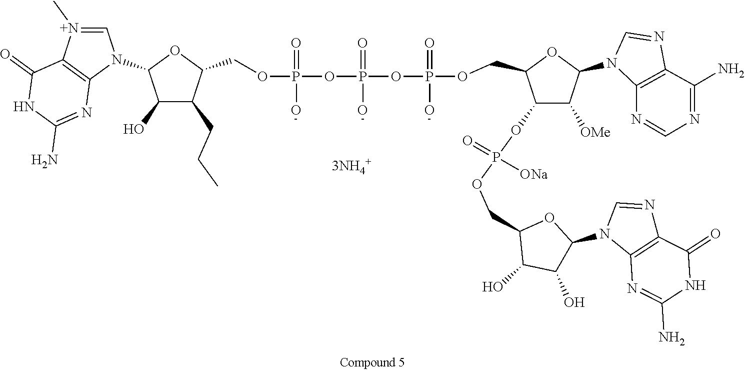

According to the above synthetic route and referring to the synthetic method of compound 1-11, compound 5-9 was synthesized by using propyltriphenylphosphonium bromide instead of methyltriphenylphosphonium bromide.

The characterization data of compound 5-9 are: MS (m/z): 310.01 [M+H]⁺; ¹H NMR (500 MHz, DMSO) δ 10.59 (s, 1H), 8.02 (s, 1H), 6.45 (s, 2H), 5.73 (s, 1H), 5.58 (d, J=5.6 Hz, 1H), 5.02 (t, J=5.3 Hz, 1H), 4.20 (t, J=5.2 Hz, 1H), 3.86-3.82 (m, 1H), 3.76-3.71 (m, 1H), 3.56-3.50 (m, 1H), 2.27-2.20 (m, 1H), 1.56-1.47 (m, 1H), 1.44-1.36 (m, 1H), 1.32-1.26 (m, 1H), 1.24-1.19 (m, 1H), 0.88 (t, J=7.2 Hz, 3H).

According to the above synthetic route and referring to the synthetic method of compound 1, compound 5-9 was used instead of compound 1-11 to synthesize the ammonium salt of compound 5.

The characterization data of the ammonium salt of compound 5 are: MS (m/z): 1170.05 [M−1]⁻; ¹H NMR (500 MHz, D₂O) δ 9.10 (s, 1H), 8.49 (s, 1H), 8.22 (s, 1H), 7.97 (s, 1H), 6.06 (d, J=5.3 Hz, 1H), 5.80 (d, J=5.8 Hz, 1H), 5.77 (s, 1H), 4.96-4.93 (m, 1H), 4.54-4.42 (m, 2H), 4.49-4.42 (m, 3H), 4.34 (m, 2H), 4.26-4.17 (m, 3H), 4.16-4.13 (m, 1H), 4.12-4.09 (m, 1H), 4.00 (s, 3H), 3.46 (s, 3H), 2.24-2.19 (m, 1H), 1.43-1.19 (m, 4H), 0.83 (t, J=6.9 Hz, 3H); ¹P NMR (202 MHz, D₂O) S6-0.92 (s, 1P), −11.61 (m, 2P), −22.81 (m, 1P).

### Example 4: Synthes of Compound 19

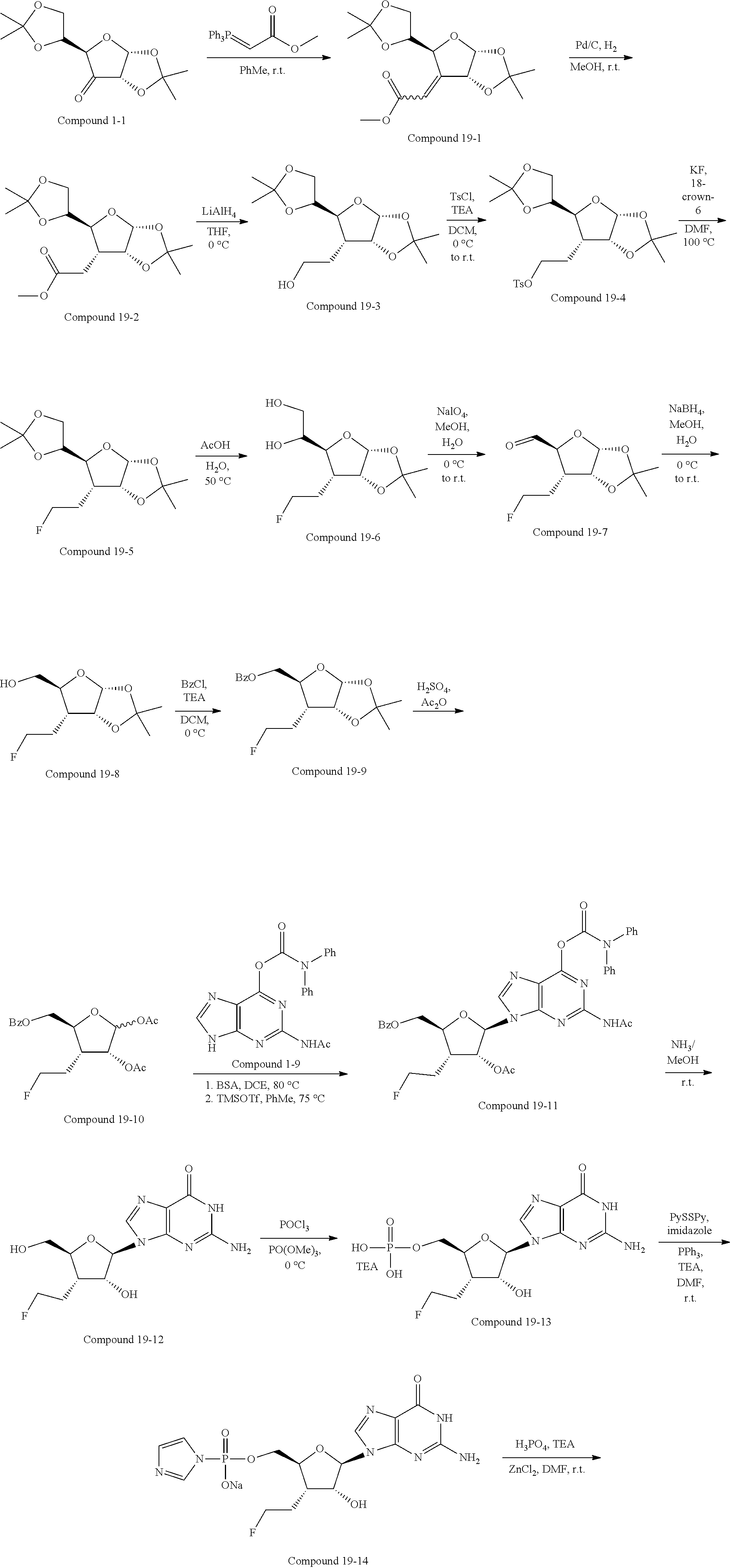

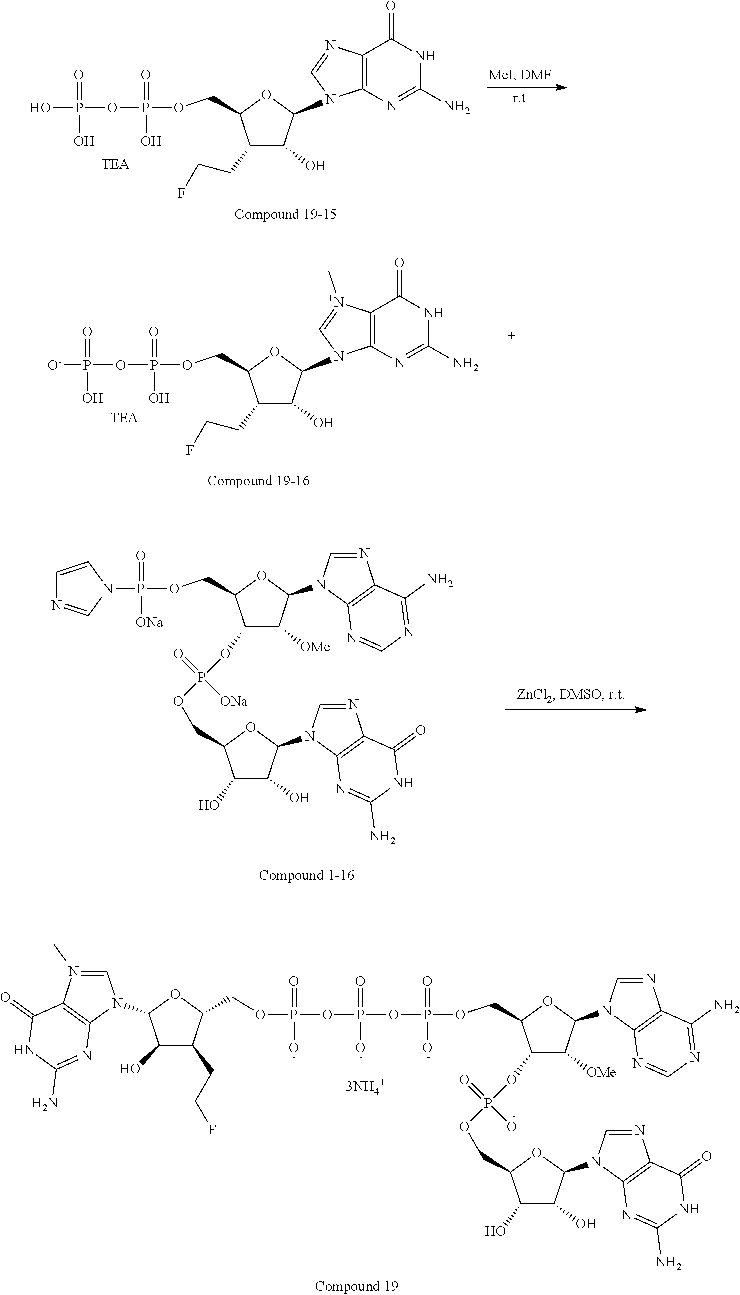

Compound 1-1 (18 g, 69.69 mmol) was dissolved in toluene (120 mL), and methyl (triphenylphosphoranylidene)acetate (25.63 g, 76.66 mmol) was added thereto, and then the reaction mixture was reacted overnight at room temperature. TLC showed that the reaction was completed. The reaction mixture was added with water (100 mL) and extracted with ethyl acetate (100 mL×3). The organic phases were combined, washed with saturated brine, dried over anhydrous sodium sulfate, concentrated under reduced pressure, and purified by column chromatography to obtain 19.77 g of compound 19-1 as a light yellow oil.

Compound 19-1 (19.77 g, 62.96 mmol) was dissolved in tetrahydrofuran (200 mL), and wet palladium on carbon (2 g, 10%) was added and reacted under hydrogen pressure overnight. TLC showed that the reaction was completed. The reaction was stopped, and the insoluble material was removed by filtration. The filtrate was concentrated under reduced pressure to obtain 20.4 g of compound 19-2 as a colorless and transparent oil with a yield of 102.5%.

LiAlH₄ (2.65 g, 69.80 mmol) was added to anhydrous tetrahydrofuran (100 mL), cooled to 0 °C, and a solution of compound 19-2 (18.4 g, 58.16 mmol) in tetrahydrofuran (100 mL) was added dropwise. After 3 hours of reaction, TLC showed that the reaction was completed, and the reaction was stopped. Ethyl acetate (6 mL), water (10 mL) and anhydrous sodium sulfate were sequentially added thereto, stirred for 2 minutes, filtered and concentrated under reduced pressure to obtain 15.1 g of compound 19-3 as a colorless oil with a yield of 90%.

Compound 19-3 (8.2 g, 28.47 mmol) was dissolved in dichloromethane (80 mL), then triethylamine (5.76 g, 56.94 mmol) and DMAP (348 mg, 2.85 mmol) were added thereto, cooled to 0 °C, and a solution of p-toluenesulfonyl chloride (5.97 g, 31.32 mmol) in dichloromethane (80 mL) was added dropwise. After the dropwise addition was completed, the reaction mixture was transferred to room temperature and reacted overnight. TLC showed that a small amount of starting material remained, then the reaction was stopped, and water (100 mL) was added thereto and stirred for 5 minutes. The phases were separated, and the aqueous phase was extracted with dichloromethane (100 mL×2). The organic phases were combined, concentrated under reduced pressure, and purified by column chromatography to obtain 9.17 g of compound 19-4 with a yield of 76%.

Compound 19-4 (6.72 g, 15.20 mmol) was dissolved in DMF (70 mL), then potassium fluoride (4.41 g, 76 mmol) and 18-crown-6-ether (10 g, 38 mmol) were added thereto, and the reaction mixture was heated to 100 °C and reacted overnight under a nitrogen atmosphere. TLC showed that the reaction was completed, and the reaction was stopped and cooled to room temperature. The reaction mixture was concentrated under reduced pressure to remove DMF, added with water (100 mL), and extracted with ethyl acetate (50 mL×3). The organic phases were combined, concentrated under reduced pressure, and purified by column chromatography to obtain 2.54 g of compound 19-5 with a yield of 57.6%.

According to the above synthetic route and referring to the synthetic method of compound 1-11, compound 19-5 was used instead of compound 1-3 to synthesize compound 19-12.

The characterization data of compound 19-12 are: MS (m/z): 314.12 [M+H]⁺; ¹H NMR (500 MHz, DMSO) δ 10.59 (s, 1H), 8.01 (s, 1H), 6.45 (s, 2H), 5.74-5.73 (m, 2H), 5.05 (t, J=5.3 Hz, 1H), 4.62-4.52 (m, 1H), 4.52-4.42 (m, 1H), 4.26 (t, J=5.2 Hz, 1H), 3.90 (d, J=9.7 Hz, 1H), 3.78-3.70 (m, 1H), 3.62-3.48 (m, 1H), 2.37 (m, 1H), 1.94 (m, 1H), 1.73 (m, 1H).

According to the above synthetic route and referring to the synthetic method of compound 1, compound 19-12 was used instead of compound 1-11 to synthesize the ammonium salt of compound 19.

The characterization data of the ammonium salt of compound 19 are: MS (m/z): 1174.14 [M−1]⁻; ¹H-NMR (500 MHz, D₂O): δ 9.13 (s, 1H), 8.55 (s, 1H), 8.27 (s, 1H), 8.00 (s, 1H), 6.09 (t, J=4.2 Hz, 1H), 5.82 (d, J=4.1 Hz, 1H), 5.80 (d, J=5.8 Hz, 1H), 4.96-4.92 (m, 1H), 4.64-4.59 (m, 2H), 4.53-4.44 (m, 5H), 4.34 (br, 2H), 4.27-4.23 (m, 2H), 4.19-4.15 (m, 3H), 4.02 (s, 3H), 3.46 (s, 3H), 2.45-2.39 (m, 1H), 2.02-1.91 (m, 1H), 1.89-1.77 (m, 1H). ³¹P-NMR (200 MHz, D₂O): δ −0.90 (s, 1P), −11.62(m, 2P), −22.81(m, 1P).

### Example 5: Synthes of Compound 193

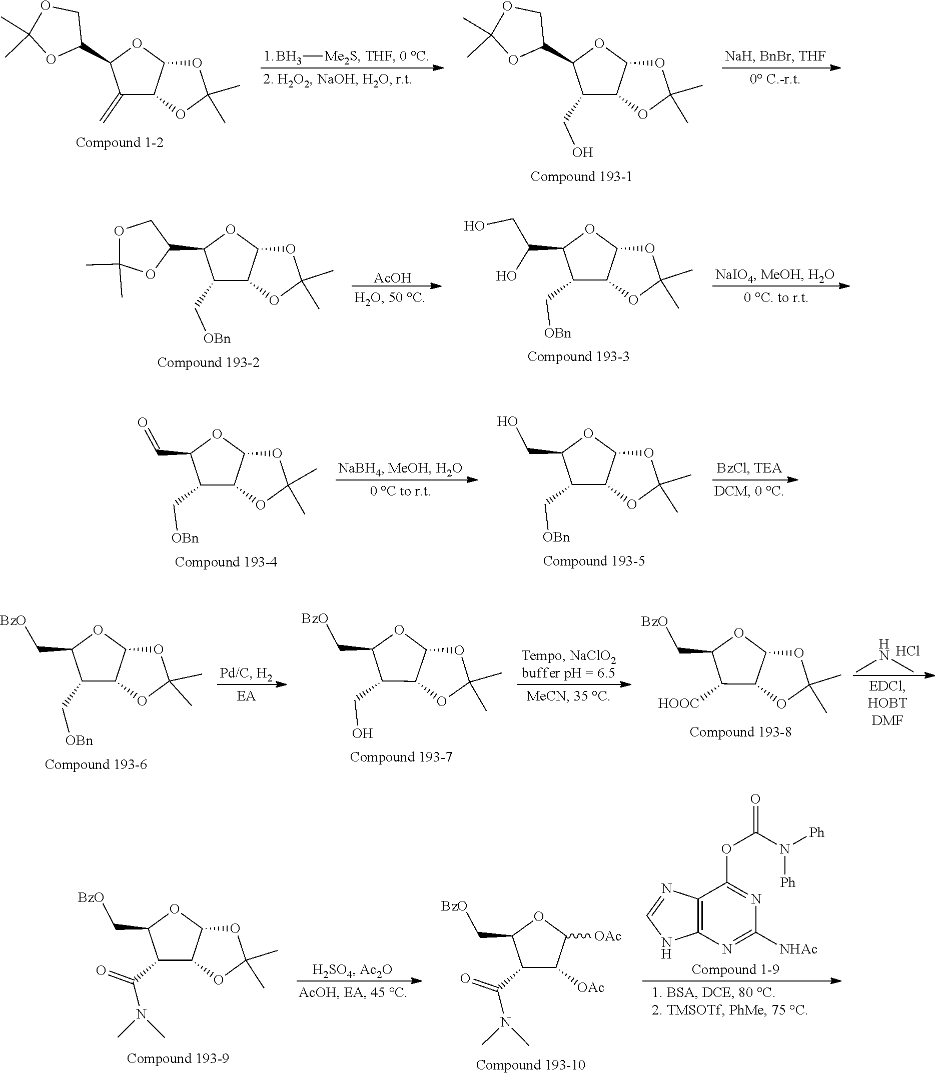

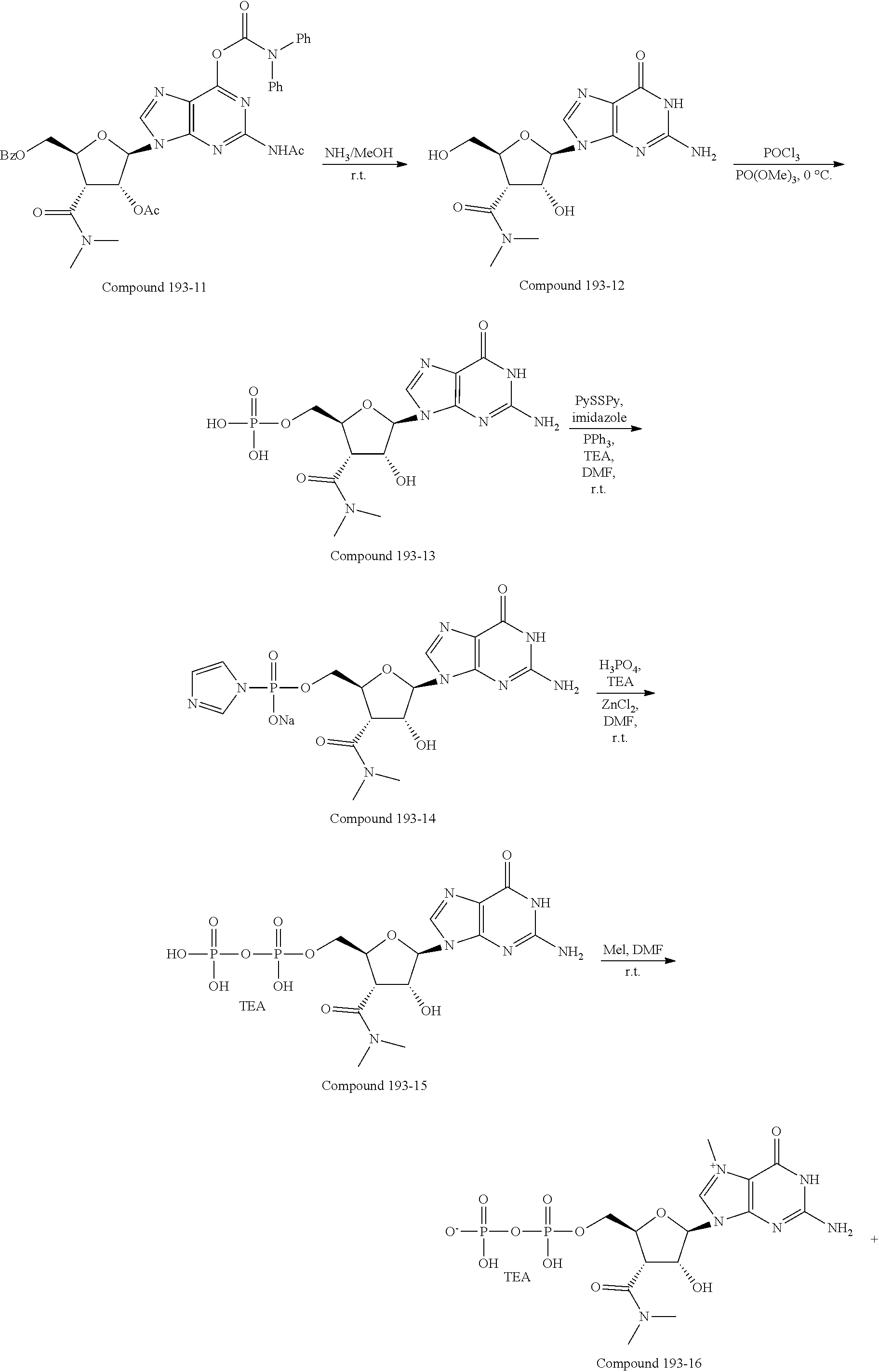

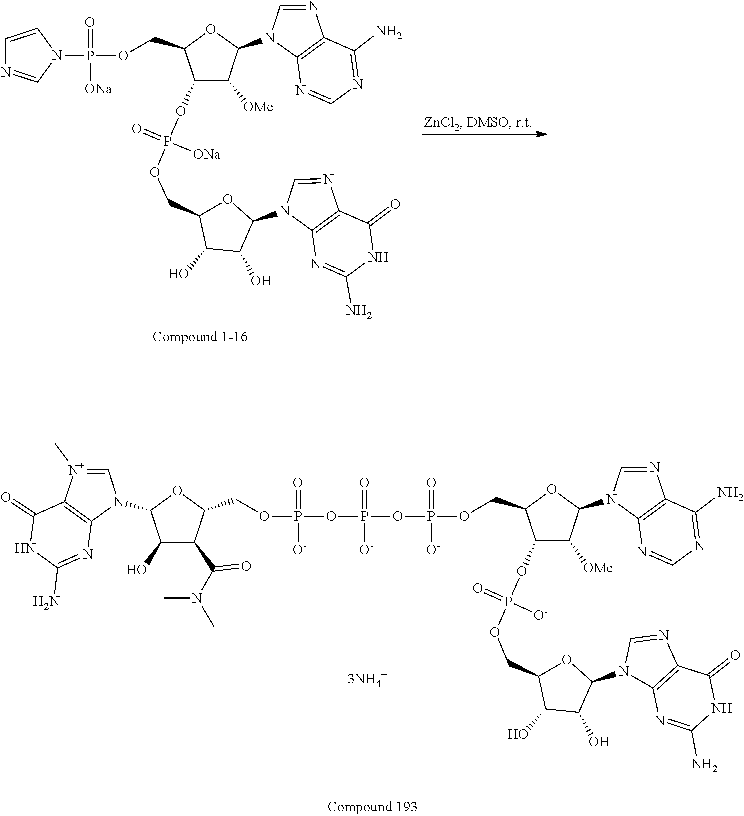

According to the above synthesis route, compound 1-2 (30.0 g, 117 mmol) was dissolved in anhydrous tetrahydrofuran (300 mL) at room temperature under a nitrogen atmosphere, cooled to 0 °C, and then a borane dimethyl sulfide solution (23.4 mL, 234 mmol, 10 M) was added dropwise. After the dropwise addition was completed, the reaction mixture was reacted at 0 °C for 20 hours. Sodium hydroxide solution (28 g, 702 mmol, 350 mL of water) was then added dropwise, followed by the dropwise addition of hydrogen peroxide (80 mL, 30% in water), and the reaction mixture was stirred and reacted for 2 hours at room temperature. Ethyl acetate (200 mL) was added for phase separation. The organic phase was sequentially washed with water (100 mL×2) and saturated brine, dried over anhydrous sodium sulfate, and concentrated under reduced pressure to obtain 20.5 g of compound 193-1, which was directly used in the next reaction without purification.

Sodium hydride (2.18 g, 54.70 mmol) was added to a solution of compound 193-1 (10 g, 36.47 mmol) in tetrahydrofuran (100 mL) in an ice bath under a nitrogen atmosphere, and the reaction mixture was transferred to room temperature, stirred and reacted for 30 minutes. Benzyl bromide (6.5 mL, 54.70 mmol) was added dropwise to the system under an ice bath and the reaction mixture was stirred and reacted at room temperature for 1 hour. Under an ice bath, ice acetic acid (4.2 mL, 72.94 mmol) was added to the system with stirring, and transferred to room temperature. Ethyl acetate (70 mL) and water (100 mL) were added thereto, and the phases were separated. The organic phase was sequentially washed with water (100 mL×3) and saturated brine, dried over anhydrous sodium sulfate, and the organic phase was concentrated under reduced pressure. The residue was purified by silica gel column chromatography (petroleum ether/ethyl acetate=8/1) to obtain 10.89 g of compound 193-2.

Referring to the synthetic method of compound 1-7, compound 193-2 was used instead of compound 1-3 to synthesize compound 193-6.

10% palladium on carbon (0.60 g) was added to a solution of compound 193-6 (8.50 g, 39.04 mmol) in methanol (60 mL) at room temperature, and the reaction mixture was stirred and reacted overnight under a hydrogen atmosphere. The reaction mixture was subjected to suction filtration, and the filtrate was concentrated under reduced pressure to obtain 6.80 g of compound 193-7.

At room temperature, 2,2,6,6-tetramethylpiperidine 1-oxyl (223 mg, 1.43 mmol) was added to a solution of compound 193-7 (6.3 g, 20.45 mmol) in acetonitrile (100 mL) and a buffer solution (75 mL) of pH=6.7, and the reaction mixture was heated to 35 °C, stirred and reacted for 30 minutes. Sodium hypochlorite (0.5 mL, 0.41 mmol) solution (10 mL), sodium chlorite (3.70 g, 40.90 mmol) solution (20 mL) were added to the system, and the reaction mixture was stirred and reacted at room temperature overnight. Water (200 mL) was added to the system, and the pH was adjusted to 3 with dilute hydrochloric acid. The mixture was extracted with dichloromethane/methanol=8/1 (200 mL×5), dried over anhydrous sodium sulfate, and concentrated under reduced pressure to obtain 5.66 g of compound 193-8.

At room temperature, under a nitrogen atmosphere, 1-ethyl-(3-dimethylaminopropyl)carbodiimide hydrochloride (6.73 g, 35.14 mmol) was added to a solution of compound 193-8 (5.66 g, 17.57 mmol), dimethylamine hydrochloride (2.15 g, 26.35 mmol), 1-hydroxybenzotriazole (0.47 g, 3.51 mmol) and triethylamine (7.3 mL, 52.71 mmol) in N,N-dimethylformamide (74 mL), and the reaction mixture was stirred and reacted at room temperature. Water (100 mL) and ethyl acetate (200 mL) were added to the system. The phases were separated, and the aqueous phase was washed with dichloromethane (100 mL×3), then the organic phase was concentrated under reduced pressure. The residue was purified by silica gel column chromatography (petroleum ether/ethyl acetate=1/2) to obtain 3.80 g of compound 193-9.

Referring to the synthetic method of compound 1-11, compound 193-9 was used instead of compound 1-7 to synthesize compound 193-12.

The characterization data of compound 193-12 are: MS (m/z): 339.10 [M+H]⁺; ¹H NMR (500 MHz, DMSO) δ 9.15 (br, 1H), 7.96 (s, 1H), 6.51 (s, 2H), 5.85 (br, 1H), 5.72 (d, J=3.9 Hz, 1H), 4.99 (br, 1H), 4.71 (m, 1H), 4.46-4.43 (m, 1H), 3.64-3.61 (m, 2H), 3.45 (d, J=9.4 Hz, 1H), 3.02 (s, 3H), 2.85 (s, 3H).

According to the above synthetic route and referring to the synthetic method of compound 1, compound 193-12 was used instead of compound 1-11 to synthesize the ammonium salt of compound 193.

The characterization data of the ammonium salt of compound 193 are: MS (m/z): 1199.08 [M−1]⁻; ¹H-NMR (500 MHz, D₂O): δ 8.38 (s, 1H), 8.10 (s, 1H), 7.98 (s, 1H), 6.01 (d, J=5.8 Hz, 1H), 5.88 (d, J=2.5 Hz, 1H), 5.82 (d, J=5.5 Hz, 1H), 4.94-4.91 (m, 2H), 4.89-4.88 (m, 1H), 4.51-4.49 (m, 2H), 4.43-4.40 (m, 2H), 4.34 (s, 1H), 4.29-4.23 (m, 2H), 4.21-4.18 (m, 2H), 4.11-4.07 (m, 1H), 4.01 (s, 3H), 3.81 (t, J=6.8 Hz, 1H), 3.43 (s, 3H), 3.06 (s, 3H), 2.94 (s, 3H). ³¹P-NMR (200 MHz, D₂O): δ −0.94 (s, 1P), −11.36 (d, J=18.4 Hz, 1P), −11.57 (d, J=17.6 Hz, 1P), −22.69 (t, J=18.0 Hz, 1P).

### Example 6: Synthes of Compound 289

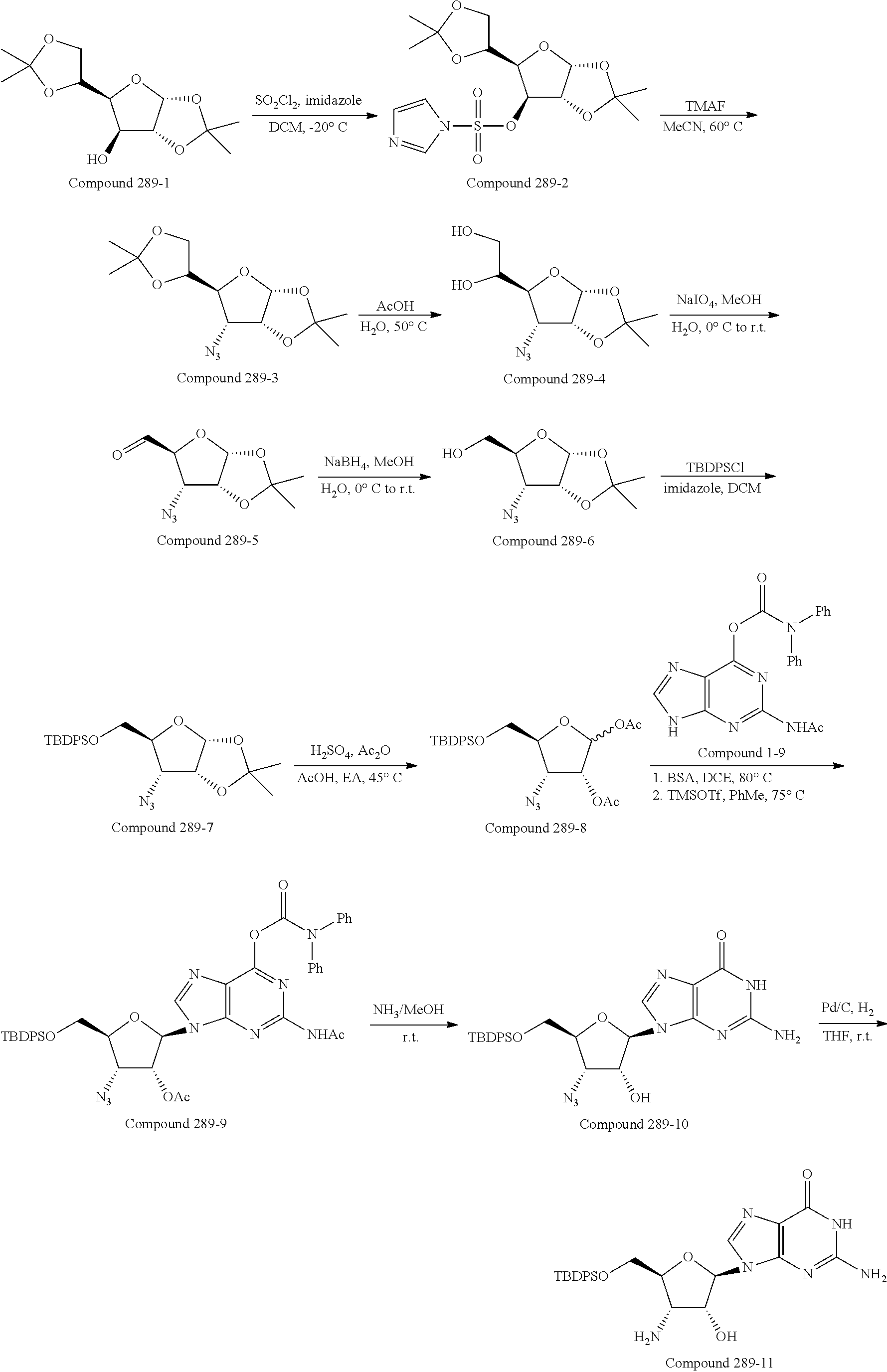

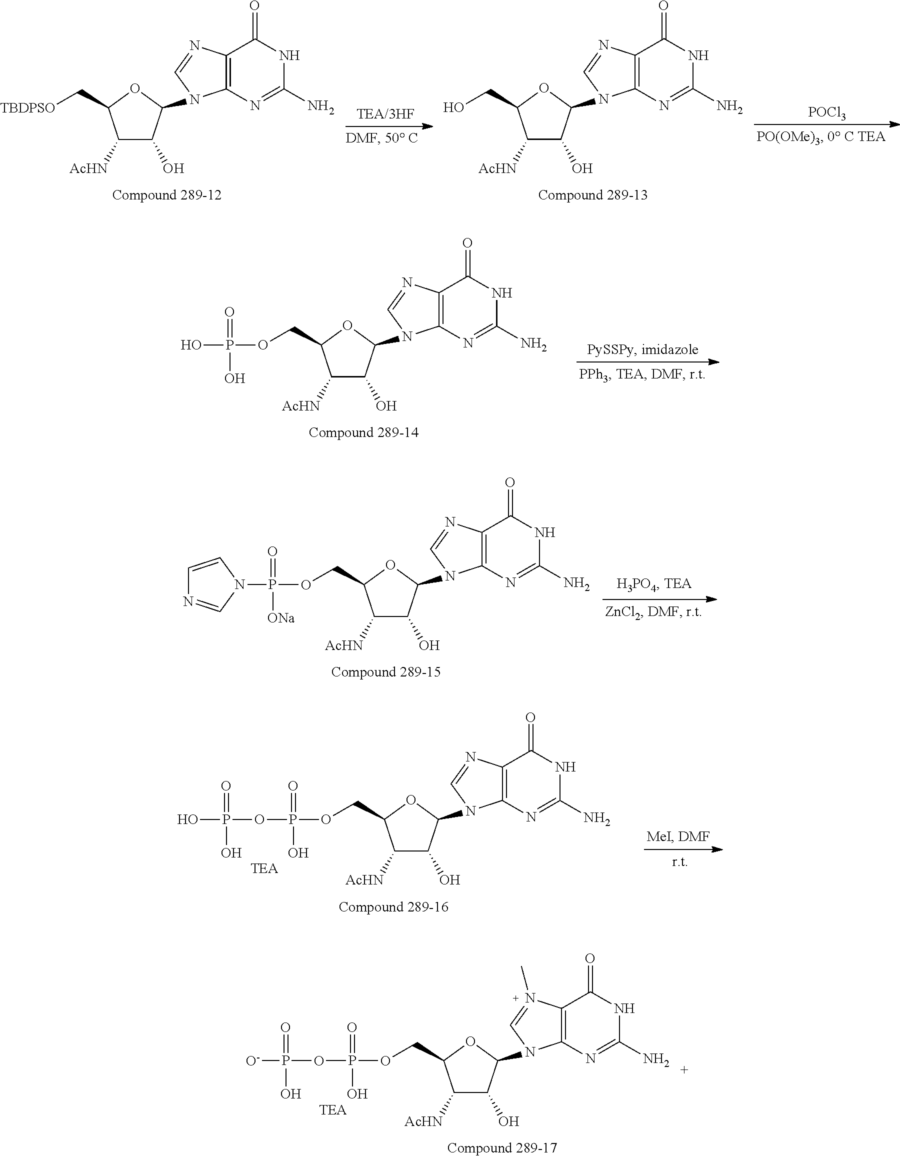

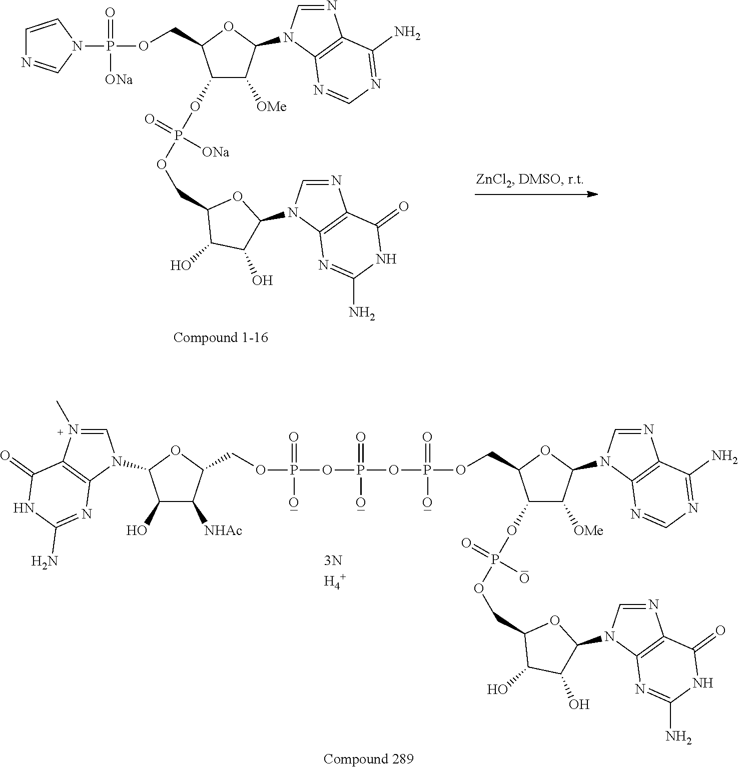

According to the above synthetic route, compound 289-1 (260.28 g, 1 mol) and imidazole (340 g, 5 mol) were dissolved in 2 L of DCM at room temperature and cooled to −20 °C. Sulfonyl chloride (175 g, 1.3 mol, diluted in 50 mL of DCM) was added dropwise, and the reaction mixture was stirred and reacted at −20 °C for 2 hours after the dropwise addition was completed, then slowly warmed to room temperature, stirred and reacted overnight. The organic phase was washed once with water (1 L), saturated sodium bicarbonate and saturated sodium chloride, respectively, and the organic phase was dried, then concentrated to about 600 mL, then 600 mL of petroleum ether was added dropwise, and the mixture was filtered to remove the solid by-products. The organic phase was further concentrated to dryness, and then slurried with DCM (500 mL) and petroleum ether (1.3 L) to obtain 280 g of compound 289-2.

Compound 289-2 (83.0 g, 212.82 mmol) and sodium azide (55.3 g, 850.6 mmol) were dissolved in 830 mL DMF at room temperature. Then the reaction mixture was stirred and reacted at 50 °C for 2 days, cooled to room temperature and filtered. The filtrate was concentrated under reduced pressure, and the residue was purified by silica gel column chromatography (petroleum ether/ethyl acetate=5/1) to obtain 27.0 g of compound 289-3.

Referring to the synthetic method of compound 1-6, compound 289-3 was used instead of compound 1-3 to synthesize compound 289-6.

In an ice bath, under a nitrogen atmosphere, TBDPSCl (26.0 g, 94.6 mmol) was slowly added dropwise to a solution of compound 289-6 (16.00 g, 74.35 mmol) and imidazole (10.12 g, 148.70 mmol) in dichloromethane (160 mL). After the addition was completed, the reaction mixture was stirred and reacted at room temperature overnight. Water (200 mL) was added to the system, and the mixture was extracted with dichloromethane (100 mL×3). The organic phases were combined and concentrated under reduced pressure. The residue was purified by silica gel column chromatography (petroleum ether/ethyl acetate=5/1) to obtain 35.00 g of compound 289-7.

Referring to the synthetic method of compound 1-10, compound 289-7 was used instead of compound 1-7 to synthesize compound 289-10.

At room temperature, compound 289-10 (5.00 g, 9.15 mmol) was dissolved in 10 mL of THF, then Pd/C (500 mg) was added thereto. The reaction system was replaced with hydrogen, and the reaction mixture was stirred and reacted overnight at room temperature. 100 mL of DMF was added thereto, and the mixture was filtered. The filtrate was concentrated under reduced pressure to obtain 4.8 g of compound 289-11.

At room temperature, compound 289-11 (2.08 g, 4.00 mmol) was dissolved in 15 mL of DMF, cooled to 0 °C, and added dropwise with acetic anhydride. After the dropwise addition was completed, the reaction mixture was slowly warmed to room temperature, stirred and reacted overnight. The reaction mixture was concentrated under reduced pressure, and the residue was purified by silica gel column chromatography (dichloromethane/methanol=1/10) to obtain 1.50 g of compound 289-12.

Compound 289-12 (1.50 g, 2.70 mmol) was dissolved in 15 mL of DMF at room temperature, then triethylamine hydrofluoride (869 mg, 5.40 mmol) and triethylamine (545 mg, 5.4 mmol) were added thereto, and the reaction mixture was stirred and reacted at 50 °C overnight. The reaction mixture was concentrated under reduced pressure, and the residue was subjected to preparative separation under medium pressure to obtain 350 mg of compound 289-13.

The characterization data of compound 289-13 are: MS (m/z): 325.11 [M+H]⁺; ¹H NMR (500 MHz, DMSO) δ 10.62 (s, 1H), 7.99 (s, 1H), 7.95 (d, J=7.6 Hz, 1H), 6.44 (s, 2H), 5.85 (d, J=4.9 Hz, 1H), 5.73 (d, J=2.8 Hz, 1H), 5.07 (t, J=5.3 Hz, 1H), 4.37-4.30 (m, 2H), 3.94-3.91 (m, 1H), 3.70-3.66 (m, 1H), 3.53-3.48 (m, 1H), 1.88 (s, 3H).

According to the above synthetic route and referring to the synthetic method of compound 1, compound 289-13 was used instead of compound 1-11 to synthesize the ammonium salt of compound 289.

The characterization data of the ammonium salt of compound 289 are: MS (m/z): 1185.00 [M−1]⁻; ¹H-NMR (500 MHz, D₂O): ¹H NMR (500 MHz, D₂O) δ 9.06 (s, 1H), 8.45 (s, 1H), 8.18 (s, 1H), 8.00 (s, 1H), 6.05 (d, J=5.3 Hz, 1H), 5.89 (s, 1H), 5.84 (d, J=5.9 Hz, 1H), 4.97-4.94 (m, 1H), 4.76-4.73 (m, 1H), 4.63 (d, J=4.4 Hz, 1H), 4.54-4.48 (m, 3H), 4.46 (t, J=4.9 Hz, 1H), 4.38 (m, 1H), 4.35-4.32 (m, 2H), 4.29-4.26 (m, 1H), 4.24-4.20 (m, 2H), 4.17-4.13 (m, 1H), 4.00 (s, 3H), 3.53 (m, 1H), 3.45 (s, 3H), 2.01 (s, 3H); ³¹P NMR (202 MHz, D₂O) 8-0.95 (s, 1P), −11.58 (m, 2P), −22.62 (m, 1P).

### Example 7: Synthes of Compound 2

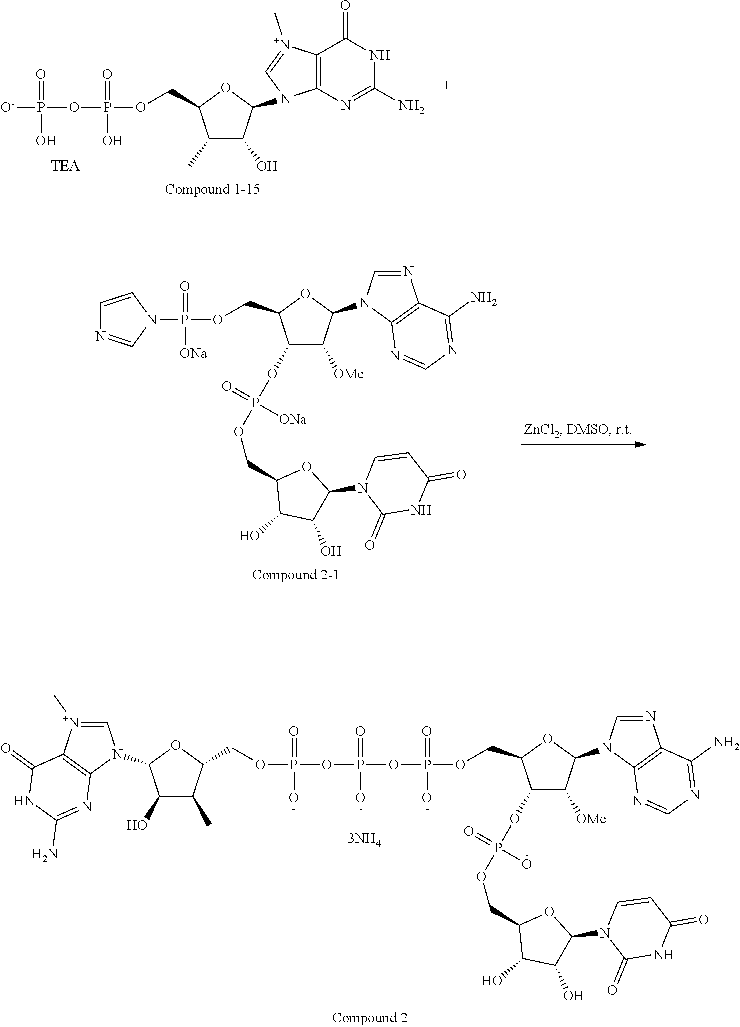

According to the above synthetic route and referring to the synthetic method of compound 1, compound 2-1 was used instead of compound 1-16 to synthesize the ammonium salt of compound 2.

The characterization data of the ammonium salt of compound 2 are: MS (m/z): 1102.96 [M−1]⁻; ¹H NMR (500 MHz, D₂O) δ 9.02 (s, 1H), 8.40 (s, 1H), 8.13 (s, 1H), 7.86 (d, J=8.1 Hz, 1H), 6.02 (d, J=5.6 Hz, 1H), 5.80 (d, J=5.9 Hz, 1H), 5.80-5.78 (m, 2H), 4.96-4.91 (m, 1H), 4.52-4.48 (m, 2H), 4.47-4.41 (m, 3H), 4.34 (s, 1H), 4.30-4.23 (m, 2H), 4.19-4.16 (m, 2H), 4.15-4.12 (m, 2H), 4.00 (s, 3H), 3.43 (s, 3H), 2.36-2.30 (m, 1H), 1.02 (d, J=6.7 Hz, 3H); ³¹P NMR (202 MHz, D₂O) δ −1.05 (s, 1P), −11.63 (m, 2P), −22.92 (m, 1P).

### Example 8: Synthes of Compound 716

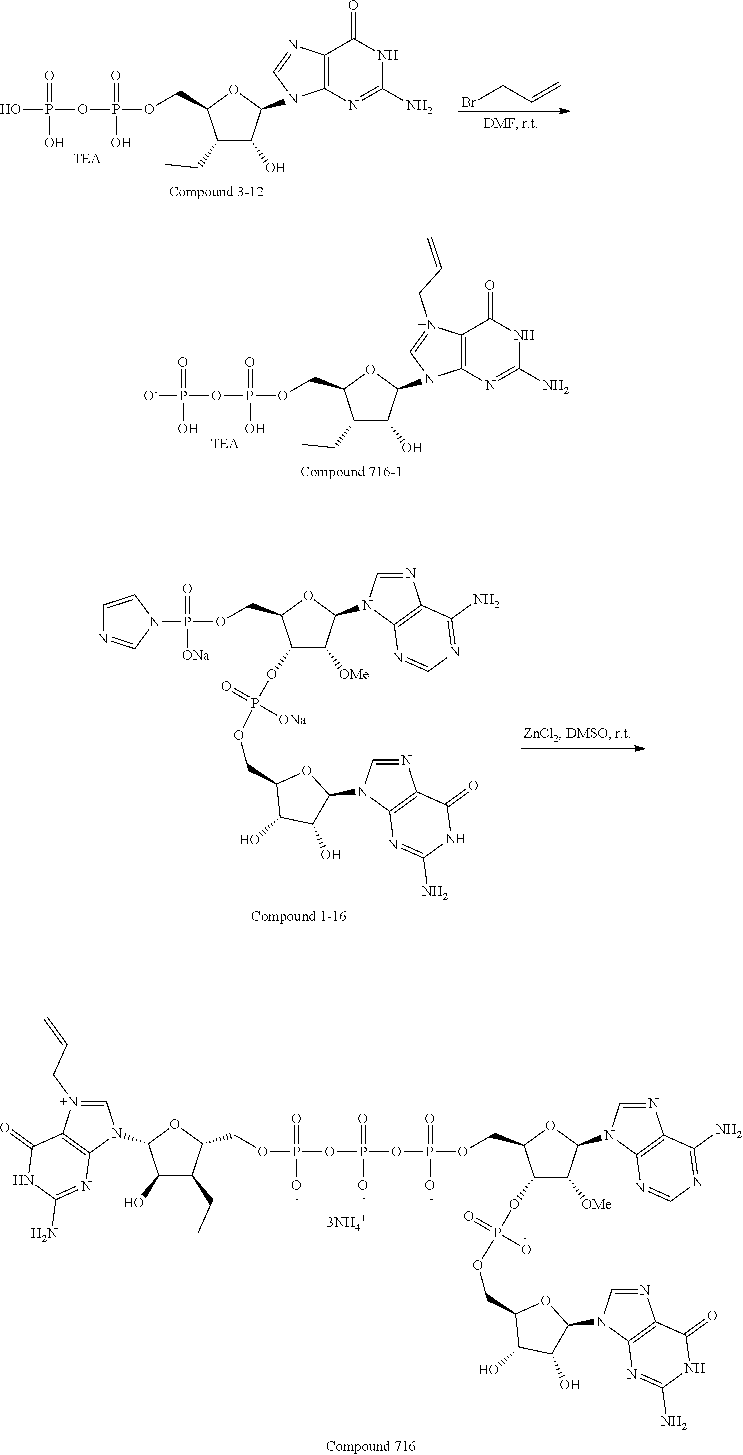

According to the above synthetic route and referring to the synthesis method of compound 1, compound 3-12 was used instead of compound 1-14, and 3-bromopropene was used instead of iodomethane to synthesize the ammonium salt of compound 716.

The characterization data of the ammonium salt of compound 716 are: MS (m/z): 1182.02 [M−1]⁻; ¹H NMR (500 MHz, D₂O) δ 9.13 (s, 1H), 8.46 (s, 1H), 8.19 (s, 1H), 7.96 (s, 1H), 6.06-5.98 (m, 2H), 5.81 (d, J=6.0 Hz, 1H), 5.78 (s, 1H), 5.34 (s, 1H), 5.31 (d, J=5.6 Hz, 1H), 4.97 (d, J=5.7 Hz, 2H), 4.95-4.92 (m, 1H), 4.83-4.81 (m, 1H), 4.58 (d, J=4.4 Hz, 1H), 4.50-4.48 (m, 2H), 4.46-4.42 (m, 2H), 4.35-4.33 (m, 1H), 4.32-4.28 (m, 1H), 4.25-4.11 (m, 5H), 3.43 (s, 3H), 2.15-2.12 (m, 1H), 1.46-1.40 (m, 2H), 0.89 (t, J=7.4 Hz, 3H); ³¹P NMR (202 MHz, D₂O) δ −0.94 (s, 1P), −11.61 (m, 2P), −22.96 (m, 1P).

### Example 9: Synthes of Compound 717

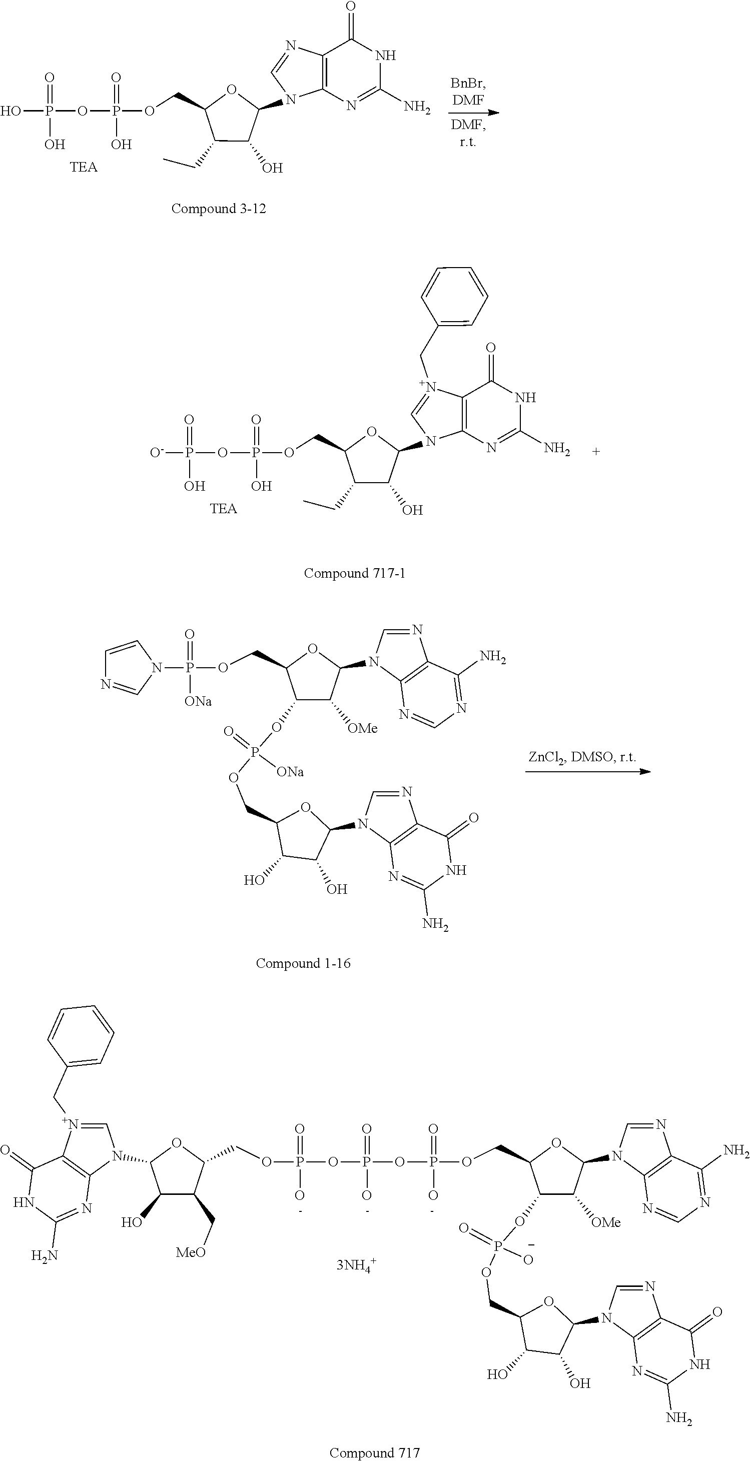

According to the above synthetic route and referring to the synthesis method of compound 1, compound 3-12 was used instead of compound 1-14, and benzyl bromide was used instead of iodomethane to synthesize the ammonium salt of compound 717.

The characterization data of the ammonium salt of compound 717 are: MS (m/z): 1232.05 [M−1]⁻; ¹H NMR (500 MHz, D₂O) δ 9.38 (s, 1H), 8.40 (s, 1H), 8.14 (s, 1H), 7.93 (s, 1H), 7.32-7.20 (m, 5H), 5.93-5.92 (m, 1H), 5.80-5.79 (m, 2H), 5.54-5.50 (m, 2H), 4.94-4.92 (m, 1H), 4.64-4.60 (m, 1H), 4.49-4.45 (m, 3H), 4.40 (m, 1H), 4.33-4.29 (m, 2H), 4.22-4.15 (m, 5H), 3.41 (s, 3H), 2.21-2.17 (m, 1H), 1.49-1.43 (m, 2H), 0.90 (t, J=7.3 Hz, 3H); ³¹P NMR (202 MHz, D₂O) δ −0.91 (s, 1P), −11.48 (m, 2P), −22.91 (m, 1P).

### Example 10: Synthes of Compound 721

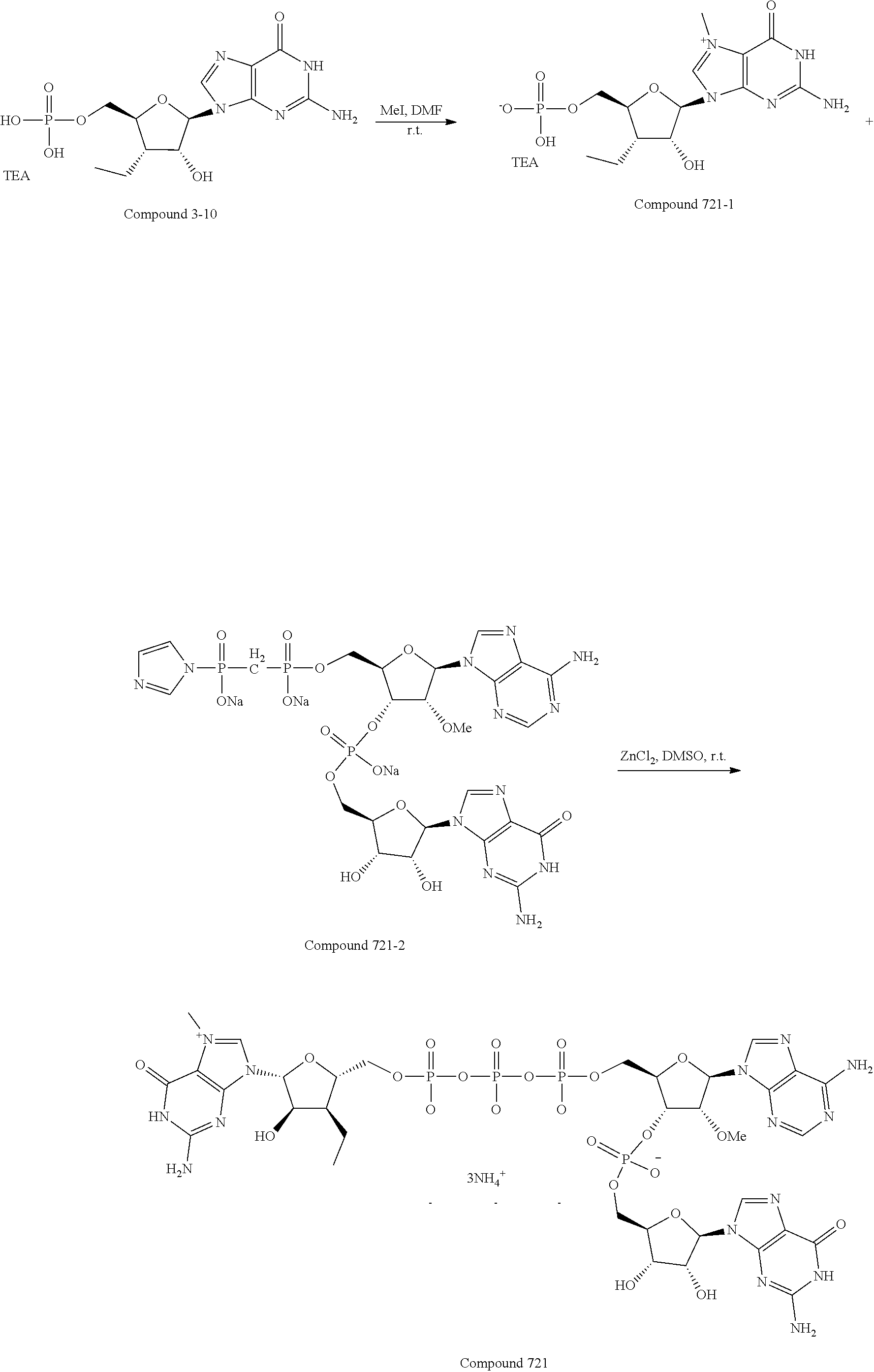

According to the above synthetic route and referring to the synthesis method of compound 1, compound 3-10 was used instead of compound 1-14, and compound 721-2 was used instead of compound 1-16 to synthesize the ammonium salt of compound 721.

The characterization data of the ammonium salt of compound 721 are: MS (m/z): 1154.04 [M−1]⁻; ¹H NMR (500 MHz, D₂O) δ 8.42 (s, 1H), 8.12 (s, 1H), 7.96 (s, 1H), 6.03 (d, J=5.8 Hz, 1H), 5.85-5.83 (m, 2H), 4.95-4.92 (m, 1H), 4.58 (d, J=4.4 Hz, 1H), 4.53-4.50 (m, 2H), 4.46-4.44 (m, 2H), 4.35 (m, 1H), 4.24-4.21 (m, 5H), 4.14-4.11 (m, 1H), 4.02 (s, 3H), 3.44 (s, 3H), 2.47-2.37 (m, 2H), 2.20-2.14 (m, 1H), 1.51-1.45 (m, 2H), 0.91 (t, J=7.4 Hz, 3H); ³¹P NMR (202 MHz, D₂O) δ 16.77 (d, J=8.0 Hz, 1P), 7.51-7.38 (m, 1P), −0.89 (s, 1P), −11.37 (d, J=26.3 Hz, 1P).

### Example 11: Synthes of Compound 718

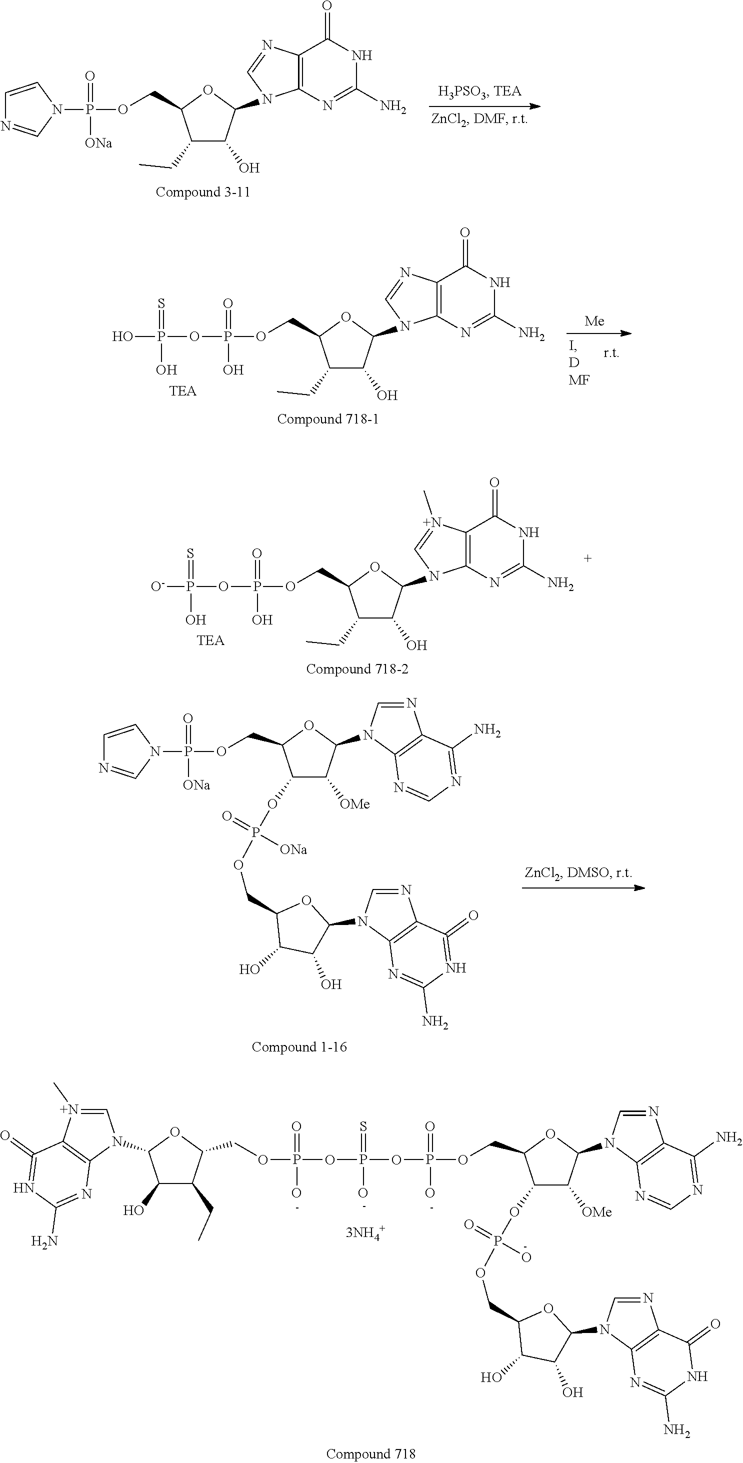

According to the above synthetic route and referring to the synthesis method of compound 1, compound 3-11 was used instead of compound 1-13, and thiophosphoric acid was used instead of phosphoric acid to synthesize the ammonium salt of compound 718.

The characterization data of the ammonium salt of compound 718 are: MS (m/z): 1172.08 [M−1]⁻; ¹H NMR (500 MHz, D₂O) δ 9.09 (s, 1H), 8.44-8.43 (m, 1H), 8.15-8.14 (m, 1H), 7.95-7.94 (m, 1H), 6.03-6.01 (m, 1H), 5.82-5.81 (m, 1H), 5.77 (s, 1H), 4.99-4.97 (m, 1H), 4.57-4.47 (m, 5H), 4.34 (m, 1H), 4.29-4.26 (m, 2H), 4.20-4.09 (m, 4H), 4.03-4.00 (m, 3H), 3.43-3.42 (m, 3H), 2.18-2.15 (m, 1H), 1.44-1.41 (m, 2H), 0.91-0.86 (m, 3H); ³¹P NMR (202 MHz, D₂O) δ 29.99 (m, 1P), −0.93 (d, J=12.7 Hz, 1P), −12.49 (m, 2P).

### Example 12: Synthes of Compound 719

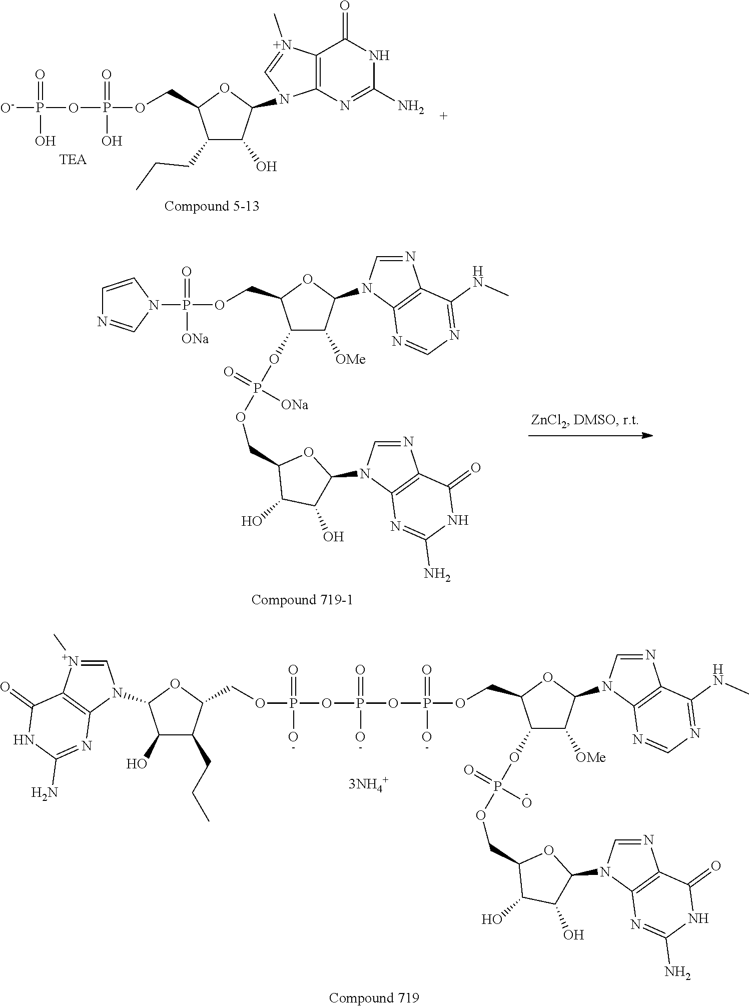

According to the above synthetic route and referring to the synthesis method of compound 1, compound 5-13 was used instead of compound 1-15, and compound 719-1 was used instead of compound 1-16 to synthesize the ammonium salt of compound 719.

The characterization data of the ammonium salt of compound 719 are: MS (m/z): 1184.03 [M−1]⁻; ¹H NMR (500 MHz, D₂O) δ 9.09 (s, 1H), 8.45 (s, 1H), 8.20 (s, 1H), 7.98 (s, 1H), 6.05 (d, J=5.2 Hz, 1H), 5.81-5.80 (m, 2H), 4.96-4.93 (m, 1H), 4.55 (d, J=4.4 Hz, 1H), 4.52 (s, 1H), 4.49-4.44 (m, 3H), 4.34-4.32 (m, 2H), 4.26-4.23 (m, 1H), 4.20-4.19 (m, 2H), 4.16-4.09 (m, 2H), 4.01 (s, 3H), 3.46 (s, 3H), 3.14 (br, 3H), 2.23-2.20 (m, 1H), 1.43-1.20 (m, 4H), 0.84 (t, J=6.8 Hz, 3H); ³¹P NMR (202 MHz, D₂O) δ −0.89 (s, 1P), −11.61 (m, 2P), −22.88 (m, 1P).

### Example 13: Synthes of Compound 720

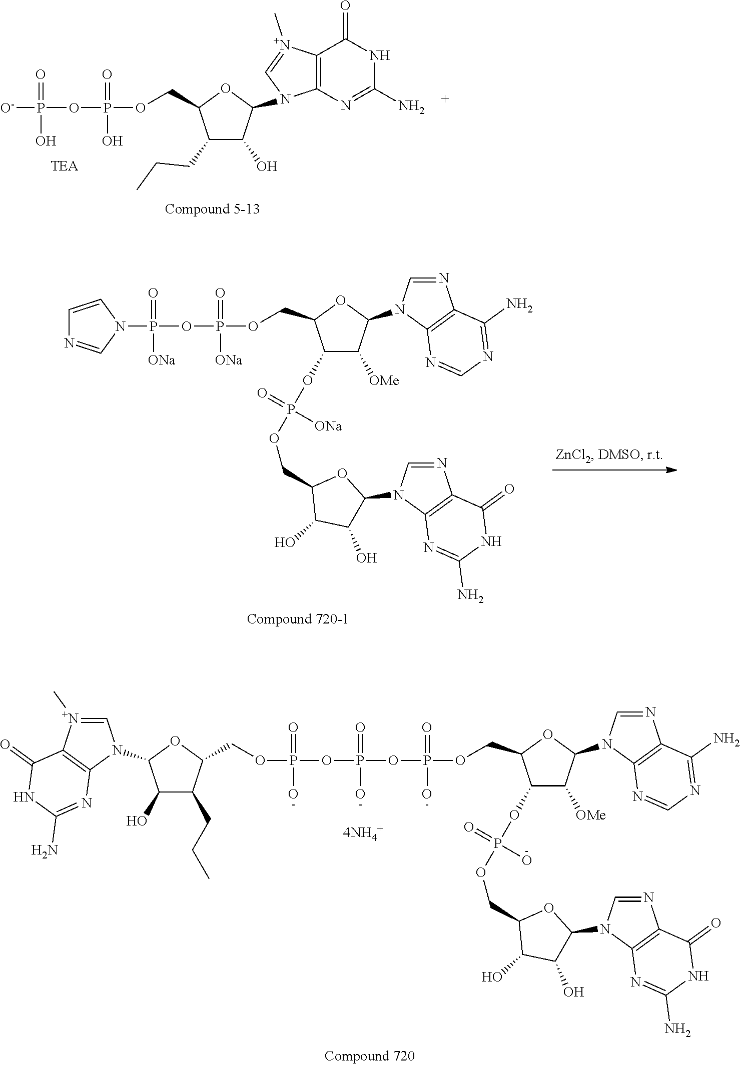

According to the above synthetic route and referring to the synthesis method of compound 1, compound 5-13 was used instead of compound 1-15, and compound 720-1 was used instead of compound 1-16 to synthesize the ammonium salt of compound 720.

The characterization data of the ammonium salt of compound 720 are: MS (m/z): 1250.03 [M−1]⁻; ¹H NMR (500 MHz, D₂O) δ 8.36 (s, 1H), 8.06 (s, 1H), 7.85 (s, 1H), 5.96 (d, J=5.4 Hz, 1H), 5.77-5.75 (m, 2H), 4.88-4.87 (m, 1H), 4.48-4.44 (m, 4H), 4.37-4.35 (m, 1H), 4.31-4.28 (m, 2H), 4.22-4.17 (m, 3H), 4.14-4.12 (m, 2H), 4.00 (s, 3H), 3.40 (s, 3H), 2.20-2.16 (m, 1H), 1.43-1.14 (m, 4H), 0.81 (t, J=6.8 Hz, 3H); ³¹P NMR (202 MHz, D₂O) δ −0.98 (s, 1P), −11.50 (m, 2P), −22.75 (m, 2P).

### Example 14: Synthes of Compound 103

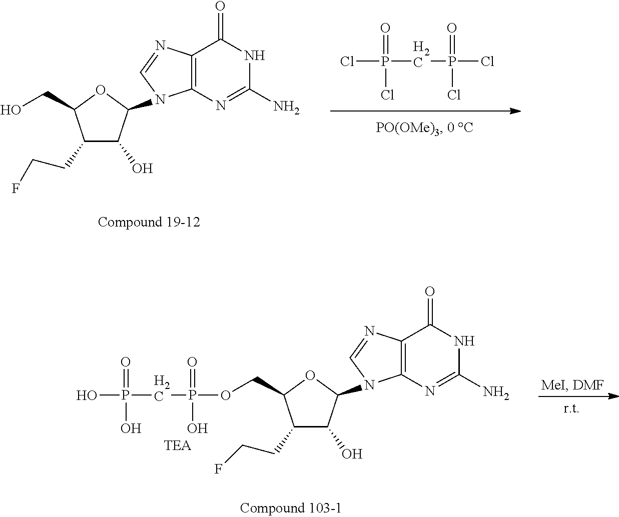

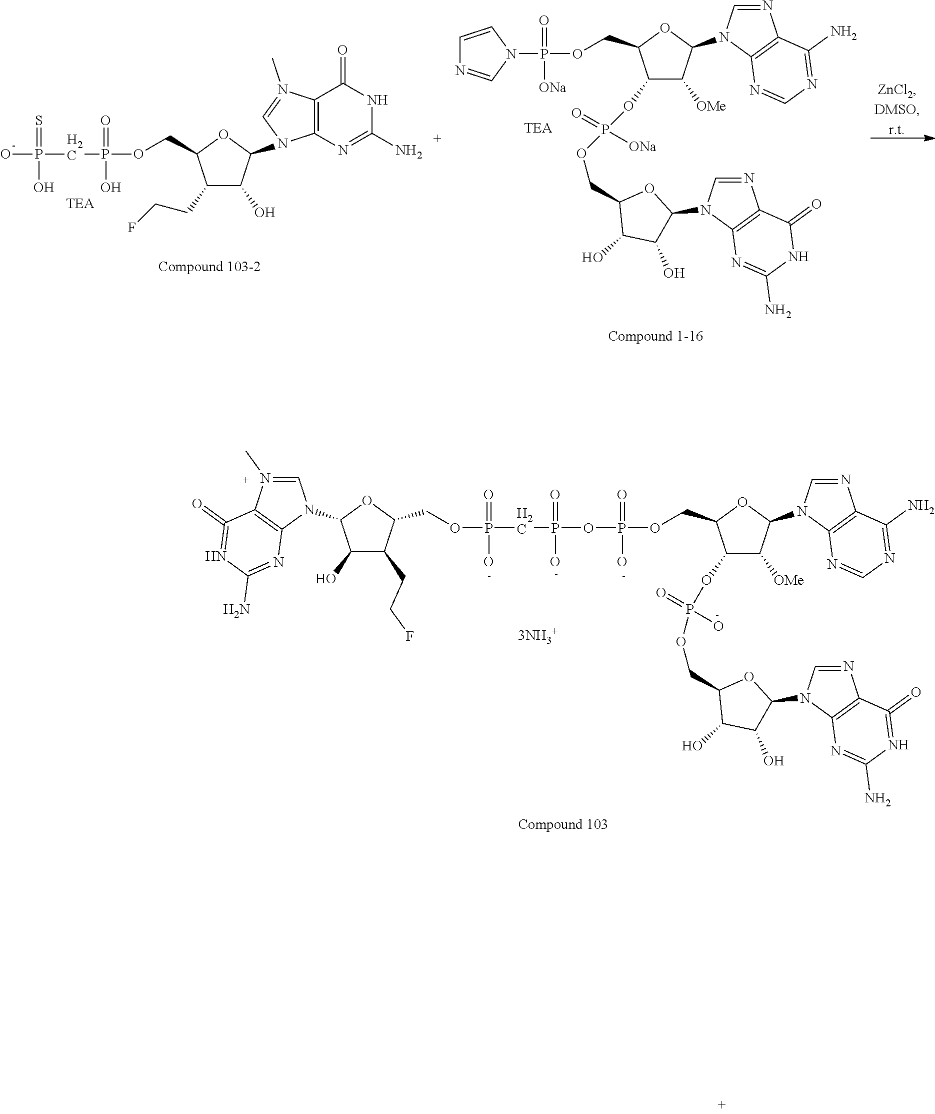

According to the above synthetic route and referring to the synthesis method of compound 1, compound 19-12 was used instead of compound 1-11, and methylenebis(phosphonic dichloride) was used instead of trimethyl phosphate to synthesize the triethylamine salt of compound 103-1.

The ammonium salt of compound 103 was synthesized by replacing compound 1-14 with compound 103-1.

The characterization data of the ammonium salt of compound 103 are: MS (m/z): 1172.07 [M−1]⁻; ¹H NMR (500 MHz, D₂O) δ 8.43 (s, 1H), 8.10 (s, 1H), 7.94 (s, 1H), 6.04 (d, J=5.6 Hz, 1H), 5.86 (s, 1H), 5.81 (d, J=5.6 Hz, 1H), 4.93-4.89 (m, 1H), 4.62-4.58 (m, 2H), 4.53-4.49 (m, 3H), 4.43-4.39 (m, 2H), 4.33 (s, 1H), 4.27-4.19 (m, 5H), 4.14-4.10 (m, 1H), 3.99 (s, 3H), 3.43 (s, 3H), 2.50-2.34 (m, 3H), 2.01-1.78 (m, 2H); ³¹P NMR (202 MHz, D₂O) δ 16.69 (d, J=9.5 Hz, 1P), 7.61-7.44 (m, 1P), −0.90 (s, 1P), −11.61 (d, J=25.0 Hz, 1P).

### Example 15: Synthes of Compound 399

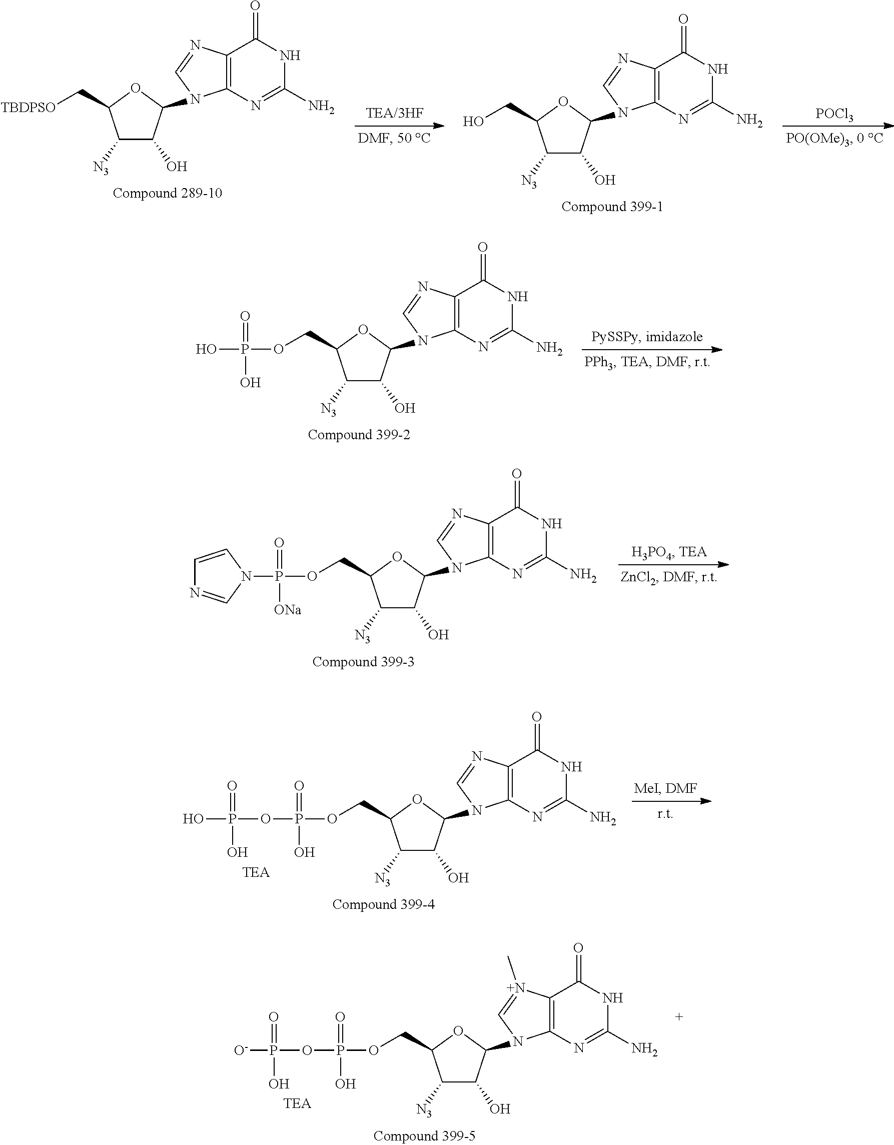

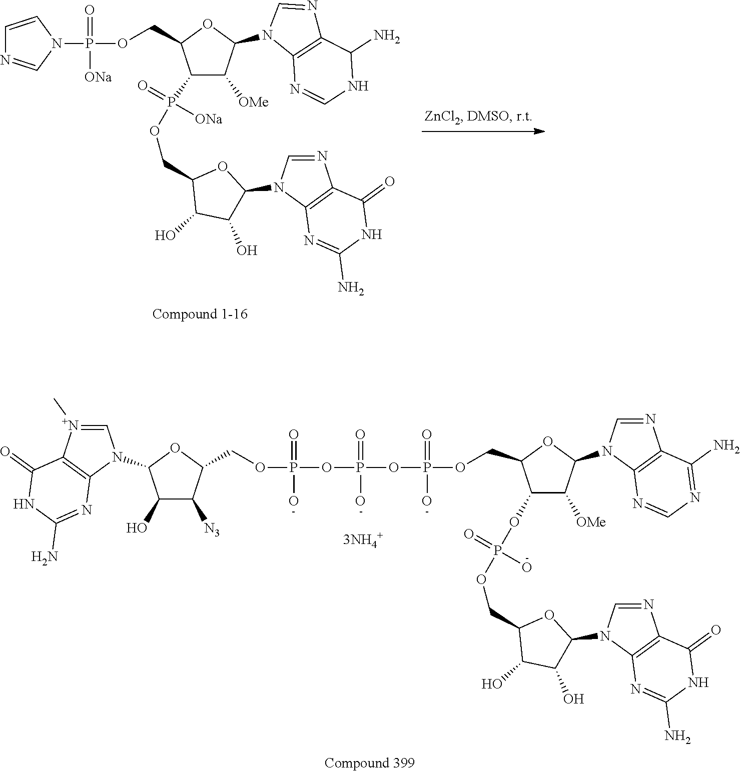

According to the above synthetic route and referring to the synthetic method of compound 289-13, compound 289-10 was used instead of compound 289-12 to synthesize compound 399-1.

According to the above synthetic route and referring to the synthetic method of compound 1, compound 399-1 was used instead of compound 1-11 to synthesize the ammonium salt of compound 399.

The characterization data of the ammonium salt of compound 399 are: MS (m/z): 1169.10 [M−1]⁻; ¹H-NMR (500 MHz, D₂O): ¹H NMR (500 MHz, D₂O) δ 8.29 (s, 1H), 8.04 (s, 1H), 7.89 (s, 1H), 5.93 (d, J=5.1 Hz, 1H), 5.79 (d, J=5.6 Hz, 1H), 5.75 (d, J=3.5 Hz, 1H), 4.88-4.86 (m, 1H), 4.74-4.70 (m, 2H), 4.46-4.44 (m, 2H), 4.34-4.28 (m, 6H), 4.21-4.17 (m, 4H), 3.99 (s, 3H), 3.41 (m, 3H); ³¹P NMR (202 MHz, D₂O) δ −0.95 (s, 1P), −11.56 (m, 2P), −22.93 (m, 1P).

### Example 16: Synthes of Compound 291

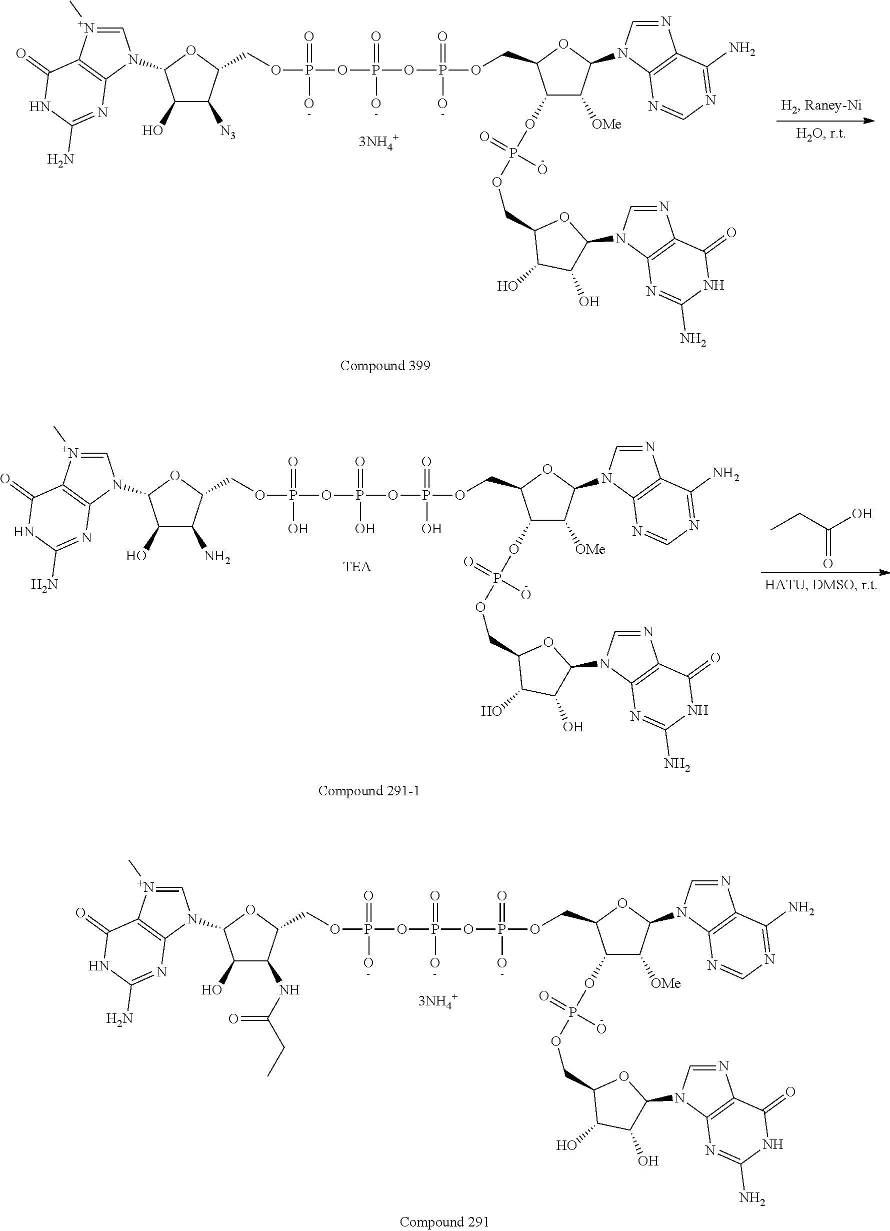

According to the above synthesis route, compound 399 (500 mg, 0.41 mmol) was dissolved in water (5 mL), and Raney nickel (500 mg) was added thereto. The reaction system was replaced with hydrogen, and the reaction mixture was stirred at room temperature until the reaction of raw materials was completed. The reaction mixture was filtered, and the filter cake was washed with water. The filtrates were combined, purified by an ion column and freeze-dried to obtain the triethylamine salt of compound 291-1 (520 mg).

n-Propionic acid (26 mg, 0.35 mmol, eq), DMSO (2 mL) and HATU (160 mg, 0.42 mmol, 1.2 eq) were added to a 25 mL single-necked flask, and stirred for 30 minutes, then the reaction mixture was added dropwise to a solution of the triethylamine salt of compound 291-1 (520 mg, 0.35 mmol) in DMSO (5 mL), stirred at room temperature overnight, purified by an ion column, and freeze-dried to obtain the ammonium salt of compound 291 (220 mg).

The characterization data of the ammonium salt of compound 291 are: MS (m/z): 1199.02 [M−1]⁻; ¹H-NMR (500 MHz, D₂O): ¹H NMR (500 MHz, D₂O) δ 9.10 (s, 1H), 8.39 (s, 1H), 8.14 (s, 1H), 7.93 (s, 1H), 6.02 (d, J=5.3 Hz, 1H), 5.83 (s, 1H), 5.79 (d, J=5.7 Hz, 1H), 4.93-4.89 (m, 1H), 4.56 (d, J=4.4 Hz, 1H), 4.49-4.43 (m, 4H), 4.41-4.38 (m, 2H), 4.32-4.29 (m, 2H), 4.24-4.22 (m, 1H), 4.19-4.16 (m, 2H), 4.10-4.08 (m, 1H), 4.01 (s, 3H), 3.43 (m, 3H), 2.28 (q, J=7.6 Hz, 2H), 1.07 (t, J=7.6 Hz, 3H); ³¹P NMR (202 MHz, D₂O) δ −0.92 (s, 1P), −11.68 (m, 2P), −22.85 (m, 1P).

### Example 17: Synthes of Compound 293

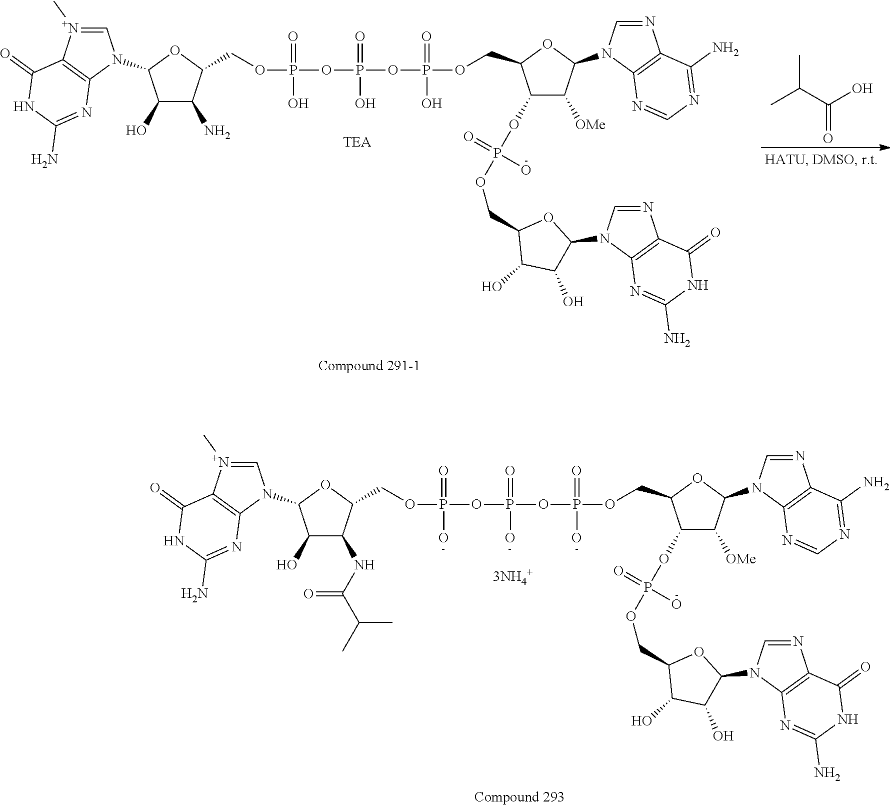

According to the above synthetic route and referring to the synthetic method of compound 291, isobutyric acid was used instead of n-propionic acid to synthesize the ammonium salt of compound 293.

The characterization data of the ammonium salt of compound 293 are: MS (m/z): 1213.05 [M−1]⁻; ¹H-NMR (500 MHz, D₂O): ¹H NMR (500 MHz, D₂O) δ 8.34 (s, 1H), 8.10 (s, 1H), 7.92 (s, 1H), 5.99 (d, J=5.5 Hz, 1H), 5.84 (s, 1H), 5.80 (d, J=5.8 Hz, 1H), 4.91-4.90 (m, 1H), 4.55 (d, 1H), 4.50-4.45 (m, 3H), 4.43-4.42 (m, 2H), 4.39-4.37 (m, 1H), 4.33-4.28 (m, 2H), 4.24-4.18 (m, 3H), 4.09-4.07 (m, 1H), 4.01 (s, 3H), 3.42 (m, 3H), 2.58-2.53 (m, 1H), 1.08-1.06 (m, 6H); ³¹P NMR (202 MHz, D₂O) δ −0.92 (s, 1P), −11.67 (m, 2P), −22.86 (m, 1P).

### Example 18: Synthes of Compound 722

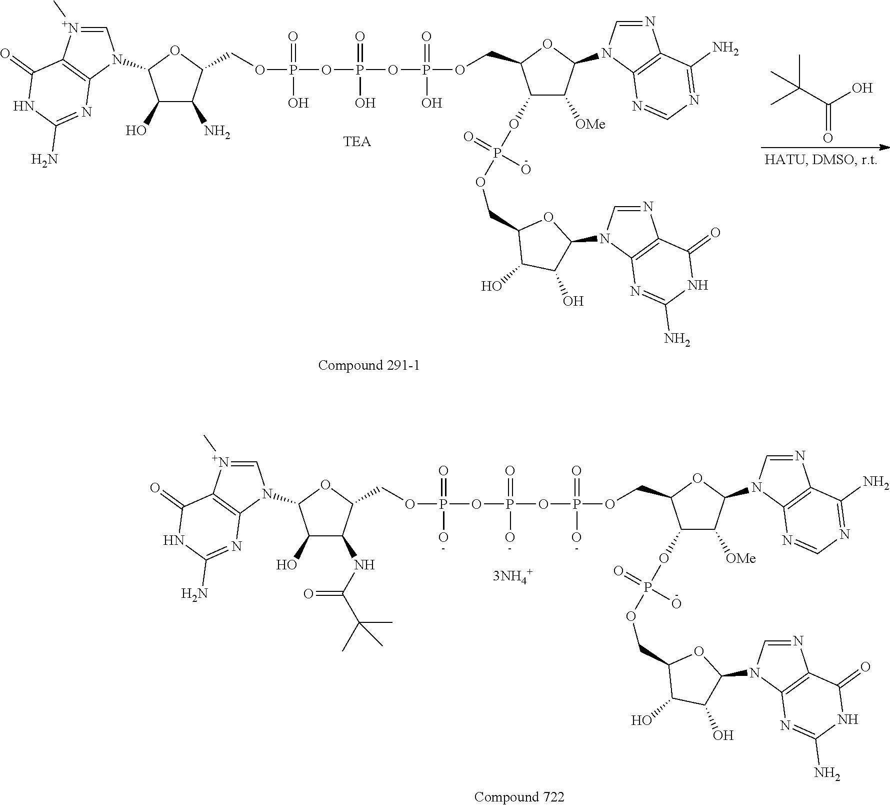

According to the above synthetic route and referring to the synthetic method of compound 291, pivalic acid was used instead of n-propionic acid to synthesize the ammonium salt of compound 722.

The characterization data of the ammonium salt of compound 722 are: MS (m/z): 1227.03 [M−1]⁻; ¹H-NMR (500 MHz, D₂O): ¹H NMR (500 MHz, D₂O) δ 9.16 (s, 1H), 8.47 (s, 1H), 8.22 (s, 1H), 7.97 (s, 1H), 6.07 (d, J=5.1 Hz, 1H), 5.89 (s, 1H), 5.82 (d, J=5.7 Hz, 1H), 4.95-4.93 (m, 1H), 4.59 (s, 1H), 4.52-4.48 (m, 5H), 4.43-4.42 (m, 1H), 4.35-4.33 (m, 2H), 4.27-4.24 (m, 1H), 4.21-4.19 (m, 2H), 4.12-4.10 (m, 1H), 4.04 (s, 3H), 3.46 (m, 3H), 1.19 (s, 9H); ³¹P NMR (202 MHz, D₂O) δ −0.90 (s, 1P), −11.62 (m, 2P), −22.80 (m, 1P).

### Example 19: Synthes of Compound 297

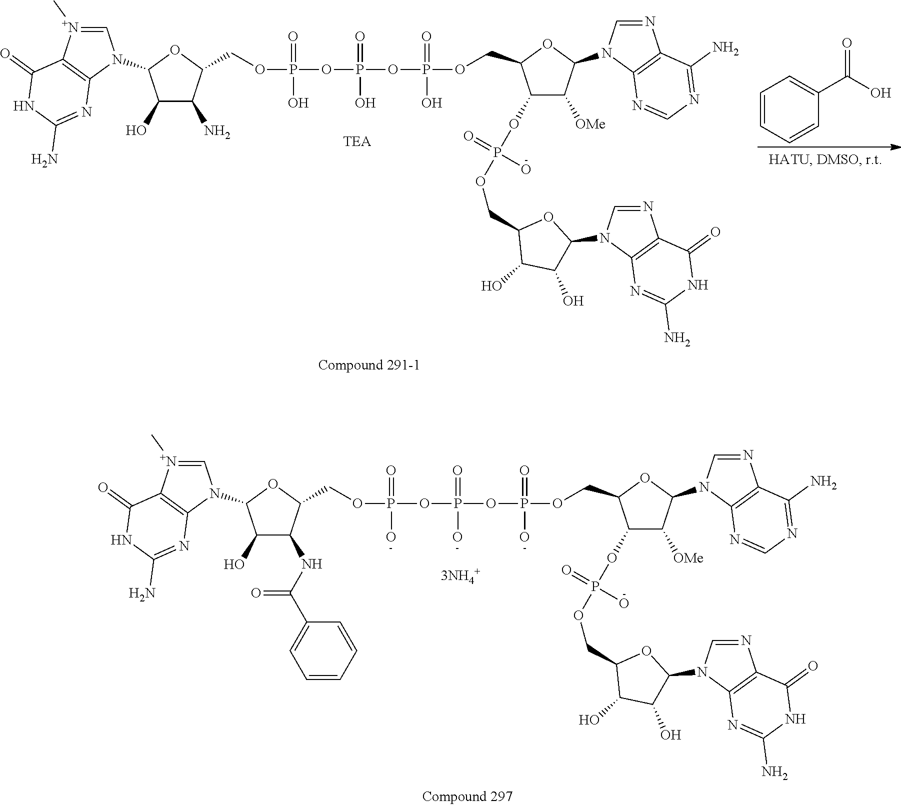 [SMILES: CO[C@@H]1[C@H](OP(=O)(O)OC[C@H]2O[C@@H](n3cnc4c(=O)[nH]c(N)nc43)[C@H](O)[C@@H]2O)[C@@H](COP(=O)(O)OP(=O)(O)OP(=O)(O)OC[C@H]2O[C@@H](n3c[n+](C)c4c(=O)[nH]c(N)nc43)[C@H](O)[C@@H]2N)O[C@H]1n1cnc2c(N)ncnc21 ; O=C(O)c1ccccc1 ; CO[C@@H]1[C@H](OP(=O)(O)OC[C@H]2O[C@@H](n3cnc4c(=O)[nH]c(N)nc43)[C@H](O)[C@@H]2O)[C@@H](COP(=O)(O)OP(=O)(O)OP(=O)(O)OC[C@H]2O[C@@H](n3c[n+](C)c4c(=O)[nH]c(N)nc43)[C@H](O)[C@@H]2NC(=O)c2ccccc2)O[C@H]1n1cnc2c(N)ncnc21]

According to the above synthetic route and referring to the synthetic method of compound 291, benzoic acid was used instead of n-propionic acid to synthesize the ammonium salt of compound 297.

The characterization data of the ammonium salt of compound 297 are: MS (m/z): 1247.03 [M−1]⁻; ¹H-NMR (500 MHz, D₂O): ¹H NMR (500 MHz, D₂O) δ 8.36 (s, 1H), 8.07 (s, 1H), 7.93 (s, 1H), 7.71 (d, J=8.1 Hz, 2H), 7.54 (t, J=7.4 Hz, 1H), 7.44 (t, J=7.8 Hz, 2H), 5.97 (d, J=5.3 Hz, 1H), 5.94 (s, 1H), 5.82 (d, J=5.8 Hz, 1H), 4.93-4.91 (m, 1H), 4.71 (s, 1H), 4.66-4.62 (m, 2H), 4.55-4.52 (m, 2H), 4.49 (t, J=4.3 Hz, 1H), 4.40 (t, J=4.8 Hz, 1H), 4.35-4.31 (m, 2H), 4.27-4.23 (m, 2H), 4.21-4.19 (m, 2H), 4.08 (s, 3H), 3.43 (m, 3H); ³¹P NMR (202 MHz, D₂O) δ −0.92 (s, 1P), −11.55 (m, 2P), −22.78 (m, 1P).

### Example 20: Synthes of Compound 710

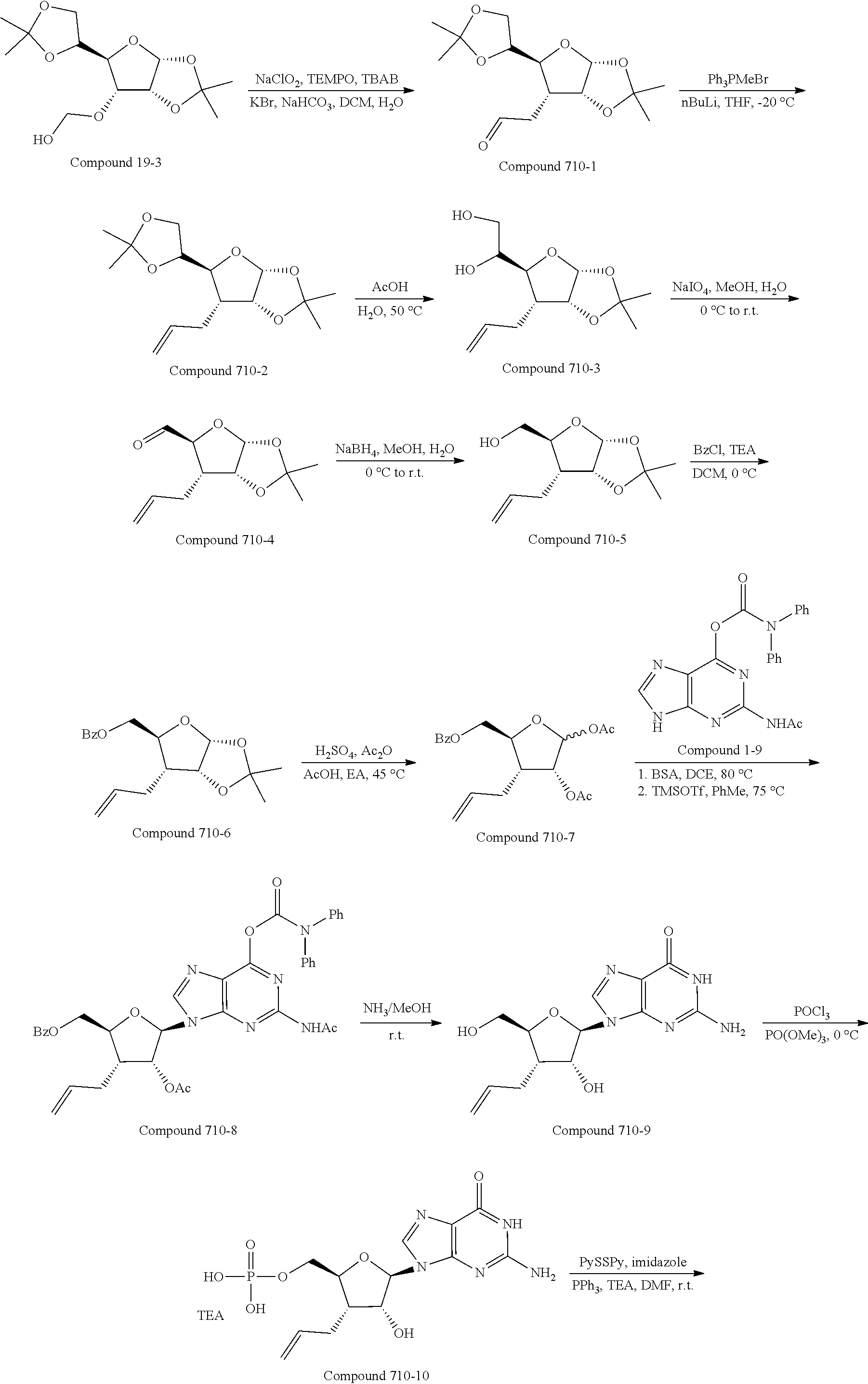

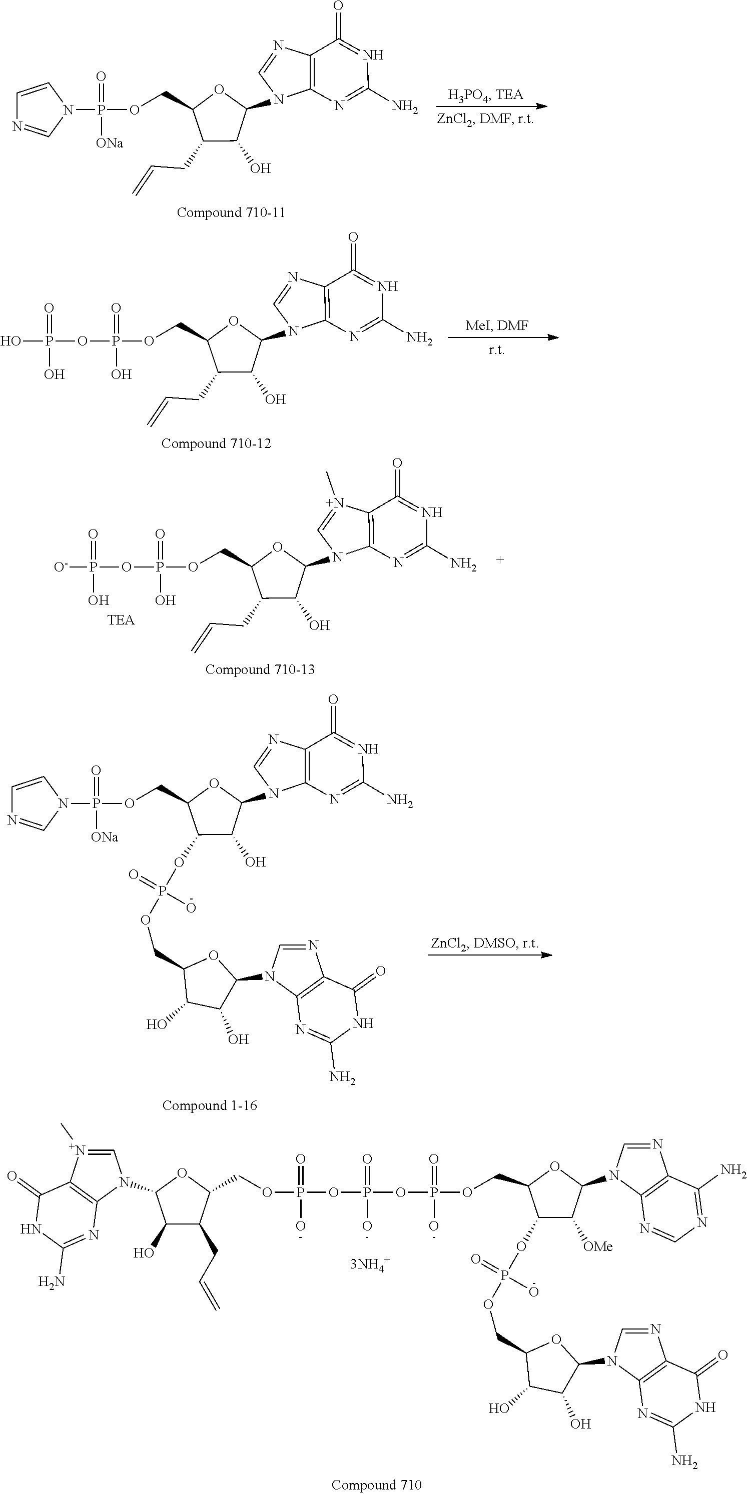

According to the above synthetic route, compound 19-3 (20 g, 69.4 mmol) was dissolved in dichloromethane (100 mL), and then potassium bromide (1.65 g, 13.9 mmol, 0.2 eq), TBAB (1.12 g, 3.5 mmol, 0.05 eq), sodium bicarbonate (11.65 g, 138.7 mmol, 2 eq) and water (20 mL) were added thereto, cooled to −5 °C. Tempo (0.11 g, 0.7 mmol, 0.01 eq) was added thereto, and sodium hypochlorite solution (83 g, 8% in water) was added dropwise, and the temperature was kept no higher than 0 °C. After TLC showed that the reaction was completed, sodium bisulfite (3.02 g) was added thereto and stirred for 10 min. The phases were separated, and the organic phase was washed with saturated brine, dried over anhydrous sodium sulfate, filtered, and concentrated under reduced pressure to obtain 20.07 g of the crude product of compound 710-1.

Referring to the synthetic method of compound 1-2, compound 710-1 was used instead of compound 1-1 to synthesize compound 710-2.

Referring to the synthetic method of compound 1-11, compound 710-2 was used instead of compound 1-3 to synthesize compound 710-9.

The characterization data of compound 710-9 are: MS (m/z): 308.14 [M+H]⁺; ¹H NMR (500 MHz, DMSO): δ 10.71 (s, 1H), 7.97 (s, 1H), 6.44 (s, 2H), 5.85-5.82 (m, 1H), 5.70 (s, 1H), 5.52 (d, J=5.5 Hz, 1H), 5.15-5.12 (m, 1H), 4.96 (t, J=5.5 Hz, 1H), 4.90-4.87 (m, 1H), 4.19 (t, J=5.0 Hz, 1H), 3.77 (d, J=9.3 Hz, 1H), 3.73-3.67 (m, 1H), 3.58-3.45 (m, 1H), 2.47-2.37 (m, 3H).

Referring to the synthetic method of compound 1, compound 710-9 was used instead of compound 1-11 to synthesize the ammonium salt of compound 710.

The characterization data of the ammonium salt of compound 710 are: MS (m/z): 1168.02 [M−1]⁻; ¹H NMR (500 MHz, D₂O) δ 8.36 (s, 1H), 8.11 (s, 1H), 7.98 (s, 1H), 6.04 (d, J=5.6 Hz, 1H), 5.88 (s, 1H), 5.82 (d, J=6.0 Hz, 1H), 5.53 (s, 1H), 5.30 (s, 1H), 5.22 (d, J=5.6 Hz, 1H), 4.97-4.93 (m, 1H), 4.83-4.81 (m, 1H), 4.58-4.48 (m, 4H), 4.46-4.40 (m, 2H), 4.35-4.28 (m, 1H), 4.25-4.10 (m, 4H), 4.00 (s, 3H), 3.41 (s, 3H), 2.25-2.15 (m, 3H); ³¹P NMR (202 MHz, D₂O) δ −0.92 (s, 1P), −11.55 (m, 2P), −22.83 (m, 1P).

### Example 21: Synthes of Compound 191

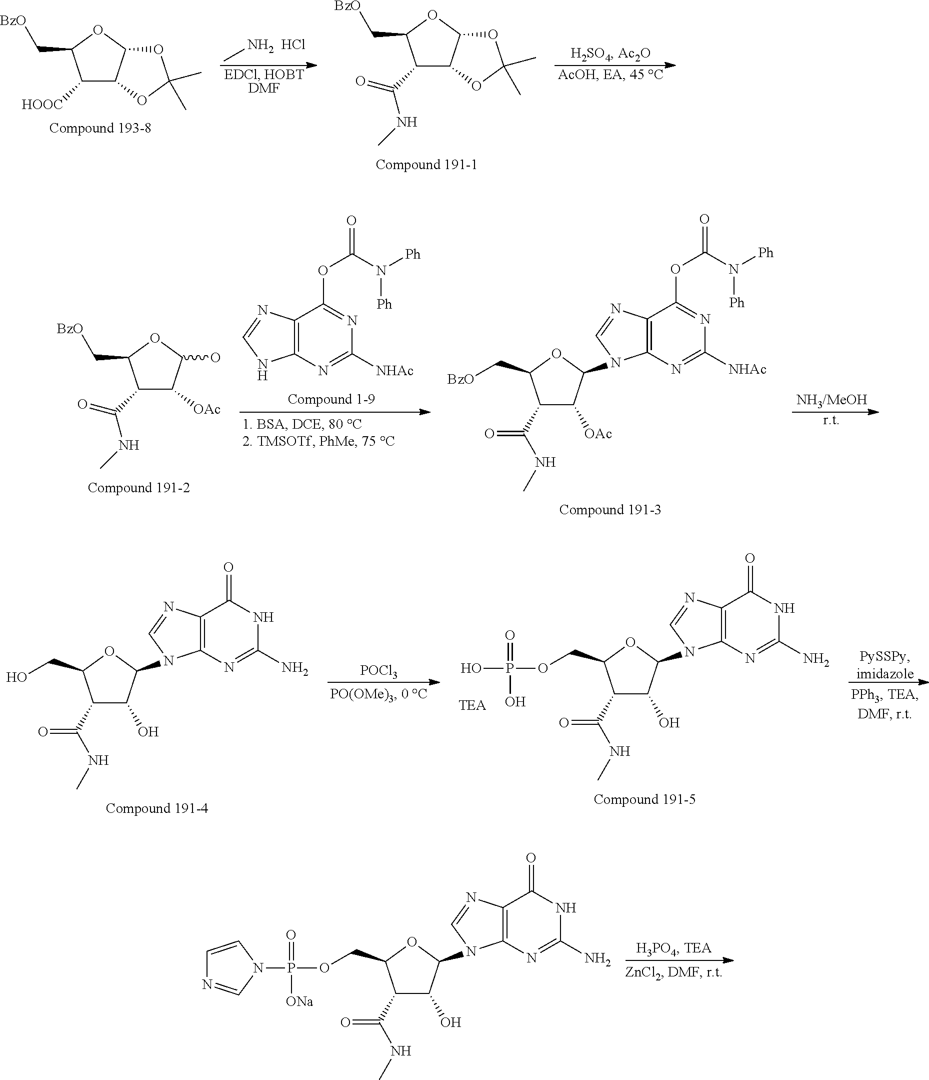

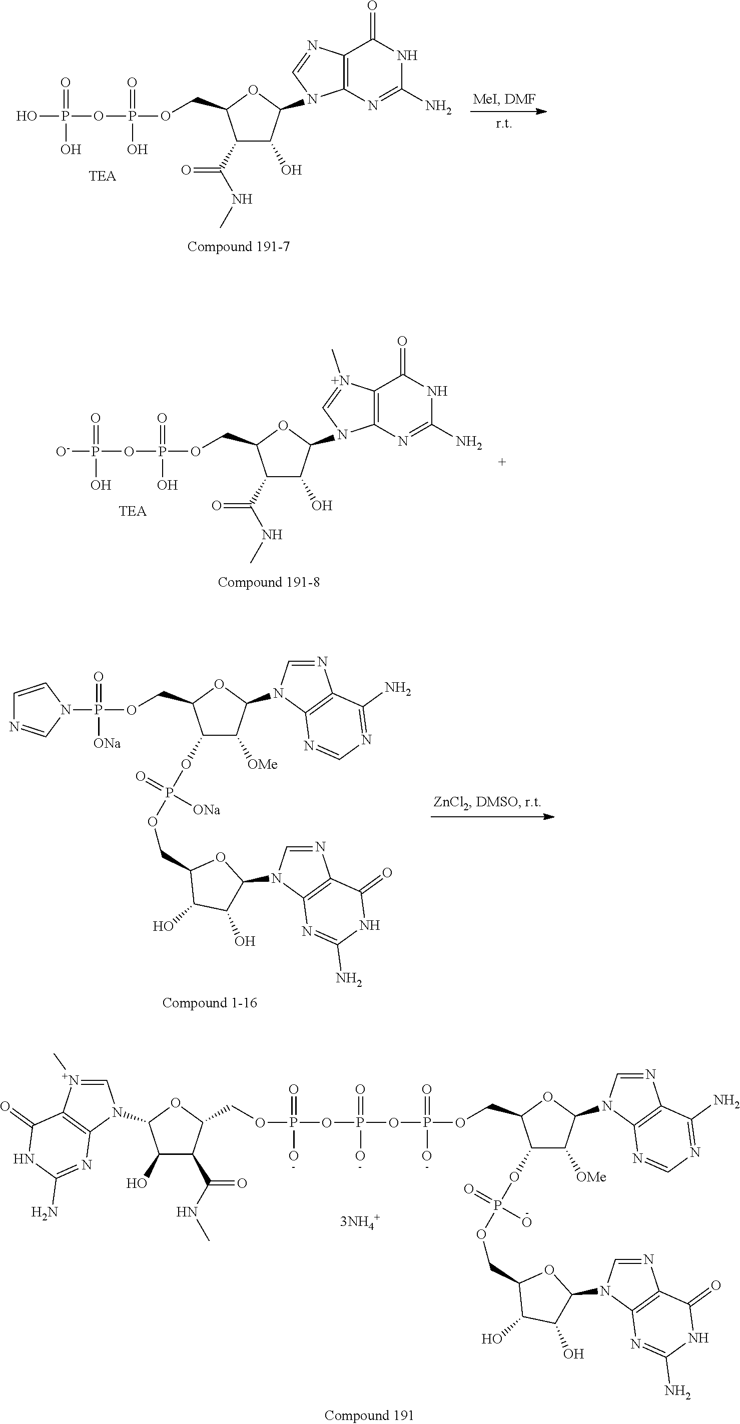

According to the above synthetic route and referring to the synthetic method of compound 193, methylamine hydrochloride was used instead of dimethylamine hydrochloride to synthesize the ammonium salt of compound 191.

The characterization data of the ammonium salt of compound 191 are: MS (m/z): 1185.05 [M−1]⁻; ¹H-NMR (500 MHz, D₂O): δ 9.00 (s, 1H), 8.32 (s, 1H), 8.11 (s, 1H), 7.98 (s, 1H), 6.01 (d, J=5.8 Hz, 1H), 5.88 (d, J=2.5 Hz, 1H), 5.82 (d, J=5.5 Hz, 1H), 4.94-4.91 (m, 2H), 4.89-4.88 (m, 1H), 4.51-4.49 (m, 2H), 4.43-4.40 (m, 2H), 4.34 (s, 1H), 4.29-4.23 (m, 2H), 4.21-4.18 (m, 2H), 4.11-4.07 (m, 1H), 4.01 (s, 3H), 3.81 (t, J=6.8 Hz, 1H), 3.41 (s, 3H), 3.02 (s, 3H). ³¹P-NMR (200 MHz, D₂O): 6-0.90 (s, 1P), −11.62 (m, 2P), −22.89 (m, 1P).

### Example 22: Synthes of Compound 205

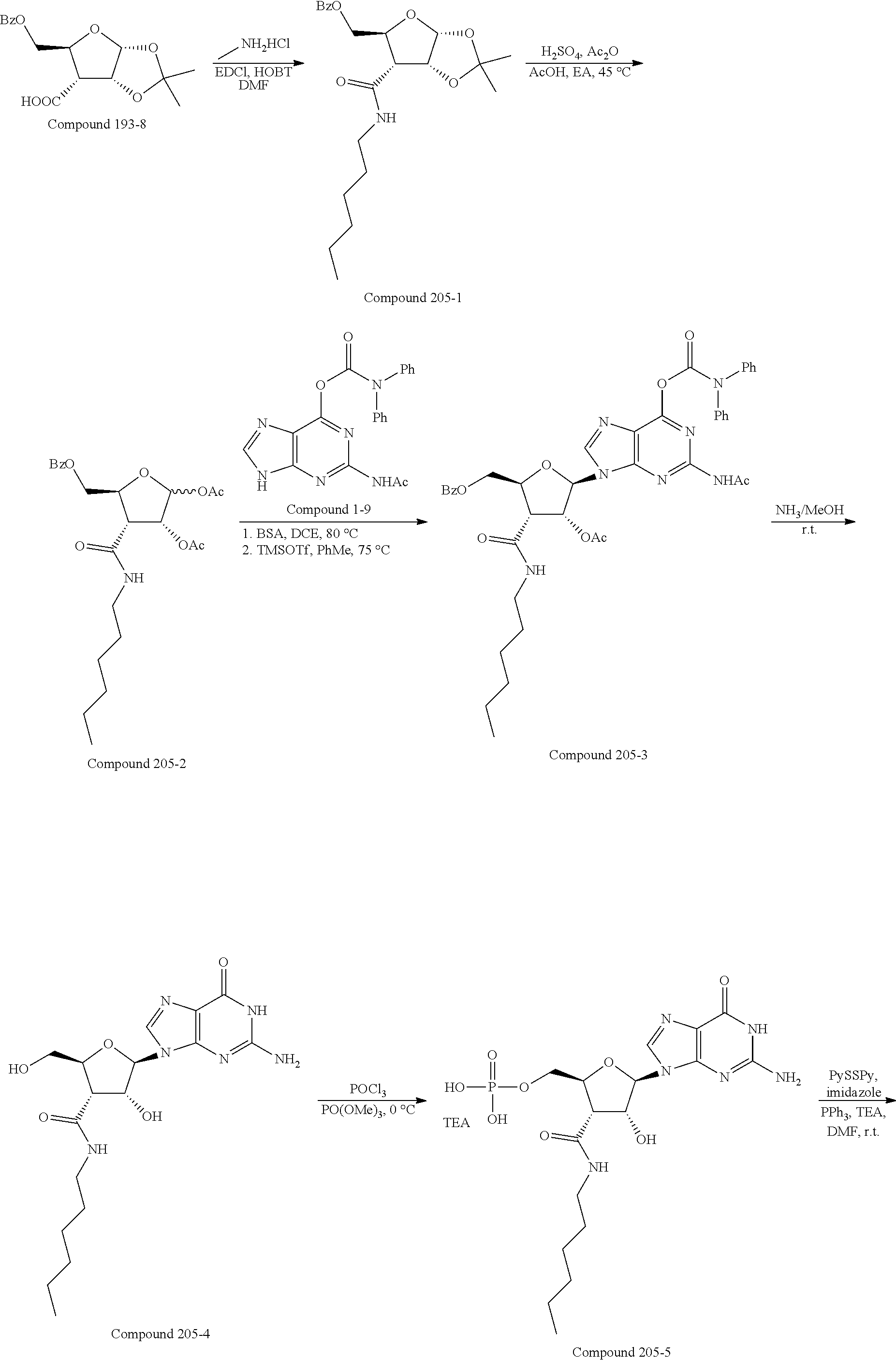

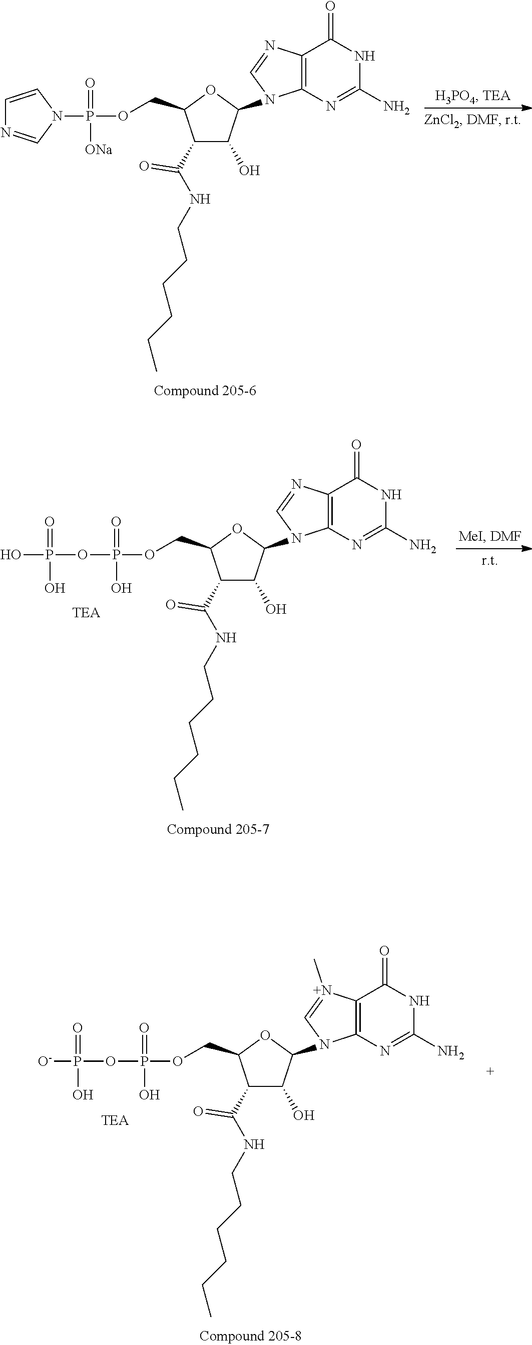

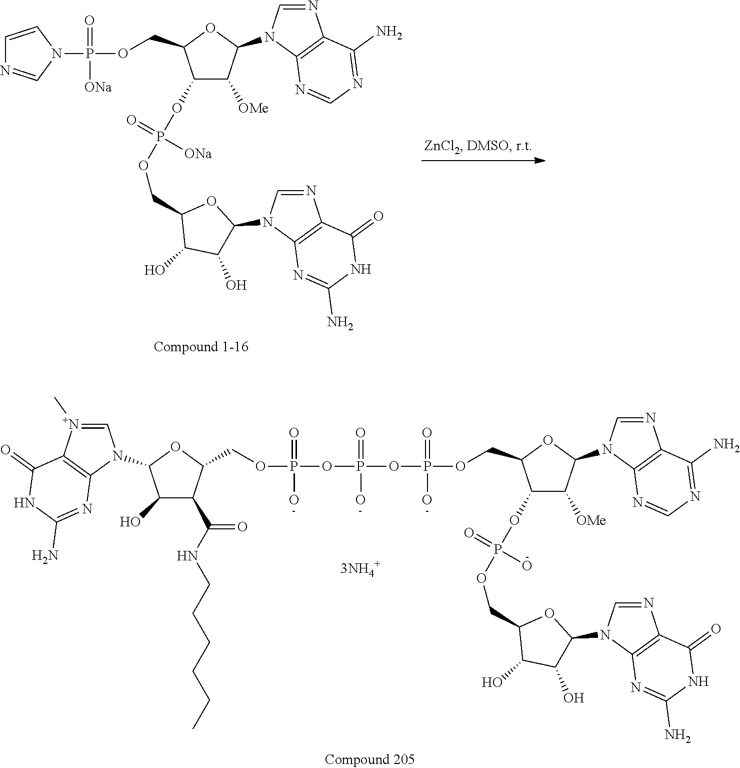

According to the above synthetic route and referring to the synthetic method of compound 193, n-hexylamine was used instead of dimethylamine hydrochloride to synthesize the ammonium salt of compound 205.

The characterization data of the ammonium salt of compound 205 are: MS (m/z): 1255.04 [M−1]⁻; ¹H-NMR (500 MHz, D₂O): δ 8.38 (s, 1H), 8.10 (s, 1H), 7.98 (s, 1H), 6.01 (d, J=5.8 Hz, 1H), 5.88 (d, J=2.5 Hz, 1H), 5.82 (d, J=5.5 Hz, 1H), 4.94-4.91 (m, 2H), 4.89-4.88 (m, 1H), 4.51-4.49 (m, 2H), 4.43-4.40 (m, 2H), 4.34 (s, 1H), 4.29-4.23 (m, 2H), 4.21-4.18 (m, 2H), 4.11-4.07 (m, 1H), 4.01 (s, 3H), 3.81 (t, J=6.8 Hz, 1H), 3.43 (s, 3H), 3.08 (m, 2H), 2.67 (s, 1H), 1.38 (m, 2H), 1.13 (m, 6H), 0.75 (m, 3H). ³¹P-NMR (200 MHz, D₂O): δ −0.91 (s, 1P), −11.46 (m, 2P), −22.90 (t, J=18.0 Hz, 1P).

### Example 23: Synthes of Compound 711

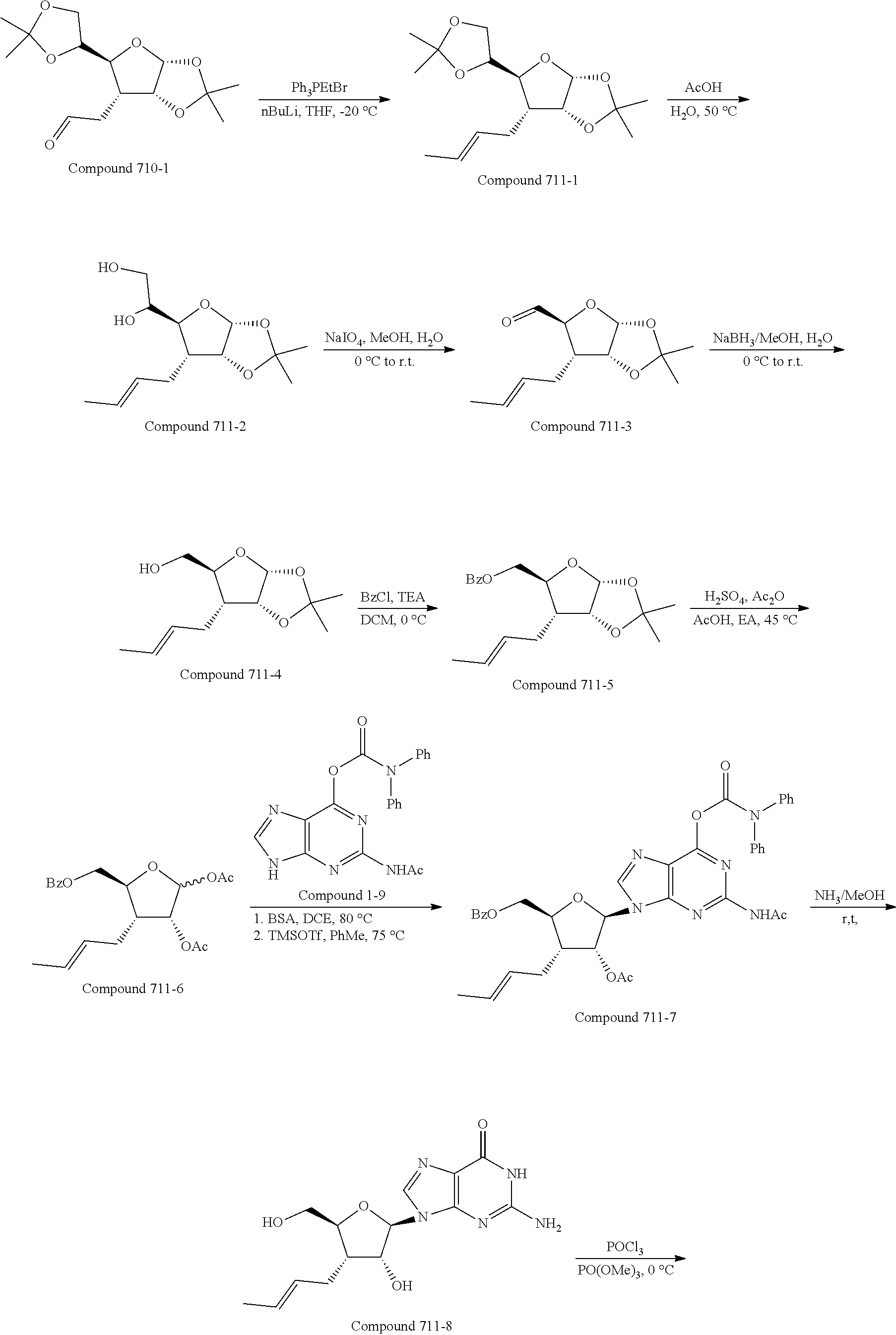

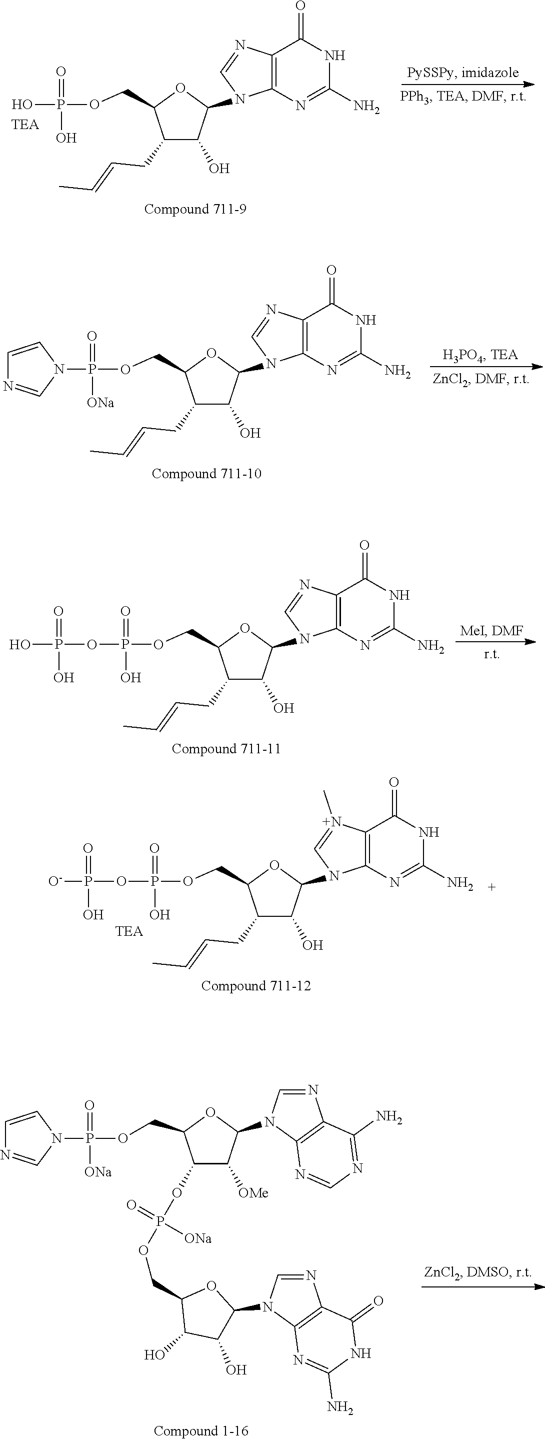

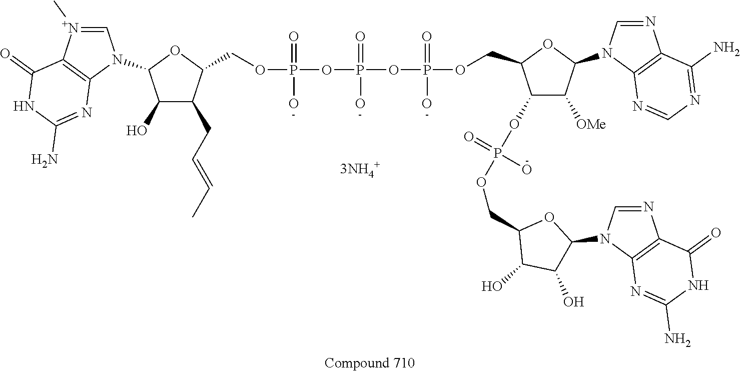 [SMILES: C/C=C/C[C@H]1[C@@H](O)[C@H](n2c[n+](C)c3c(=O)[nH]c(N)nc32)O[C@@H]1COP(=O)(O)OP(=O)(O)OP(=O)(O)OC[C@H]1O[C@@H](n2cnc3c(N)ncnc32)[C@H](OC)[C@@H]1OP(=O)(O)OC[C@H]1O[C@@H](n2cnc3c(=O)[nH]c(N)nc32)[C@H](O)[C@@H]1O]

According to the above synthetic route and referring to the synthetic method of compound 710, ethyltriphenylphosphonium bromide was used instead of methyltriphenylphosphonium bromide to synthesize the ammonium salt of compound 711.

The characterization data of the ammonium salt of compound 711 are: MS (m/z): 1182.09 [M−1]⁻; ¹H NMR (500 MHz, D₂O) δ 9.01 (s, 1H), 8.30 (s, 1H), 8.08 (s, 1H), 7.96 (s, 1H), 6.04 (d, J=5.6 Hz, 1H), 5.88 (s, 1H), 5.82 (d, J=6.0 Hz, 1H), 5.53 (s, 1H), 5.22 (d, J=5.6 Hz, 1H), 4.97-4.93 (m, 1H), 4.83-4.81 (m, 1H), 4.58-4.48 (m, 4H), 4.46-4.40 (m, 2H), 4.35-4.28 (m, 1H), 4.25-4.10 (m, 4H), 4.00 (s, 3H), 3.41 (s, 3H), 2.25-2.15 (m, 3H), 1.63 (d, d, J=6.5 Hz, 3H); ³¹P NMR (202 MHz, D₂O) δ −0.90 (s, 1P), −11.65 (m, 2P), −22.89 (m, 1P).

### Example 24: Synthes of Compound 57

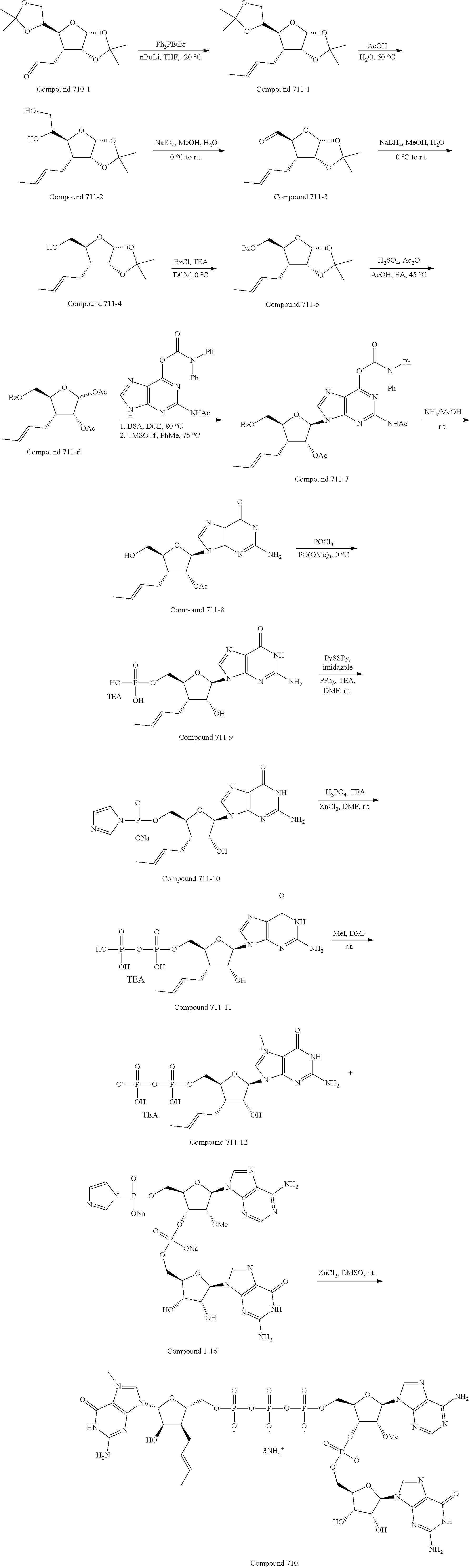

According to the above synthetic route and referring to the synthetic method of compound 1, compound 57-1 was used instead of compound 1-16 to synthesize the ammonium salt of compound 57.

The characterization data of the ammonium salt of compound 57 are MS (m/z): 1092.08 [M−1]⁻; ¹H NMR (500 MHz, D₂O) δ 8.25 (s, 1H), 7.99 (s, 1H), 7.96 (s, 1H), 5.90 (d, J=5.2 Hz, 1H), 5.81 (d, J=5.9 Hz, 1H), 5.79 (s, 1H), 5.13 (s, 1H), 4.95-4.87 (m, 2H), 4.49-4.38 (m, 5H), 4.34 (s, 1H), 4.30-4.25 (m, 2H), 4.19-4.17 (m, 2H), 4.15-4.13 (m, 2H), 4.01 (s, 3H), 3.41 (s, 3H), 2.36-2.32 (m, 1H), 1.02 (d, J=6.7 Hz, 3H); ³¹P NMR (202 MHz, D₂O) δ −11.12 (m, 2P), −22.81 (m, 1P).

### Example 25: Synthes of Compound 59

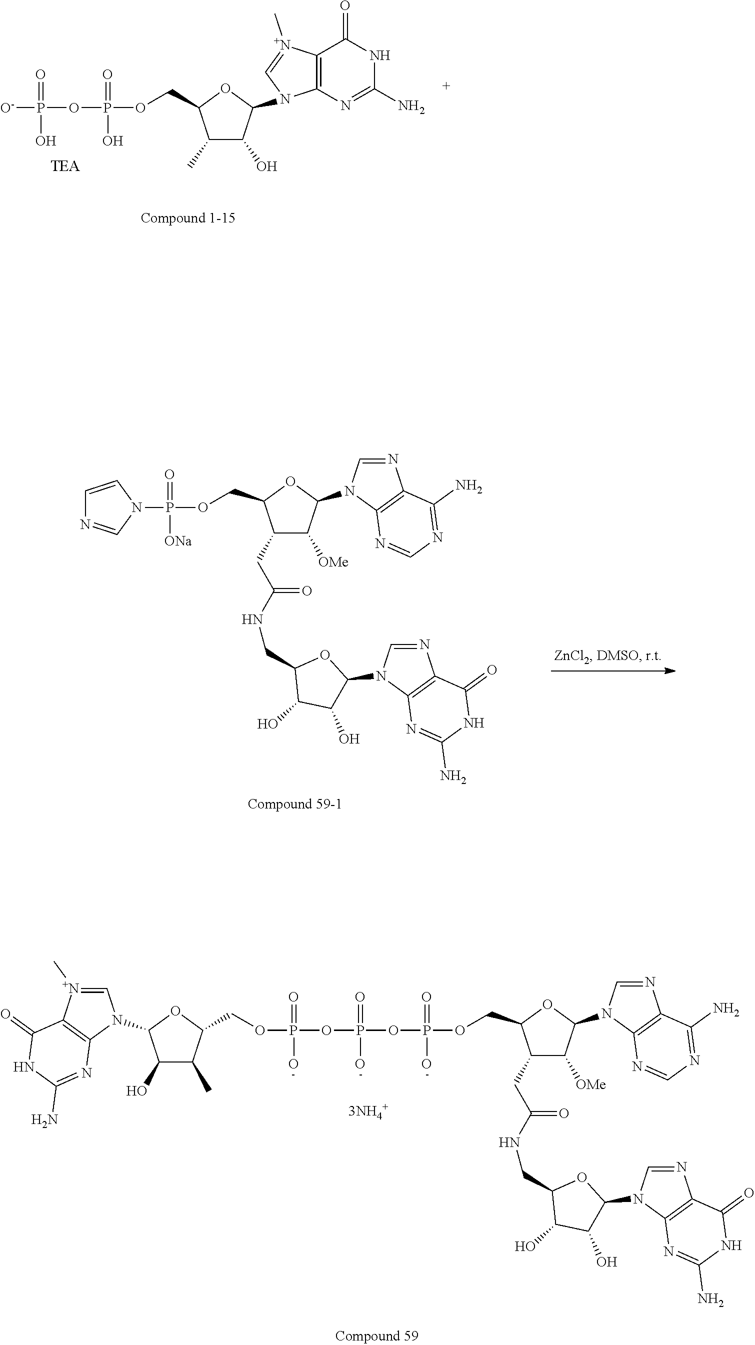

According to the above synthetic route and referring to the synthetic method of compound 1, compound 59-1 was used instead of compound 1-16 to synthesize the ammonium salt of compound 59.

The characterization data of the ammonium salt of compound 1 are: MS (m/z): 1103.05 [M−1]⁻; ¹H NMR (500 MHz, D₂O) δ 8.42 (s, 1H), 8.24 (s, 1H), 7.82 (s, 1H), 6.11 (d, J=5.6 Hz, 1H), 5.81 (d, J=5.9 Hz, 1H), 5.70 (s, 1H), 4.95-4.91 (m, 1H), 4.51-4.48 (m, 2H), 4.47-4.41 (m, 2H), 4.30-4.25 (m, 2H), 4.19-4.17 (m, 1H), 4.15-4.13 (m, 2H), 3.99 (s, 3H), 3.58-3.53 (m, 2H), 3.43 (s, 3H), 2.89-2.87 (m, 1H), 2.61-2.53 (m, 2H), 2.36-2.30 (m, 1H), 1.02 (d, J=6.7 Hz, 3H); ³¹P NMR (202 MHz, D₂O) δ −11.52 (m, 2P), −22.90 (m, 1P).

### EXPERIMENTAL EXAMPLE

1. Detecting the Efficiency of mRNA Capping Synthesis

### 1.1 Experimental Method:

1) The plasmids were linearized and the DNA templates were purified.

2) The mRNA was synthesized by in vitro transcription, using 11 cap structures of the present disclosure, positive control CleanCap®AG (3′OMe) (Trilink, N-7413), negative control (uncapped mRNA) and blank control (DNase/RNase-Free water), respectively. The reaction system is shown in Table 1:

TABLE 1
Reaction system

| | Final | | | |
|---|---|---|---|---|
| Component | concentration | | Dosage | |
| DNase/RNase-Free water | | | Up to 20 μL | |
| ATP Solution (100 mM) | 5 | mM | 1 | μL |
| CTP Solution (100 mM) | 5 | mM | 1 | μL |
| GTP Solution (100 mM) | 5 | mM | 1 | μL |
| UTP Solution (100 mM) | 5 | mM | 1 | μL |
| Cap1 Analog (100 mM) | 4 | mM | 0.8 | μL |
| 10× Transcription buffer | 1× | | 2 | μL |
| DNA template | 50 | μg/mL | 1 | μg |
| Murine RNase Inhibitor (40 U/μL) | 1 | U/μL | 0.5 | μL |
| Inorganic Pyrophosphatase (0.1 U/μL) | 0.002 | U/μL | 0.4 | μL |
| T7 RNA Polymerase (50 U/μL) | 8 | U/μL | 3.2 | μL |

| | |
|---|---|
| Total volume | 20 μL |

The reaction was carried out at 37 °C for 2 to 3 hours. TURBO DNase digestion was carried out for 15 minutes. The mRNA was precipitated by LiC for 30 min or more or overnight, and the mRNA precipitate was washed with 75% ethanol, dried briefly to evaporate the ethanol, and the mRNA was redissolved with RNase-Free water.

3) The transcription product was purified, and the reaction yield was calculated to provide the test results of some compounds, as shown in Table 2.

TABLE 2
Final product mass (μg) obtained from the final product
of 20 μL mRNA synthesis reaction system

| Corresponding | | Final product |
|---|---|---|
| example | Compound | mass |
| 1 | Compound 1 | 96 |
| 2 | Compound 3 | 110 |
| 3 | Compound 5 | 117 |
| 4 | Compound 19 | 137 |
| 5 | Compound 193 | 105 |
| 6 | Compound 289 | 113 |
| 8 | Compound 716 | 103 |
| 9 | Compound 717 | 97 |
| 10 | Compound 721 | 95 |
| 11 | Compound 718 | 106 |
| 12 | Compound 719 | 97 |
| 13 | Compound 720 | 92 |
| 14 | Compound 103 | 94 |
| 15 | Compound 399 | 117 |
| 16 | Compound 291 | 106 |
| 17 | Compound 293 | 103 |
| 18 | Compound 722 | 103 |
| 19 | Compound 297 | 110 |
| 20 | Compound 710 | 108 |
| 21 | Compound 191 | 100 |
| 22 | Compound 205 | 98 |
| 23 | Compound 711 | 106 |
| 24 | Compound 57 | 90 |
| 25 | Compound 59 | 91 |
| | N-7413 | 114 |
| | Negative control | 118 |
| | Blank control | 0 |

4) The obtained mRNA was subjected to an annealing reaction with the probe and the reaction system is shown in Table 3.

TABLE 3
Annealing reaction system

| Component | Dosage | |
|---|---|---|
| RNase H probe | 100 | pmol |
| mRNA | 100 | pmol |
| 10x RNase H reaction buffer | 12 | μL |

| | |
|---|---|
| DNase/RNase-Free water | Up to 120 μL |

Gradient annealing was performed in a PCR instrument:

95 °C 5 min; 65 °C 2 min; 55 °C 2 min; 40 °C 2 min; 22 °C 2 min.

5) Magnetic beads were pre-treated and bound to probes.

100 μL of magnetic beads were taken and placed on a magnetic stand. The storage solution was discarded, and 100 μL of 1*BW solution was added thereto. After mixing well, the mixture was placed on the magnetic stand, and the supernatant was discarded. The wash was carried out three times in the same way. 100 μL of wash A buffer was added thereto. After mixing well, the mixture was placed on the magnetic stand, and the supernatant was discarded. The wash was carried out three times in the same way. 100 μL of wash B buffer was added thereto. After mixing well, the mixture was placed on the magnetic stand, and the supernatant was discarded. The wash was carried out three times in the same way. 120 μL of the sample was added to the magnetic bead solution and incubated for 30 min at room temperature with gentle mixing while incubating.

6) The mRNA was cut to obtain the 5′ single-stranded sequence of mRNA bound to probes.

20 μL of RNase H (5 U/μL) was added thereto, incubated at 37 °C for 3 hours, and mixed well every half hour. 100 μL of 1*BW solution was added thereto. After mixing well, the mixture was placed on the magnetic stand, and the supernatant was discarded. The wash was carried out three times in the same way. 100 μL of deionized water solution was added thereto. After mixing well, the mixture was placed on the magnetic stand, and the supernatant was discarded. The wash was carried out three times in the same way. 100 μL of 75% methanol was added thereto at 80 °C, then the mixture was heated to 80 °C on a heating plate and held for 3 min, placed on the magnetic stand, and the supernatant was aspirated. The sample was dried at room temperature for 45 min to 10 μL using an evaporative centrifuge and then resuspended in 50 μL of 100 μM EDTA/1% MeOH for LC-MS analysis.

### 1.2 Experimental Results

LC-MS was used to analyze the capping efficiency of mRNA synthesized by analogues with different cap structures. The capping efficiency results of some compounds in the detection results are now provided. The results are shown in Table 4.

TABLE 4
mRNA capping efficiency (%) when using different cap structures

| Corresponding example | Compound | Capping efficiency |
|---|---|---|
| 1 | Compound 1 | A |
| 2 | Compound 3 | A |
| 3 | Compound 5 | A |
| 4 | Compound 19 | A |
| 5 | Compound 193 | A |
| 6 | Compound 289 | A |
| 9 | Compound 425 | A |
| 10 | Compound 691 | A |
| 20 | Compound 710 | A |
| 24 | Compound 57 | C |
| | Negative control | 0 |
| | Blank control | 0 |

Note:
The range of capping efficiency is: 100% > A ≥ 95%, 95% > B ≥ 90%, 90% > C ≥ 80%.

As can be seen from the above table, the compounds of the present disclosure all exhibit good capping efficiency, with high capping efficiency (>95%) for multiple cap structures in some of the results shown.

2. Evaluation of Expression Efficiency of Different Capped Luciferase mRNAs in HEK293T Cells

### 2.1 Experimental Method:

1) HNE293T cells were cultured in DMEM medium containing 10% FBS and penicillin/streptomycin at 37 °C and 5% CO₂.

2) The cultured HEK293T cells were spread in a 96-well plate with 1.25×10⁴ cells per well.

3) After the cells completely adhered to the wall, 0.5 μg of a mixture of mRNA sample and Polyplus jetMESSENGER was added to each well of cells, and cultured at 37 °C and 5% CO₂ for 6 hours.

4) The growth medium was removed from the cells to be tested and the cells were rinsed with PBS. After centrifugation to remove PBS, 50 μL of 1× lysis buffer was added thereto, and cells and all liquid were transferred to a microcentrifuge tube, and then centrifuged at 12000×g and 4 °C for 2 min.

5) The supernatant was transferred to a new tube. Fluorescence readings were detected using the ONE-Glo Luciferase Assay System Kit.

3. All compounds, N-7413, negative control and blank control were tested under the same conditions, and some experimental results in the test results are shown in Table 5.

TABLE 5
Relative fluorescence readings for capped mRNAs

| | | Relative |
|---|---|---|
| Corresponding | | fluorescence |
| example | Compound | reading |
| 1 | Compound 1 | 1.74 |
| 2 | Compound 3 | 1.72 |
| 3 | Compound 5 | 1.85 |
| 4 | Compound 19 | 1.52 |
| 5 | Compound 193 | 1.64 |
| 6 | Compound 289 | 1.70 |
| 8 | Compound 716 | 1.50 |
| 9 | Compound 717 | 2.31 |
| 10 | Compound 721 | 1.18 |
| 11 | Compound 718 | 1.66 |
| 12 | Compound 719 | 1.69 |
| 13 | Compound 720 | 1.43 |
| 14 | Compound 103 | 1.45 |
| 15 | Compound 399 | 1.51 |
| 16 | Compound 291 | 1.60 |
| 17 | Compound 293 | 1.68 |
| 18 | Compound 722 | 1.74 |
| 19 | Compound 297 | 1.79 |
| 20 | Compound 710 | 1.90 |
| 21 | Compound 191 | 1.68 |
| 22 | Compound 205 | 1.52 |
| 23 | Compound 711 | 1.86 |
| 24 | Compound 57 | 0.88 |
| 25 | Compound 59 | 0.71 |
| | N-7413 | 1.00 |
| | Negative control | 0 |
| | Blank control | 0 |

Note:
The fluorescence reading of N-7413 is used as 1 for calibration, and the ratio of the fluorescence reading of other capped mRNA transfected to the fluorescence reading of N-7413 is the relative fluorescence reading.

The relative fluorescence reading results (6 h) after transfection of capped mRNA into 293T cells are now provided, as shown in Table 5. It can be seen from the FIGURE that the mRNA capped by the compound of the present disclosure has a high expression level, and some cap structures can enable higher expression efficiency of mRNA compared to N-7413.

The technical features of the above examples can be combined arbitrarily. To simplify the description, not all possible combinations of the technical features in the above examples are described. However, as long as there is no contradiction in the combination of these technical features, they should be considered to be within the scope of the present specification.

The above examples only express several embodiments of the present disclosure, and their descriptions are relatively specific and detailed, but they should not be understood as limiting the scope of the present disclosure. It should be noted that, for those of ordinary skill in the art, several modifications and improvements can be made without departing from the concept of the present disclosure, and these all belong to the protection scope of the present disclosure. Therefore, the scope of protection of the patent of the present disclosure should be determined by the appended claims.

1-19. (canceled)

20. A compound or pharmaceutically acceptable salt, wherein the compound is the following compound:

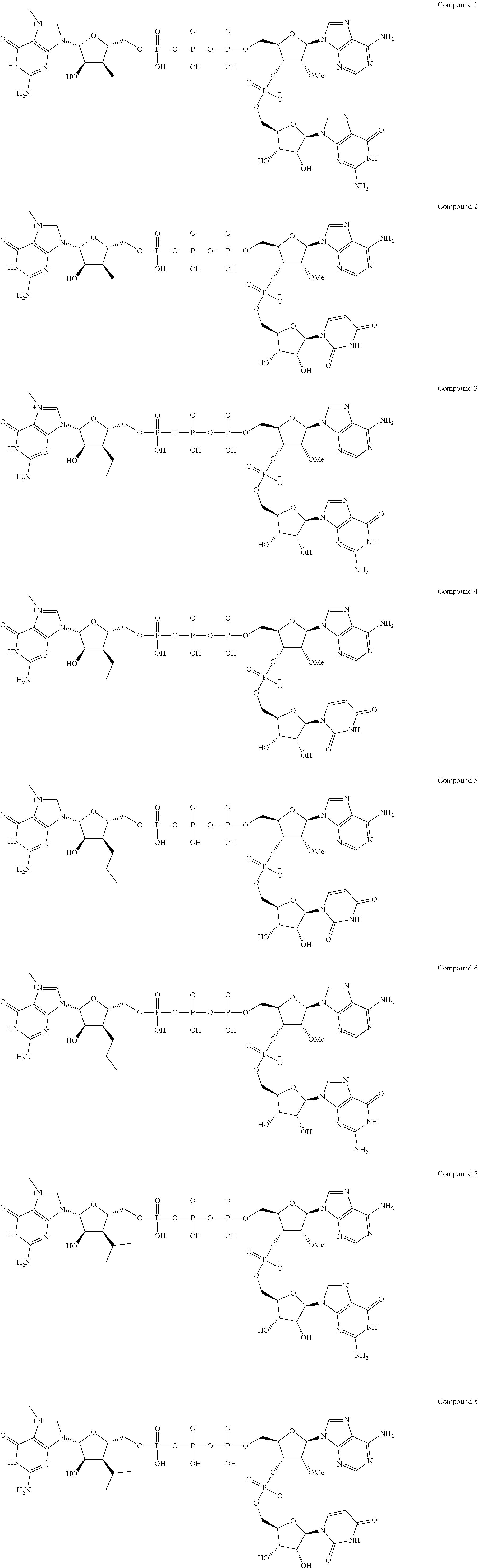

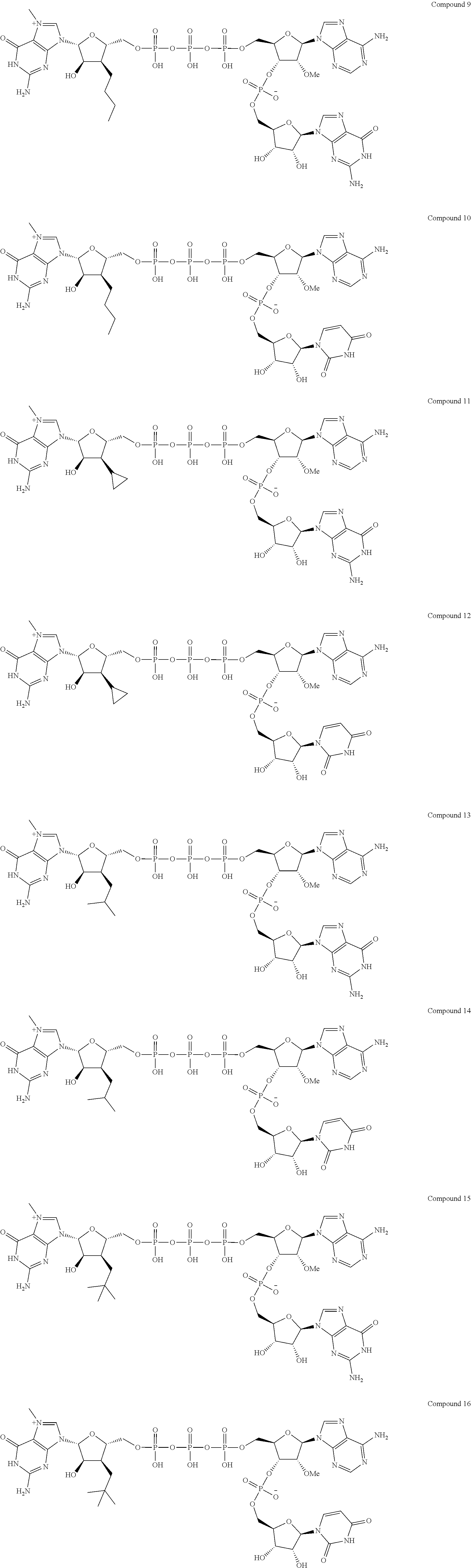

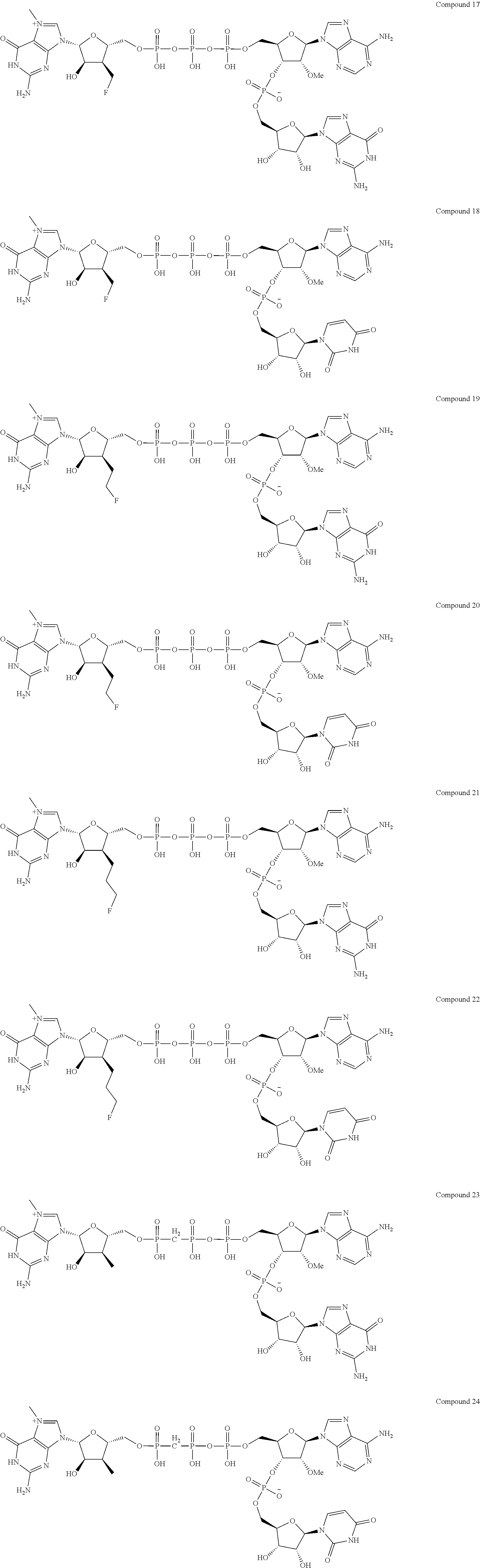

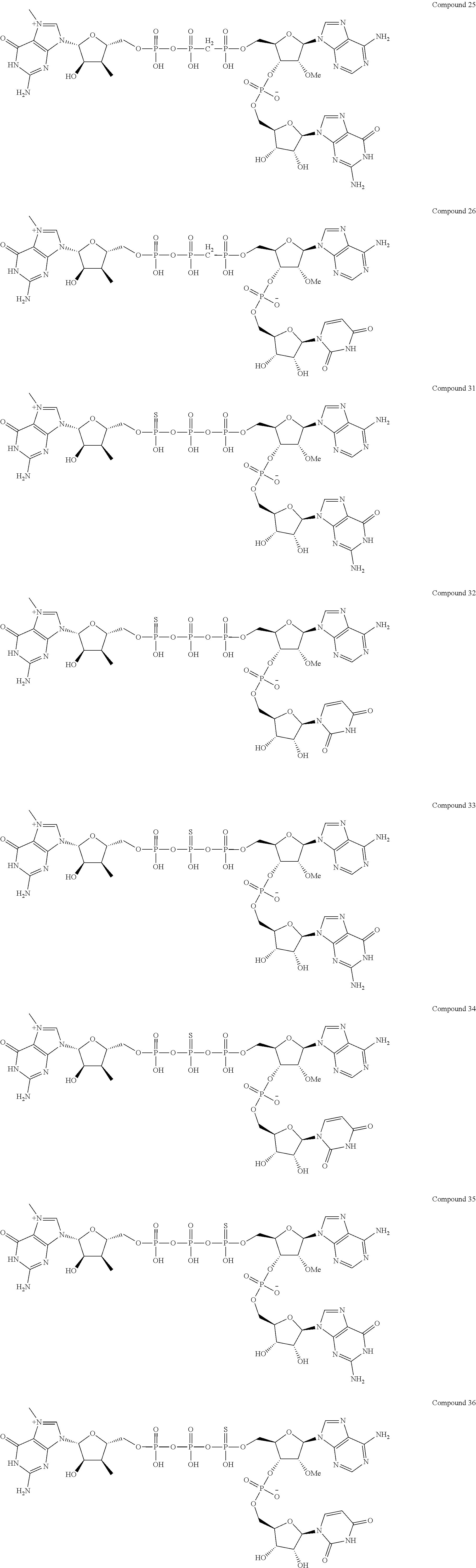

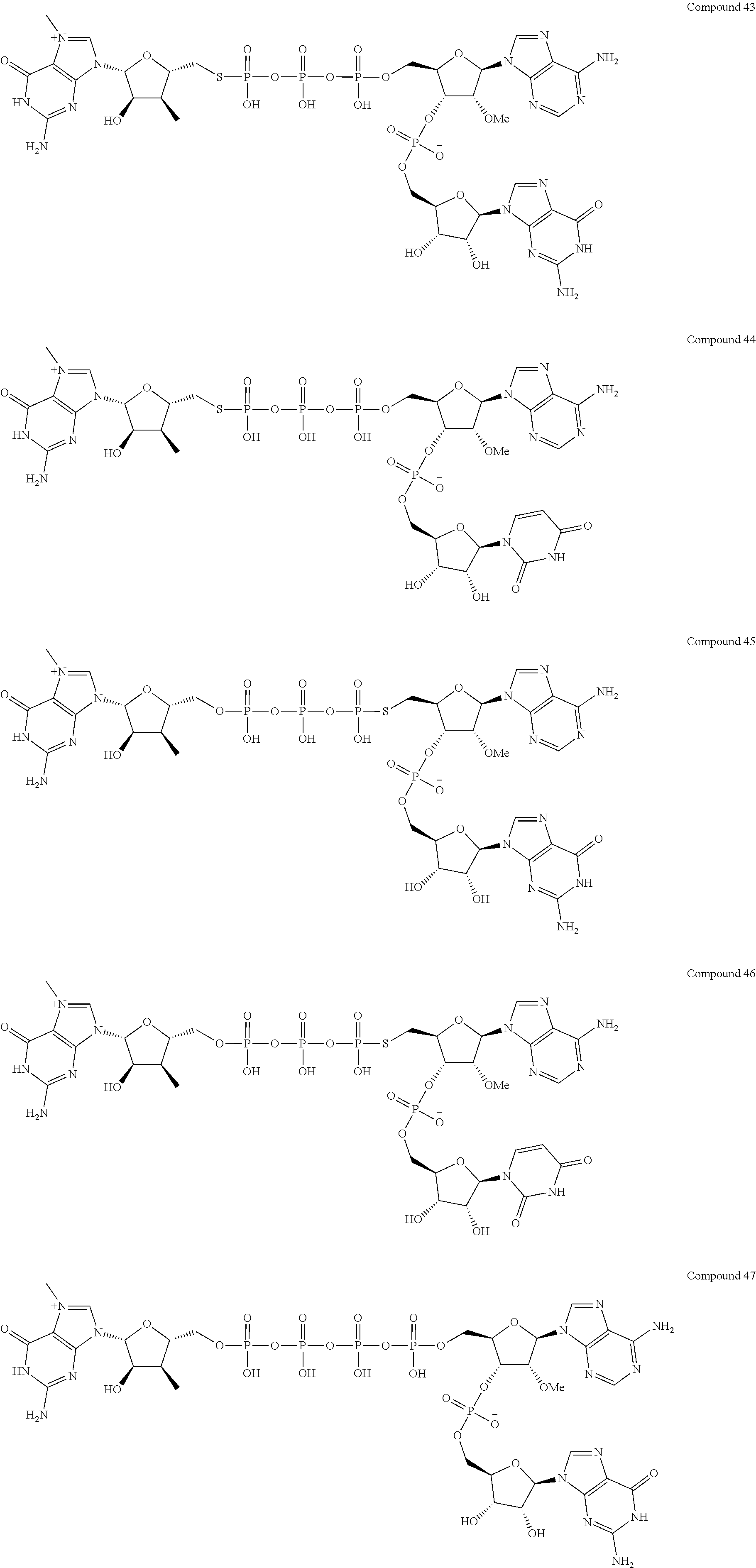

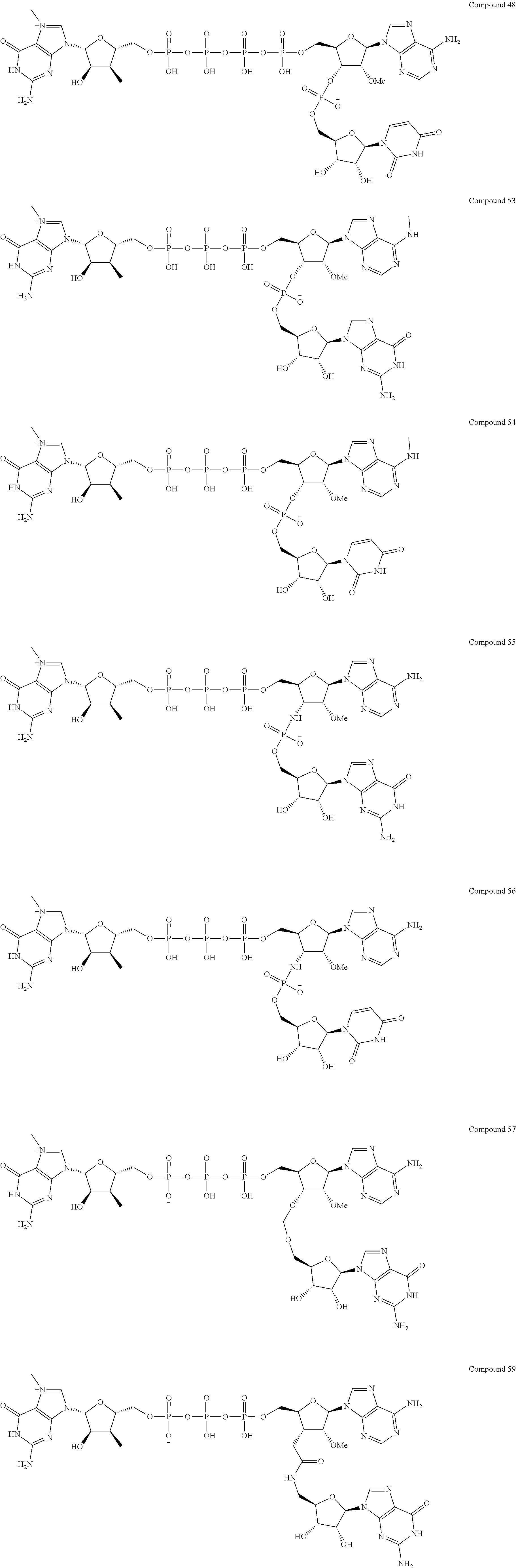

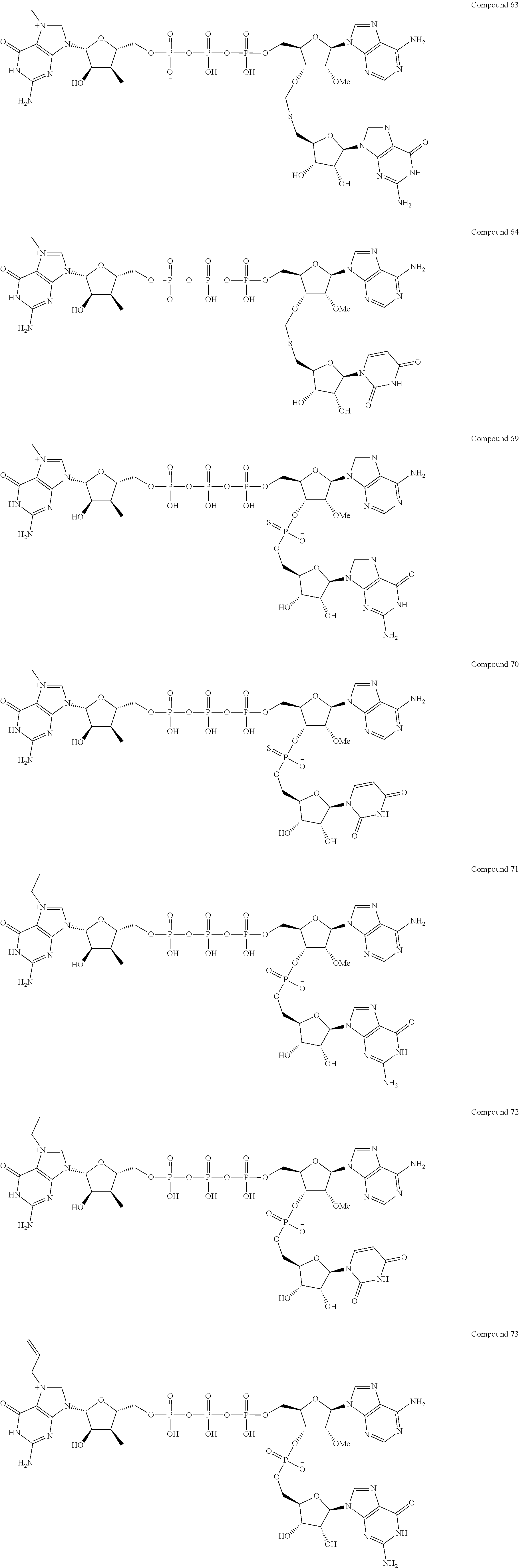

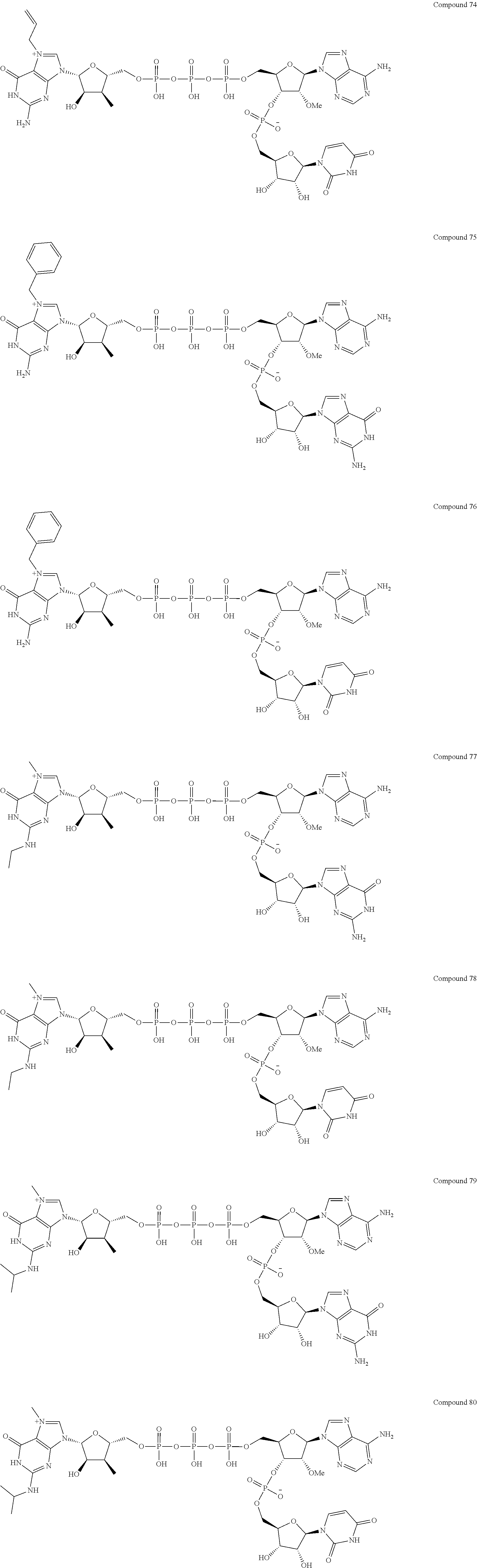

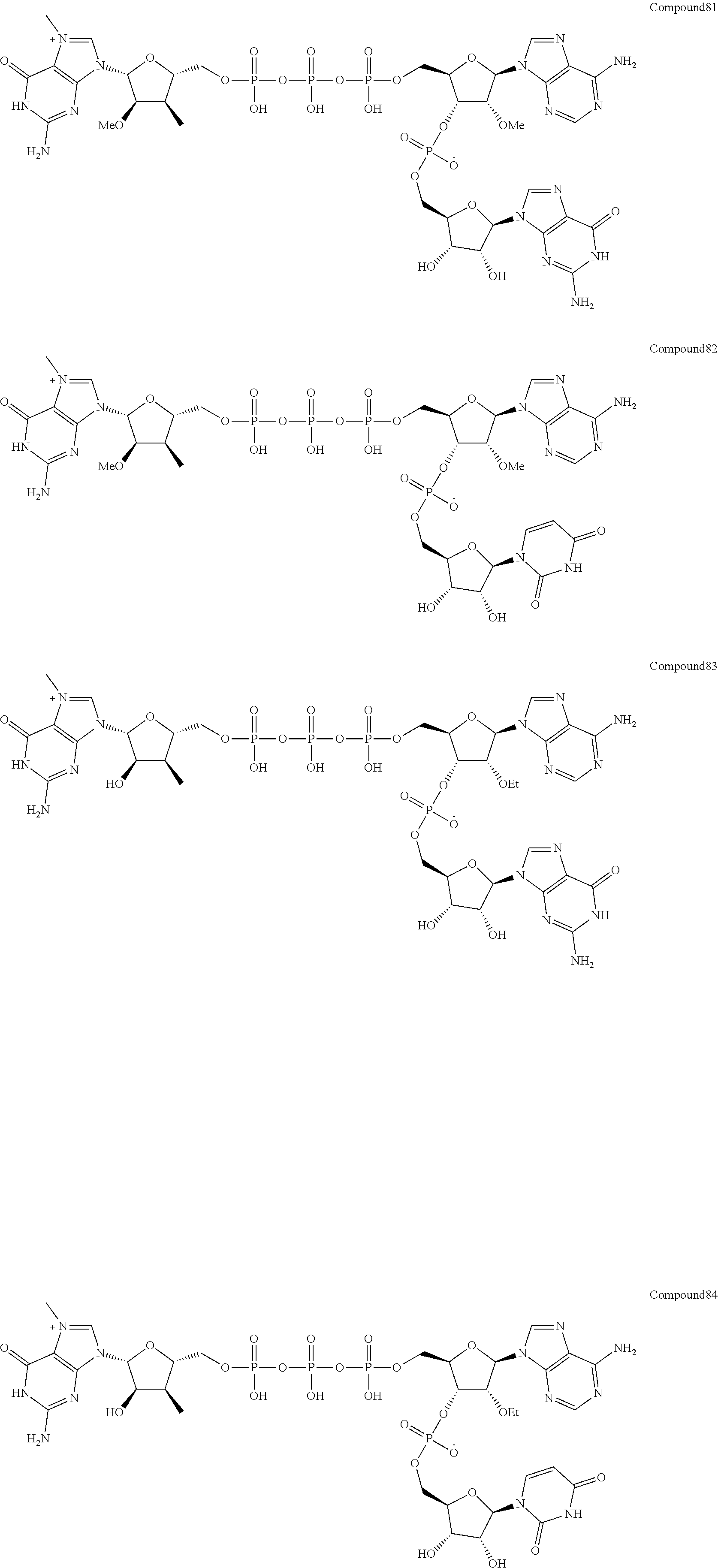

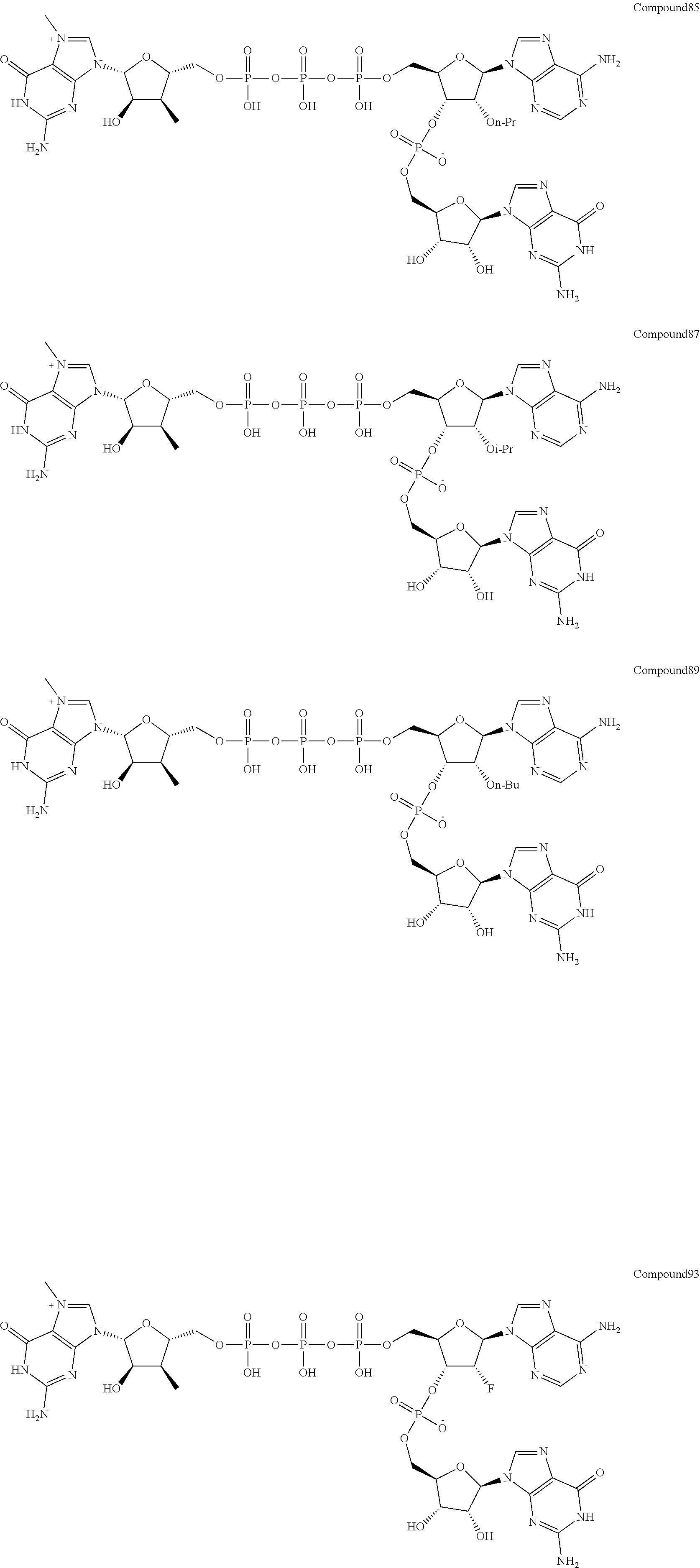

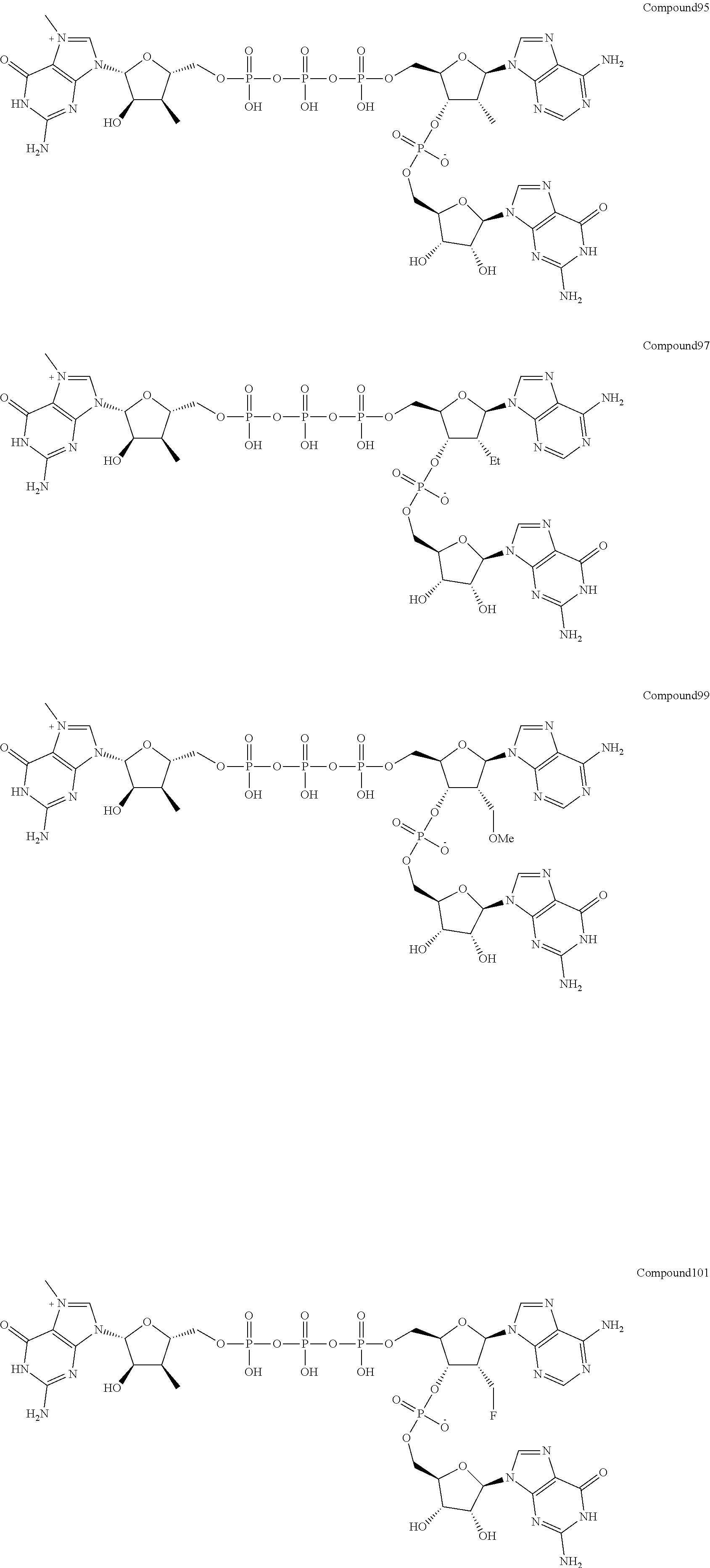

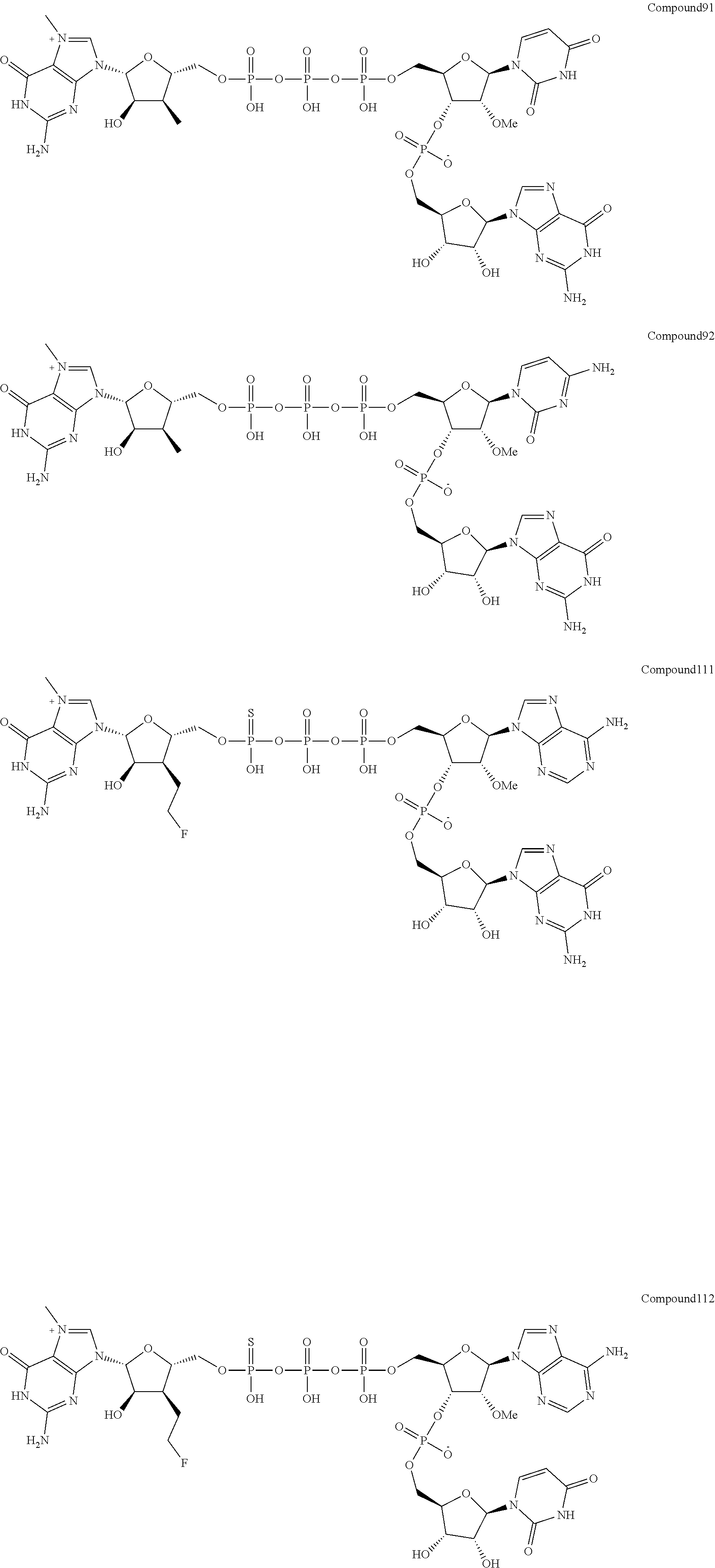

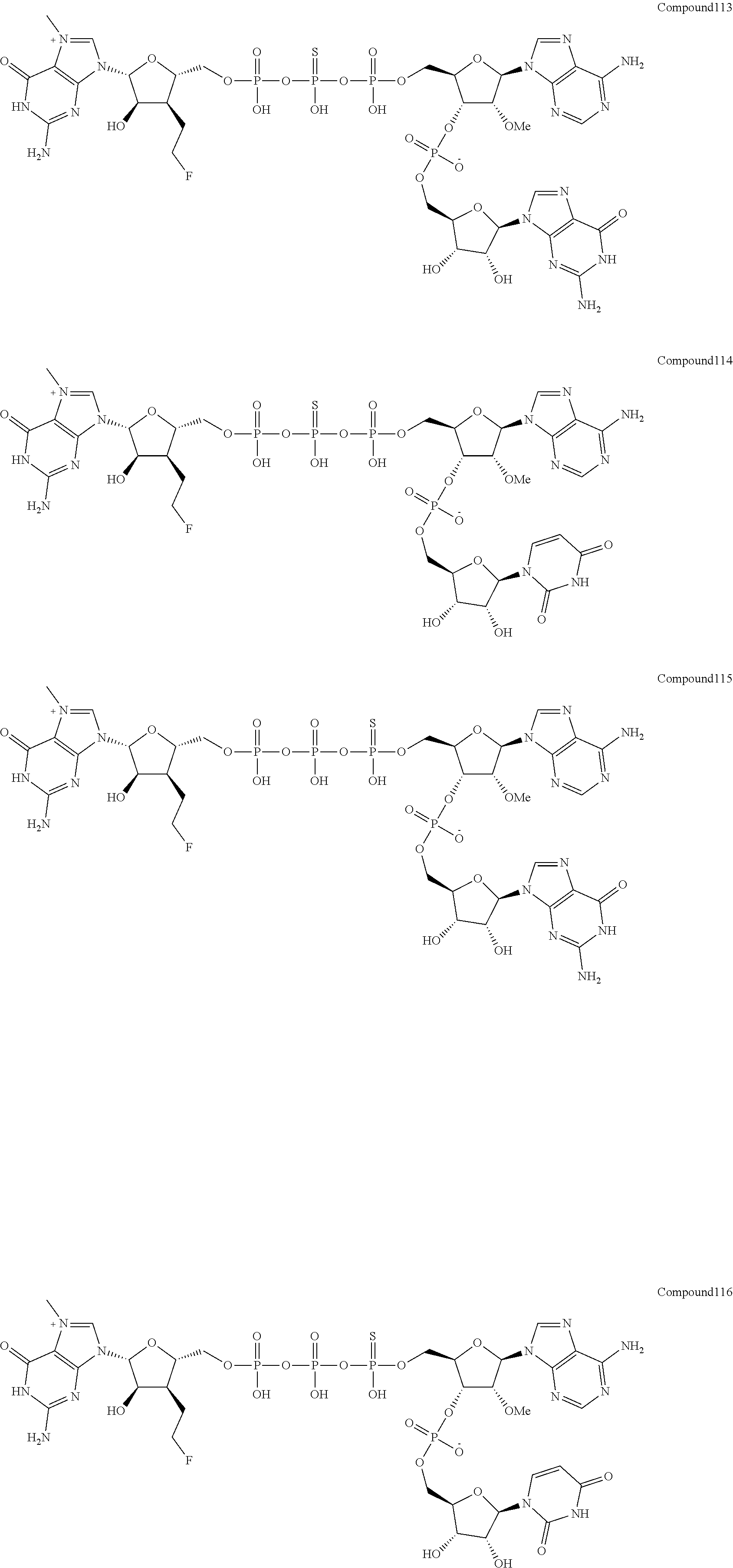

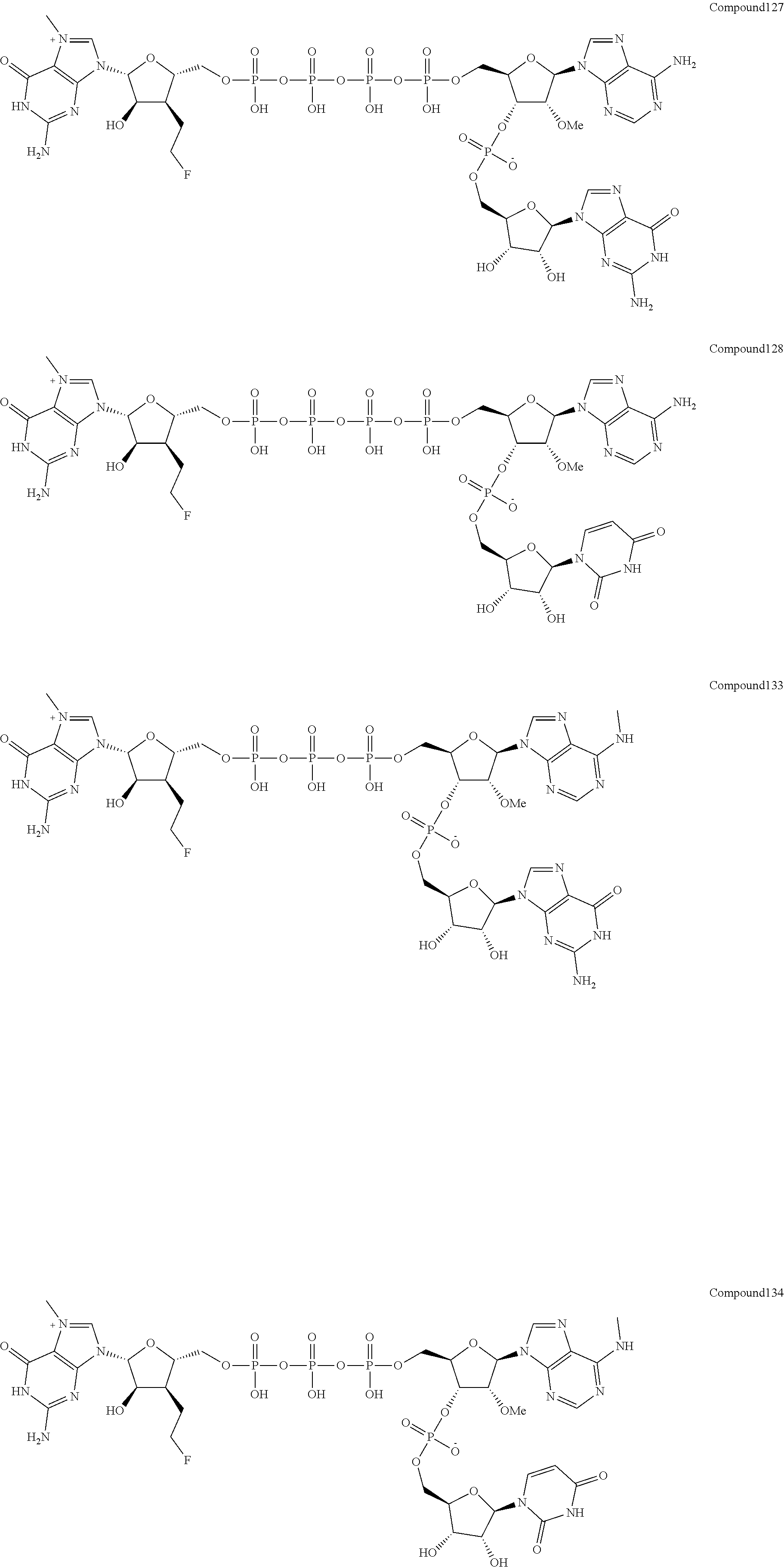

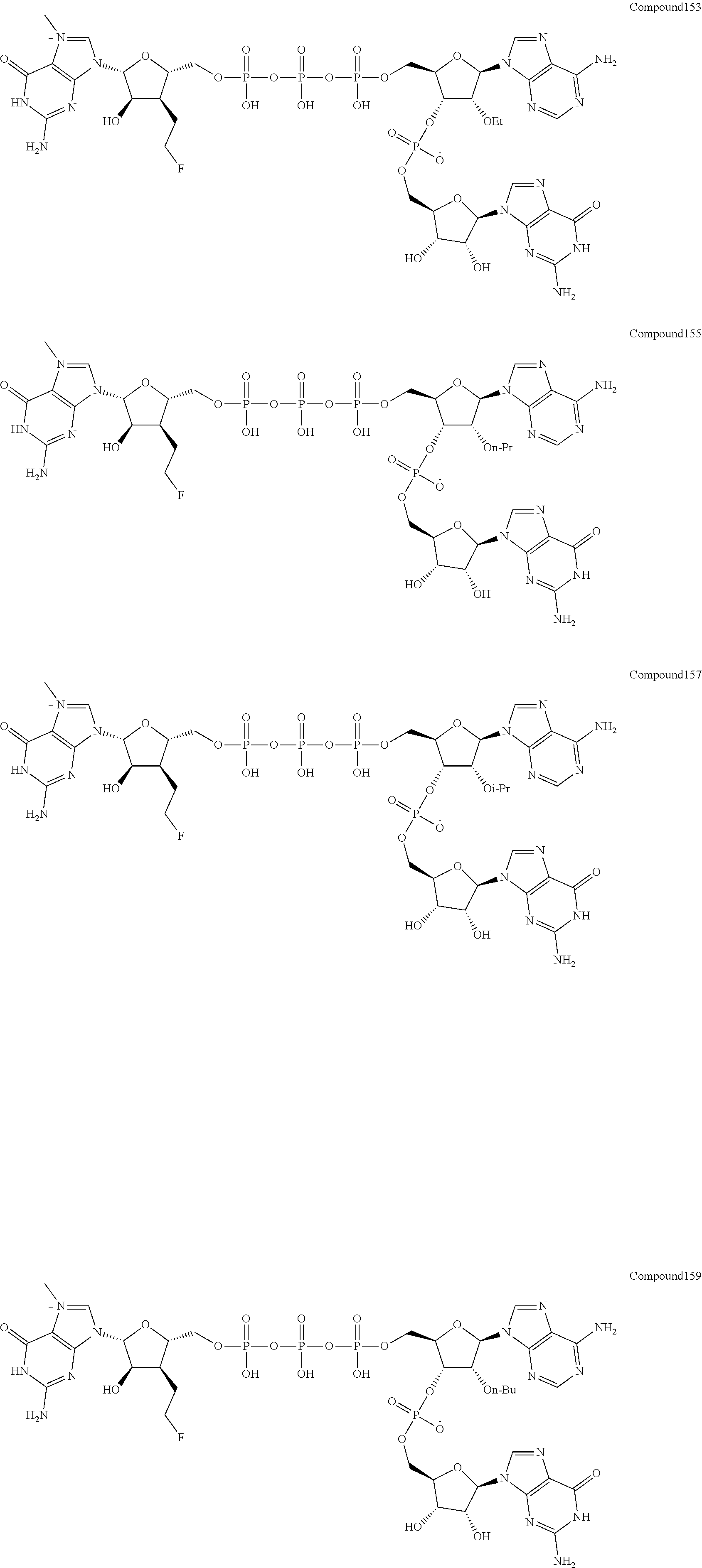

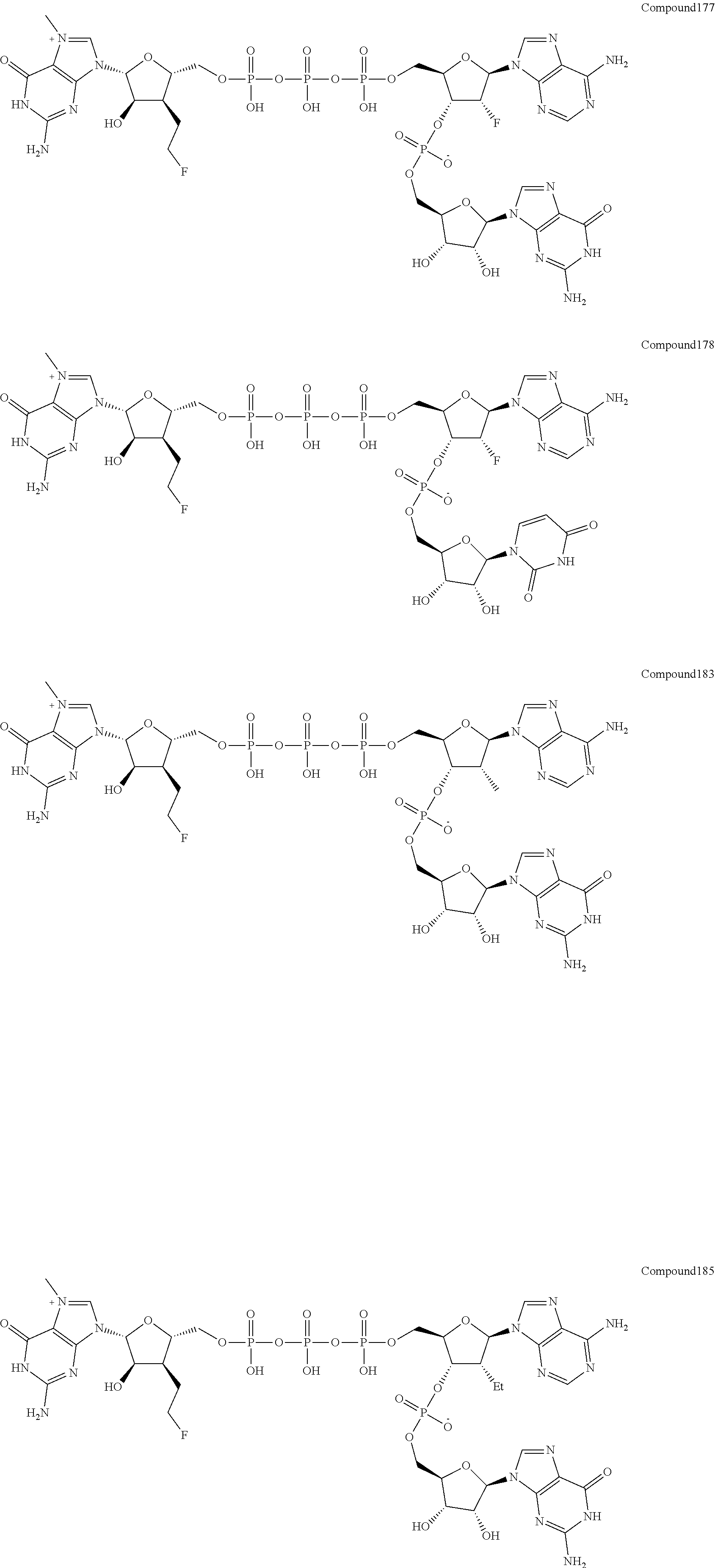

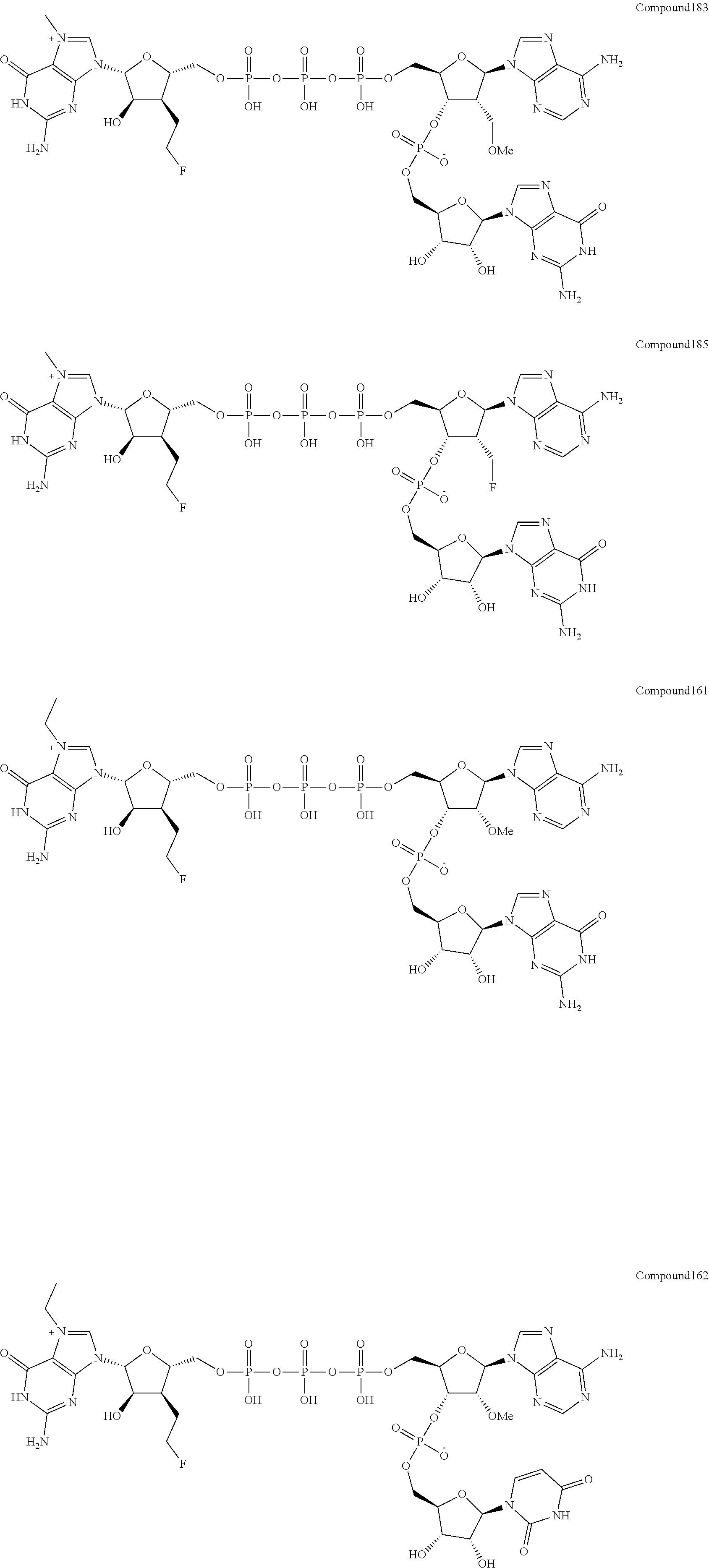

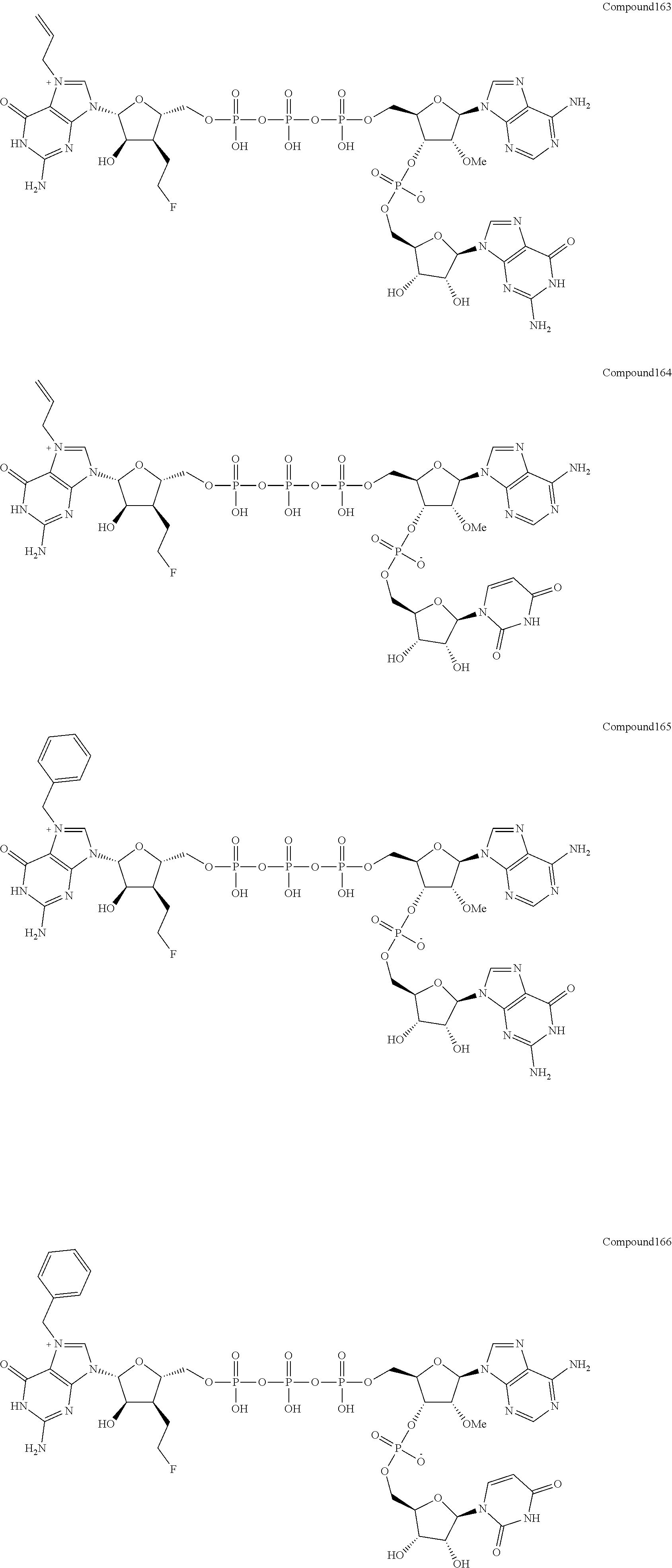

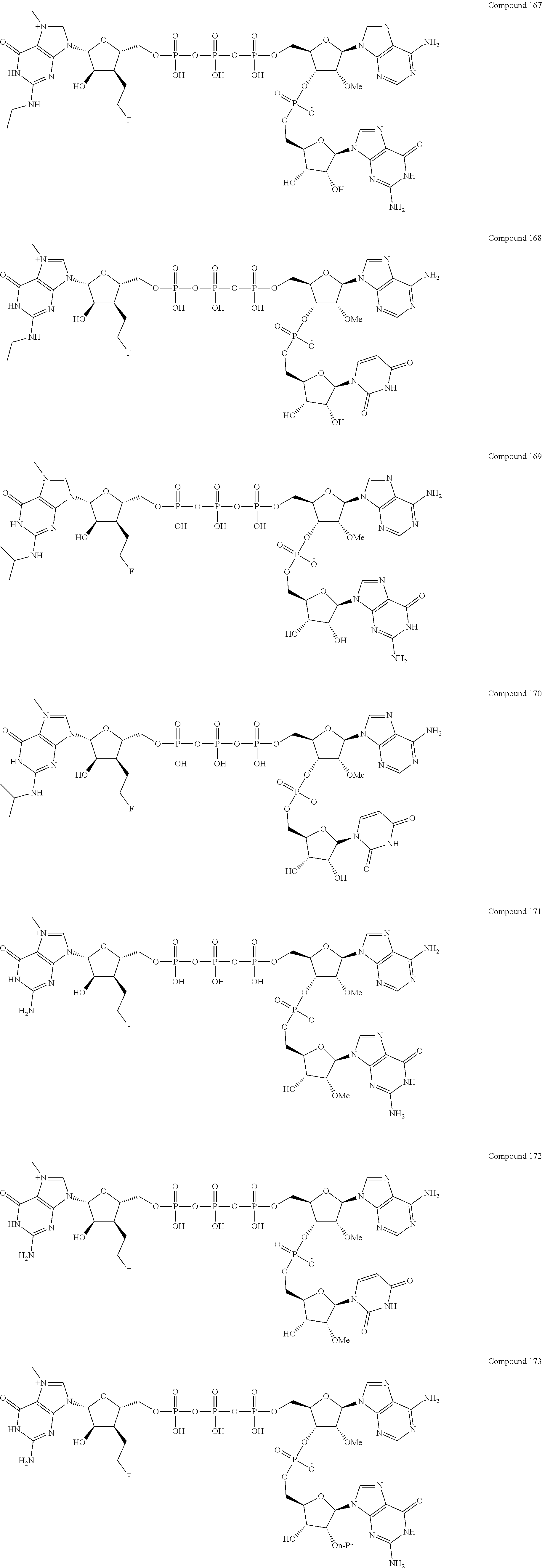

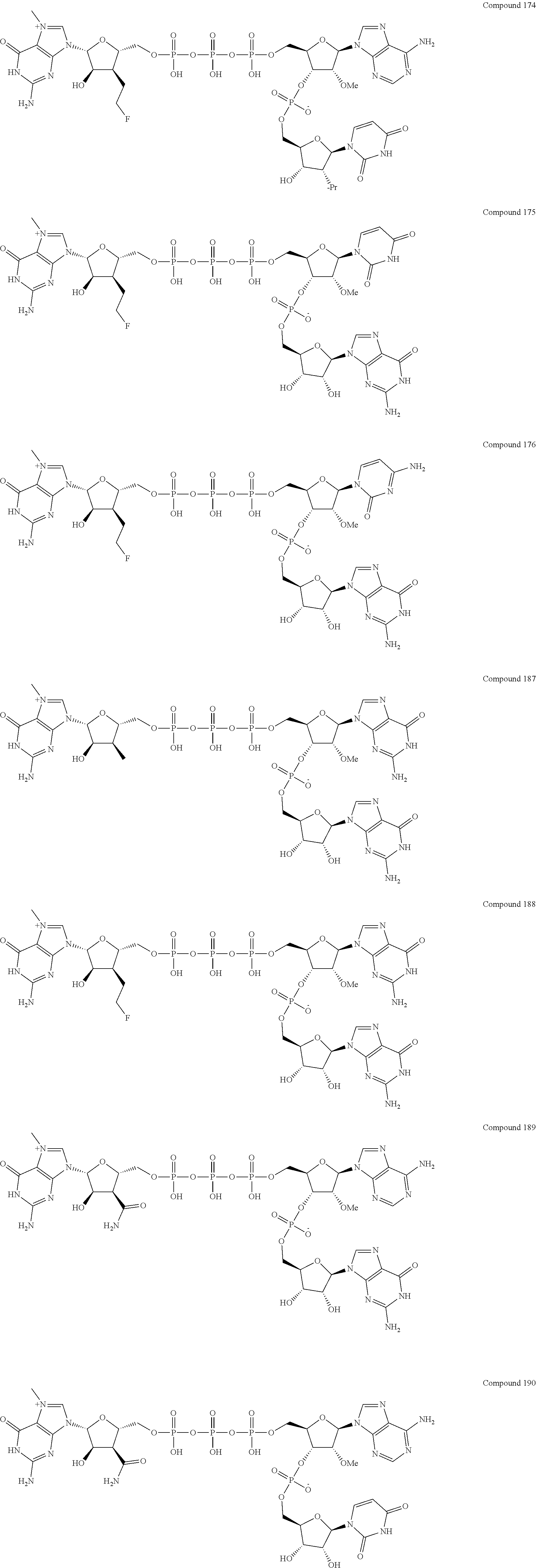

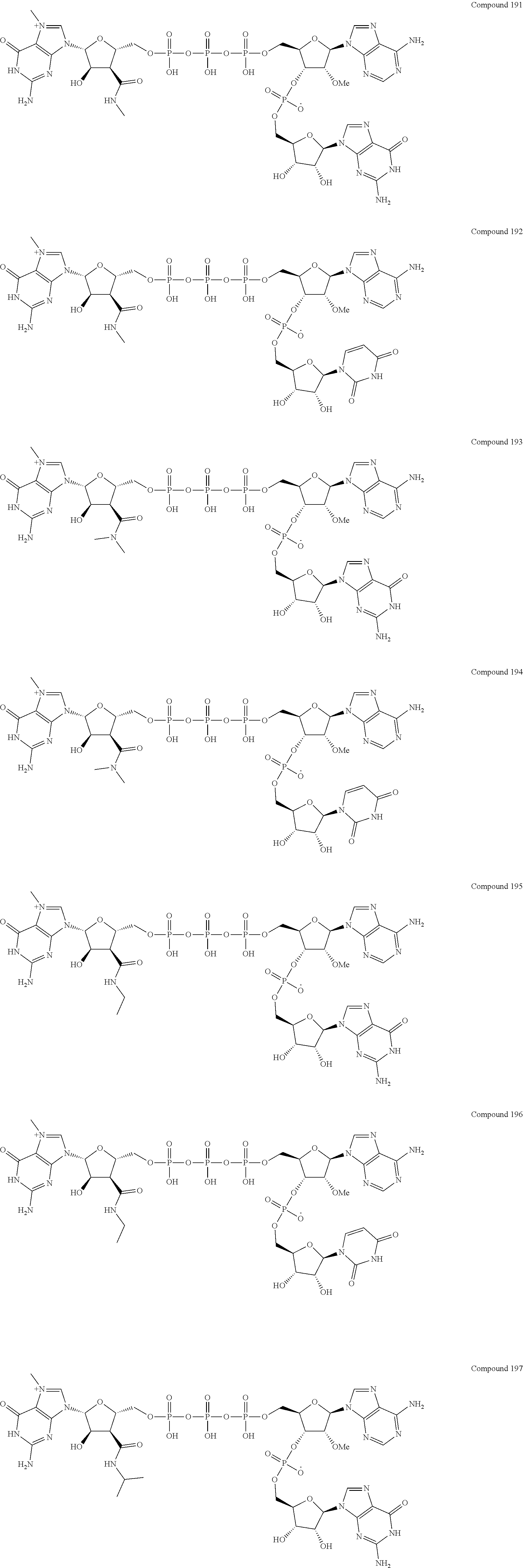

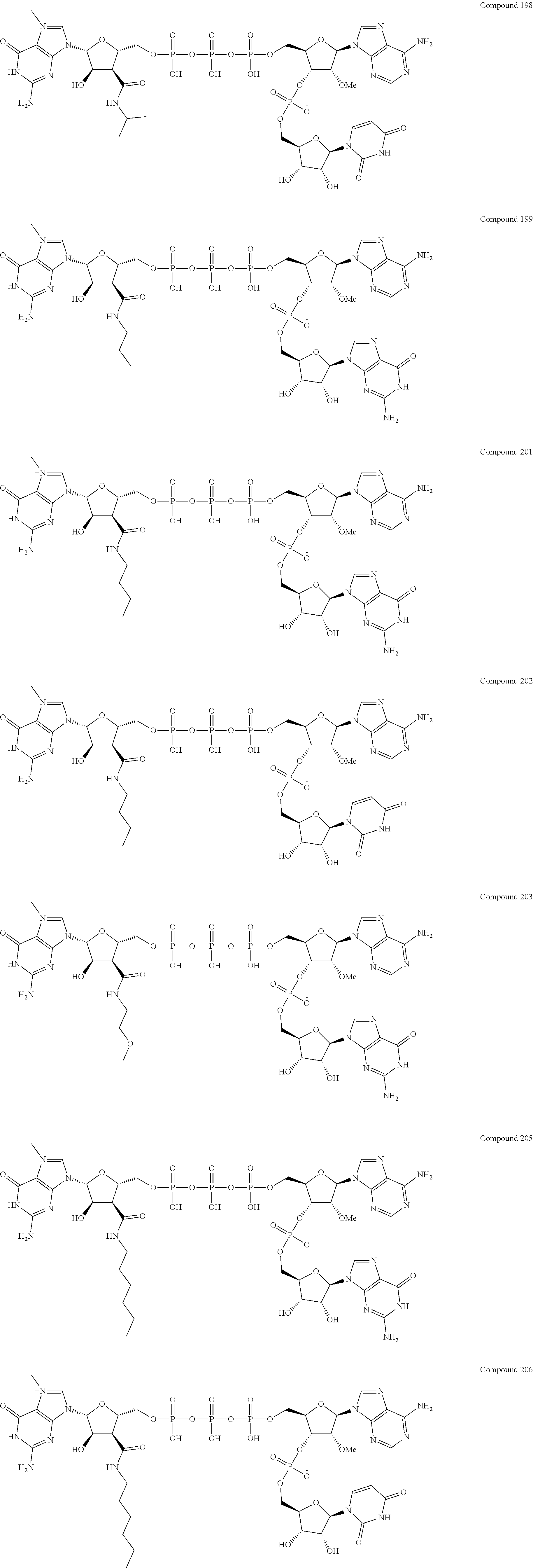

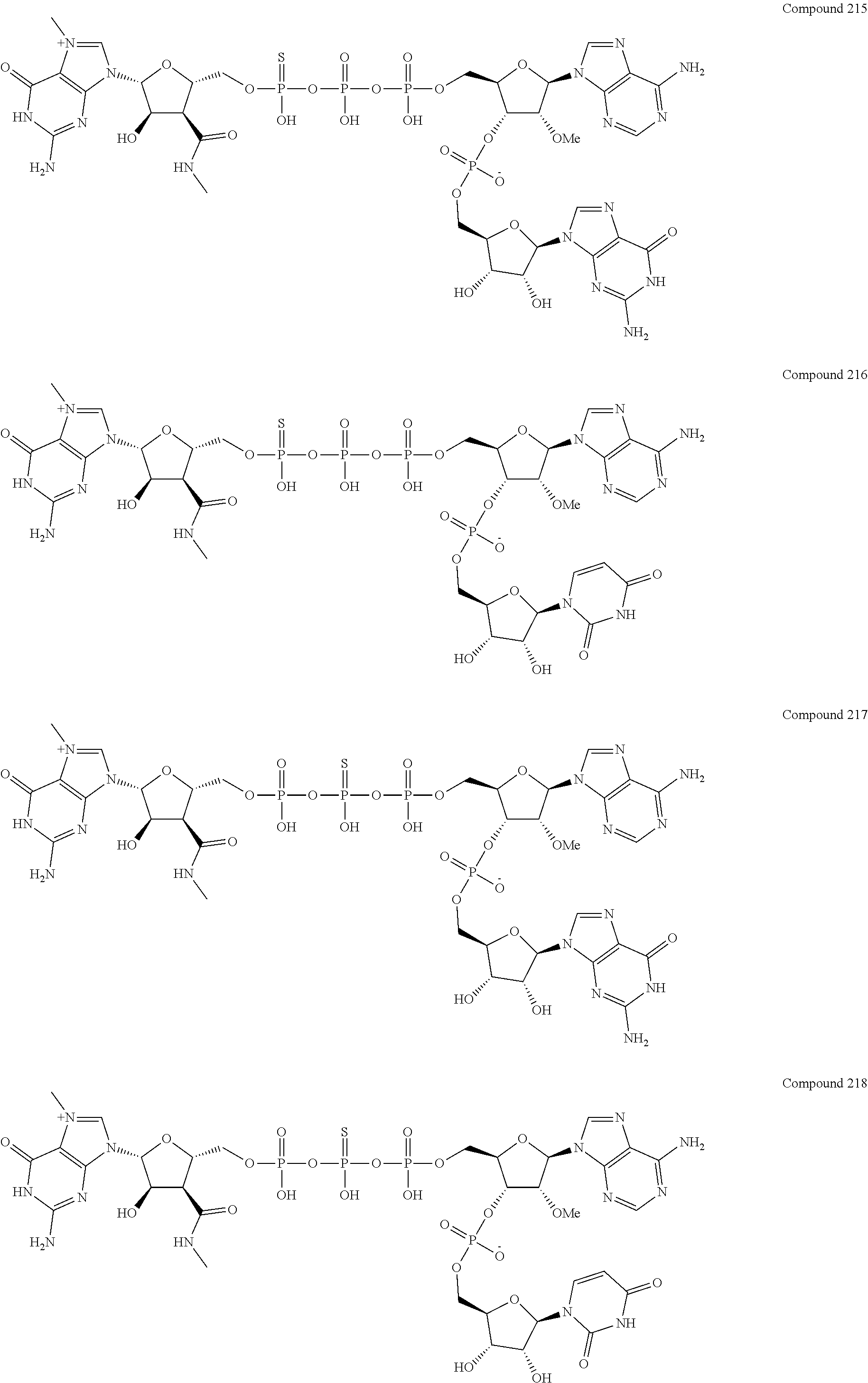

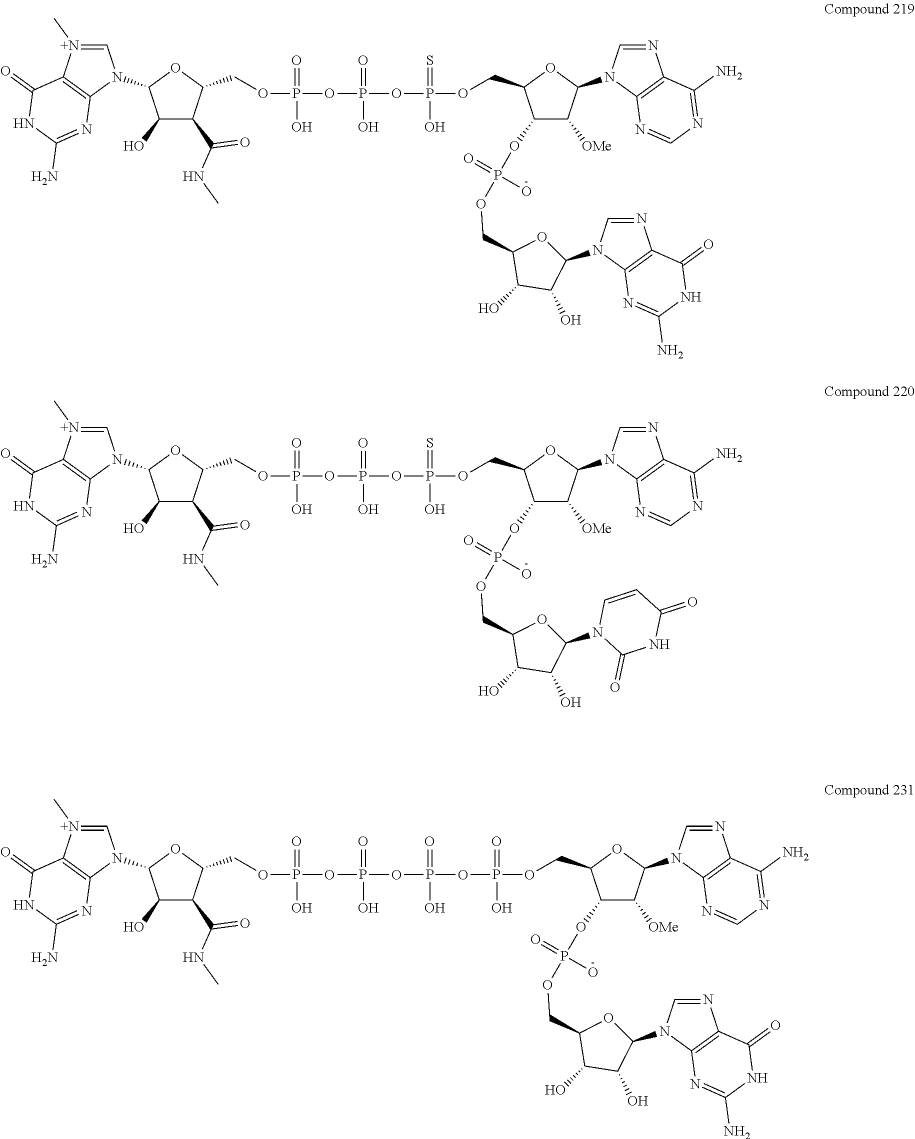

[chemical structure]

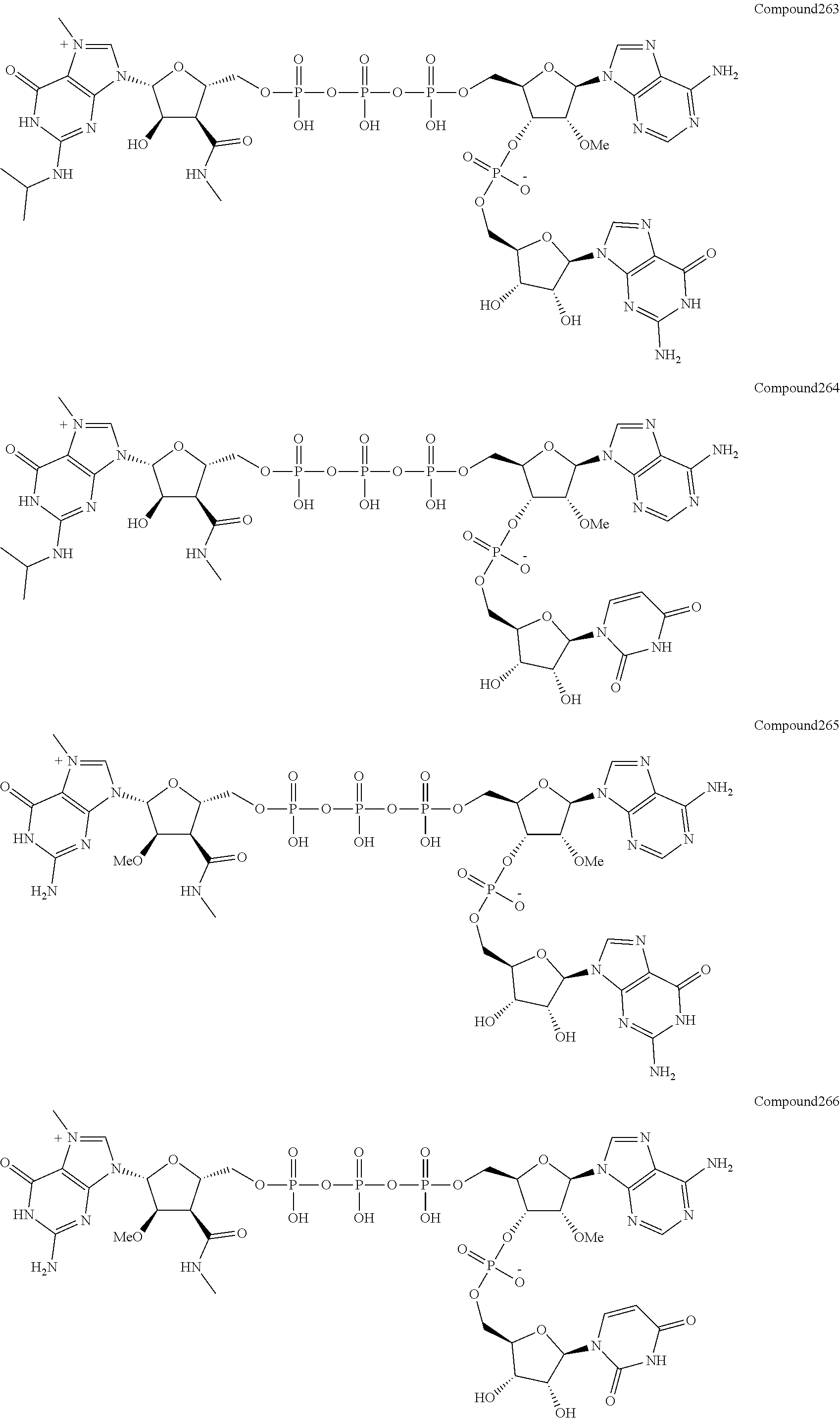

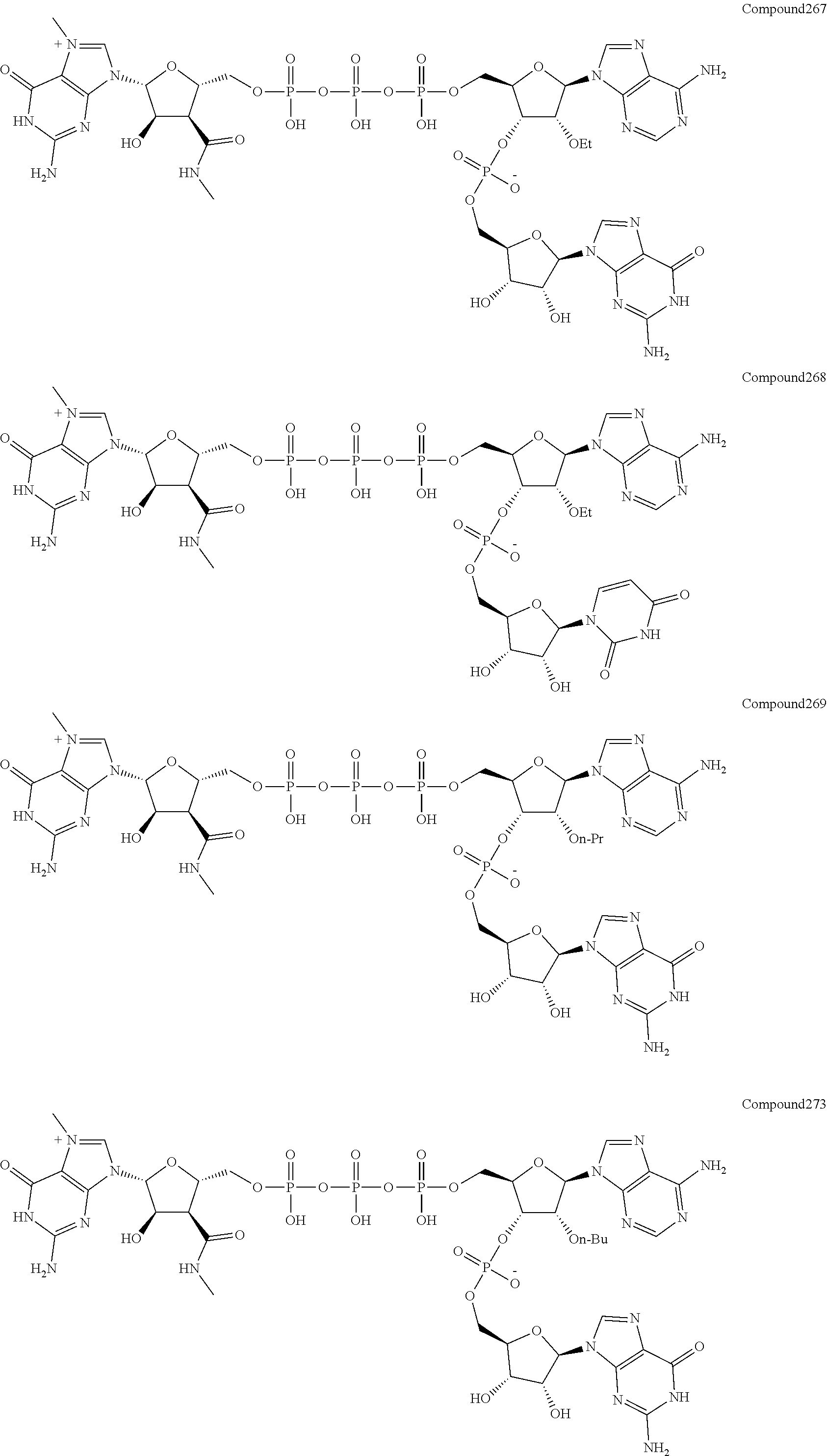

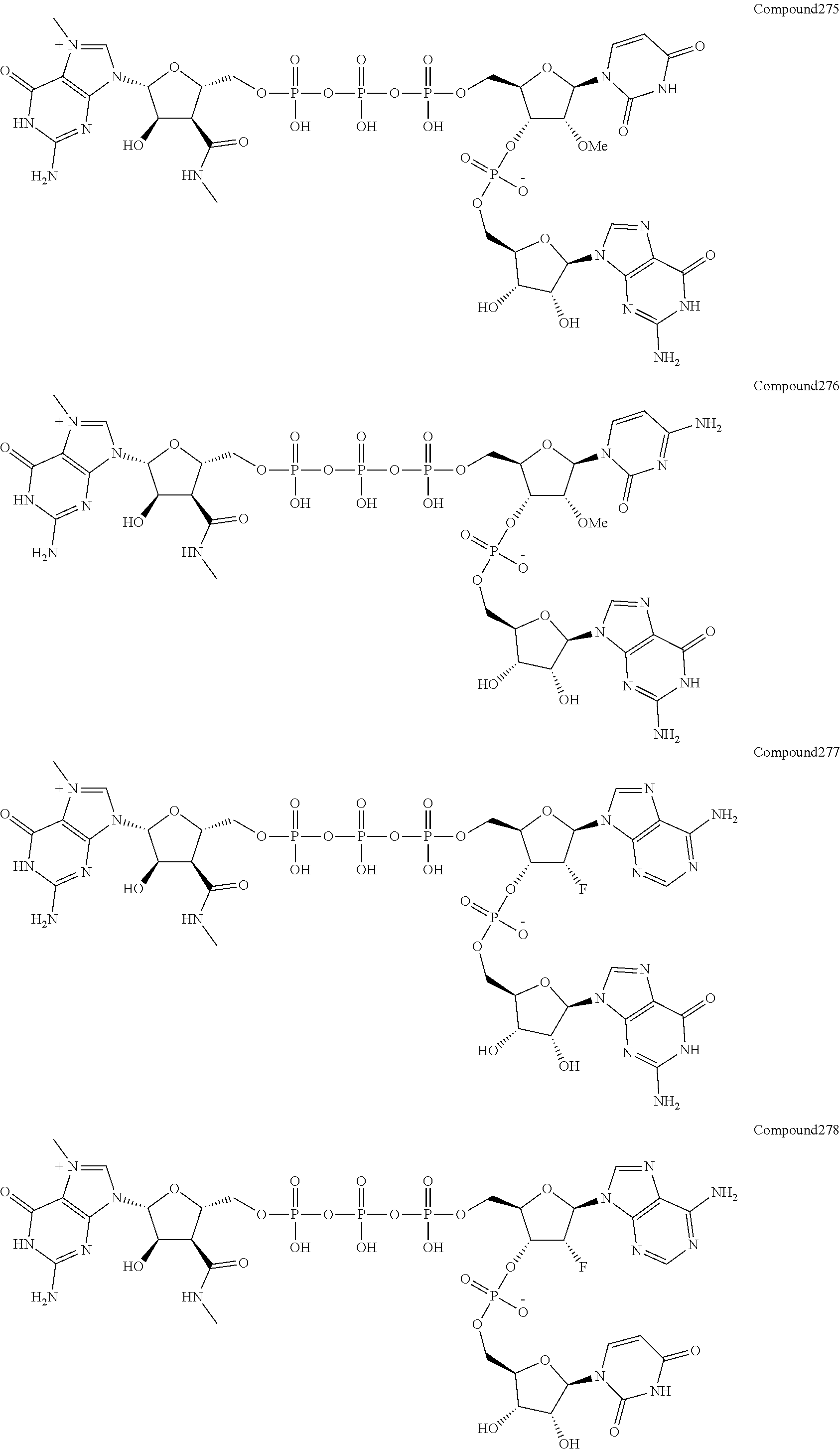

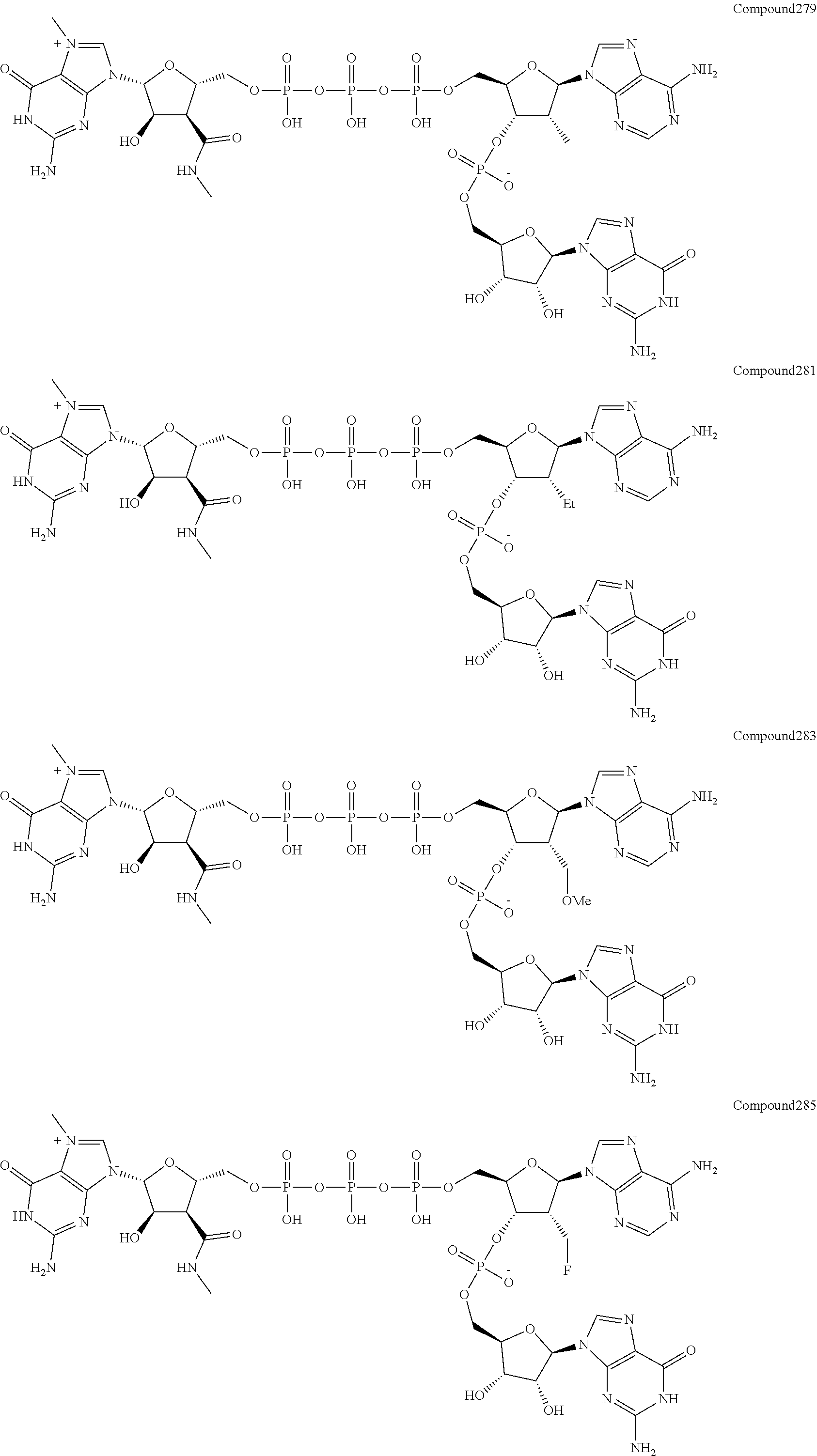

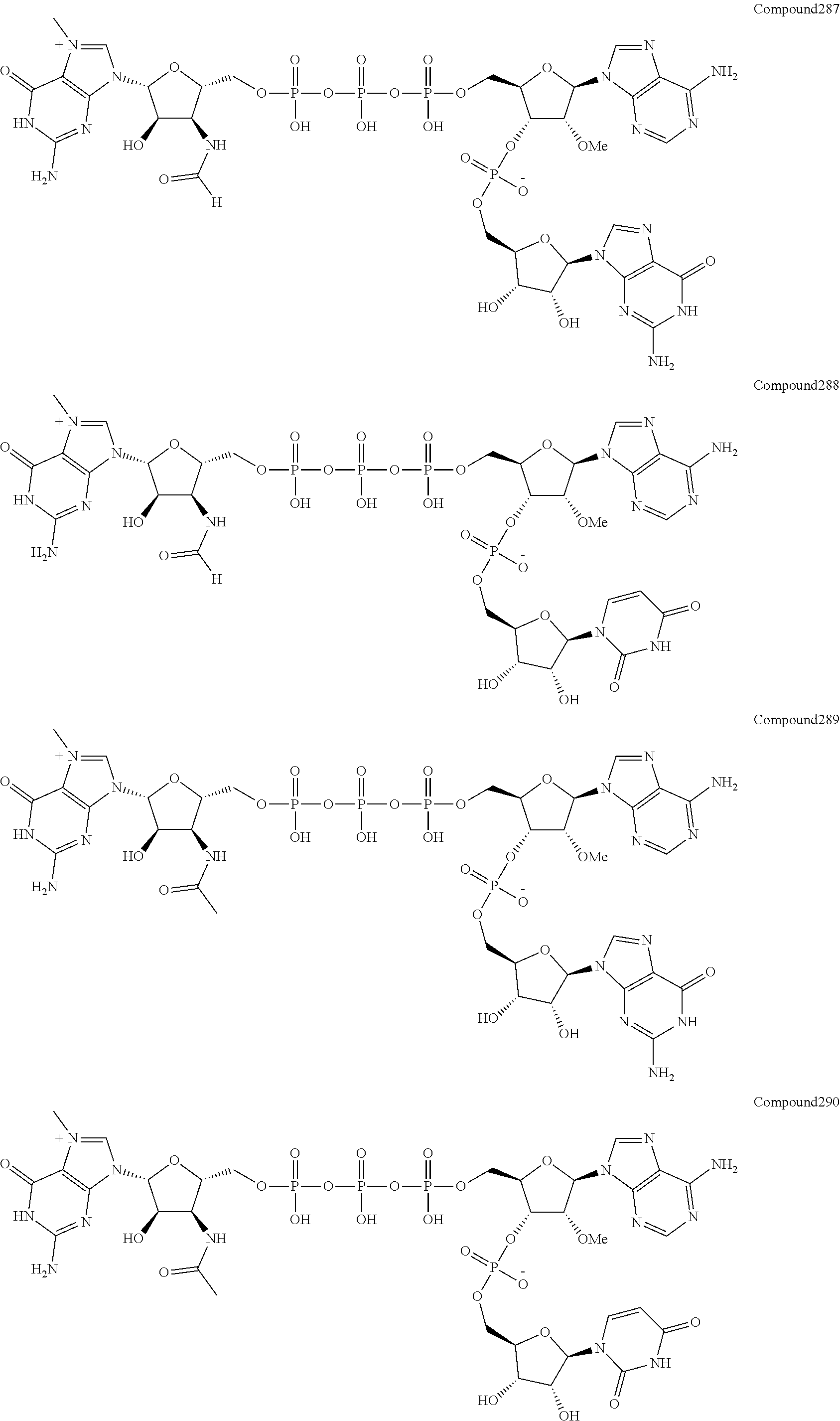

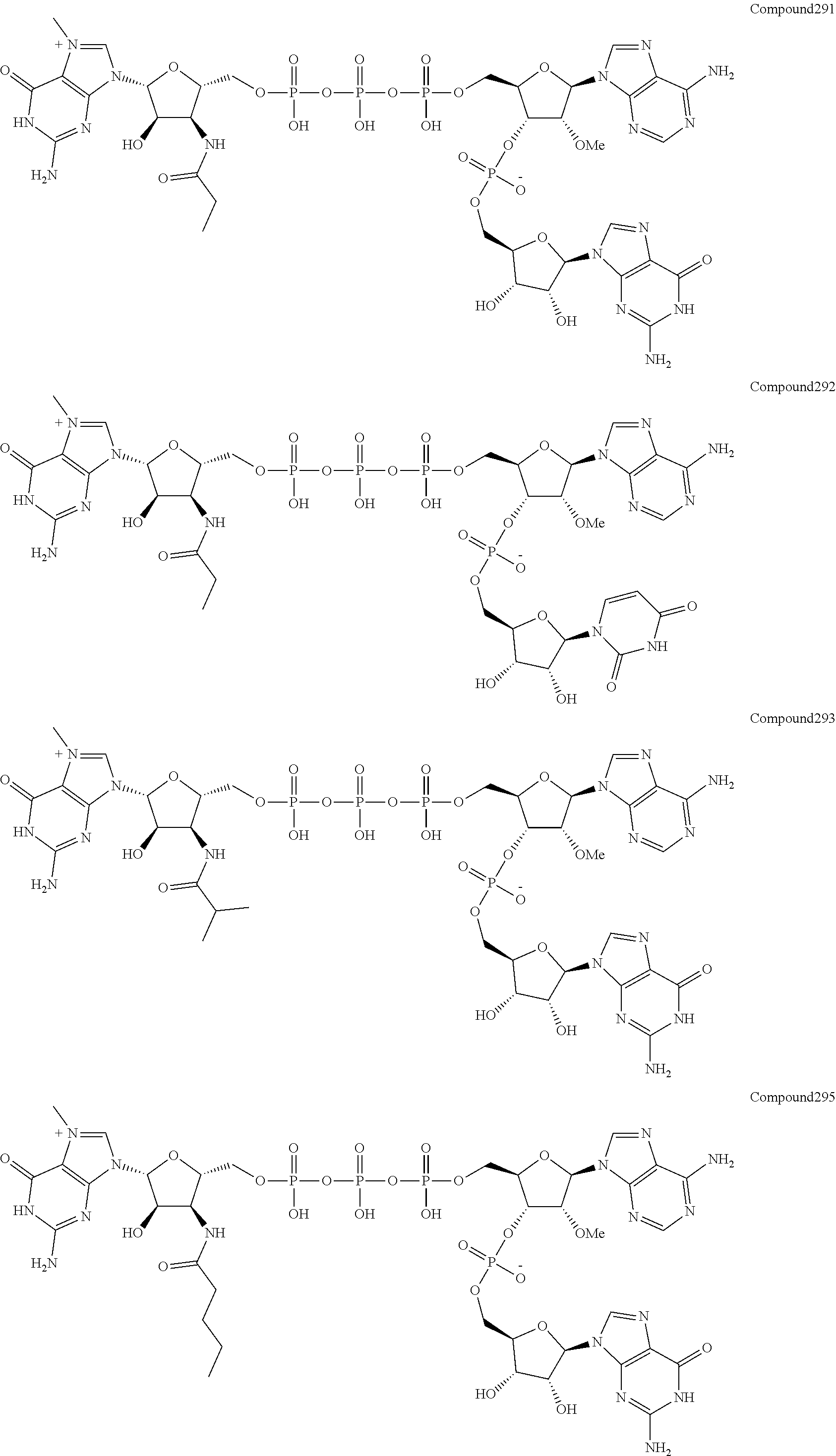

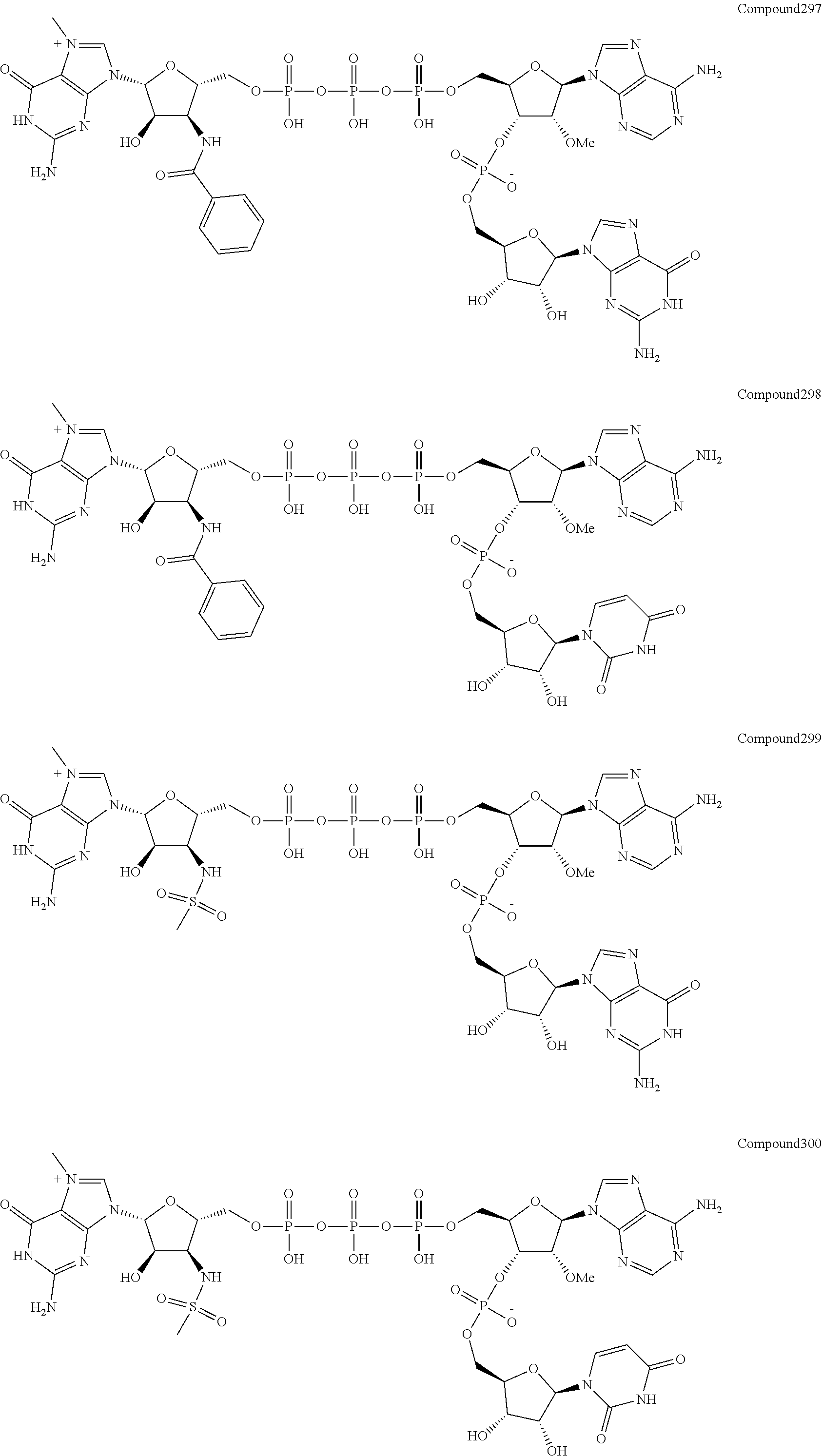

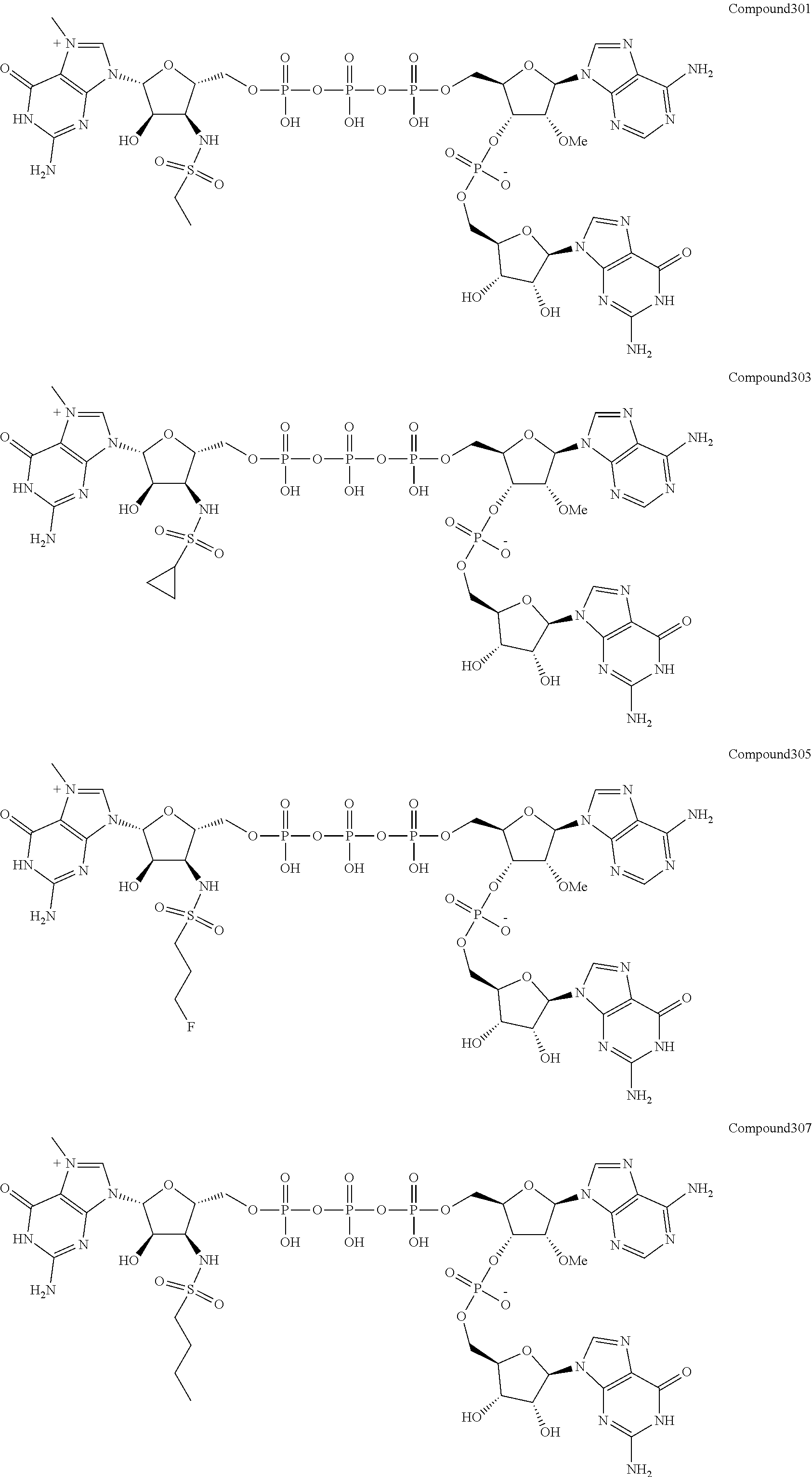

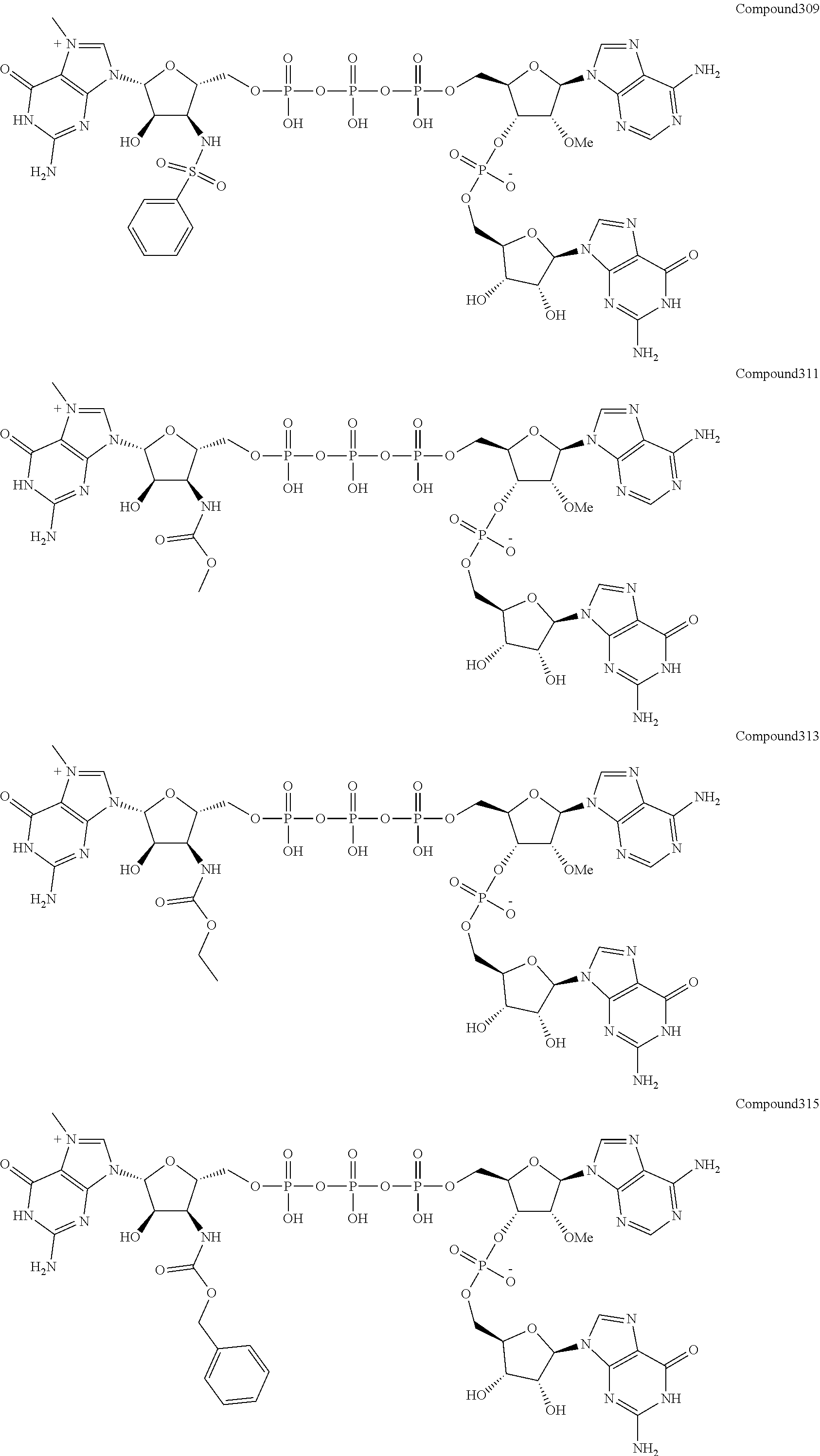

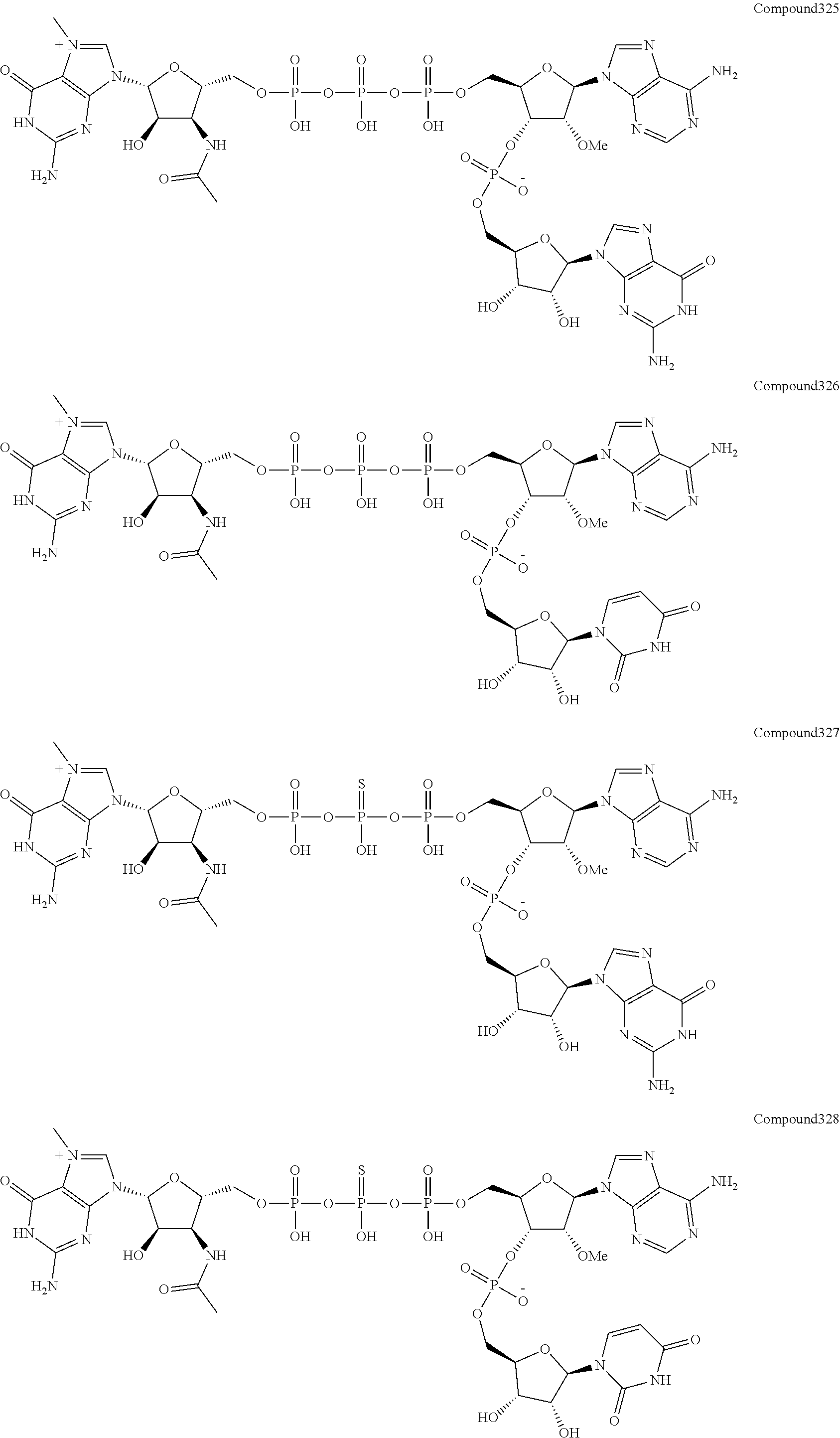

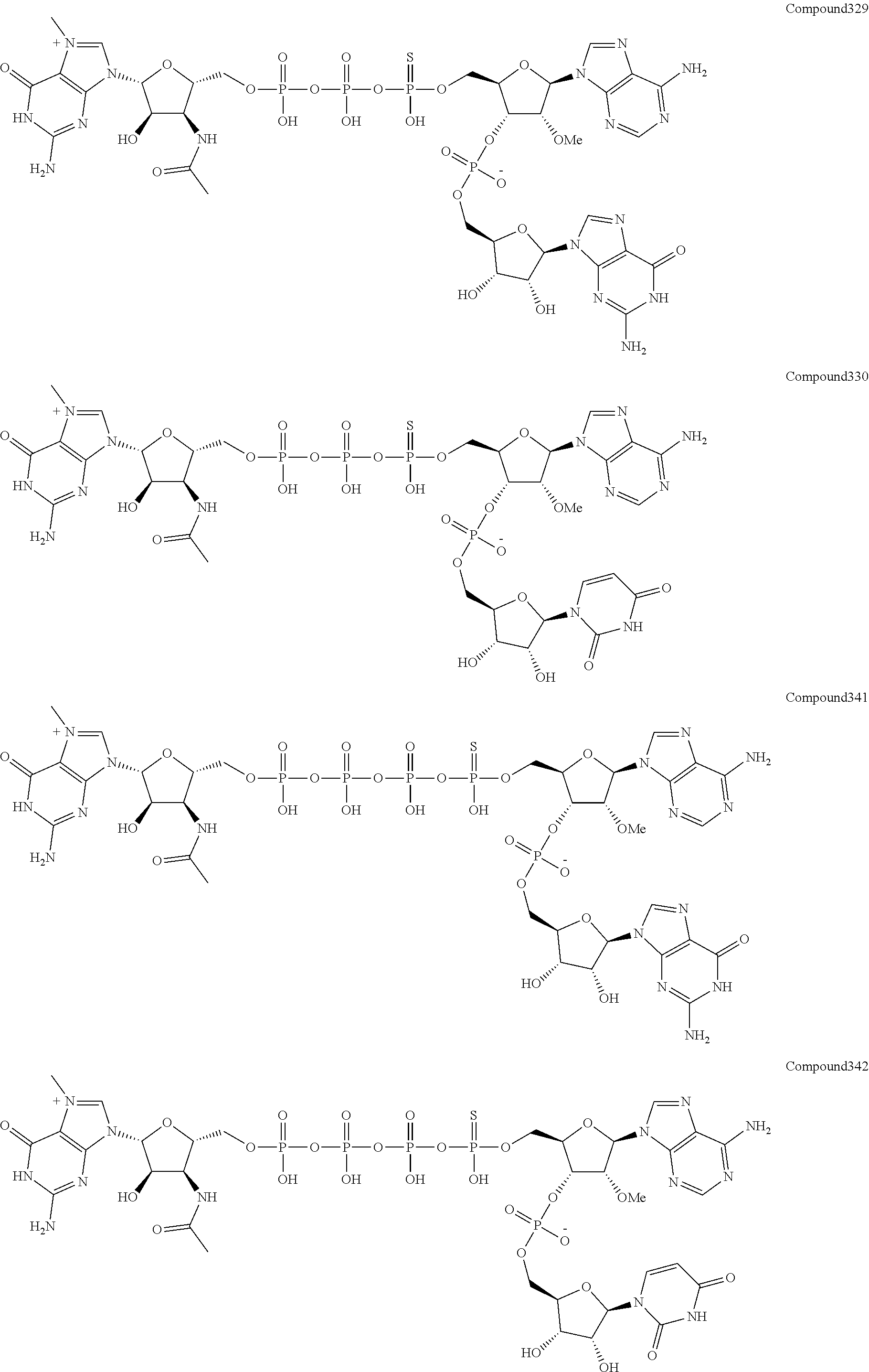

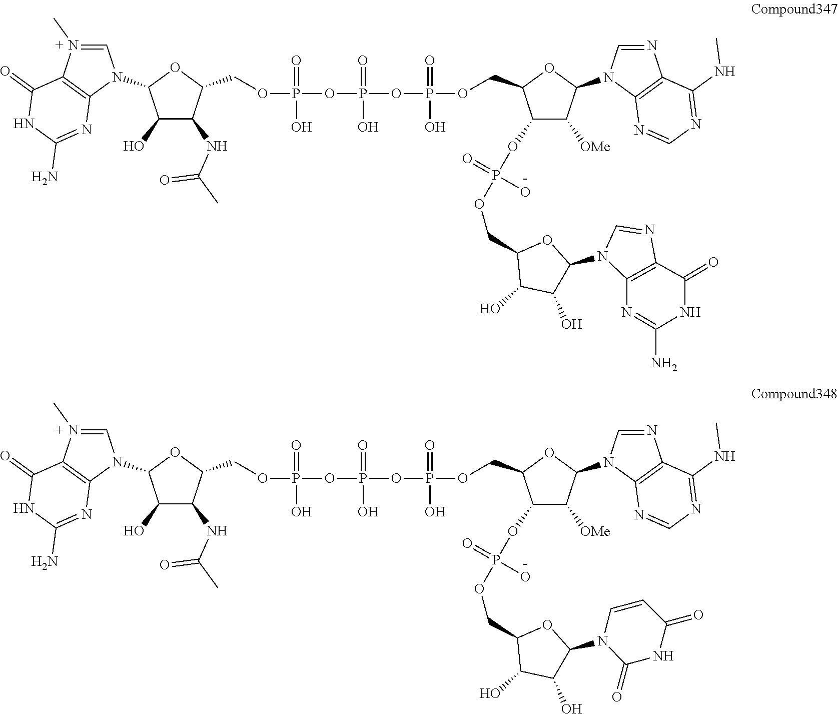

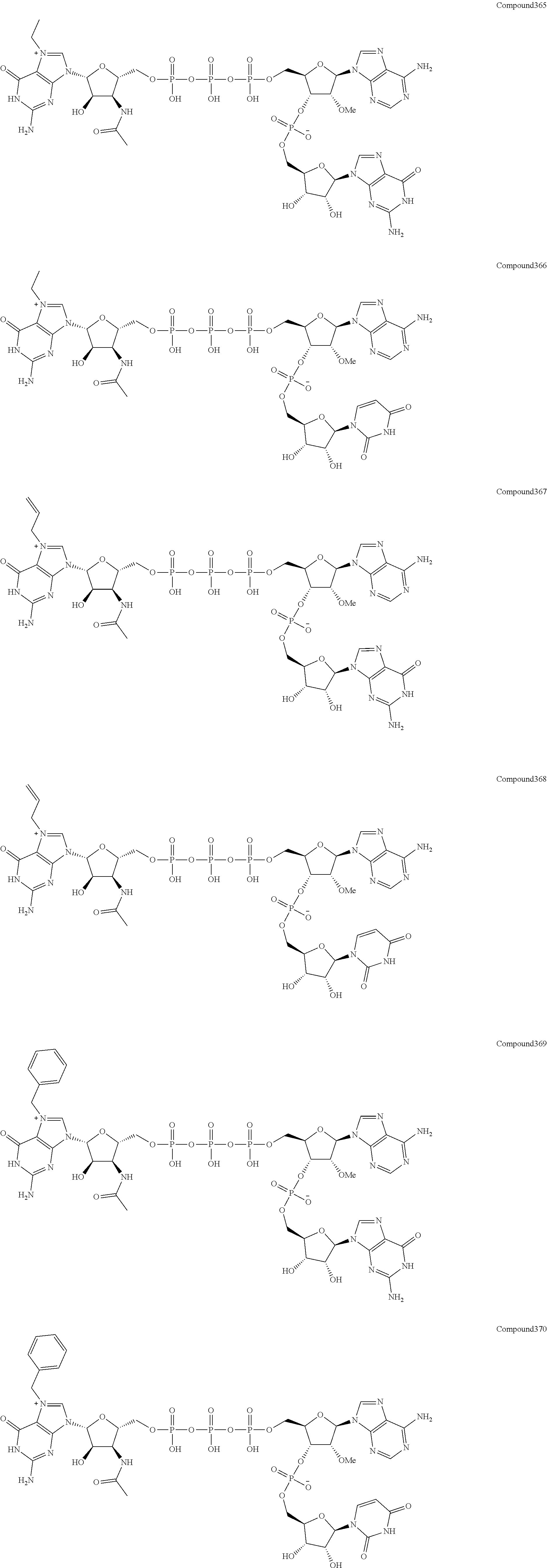

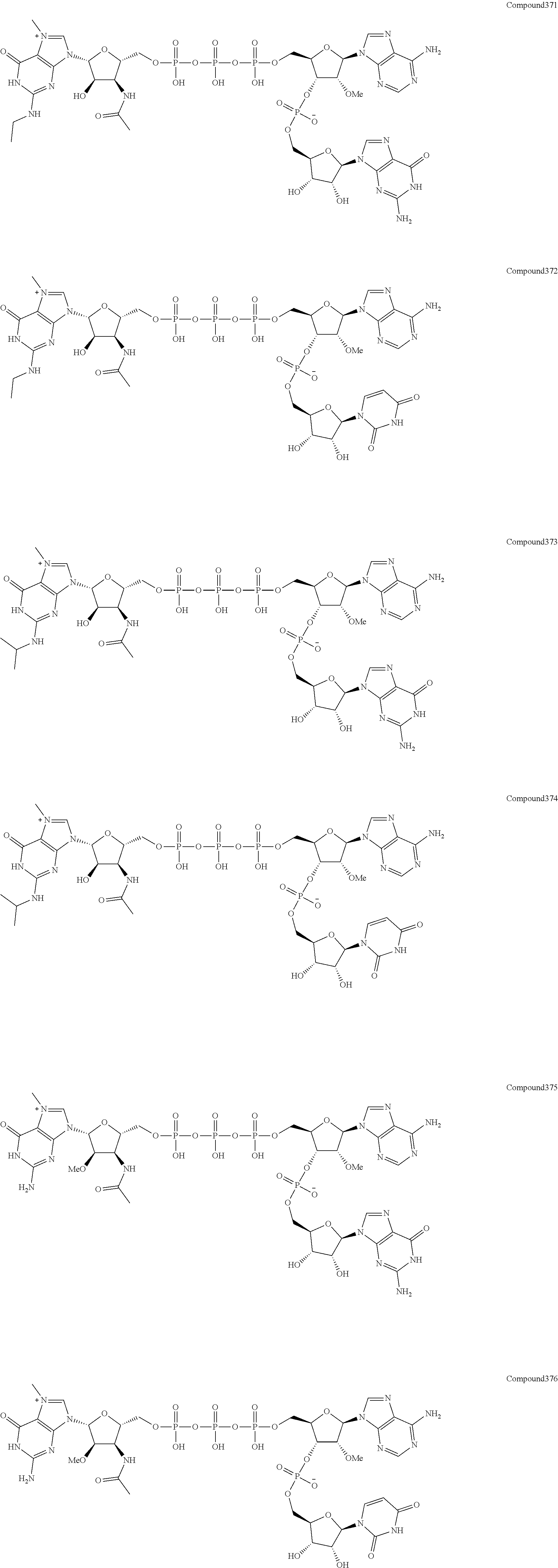

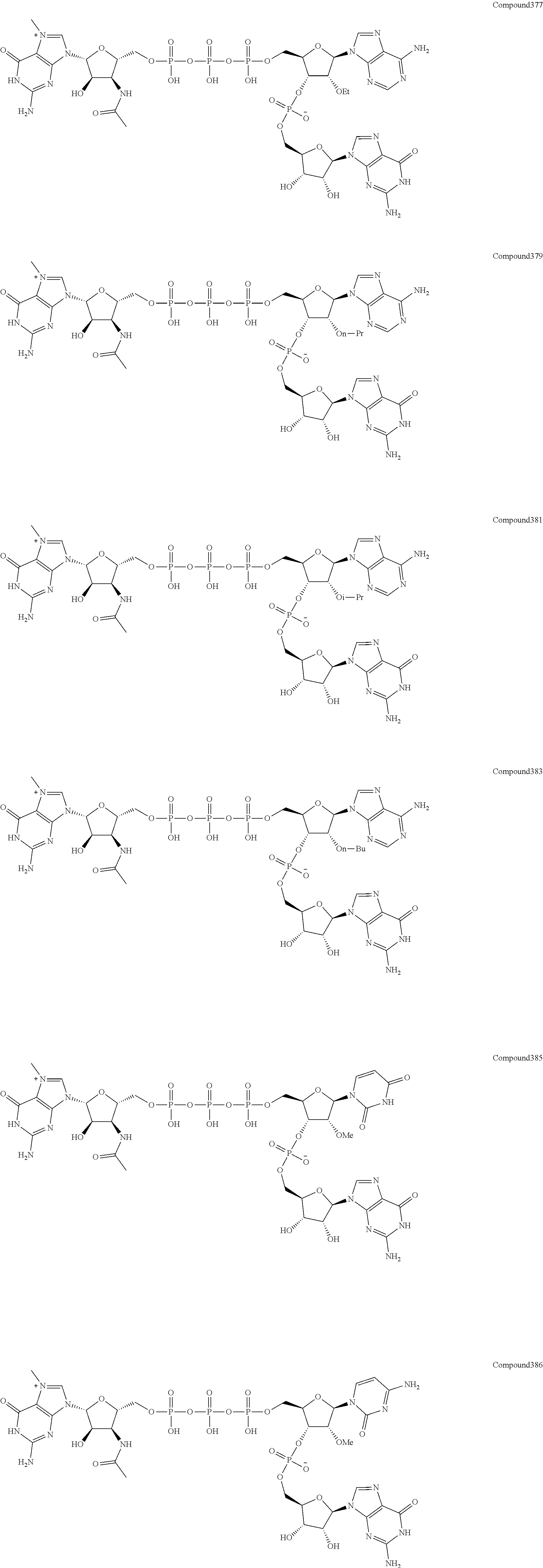

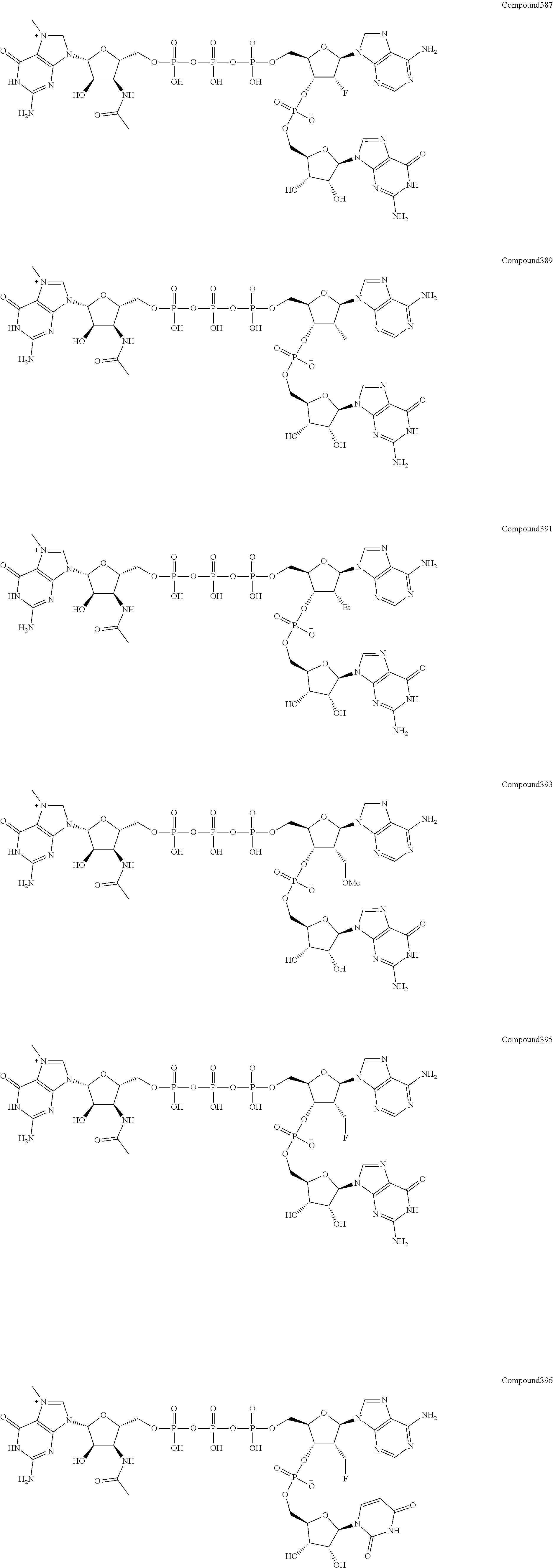

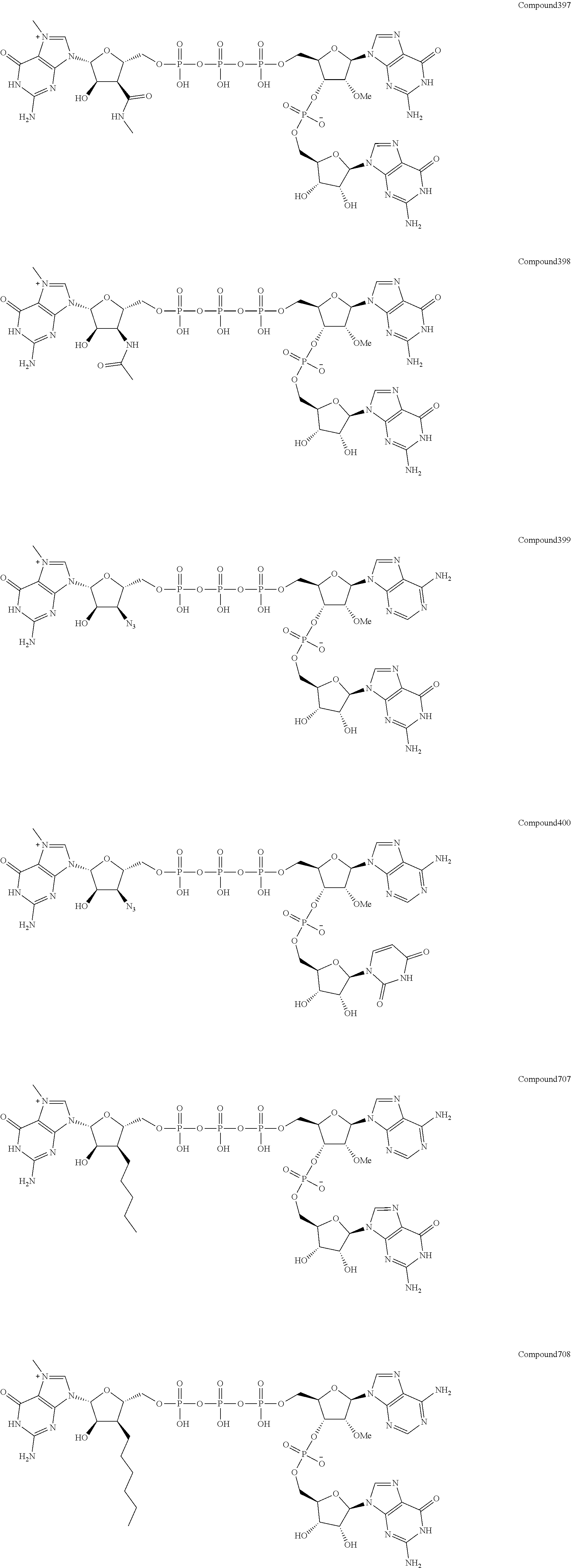

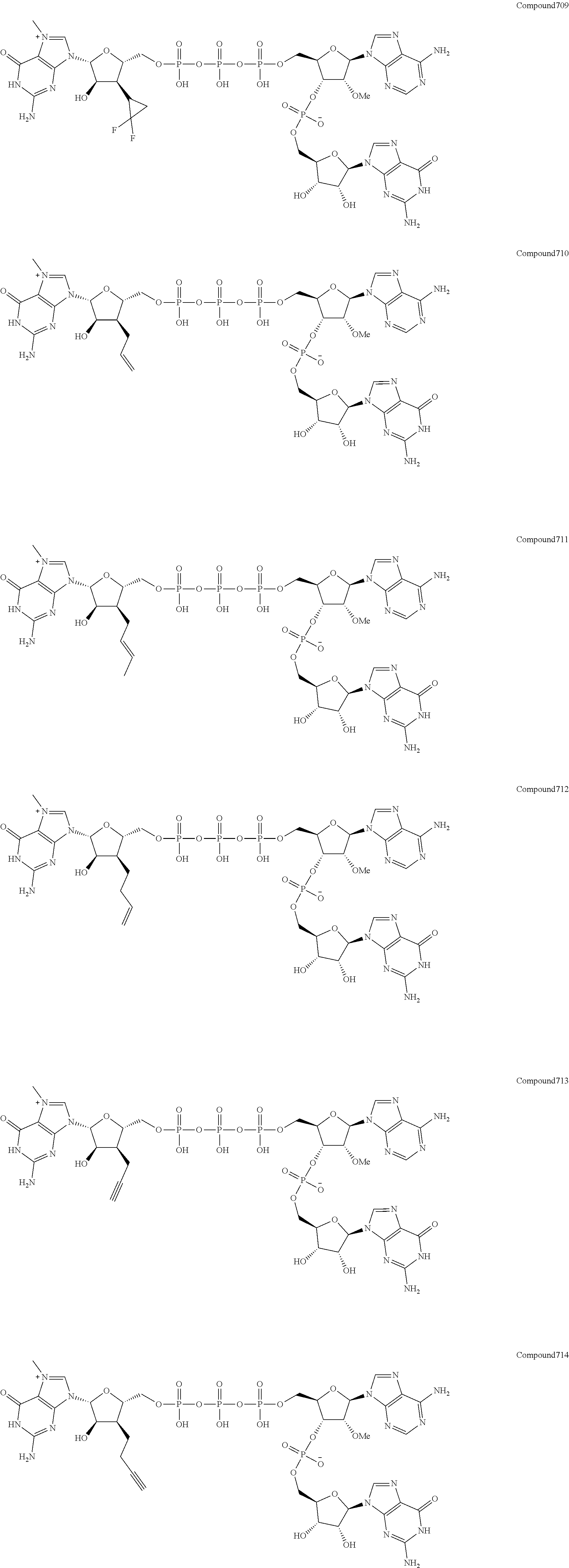

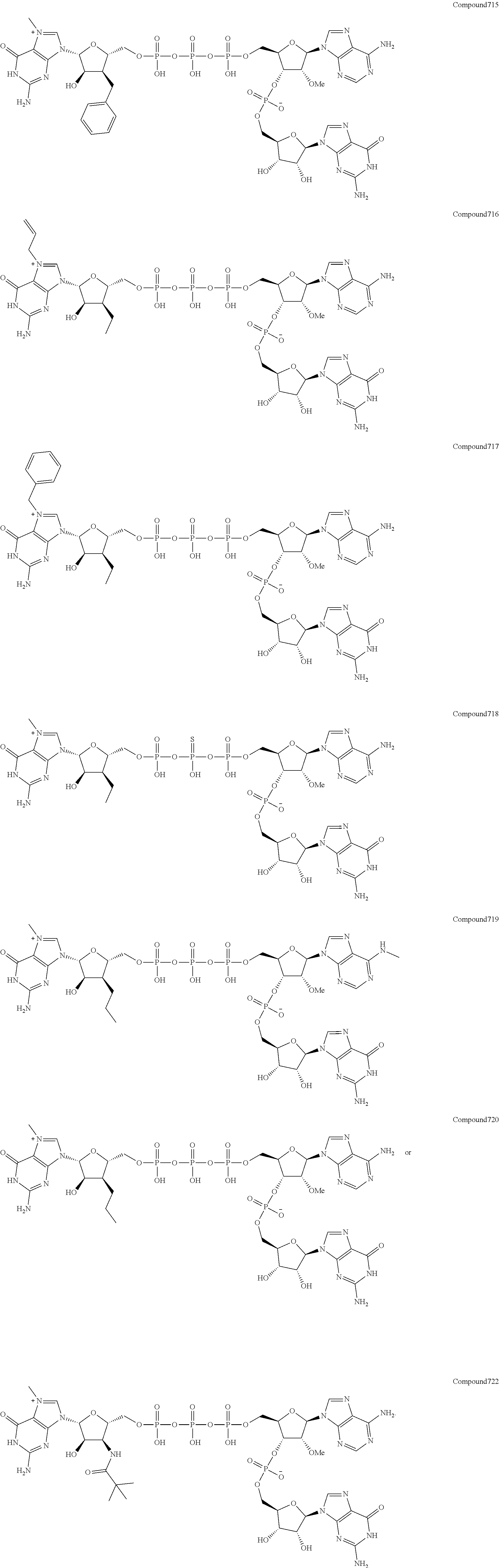

21. An in vitro co-transcriptional RNA capping reagent, comprising the compound as defined in claim 20.

22. An RNA molecule comprising the compound as defined in claim 20 as a cap structure or a cap structure fragment.

23. A pharmaceutical composition comprising the RNA molecule as defined in claim 22 and a pharmaceutically acceptable carrier.

24. A method for synthesizing an RNA molecule, comprising the following step:
co-incubating the compound as defined in claim 20 with a polynucleotide template for template transcription.

25. A capped RNA transcription reaction system comprising: a polynucleotide template, the compound as defined in claim 20, NTPs, an RNA polymerase.

26. A compound having a structure represented by formula I-a11, or a stereoisomer, pharmaceutically acceptable salt or solvate thereof: 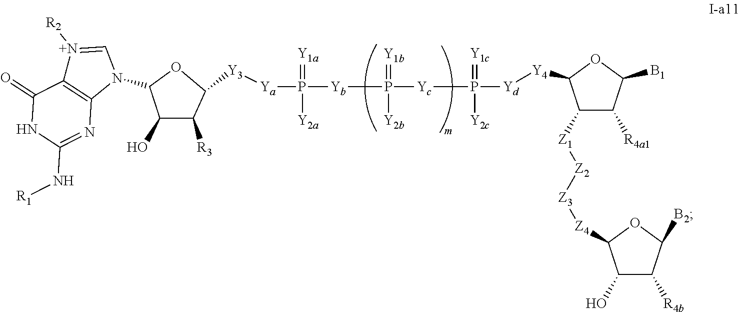
wherein
R₁ is H, C₁-C₆ alkyl, C₃-C₆ cycloalkyl, benzyl, R₅-substituted C₁-C₆ alkyl, or R₅-substituted benzyl;
R₂ is C₁-C₆ alkyl, C₂-C₆ alkenyl, C₂-C₆ alkynyl, benzyl or R₅-substituted benzyl;
R₃ is C₁-C₆ alkyl, C₂-C₆ alkenyl, C₃-C₆ cycloalkyl, benzyl, R₈-substituted C₁-C₆ alkyl, R₈-substituted C₂-C₆ alkenyl, R₈-substituted C₃-C₆ cycloalkyl, R₈-substituted benzyl, —C(O)NR₇R₈ or —NR₇SO₂R₈;
R₄ₐ₁ and R_{4ab} are independently H, OH, C₁-C₆ alkyl, C₁-C₆ alkoxy, R₈-substituted C₁-C₆ alkyl, R₈-substituted C₁-C₆ alkoxy or halogen;
Yₐ, Y_{b}, Y_{c} and Y_{d} are O;
Y₁ₐ, Y_{1b} and Y_{1c} are independently O or S, and Y₁ₐ, Y_{1b} and Y_{1c} are all O or at most one is S;
Y₂ₐ, Y_{2b} and Y_{2c} are independently OH, SH or BH₃, and Y₂ₐ, Y_{2b} and Y_{2c} are all OH or at most one is SH or BH₃;
Y₃ and Y₄ are CH₂;
Z₁ is O;
Z₂ is PO(OH);
Z₃ is O;
Z₄ is CH₂;
B₁ and B₂ are independently a natural or modified cytosine nucleotide base, a natural or modified uracil nucleotide base, a natural or modified adenine nucleotide base or a natural or modified guanine nucleotide base;
R₅ is halogen;
R₇ is H, C₁-C₈ alkyl, C₂-C₈ alkenyl or C₂-C₈ alkynyl;
R₈ is H, halogen, C₁-C₈ alkyl, phenyl, benzyl, C₁-C₈ haloalkyl, halophenyl or halobenzyl;
m is 1 or 2.

27. The compound according to claim 26, wherein R₃ is C₁-C₄ alkyl, C₂-C₆ alkenyl, C₃-C₆ cycloalkyl, benzyl, C₁-C₆ haloalkyl, C₂-C₆ haloalkenyl, C₃-C₆ halocycloalkyl, halobenzyl, —C(O)NR₇R₈ or —NR₇C(O)R₈;
R₈ is H, halogen, C₁-C₆ alkyl, phenyl, benzyl, C₁-C₆ haloalkyl, halophenyl or halobenzyl.

28. The compound according to claim 27, wherein R₃ is C₁-C₄ alkyl, C₂-C₄ alkenyl, C₃-C₆ cycloalkyl, C₁-C₄ haloalkyl, C₂-C₄ haloalkenyl or C₃-C₆ halocycloalkyl.

29. The compound according to claim 28, wherein R₃ is C₁-C₄ alkyl, C₃-C₆ cycloalkyl, C₁-C₄ haloalkyl or C₃-C₆ halocycloalkyl.

30. The compound according to claim 26, wherein R₇ is H, C₁-C₄ alkyl or C₂-C₄ alkenyl.

31. The compound according to claim 26, wherein R₃ is methyl, ethyl, propyl, isopropyl, cyclopropyl, n-butyl, sec-butyl, tert-butyl, propenyl, butenyl, halomethyl, haloethyl, halopropyl, haloisopropyl, halocyclopropyl, halo-n-butyl, halo-sec-butyl, halo-tert-butyl, halopropenyl, halobutenyl, benzyl, halobenzyl, —C(O)NR₇R₈ or —NR₇C(O)R₈;
R₇ is H, methyl, ethyl, propyl or isopropyl;
R₈ is H, methyl, ethyl, propyl, isopropyl, cyclopropyl, n-butyl, tert-butyl, phenyl or benzyl.

32. The compound according to claim 26, wherein R₁ is H, C₁-C₄ alkyl, benzyl, C₁-C₄ haloalkyl or halobenzyl;
R₂ is H, C₁-C₄ alkyl, C₂-C₄ alkenyl, benzyl or halobenzyl.

33. The compound according to claim 32, wherein R₁ is H, methyl, ethyl, n-propyl, isopropyl, butylhalomethyl, haloethyl, halo-n-propyl, haloisopropyl, halobutyl benzyl or halobenzyl;
R₂ is H, methyl, ethyl, n-propyl, isopropyl, butyl, vinyl, propenyl, butenyl, benzyl or halobenzyl.

34. The compound according to claim 26, wherein the definition of R₄ₐ₁ or R_{4b} is any one of the following (1), (2) and (3):
(1) R₄ₐ₁ and R_{4b} are independently H, OH, halogen, C₁-C₄ alkyl, C₁-C₄ alkoxy, C₁-C₄ haloalkyl or C₁-C₄ haloalkoxy;
(2) R₄ₐ₁ and R_{4ab} are independently H, OH, halogen, C₁-C₄ alkyl or C₁-C₄ alkoxy;
(3) R₄ₐ₁ and R_{4b} are independently OH, halogen, methyl, ethyl, propyl, isopropyl, methoxy, ethoxy, propoxy, isopropoxy or n-butoxy.

35. An in vitro co-transcriptional RNA capping reagent, wherein comprising the compound as defined in claim 26.

36. An RNA molecule comprising the compound as defined in claim 26 as a cap structure or a cap structure fragment.

37. A pharmaceutical composition comprising the RNA molecule as defined in claim 36 and a pharmaceutically acceptable carrier.

38. A method for synthesizing an RNA molecule, comprising the following step:
co-incubating the compound as defined in claim 26 with a polynucleotide template for template transcription.

39. A capped RNA transcription reaction system comprising: a polynucleotide template, the compound as defined in claim 26, NTPs, an RNA polymerase.
